(19) United States
(12) Patent Application Publication
(10) Pub. No.: US 2010/0120798 A1
(43) Pub. Date: May 13, 2010

(54) SUBSTITUTED PIPERIDINES CONTAINING A HETEROARYLAMIDE OR HETEROARYLPHENYL MOIETY

(75) Inventors: Steven John Woodhead, Cambridge (GB); Christopher Hamlett, Cambridge (GB); Marinus Leendert Verdonk, Burwell (GB); Hannah Fiona Sore, Cambridge (GB); David Winter Walker, Cambridge (GB); Ian Collins, Redhill (GB); Kwai Ming Cheung, Staines (GB); John Caldwell, Sutton (GB); Tatiana Faria Da Fonseca McHardy, Sunbury-on-Thames (GB); Richard William Arthur Luke, Cheshire (GB); Zbigniew Stanley Matusiak, Cheshire (GB); Gregory Richard Carr, Cheshire (GB); Jeffrey James Morris, Cheshire (GB)

Correspondence Address: HESLIN ROTHENBERG FARLEY & MESITI PC, 5 COLUMBIA CIRCLE, ALBANY, NY, 12203, US

(73) Assignees: ASTEX THERAPEUTICS LIMITED, Cambridge (GB); CANCER RESEARCH TECHNOLOGY LIMITED, London (GB); ASTRAZENECA AB, Södertälje (SE); THE INSTITUTE OF CANCER RESEARCH, LONDON (GB)

(21) Appl. No.: 12/519,993

(22) PCT Filed: Dec. 20, 2007
(86) PCT No.: PCT/GB07/50777

§ 371 Date: Oct. 6, 2009

Related U.S. Application Data
(60) Provisional application No. 60/871,355, filed on Dec. 21, 2006.
(60) Provisional application No. 60/982,636, filed on Oct. 25, 2007.
(60) Provisional application No. 60/986,150, filed on Nov. 7, 2007.

(30) Foreign Application Priority Data
Dec. 21, 2006 (GB) 0625682.0

(51) Int. Cl. A61K 31/522; A61K 31/52; C07D 473/34; A61P 37/00; A61P 9/00; A61P 11/06; A61P 25/00; A61P 29/00; A61P 35/00

(52) U.S. Cl. 514/263.22; 514/265.1; 544/276; 544/277; 544/280

(57) ABSTRACT

The invention provides compounds of the formula (I) having PKA and PKB kinase inhibiting compounds of the formula (I): GP 1 J T 2 J N 4 R N H (I) or salts, solvates, tautomers or N-oxides thereof, wherein (1) GP is a group GP1: HET 2a a Q G (HNCO)f 7 (R)x N * (GP1) (2) GP is a group GP2: 10 (R)r O 2a a QG (CH2)w N V H N * (GP2) wherein HET is a monocyclic or bicyclic heterocyclic group containing up to 4 heteroatom ring members; the ring V is a monocyclic or bicyclic heteroaryl group of 5 to 10 ring members; and J1, J2, R4, R7, R10, Q2a, Ga, x, w and f are as defined in the claims

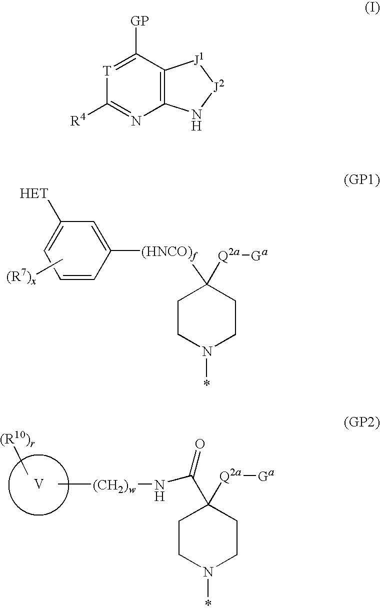

This invention relates to purine, purinone and deazapurine and deazapurinone compounds or structural isomers thereof that inhibit or modulate the activity of protein kinase B (PKB) and/or protein kinase A (PKA), to the use of the compounds in the treatment or prophylaxis of disease states or conditions mediated by PKB and/or PKA, and to novel compounds having PKB and/or PKA inhibitory or modulating activity. Also provided are pharmaceutical compositions containing the compounds and novel chemical intermediates.

### BACKGROUND OF THE INVENTION

Protein kinases constitute a large family of structurally related enzymes that are responsible for the control of a wide variety of signal transduction processes within the cell (Hardie, G. and Hanks, S. (1995) The Protein Kinase Facts Book. I and II, Academic Press, San Diego, Calif.). The kinases may be categorized into families by the substrates they phosphorylate (e.g., protein-tyrosine, protein-serine/threonine, lipids, etc.). Sequence motifs have been identified that generally correspond to each of these kinase families (e.g., Hanks, S. K., Hunter, T., FASEB J., 9:576-596 (1995); Knighton, et al., Science, 253:407-414 (1991); Hiles, et al., Cell, 70:419-429 (1992); Kunz, et al., Cell, 73:585-596 (1993); Garcia-Bustos, et al., EMBO J., 13:2352-2361 (1994)).

Protein kinases may be characterized by their regulation mechanisms. These mechanisms include, for example, autophosphorylation, transphosphorylation by other kinases, protein-protein interactions, protein-lipid interactions, and protein-polynucleotide interactions. An individual protein kinase may be regulated by more than one mechanism.

Kinases regulate many different cell processes including, but not limited to, proliferation, differentiation, apoptosis, motility, transcription, translation and other signalling processes, by adding phosphate groups to target proteins. These phosphorylation events act as molecular on/off switches that can modulate or regulate the target protein biological function. Phosphorylation of target proteins occurs in response to a variety of extracellular signals (hormones, neurotransmitters, growth and differentiation factors, etc.), cell cycle events, environmental or nutritional stresses, etc. The appropriate protein kinase functions in signalling pathways to activate or inactivate (either directly or indirectly), for example, a metabolic enzyme, regulatory protein, receptor, cytoskeletal protein, ion channel or pump, or transcription factor. Uncontrolled signalling due to defective control of protein phosphorylation has been implicated in a number of diseases, including, for example, inflammation, cancer, allergy/asthma, diseases and conditions of the immune system, diseases and conditions of the central nervous system, and angiogenesis.

Apoptosis or programmed cell death is an important physiological process which removes cells no longer required by an organism. The process is important in early embryonic growth and development allowing the non-necrotic controlled breakdown, removal and recovery of cellular components. The removal of cells by apoptosis is also important in the maintenance of chromosomal and genomic integrity of growing cell populations. There are several known checkpoints in the cell growth cycle at which DNA damage and genomic integrity are carefully monitored. The response to the detection of anomalies at such checkpoints is to arrest the growth of such cells and initiate repair processes. If the damage or anomalies cannot be repaired then apoptosis is initiated by the damaged cell in order to prevent the propagation of faults and errors. Cancerous cells consistently contain numerous mutations, errors or rearrangements in their chromosomal DNA. It is widely believed that this occurs in part because the majority of tumours have a defect in one or more of the processes responsible for initiation of the apoptotic process. Normal control mechanisms cannot kill the cancerous cells and the chromosomal or DNA coding errors continue to be propagated. As a consequence restoring these pro-apoptotic signals or suppressing unregulated survival signals is an attractive means of treating cancer.

The signal transduction pathway containing the enzymes phosphatidylinositol 3-kinase (PI3K), PDK1 and PKB amongst others, has long been known to mediate increased resistance to apoptosis or survival responses in many cells. There is a substantial amount of data to indicate that this pathway is an important survival pathway used by many growth factors to suppress apoptosis. The enzymes of the PI3K family are activated by a range of growth and survival factors e.g. EGF, PDGF and through the generation of polyphosphatidylinositols, initiates the activation of the downstream signalling events including the activity of the kinases PDK1 and protein kinase B (PKB) also known as akt. This is also true in host tissues, e.g. vascular endothelial cells as well as neoplasias. PKB is a protein ser/thr kinase consisting of a kinase domain together with an N-terminal PH domain and C-terminal regulatory domain. The enzyme PKBₐₗₚₕₐ (akt1) itself is phosphorylated on Thr 308 by PDK1 and on Ser 473 by ‘PDK2’ now believed to be constituted from the target of rapamycin (TOR) kinase and its associated protein rictor. Full activation requires phosphorylation at both sites whilst association between PIP3 and the PH domain is required for anchoring of the enzyme to the cytoplasmic face of the lipid membrane providing optimal access to substrates.

At least 10 kinases have been suggested to function as a Ser 473 kinase including mitogen-activated protein (MAP) kinase-activated protein kinase-2 (MK2), integrin-linked kinase (ILK), p38 MAP kinase, protein kinase Calpha (PKCalpha), PKCbeta, the NIMA-related kinase-6 (NEK6), the mammalian target of rapamycin (mTOR), the double-stranded DNA-dependent protein kinase (DNK-PK), and the ataxia telangiectasia mutated (ATM) gene product. Available data suggest that multiple systems may be used in cells to regulate the activation of PKB. Full activation of PKB requires phosphorylation at both sites whilst association between PIP3 and the PH domain is required for anchoring of the enzyme to the cytoplasmic face of the lipid membrane providing optimal access to substrates.

Recently, it has been reported that somatic mutations within the PI3K catalytic subunit, PIK3CA, are common (25-40%) among colorectal, gastric, breast, ovarian cancers, and high-grade brain tumors. PIK3CA mutations are a common event that can occur early in bladder carcinogenesis. In invasive breast carcinomas, PIK3CA alterations are mainly present in lobular and ductal tumours. The PI3K pathway is extensively activated in endometrial carcinomas, and that combination of PIK3CA/PTEN alterations might play an important role in development of these tumors. Tumours activated by mutations of PI3 kinase and loss of PTEN will have sustained activation of PKB and will be as a result disproportionately sensitive to inhibition by PKA/PKB inhibitors.

Activated PKB in turns phosphorylates a range of substrates contributing to the overall survival response. Whilst we cannot be certain that we understand all of the factors responsible for mediating the PKB dependent survival response, some important actions are believed to be phosphorylation and inactivation of the pro-apoptotic factor BAD and caspase 9, phosphorylation of Forkhead transcription factors e.g. FKHR leading to their exclusion from the nucleus, and activation of the NfkappaB pathway by phosphorylation of upstream kinases in the cascade.

In addition to the anti-apoptotic and pro-survival actions of the PKB pathway, the enzyme also plays an important role in promoting cell proliferation. This action is again likely to be mediated via several actions, some of which are thought to be phosphorylation and inactivation of the cyclin dependent kinase inhibitor of p21^{Cip1/WAF1}, and phosphorylation and activation of mTOR, a kinase controlling several aspects of cell size, growth and protein translation.

The phosphatase PTEN which dephosphorylates and inactivates polyphosphatidyl-inositols is a key tumour suppressor protein which normally acts to regulate the PI3K/PKB survival pathway. The significance of the PI3K/PKB pathway in tumourigenesis can be judged from the observation that PTEN is one of the most common targets of mutation in human tumours, with mutations in this phosphatase having been found in ˜50% or more of melanomas (Guldberg et al 1997, Cancer Research 57, 3660-3663) and advanced prostate cancers (Cairns et al 1997 Cancer Research 57, 4997). These observations and others suggest that a wide range of tumour types are dependent on the enhanced PKB activity for growth and survival and would respond therapeutically to appropriate inhibitors of PKB.

There are 3 closely related isoforms of PKB called alpha, beta and gamma (AKT1, 2 and 3), which genetic studies suggest have distinct but overlapping functions. Evidence suggests that they can all independently play a role in cancer. For example PKB beta has been found to be over-expressed or activated in 10-40% of ovarian and pancreatic cancers (Bellacosa et al 1995, Int. J. Cancer 64, 280-285; Cheng et al 1996, PNAS 93, 3636-3641; Yuan et al 2000, Oncogene 19, 2324-2330), PKB alpha is amplified in human gastric, prostate and breast cancer (Staal 1987, PNAS 84, 5034-5037; Sun et al 2001, Am. J. Pathol. 159, 431-437) and increased PKB gamma activity has been observed in steroid independent breast and prostate cell lines (Nakatani et al 1999, J. Biol. Chem. 274, 21528-21532).

The PKB pathway also functions in the growth and survival of normal tissues and may be regulated during normal physiology to control cell and tissue function. Thus disorders associated with undesirable proliferation and survival of normal cells and tissues may also benefit therapeutically from treatment with a PKB inhibitor. Examples of such disorders are disorders of immune cells associated with prolonged expansion and survival of cell population leading to a prolonged or up regulated immune response. For example, T and B lymphocyte response to cognate antigens or growth factors such as interferon gamma activates the PI3K/PKB pathway and is responsible for maintaining the survival of the antigen specific lymphocyte clones during the immune response. Under conditions in which lymphocytes and other immune cells are responding to inappropriate self or foreign antigens, or in which other abnormalities lead to prolonged activation, the PKB pathway contributes an important survival signal preventing the normal mechanisms by which the immune response is terminated via apoptosis of the activated cell population. There is a considerable amount of evidence demonstrating the expansion of lymphocyte populations responding to self antigens in autoimmune conditions such as multiple sclerosis and arthritis. Expansion of lymphocyte populations responding inappropriately to foreign antigens is a feature of another set of conditions such as allergic responses and asthma. In summary inhibition of PKB could provide a beneficial treatment for immune disorders.

Other examples of inappropriate expansion, growth, proliferation, hyperplasia and survival of normal cells in which PKB may play a role include but are not limited to atherosclerosis, cardiac myopathy and glomerulonephritis.

In addition to the role in cell growth and survival, the PKB pathway functions in the control of glucose metabolism by insulin. Available evidence from mice deficient in the alpha and beta isoforms of PKB suggests that this action is mediated by the beta iso form primarily. As a consequence, modulators of PKB activity may also find utility in diseases in which there is a dysfunction of glucose metabolism and energy storage such as diabetes, metabolic disease and obesity.

Cyclic AMP-dependent protein kinase (PKA) is a serine/threonine protein kinase that phosphorylates a wide range of substrates and is involved in the regulation of many cellular processes including cell growth, cell differentiation, ion-channel conductivity, gene transcription and synaptic release of neurotransmitters. In its inactive form, the PKA holoenzyme is a tetramer comprising two regulatory subunits and two catalytic subunits. PKA acts as a link between G-protein mediated signal transduction events and the cellular processes that they regulate. Binding of a hormone ligand such as glucagon to a transmembrane receptor activates a receptor-coupled G-protein (GTP-binding and hydrolyzing protein). Upon activation, the alpha subunit of the G protein dissociates and binds to and activates adenylate cyclase, which in turn converts ATP to cyclic-AMP (cAMP). The cAMP thus produced then binds to the regulatory subunits of PKA leading to dissociation of the associated catalytic subunits. The catalytic subunits of PKA, which are inactive when associated with the regulatory sub-units, become active upon dissociation and take part in the phosphorylation of other regulatory proteins.

For example, the catalytic sub-unit of PKA phosphorylates the kinase Phosphorylase Kinase which is involved in the phosphorylation of Phosphorylase, the enzyme responsible for breaking down glycogen to release glucose. PKA is also involved in the regulation of glucose levels by phosphorylating and deactivating glycogen synthase. Thus, modulators of PKA activity (which modulators may increase or decrease PKA activity) may be useful in the treatment or management of diseases in which there is a dysfunction of glucose metabolism and energy storage such as diabetes, metabolic disease and obesity.

PKA has also been established as an acute inhibitor of T cell activation Anndahl et al, have investigated the possible role of PKA type I in HIV-induced T cell dysfunction on the basis that T cells from HIV-infected patients have increased levels of cAMP and are more sensitive to inhibition by cAMP analogues than are normal T cells. From their studies, they concluded that increased activation of PKA type I may contribute to progressive T cell dysfunction in HIV infection and that PKA type I may therefore be a potential target for immunomodulating therapy. —Aandahl, E. M., Aukrust, P., Skålhegg, B. S., Müller, F., Frøland, S. S., Hansson, V., Taskén, K. Protein kinase A type I antagonist restores immune responses of T cells from HIV-infected patients. FASEB J. 12, 855-862 (1998).

It has also been recognised that mutations in the regulatory sub-unit of PKA can lead to hyperactivation in endocrine tissue.

Because of the diversity and importance of PKA as a messenger in cell regulation, abnormal responses of cAMP can lead to a variety of human diseases derived from this, such as irregular cell growth and proliferation (Stratakis, C. A.; Cho-Chung, Y. S.; Protein Kinase A and human diseases. Trends Endrocri. Metab. 2002, 13, 50-52). Over-expression of PKA has been observed in a variety of human cancer cells including those from ovarian, breast and colon patients. Inhibition of PKA would therefore be an approach to treatment of cancer (Li, Q.; Zhu, G-D.; Current Topics in Medicinal Chemistry, 2002, 2, 939-971).

For a review of the role of PKA in human disease, see for example, Protein Kinase A and Human Disease, Edited by Constantine A. Stratakis, Annals of the New York Academy of Sciences, Volume 968, 2002, ISBN 1-57331-412-9.

Several classes of compounds have been disclosed as having PKA and PKB inhibitory activity.

WO 99/65909 (Pfizer) discloses a class of pyrrolo[2,3-d]pyrimidine compounds having protein tyrosine kinase activity and which are of potential use as immunosuppressant agents.

WO 2004/074287 (AstraZeneca) discloses piperazinyl-pyridyl amides for use in treating autoimmune diseases such as arthritis. The piperazine group in the compounds can be linked to a purine group.

WO02/18348 (F. Hoffman La Roche) discloses a class of amino-quinazoline derivatives as alpha-1 adrenergic antagonists. A method for preparing the amino-quinazoline compounds involves the use of a gem-disubstituted cyclic amine such as piperidine in which one of the gem substituents is an aminomethyl group.

WO03/088908 (Bristol Myers Squibb) discloses N-heteroaryl-4,4-disubstituted piperidines as potassium channel inhibitors.

WO01/07050 (Schering) discloses substituted piperidines as nociceptin receptor ORL-1 agonists for use in treating cough.

US 2003/0139427 (OSI) discloses pyrrolidine- and piperidine-substituted purines and purine analogues having adenosine receptor binding activity.

WO 2004/043380 (Harvard College et al.) discloses technetium and rhenium labelled imaging agents containing disubstituted piperidine metal ion-chelating ligands.

WO 97/38665 (Merck) discloses gem-disubstituted piperidine derivatives having farnesyl transferase inhibitory activity.

EP 1568699 (Eisai) discloses 1,3-dihydroimidazole fused ring compounds having DPPIV-inhibiting activity. The compounds are described as having a range of potential uses including the treatment of cancer.

US 2003/0073708 and US 2003/045536 (both in the name of Castelhano et al), WO 02/057267 (OSI Pharmaceuticals) and WO 99/62518 (Cadus Pharmaceutical Corporation) each disclose a class of 4-aminodeazapurines in which the 4-amino group can form part of a cyclic amine such as azetidine, pyrrolidine and piperidine, The compounds are described as having adenosine receptor antagonist activity.

U.S. Pat. No. 6,162,804 (Merck) discloses a class of benzimidazole and aza-benzimidazole compounds that have tyrosine kinase inhibitor activity.

WO 2005/061463 (Astex) discloses pyrazole compounds having PKB and PKA inhibiting activity.

### SUMMARY OF THE INVENTION

The invention provides compounds that have protein kinase B (PKB) and/or protein kinase A (PKA) inhibiting or modulating activity, and which it is envisaged will be useful in preventing or treating disease states or conditions mediated by PKB and/or PKA.

Accordingly, in one aspect, the invention provides a compound of the formula (I):

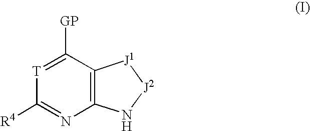

or salts, solvates, tautomers or N-oxides thereof, wherein
(1) GP is a group GP1:

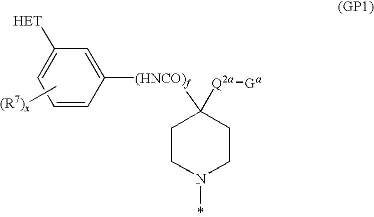

wherein f is 0 or 1, x is 0, 1, 2 or 3 and HET is a monocyclic or bicyclic heterocyclic group containing up to 4 heteroatom ring members and being optionally substituted by one or more substituents R¹¹ selected from halogen, hydroxy, trifluoromethyl, cyano, nitro, carboxy, amino, mono- or di-C₁₋₅ hydrocarbylamino, and a group R^{a}-R^{b} wherein R^{a} is a bond d, O, CO, X¹C(X²), C(X²)X¹, X¹C(X²)X¹, S, SO, SO₂, NR^{c}, SO₂NR^{c} or NR^{c}SO₂; and R^{b} is selected from hydrogen and C₁₋₅ hydrocarbyl optionally substituted by one or more substituents selected from hydroxy, oxo, halogen, cyano, nitro, carboxy, amino and mono- or di-C₁₋₄ hydrocarbylamino, and wherein one or more carbon atoms of the C₁₋₅ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₅ hydrocarbyl;
X¹ is O, S or NR^{c} and X² is ═O, ═S or ═NR^{c};
T is CH or N;
J¹-J² represents a group selected from N═CH, (R^{q})C═N, HN—C(O), H₂C—C(O), N═N and (R^{q})C═CH;
R^{q} is selected from hydrogen, methyl, chlorine and bromine;
Q^{2a} is a bond or a saturated acyclic hydrocarbon linker group containing from 1 to 3 carbon atoms;
G^{a} is C(O)NR²R³, CN, NR²R³ or OH;
R² and R³ are independently selected from hydrogen; C₁₋₅ alkyl and C₁₋₅ alkanoyl wherein the alkyl and alkanoyl groups are optionally substituted by one or more substituents selected from fluorine, hydroxy, cyano, amino, methylamino, dimethylamino and methoxy; or NR²R³ forms a saturated 4 to 7 membered heterocyclic ring optionally containing, in addition to the nitrogen atom of NR²R³ a further heteroatom selected from O, N and S, the heterocyclic ring being optionally substituted by one or more C₁₋₄ alkyl groups;
R⁴ is selected from hydrogen, halogen, C₁₋₅ saturated hydrocarbyl, cyano, CONH₂, CF₃ and NH₂; and
R⁷ is selected from hydrogen, fluorine, chlorine, trifluoromethyl, methoxy, trifluoromethoxy, difluoromethoxy and cyano; or
   (2) GP is a group GP2:

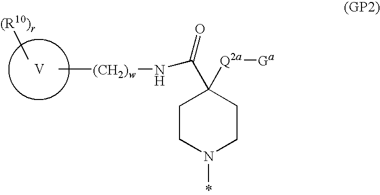

wherein
the ring V is a monocyclic or bicyclic heteroaryl group of 5 to 10 ring members containing up to 4 heteroatom ring members selected from O, N and S;
r is 0, 1, 2, 3 or 4 (e.g. r is 0, 1 or 2);
w is 0 or 1;

### T is CH or N;

J¹-J² represents a group selected from N═CH, (R^{q})C═N, HN—C(O), H₂C—C(O), N═N and (R^{q})C═CH;
R^{q} is selected from hydrogen, methyl, chlorine and bromine;
Q^{2a} is a bond or a saturated acyclic hydrocarbon linker group containing from 1 to 3 carbon atoms;
G^{a} is C(O)NR²R³, CN, NR²R³ or OH;
R² and R³ are independently selected from hydrogen; C₁₋₅ alkyl and C₁₋₅ alkanoyl wherein the alkyl and alkanoyl groups are optionally substituted by one or more substituents selected from fluorine, hydroxy, cyano, amino, methylamino, dimethylamino and methoxy;
R⁴ is selected from hydrogen, halogen, C₁₋₅ saturated hydrocarbyl, cyano, CONH₂, CF₃ and NH₂; and
   R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members, and a group R^{a}-R^{b}; wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

The invention also provides:
A compound of the formula (I) or (II) or any sub-group thereof as defined herein for use in the prophylaxis or treatment of a disease state or condition mediated by protein kinase B.
The use of a compound of formula (I) or (II) or any sub-group thereof as defined herein for the manufacture of a medicament for the prophylaxis or treatment of a disease state or condition mediated by protein kinase B.
A method for the prophylaxis or treatment of a disease state or condition mediated by protein kinase B, which method comprises administering to a subject in need thereof a compound of the formula (I) or (II) or any sub-group thereof as defined herein.
A compound of the formula (I) or any sub-group thereof as defined herein for use in treating a disease or condition comprising or arising from abnormal cell growth or abnormally arrested cell death in a mammal.
The use of a compound of formula (I) or (II) or any sub-group thereof as defined herein for the manufacture of a medicament for treating a disease or condition comprising or arising from abnormal cell growth or abnormally arrested cell death in a mammal.
A method for treating a disease or condition comprising or arising from abnormal cell growth in a mammal, which method comprises administering to the mammal a compound of the formula (I) or (II) or any sub-group thereof as defined herein in an amount effective in inhibiting abnormal cell growth or abnormally arrested cell death.
A method for alleviating or reducing the incidence of a disease or condition comprising or arising from abnormal cell growth or abnormally arrested cell death in a mammal, which method comprises administering to the mammal a compound of the formula (I) or (II) or any sub-group thereof as defined herein in an amount effective in inhibiting abnormal cell growth.
A method for treating a disease or condition comprising or arising from abnormal cell growth or abnormally arrested cell death in a mammal, the method comprising administering to the mammal a compound of the formula (I) or (II) or any sub-group thereof as defined herein in an amount effective to inhibit protein kinase B activity.
A compound of the formula (I) or (II) or any sub-group thereof as defined herein for use in inhibiting protein kinase B.
A method of inhibiting protein kinase B, which method comprises contacting the kinase with a kinase-inhibiting compound of the formula (I) or (II) or any sub-group thereof as defined herein.
A compound of the formula (I) or (II) or any sub-group thereof as defined herein for use in modulating a cellular process (for example cell division) by inhibiting the activity of a protein kinase B and/or protein kinase A.
The use of a compound of formula (I) or (II) or any sub-group thereof as defined herein for the manufacture of a medicament for modulating a cellular process (for example cell division) by inhibiting the activity of a protein kinase B and/or protein kinase A.
A method of modulating a cellular process (for example cell division) by inhibiting the activity of a protein kinase B and/or protein kinase A using a compound of the formula (I) or (II) or any sub-group thereof as defined herein.
A compound of the formula (I) or (II) or any sub-group or embodiment thereof as defined herein for use in the prophylaxis or treatment of a disease state or condition mediated by protein kinase A.
The use of a compound of formula (I) or (II) or any sub-group or embodiment thereof as defined herein for the manufacture of a medicament for the prophylaxis or treatment of a disease state or condition mediated by protein kinase A.
A method for the prophylaxis or treatment of a disease state or condition mediated by protein kinase A, which method comprises administering to a subject in need thereof a compound of the formula (I) or (II) or any sub-group or embodiment thereof as defined herein.
A method for treating a disease or condition comprising or arising from abnormal cell growth or abnormally arrested cell death in a mammal, the method comprising administering to the mammal a compound of the formula (I) or (II) or any sub-group or embodiment thereof as defined herein in an amount effective to inhibit protein kinase A activity.
A compound of the formula (I) or (II) or any sub-group or embodiment thereof as defined herein for inhibiting protein kinase A.
A method of inhibiting protein kinase A, which method comprises contacting the kinase with a kinase-inhibiting compound of the formula (I) or (II) or any sub-group or embodiment thereof as defined herein.
A method of modulating a cellular process (for example cell division) by inhibiting the activity of a protein kinase A using a compound of the formula (I) or (II) or any sub-group or embodiment thereof as defined herein.
The use of a compound of the formula (I) or (II) or any sub-group thereof as defined herein for the manufacture of a medicament for the prophylaxis or treatment of a disease state or condition arising from abnormal cell growth or abnormally arrested cell death.
A pharmaceutical composition comprising a novel compound of the formula (I) or (II) or any sub-group thereof as defined herein and a pharmaceutically acceptable carrier.
A compound of the formula (I) or (II) or any sub-group thereof as defined herein for use in medicine.
The use of a compound of the formula (I) or (II) or any sub-group thereof as defined herein for the manufacture of a medicament for the prophylaxis or treatment of any one of the disease states or conditions disclosed herein.
A method for the treatment or prophylaxis of any one of the disease states or conditions disclosed herein, which method comprises administering to a patient (e.g. a patient in need thereof) a compound (e.g. a therapeutically effective amount) of the formula (I) or (II) or any sub-group thereof as defined herein.
A method for alleviating or reducing the incidence of a disease state or condition disclosed herein, which method comprises administering to a patient (e.g. a patient in need thereof) a compound (e.g. a therapeutically effective amount) of the formula (I) or (II) or any sub-group thereof as defined herein.
A method for the diagnosis and treatment of a disease state or condition mediated by protein kinase B, which method comprises (i) screening a patient to determine whether a disease or condition from which the patient is or may be suffering is one which would be susceptible to treatment with a compound having activity against protein kinase B; and (ii) where it is indicated that the disease or condition from which the patient is thus susceptible, thereafter administering to the patient a compound of the formula (I) or (II) or any sub-group thereof as defined herein.
The use of a compound of the formula (I) or (II) or any sub-group thereof as defined herein for the manufacture of a medicament for the treatment or prophylaxis of a disease state or condition in a patient who has been screened and has been determined as suffering from, or being at risk of suffering from, a disease or condition which would be susceptible to treatment with a compound having activity against protein kinase B.
A compound of the formula (I) or (II) or any sub-group thereof as defined herein for use in the treatment or prophylaxis of a disease state or condition in a patient who has been screened and has been determined as suffering from, or being at risk of suffering from, a disease or condition which would be susceptible to treatment with a compound having activity against protein kinase B.
A method for the diagnosis and treatment of a disease state or condition mediated by protein kinase A, which method comprises (i) screening a patient to determine whether a disease or condition from which the patient is or may be suffering is one which would be susceptible to treatment with a compound having activity against protein kinase A; and (ii) where it is indicated that the disease or condition from which the patient is thus susceptible, thereafter administering to the patient a compound of the formula (I) or (II) or any sub-group or embodiment thereof as defined herein.
A compound of the formula (I) or (II) or any sub-group or embodiment thereof as defined herein for use in the treatment or prophylaxis of a disease state or condition in a patient who has been screened and has been determined as suffering from, or being at risk of suffering from, a disease or condition which would be susceptible to treatment with a compound having activity against protein kinase A.
The use of a compound of the formula (I) or (II) or any sub-group or embodiment thereof as defined herein for the manufacture of a medicament for the treatment or prophylaxis of a disease state or condition in a patient who has been screened and has been determined as suffering from, or being at risk of suffering from, a disease or condition which would be susceptible to treatment with a compound having activity against protein kinase A.
A compound of the formula (I) or (II) or any sub-group or embodiment thereof as defined herein for use as a modulator (e.g. inhibitor) of protein kinase B and/or protein kinase A.
The use of a compound of the formula (I) or (II) or any sub-group or embodiment thereof as defined herein for the manufacture of a medicament for modulating (e.g. inhibiting) protein kinase B and/or protein kinase A.
A method of modulating (e.g. inhibiting) protein kinase B and/or protein kinase A; which method comprises bringing the protein kinase B and/or protein kinase A (e.g. in a cellular environment—for example in vivo) into contact with a compound of the formula (I) or (II) or any sub-group or embodiment thereof as defined herein.

### GENERAL PREFERENCES AND DEFINITIONS

Any references to Formula (I) herein shall be taken also to refer to any sub-group of compounds within formula (I) or (II) or any embodiment or example thereof, unless the context requires otherwise.

As used herein, the term “modulation”, as applied to the activity of a kinase, is intended to define a change in the level of biological activity of the protein kinase. Thus, modulation encompasses physiological changes which effect an increase or decrease in the relevant protein kinase activity. In the latter case, the modulation may be described as “inhibition”. The modulation may arise directly or indirectly, and may be mediated by any mechanism and at any physiological level, including for example at the level of gene expression (including for example transcription, translation and/or post-translational modification), at the level of expression of genes encoding regulatory elements which act directly or indirectly on the levels of kinase activity. Thus, modulation may imply elevated/suppressed expression or over- or under-expression of a kinase, including gene amplification (i.e. multiple gene copies) and/or increased or decreased expression by a transcriptional effect, as well as hyper- (or hypo-)activity and (de)activation of the protein kinase(s) (including (de)activation) by mutation(s). The terms “modulated”, “modulating” and “modulate” are to be interpreted accordingly.

As used herein, the term “mediated”, as used e.g. in conjunction with a kinase as described herein (and applied for example to various physiological processes, diseases, states, conditions, therapies, treatments or interventions) is intended to operate limitatively so that the various processes, diseases, states, conditions, treatments and interventions to which the term is applied are those in which the kinase plays a biological role. In cases where the term is applied to a disease, state or condition, the biological role played by a kinase may be direct or indirect and may be necessary and/or sufficient for the manifestation of the symptoms of the disease, state or condition (or its aetiology or progression). Thus, kinase activity (and in particular aberrant levels of kinase activity, e.g. kinase over-expression) need not necessarily be the proximal cause of the disease, state or condition: rather, it is contemplated that the kinase mediated diseases, states or conditions include those having multifactorial aetiologies and complex progressions in which the kinase in question is only partially involved. In cases where the term is applied to treatment, prophylaxis or intervention, the role played by the kinase may be direct or indirect and may be necessary and/or sufficient for the operation of the treatment, prophylaxis or outcome of the intervention. Thus, a disease state or condition mediated by a kinase includes the development of resistance to any particular cancer drug or treatment.

In this specification, references to “the bicyclic group” shall, unless the context indicates otherwise, be taken to refer to the group:

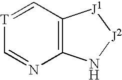

In the definition of the compounds of the formula (I) above and as used hereinafter, the term “hydrocarbyl” is a generic term encompassing aliphatic and alicyclic having an all-carbon backbone and consisting of carbon and hydrogen atoms, except where otherwise stated.

In certain cases, as defined herein, one or more of the carbon atoms making up the carbon backbone may be replaced by a specified atom or group of atoms. Examples of hydrocarbyl groups include alkyl, cycloalkyl, cycloalkenyl, alkenyl, alkynyl, cycloalkylalkyl and cycloalkenylalkyl. The examples and preferences expressed below apply to each of the hydrocarbyl substituent groups or hydrocarbyl-containing substituent groups referred to in the various definitions of substituents for compounds of the formula (I) and sub-groups thereof as defined herein unless the context indicates otherwise.

Generally by way of example, the hydrocarbyl groups can have up to five carbon atoms, unless the context requires otherwise. Within the sub-set of hydrocarbyl groups having 1 to 5 carbon atoms, particular examples are C₁₋₄ hydrocarbyl groups (e.g. C₁₋₃ hydrocarbyl groups or C₁₋₂ hydrocarbyl groups), specific examples being any individual value or combination of values selected from C₁, C₂, C₃, C₄ or C₅ hydrocarbyl groups.

The term “saturated hydrocarbyl”, whether used alone or together with a suffix such as “oxy” (e.g. as in “hydrocarbyloxy”), refers to a non-aromatic hydrocarbon group containing no multiple bonds such as C═C and C≡C.

Particular hydrocarbyl groups are saturated hydrocarbyl groups such as alkyl and cycloalkyl groups as defined herein.

The term “alkyl” as used herein covers both straight chain and branched chain alkyl groups. Examples of alkyl groups include methyl, ethyl, propyl, isopropyl, n-butyl, isobutyl, tert-butyl, n-pentyl, 2-pentyl, 3-pentyl, 2-methyl butyl and 3-methyl butyl and its isomers. Within the sub-set of alkyl groups having 1 to 5 carbon atoms, particular examples are C₁₋₄ alkyl groups (e.g. C₁₋₃ alkyl groups or C₁₋₂ alkyl groups).

Examples of cycloalkyl groups are those derived from cyclopropane, cyclobutane and cyclopentane.

Examples of alkenyl groups include, but are not limited to, ethenyl (vinyl), 1-propenyl, 2-propenyl (allyl), isopropenyl, butenyl, buta-1,4-dienyl and pentenyl. Within the sub-set of alkenyl groups the alkenyl group will have 2 to 5 carbon atoms, particular examples being C₂₋₄ alkenyl groups.

Examples of cycloalkenyl groups include cyclopropenyl, cyclobutenyl and cyclopentenyl.

Examples of alkynyl groups include, but are not limited to, ethynyl and 2-propynyl (propargyl) groups. Within the sub-set of alkynyl groups having 2 to 5 carbon atoms, particular examples are C₂₋₄ alkynyl groups.

Examples of cycloalkylalkyl groups include cyclobutylmethyl and cyclopropylmethyl groups.

The term C₁₋₈ hydrocarbyl as used herein refers to a group consisting of carbon and hydrogen atoms and having 1 to 8 carbon atoms. The term encompasses C₁₋₈ alkyl, C₂₋₈ alkenyl, C₂₋₈ alkynyl, C₃₋₈ cycloalkyl, C₃₋₈ cycloalkenyl, phenyl, benzyl and phenylethyl groups wherein the preferences for and examples of each of the aforesaid groups are as defined above. Within this definition, particular C₁₋₈ hydrocarbyl groups are alkyl groups of 1 to 6 carbon atoms (e.g. up to 5 or up to 4 or up to 3 carbon atoms), cycloalkyl groups of 3 to 7 (more preferably 3 to 6) carbon atoms, phenyl, benzyl and phenylethyl (e.g. 1-phenylethyl or 2-phenylethyl) groups, one subset of C₁₋₈ hydrocarbyl groups consisting of C₁₋₆ alkyl and C₃₋₆ cycloalkyl groups and in particular C₁₋₄ alkyl and C₃₋₆ cycloalkyl groups such as methyl, ethyl, n-propyl, isopropyl, n-butyl, isobutyl, tert-butyl, cyclopropyl and cyclobutyl.

The term C₁₋₅ hydrocarbyl defines a subset of C₁₋₈ hydrocarbyl groups and refers to a group consisting of carbon and hydrogen atoms and having 1 to 5 carbon atoms. The term encompasses C₁₋₅ alkyl, C₂₋₅ alkenyl, C₂₋₅ alkynyl, C₃₋₅ cycloalkyl, and C₃₋₅ cycloalkenyl groups wherein the preferences for and examples of each of the aforesaid groups are as defined above. Within this definition, particular C₁₋₅ hydrocarbyl groups are C₁₋₅ alkyl and C₃₋₅ cycloalkyl groups. Particular examples of C₁₋₅ alkyl and C₃₋₅ cycloalkyl groups are methyl, ethyl, n-propyl, isopropyl, n-butyl, isobutyl, tert-butyl, cyclopropyl and cyclobutyl.

The term C₁₋₄ hydrocarbyl defines a subset of C₁₋₅ hydrocarbyl groups and refers to a group consisting of carbon and hydrogen atoms and having 1 to 4 carbon atoms. The term encompasses C₁₋₄ alkyl, C₂₋₄ alkenyl, C₂₋₄ alkynyl, C₃₋₄ cycloalkyl, and C₃₋₄ cycloalkenyl groups wherein the preferences for and examples of each of the aforesaid groups are as defined above. Within this definition, particular C₁₋₄ hydrocarbyl groups are C₁₋₄ alkyl and C₃₋₄ cycloalkyl groups, such as methyl, ethyl, n-propyl, isopropyl, n-butyl, isobutyl, tert-butyl, cyclopropyl and cyclobutyl.

In certain cases as defined herein, it is stated that one or more carbon atoms of a hydrocarbyl group may optionally be replaced by O, S, SO, SO₂, NR^{c}, X¹C(X²), C(X²)X¹ or X¹C(X²)X¹ or a sub-group thereof) wherein X¹ and X² are as hereinbefore defined, provided that at least one carbon atom of the hydrocarbyl group remains. For example, 1, 2, 3 or 4 carbon atoms of the hydrocarbyl group may be replaced by one of the atoms or groups listed, and the replacing atoms or groups may be the same or different. In general, the number of linear or backbone carbon atoms replaced will correspond to the number of linear or backbone atoms in the group replacing them. Examples of groups in which one or more carbon atom of the hydrocarbyl group have been replaced by a replacement atom or group as defined above include ethers and thioethers (C replaced by O or S), amides, esters, thioamides and thioesters (C—C replaced by X¹C(X²) or C(X²)X¹), sulphones and sulphoxides (C replaced by SO or SO₂), amines (C replaced by NR^{c}), and ureas, carbonates and carbamates (C—C—C replaced by X¹C(X²)X¹).

The term C₁₋₄ acyl as used herein (whether as a discrete moiety or as part of another group such as an acylamino or acyloxy group) refers to a group containing up to 4 carbon atoms and having the formula:

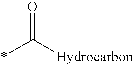

where the asterisk shows the point of attachment to the remainder the molecule and “hydrocarbon” is a hydrocarbon group of 1 to 3 carbon atoms. The hydrocarbon group can be saturated or unsaturated and can be an alkyl, alkenyl or alkynyl group as defined herein or a cyclopropyl ring. In one general embodiment, the hydrocarbon group is an alkyl or cyclopropyl group. In another general embodiment, the hydrocarbon group is an alkyl group.

Particular C₁₋₄ acyl groups are acetyl, propanoyl and isopropanoyl.

The term “aza-cycloalkyl” as used herein refers to a cycloalkyl group in which one of the carbon ring members has been replaced by a nitrogen atom. Thus examples of aza-cycloalkyl groups include piperidine and pyrrolidine. The term “oxa-cycloalkyl” as used herein refers to a cycloalkyl group in which one of the carbon ring members has been replaced by an oxygen atom. Thus examples of oxa-cycloalkyl groups include tetrahydrofuran and tetrahydropyran. In an analogous manner, the terms “diaza-cycloalkyl”, “dioxa-cycloalkyl” and “aza-oxa-cycloalkyl” refer respectively to cycloalkyl groups in which two carbon ring members have been replaced by two nitrogen atoms, or by two oxygen atoms, or by one nitrogen atom and one oxygen atom.

The definition “R^{a}-R^{b}” as used herein, either with regard to substituents present on a carbocyclic or heterocyclic moiety, or with regard to other substituents present at other locations on the compounds of the formula (I), includes inter alia compounds wherein R^{a} is selected from a bond, O, CO, OC(O), SC(O), NR^{c}C(O), OC(S), SC(S), NR^{c}C(S), OC(NR^{c}), SC(NR^{c}), NR^{c}C(NR^{c}), C(O)O, C(O)S, C(O)NR^{c}, C(S)O, C(S)S, C(S)NR^{c}, C(NR^{c})O, C(NR^{c})S, C(NR^{c})NR^{c}, OC(O)O, SC(O)O, NR^{c}C(O)O, OC(S)O, SC(S)O, NR^{c}C(S)O, OC(NR^{c})O, SC(NR^{c})O, NR^{c}C(NR^{c})O, OC(O)S, SC(O)S, NR^{c}C(O)S, OC(S)S, SC(S)S, NR^{c}C(S)S, OC(NR^{c})S, SC(NR^{c})S, NR^{c}C(NR^{c})S, OC(O)NR^{c}, SC(O)NR^{c}, NR^{c}C(O)NR^{c}, OC(S)NR^{c}, SC(S)NR^{c}, NR^{c}C(S)NR^{c}, OC(NR^{c})NR^{c}, SC(NR^{c})NR^{c}, NR^{c}C(NR^{c}NR^{c}, S, SO, SO₂, NR^{c}, SO₂NR^{c} and NR^{c}SO₂ wherein R^{c} is as hereinbefore defined.

The moiety R^{b} can be hydrogen or it can be a C₁₋₈ hydrocarbyl group optionally substituted as hereinbefore defined. Examples of hydrocarbyl groups are as set out above.

When R^{a} is O and R^{b} is a C₁₋₅ hydrocarbyl group, R^{a} and R^{b} together form a hydrocarbyloxy group. Preferred hydrocarbyloxy groups include saturated hydrocarbyloxy such as alkoxy (e.g. C₁₋₅ alkoxy, more usually C₁₋₄ alkoxy such as ethoxy and methoxy, particularly methoxy), cycloalkoxy (e.g. C₃₋₅ cycloalkoxy such as cyclopropyloxy, cyclobutyloxy and cyclopentyloxy, and cycloalkyalkoxy (e.g. cyclopropylmethoxy).

Where stated, the hydrocarbyloxy groups can be substituted by various substituents as defined herein. For example, the alkoxy groups can be substituted by halogen (e.g. as in difluoromethoxy and trifluoromethoxy), hydroxy (e.g. as in hydroxyethoxy), C₁₋₂ alkoxy (e.g. as in methoxyethoxy), hydroxy-C₁₋₂ alkyl (as in hydroxyethoxyethoxy) or a cyclic group (e.g. a cycloalkyl group as hereinbefore defined).

When R^{a} is a bond and R^{b} is a C₁₋₅ hydrocarbyl group, examples of hydrocarbyl groups R^{a}-R^{b} are as hereinbefore defined. The hydrocarbyl groups may be saturated groups such as cycloalkyl and alkyl and particular examples of such groups include methyl, ethyl and cyclopropyl. The hydrocarbyl (e.g. alkyl) groups can be substituted by various groups and atoms as defined herein. Examples of substituted alkyl groups include alkyl groups substituted by one or more halogen atoms such as fluorine and chlorine (particular examples including bromoethyl, chloroethyl, difluoromethyl, 2,2,2-trifluoroethyl and perfluoroalkyl groups such as trifluoromethyl), or hydroxy (e.g. hydroxymethyl and hydroxyethyl), C₁₋₅ acyloxy (e.g. acetoxymethyl), amino and mono- and dialkylamino (e.g. aminoethyl, methylaminoethyl, dimethylaminomethyl, dimethylaminoethyl and tert-butylaminomethyl), alkoxy (e.g. C₁₋₂ alkoxy such as methoxy—as in methoxyethyl), and cyclic groups such as cycloalkyl groups as hereinbefore defined).

When R^{a} is SO₂NR^{c}, R^{b} can be, for example, hydrogen or an optionally substituted C₁₋₅ hydrocarbyl group. Examples of R^{a}-R^{b} where R^{a} is SO₂NR^{c} include aminosulphonyl, C₁₋₄ alkylaminosulphonyl and di-C₁₋₄ alkylaminosulphonyl groups.

Examples of groups R^{a}-R^{b} where R^{a} is SO₂ include alkylsulphonyl groups. A particular examples is methylsulphonyl.

When R^{a} is NR^{c}, R^{b} can be, for example, hydrogen or an optionally substituted C₁₋₅ hydrocarbyl group. Examples of R^{a}-R^{b} where R^{a} is NR^{c} include amino, C₁₋₄ alkylamino (e.g. methylamino, ethylamino, propylamino, isopropylamino, tert-butylamino), di-C₁₋₄ alkylamino (e.g. dimethylamino and diethylamino) and cycloalkylamino (e.g. cyclopropylamino).

References to “carbocyclic” and “heterocyclic” groups as used herein shall, unless the context indicates otherwise, include both aromatic and non-aromatic ring systems. In general, such groups may be monocyclic or bicyclic and may contain, for example, 3 to 12 ring members, more usually 5 to 10 ring members. Examples of monocyclic groups are groups containing 3, 4, 5, 6, 7, and 8 ring members, more usually 3 to 7, and preferably 5 or 6 ring members. Examples of bicyclic groups are those containing 8, 9, 10, 11 and 12 ring members, and more usually 9 or 10 ring members.

The carbocyclic or heterocyclic groups can be aryl or heteroaryl groups having from 5 to 12 ring members, more usually from 5 to 10 ring members. The term “aryl” as used herein refers to a carbocyclic group having aromatic character and the term “heteroaryl” is used herein to denote a heterocyclic group having aromatic character. The terms “aryl” and “heteroaryl” embrace polycyclic (e.g. bicyclic) ring systems wherein one or more rings are non-aromatic, provided that at least one ring is aromatic. In such polycyclic systems, the group may be attached by the aromatic ring, or by a non-aromatic ring. The aryl or heteroaryl groups can be monocyclic or bicyclic groups and can be unsubstituted or substituted with one or more substituents, for example one or more groups R¹⁰ as defined herein.

The term non-aromatic group embraces unsaturated ring systems without aromatic character, partially saturated and fully saturated carbocyclic and heterocyclic ring systems. The terms “unsaturated” and “partially saturated” refer to rings wherein the ring structure(s) contains atoms sharing more than one valence bond i.e. the ring contains at least one multiple bond e.g. a C═C, C≡C or N═C bond. The term “fully saturated” refers to rings where there are no multiple bonds between ring atoms. Saturated carbocyclic groups include cycloalkyl groups as defined below. Partially saturated carbocyclic groups include cycloalkenyl groups as defined below, for example cyclopentenyl, cycloheptenyl and cyclooctenyl.

Examples of heteroaryl groups are monocyclic and bicyclic groups containing from five to twelve ring members, and more usually from five to ten ring members. The heteroaryl group can be, for example, a five membered or six membered monocyclic ring or a bicyclic structure formed from fused five and six membered rings or two fused six membered rings. Each ring may contain up to about four heteroatoms typically selected from nitrogen, sulphur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of five membered heteroaryl groups include but are not limited to pyrrole, furan, thiophene, imidazole, furazan, oxazole, oxadiazole, oxatriazole, isoxazole, thiazole, isothiazole, pyrazole, triazole and tetrazole groups.

Examples of six membered heteroaryl groups include but are not limited to pyridine, pyrazine, pyridazine, pyrimidine and triazine.

A bicyclic heteroaryl group may be, for example, a group selected from:
a) a benzene ring fused to a 5- or 6-membered ring containing 1, 2 or 3 ring heteroatoms;
b) a pyridine ring fused to a 5- or 6-membered ring containing 1, 2 or 3 ring heteroatoms;
c) a pyrimidine ring fused to a 5- or 6-membered ring containing 1 or 2 ring heteroatoms;
d) a pyrrole ring fused to a 5- or 6-membered ring containing 1, 2 or 3 ring heteroatoms;
e) a pyrazole ring fused to a 5- or 6-membered ring containing 1 or 2 ring heteroatoms;
f) a pyrazine ring fused to a 5- or 6-membered ring containing 1 or 2 ring heteroatoms;
g) an imidazole ring fused to a 5- or 6-membered ring containing 1 or 2 ring heteroatoms;
h) an oxazole ring fused to a 5- or 6-membered ring containing 1 or 2 ring heteroatoms;
i) an isoxazole ring fused to a 5- or 6-membered ring containing 1 or 2 ring heteroatoms;
j) a thiazole ring fused to a 5- or 6-membered ring containing 1 or 2 ring heteroatoms;
k) an isothiazole ring fused to a 5- or 6-membered ring containing 1 or 2 ring heteroatoms;
l) a thiophene ring fused to a 5- or 6-membered ring containing 1, 2 or 3 ring heteroatoms;
m) a furan ring fused to a 5- or 6-membered ring containing 1, 2 or 3 ring heteroatoms;
n) a cyclohexyl ring fused to a 5- or 6-membered ring containing 1, 2 or 3 ring heteroatoms; and
o) a cyclopentyl ring fused to a 5- or 6-membered ring containing 1, 2 or 3 ring heteroatoms.

Particular examples of bicyclic heteroaryl groups containing a six membered ring fused to a five membered ring include but are not limited to benzfuran, benzthiophene, benzimidazole, benzoxazole, benzisoxazole, benzthiazole, benzisothiazole, isobenzofuran, indole, isoindole, indolizine, indoline, isoindoline, purine (e.g., adenine, guanine), indazole, benzodioxole and pyrazolopyridine groups.

Particular examples of bicyclic heteroaryl groups containing two fused six membered rings include but are not limited to quinoline, isoquinoline, chroman, thiochroman, chromene, isochromene, chroman, isochroman, benzodioxan, quinolizine, benzoxazine, benzodiazine, pyridopyridine, quinoxaline, quinazoline, cinnoline, phthalazine, naphthyridine and pteridine groups.

Examples of polycyclic aryl and heteroaryl groups containing an aromatic ring and a non-aromatic ring include tetrahydronaphthalene, tetrahydroisoquinoline, tetrahydroquinoline, dihydrobenzthiene, dihydrobenzfuran, 2,3-dihydro-benzo[1,4]dioxine, benzo[1,3]dioxole, 4,5,6,7-tetrahydrobenzofuran, indoline and indane groups.

Examples of carbocyclic aryl groups include phenyl, naphthyl, indenyl, and tetrahydronaphthyl groups.

Examples of non-aromatic heterocyclic groups include unsubstituted or substituted (by one or more groups R¹⁰) heterocyclic groups having from 3 to 12 ring members, typically 4 to 12 ring members, and more usually from 5 to 10 ring members. Such groups can be monocyclic or bicyclic, for example, and typically have from 1 to 5 heteroatom ring members (more usually 1, 2, 3 or 4 heteroatom ring members) typically selected from nitrogen, oxygen and sulphur.

When sulphur is present, it may, where the nature of the adjacent atoms and groups permits, exist as —S—, —S(O)— or —S(O)₂—.

The heterocylic groups can contain, for example, cyclic ether moieties (e.g. as in tetrahydrofuran and dioxane), cyclic thioether moieties (e.g. as in tetrahydrothiophene and dithiane), cyclic amine moieties (e.g. as in pyrrolidine), cyclic amide moieties (e.g. as in pyrrolidone), cyclic urea moieties (e.g. as in imidazolidin-2-one), cyclic thiourea moieties, cyclic thioamides, cyclic thioesters, cyclic ester moieties (e.g. as in butyrolactone), cyclic sulphones (e.g. as in sulpholane and sulpholene), cyclic sulphoxides, cyclic sulphonamides and combinations thereof (e.g. morpholine and thiomorpholine and its S-oxide and S,S-dioxide).

Examples of monocyclic non-aromatic heterocyclic groups include 5-, 6- and 7-membered monocyclic heterocyclic groups. Particular examples include morpholine, thiomorpholine and its S-oxide and S,S-dioxide (particularly thiomorpholine), piperidine (e.g. 1-piperidinyl, 2-piperidinyl 3-piperidinyl and 4-piperidinyl), N-alkyl piperidines such as N-methyl piperidine, piperidone, pyrrolidine (e.g. 1-pyrrolidinyl, 2-pyrrolidinyl and 3-pyrrolidinyl), pyrrolidone, azetidine, pyran (2H-pyran or 4H-pyran), dihydrothiophene, dihydropyran, dihydropyran, dihydrothiazole, tetrahydrofuran, tetrahydrothiophene, dioxane, tetrahydropyran (e.g. 4-tetrahydro pyranyl), imidazoline, imidazolidinone, oxazoline, thiazoline, 2-pyrazoline, pyrazolidine, piperazone, piperazine, and N-alkyl piperazines such as N-methyl piperazine, N-ethyl piperazine and N-isopropylpiperazine. In general, preferred non-aromatic heterocyclic groups include piperidine, pyrrolidine, azetidine, morpholine, piperazine and N-alkyl piperazines.

Examples of non-aromatic carbocyclic groups include cycloalkane groups such as cyclohexyl and cyclopentyl, cycloalkenyl groups such as cyclopentenyl, cyclohexenyl, cycloheptenyl and cyclooctenyl, as well as cyclohexadienyl, cyclooctatetraene, tetrahydronaphthenyl and decalinyl.

Preferred non-aromatic carbocyclic groups are monocyclic rings and most preferably saturated monocyclic rings.

Typical examples are three, four, five and six membered saturated carbocyclic rings, e.g. optionally substituted cyclopentyl and cyclohexyl rings.

One sub-set of non-aromatic carbocyclic groups includes unsubstituted or substituted (by one or more groups R¹⁰) monocyclic groups and particularly saturated monocyclic groups, e.g. cycloalkyl groups. Examples of such cycloalkyl groups include cyclopropyl, cyclobutyl, cyclopentyl, cyclohexyl and cycloheptyl; more typically cyclopropyl, cyclobutyl, cyclopentyl and cyclohexyl, particularly cyclohexyl.

Further examples of non-aromatic cyclic groups include bridged ring systems such as bicycloalkanes and azabicycloalkanes although such bridged ring systems are generally less preferred. By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged ring systems include bicyclo[2.2.1]heptane, aza-bicyclo[2.2.1]heptane, bicyclo[2.2.2]octane, aza-bicyclo[2.2.2]octane, bicyclo[3.2.1]octane and aza-bicyclo[3.2.1]octane.

Where reference is made herein to carbocyclic and heterocyclic groups, the carbocyclic or heterocyclic ring can, unless the context indicates otherwise, be unsubstituted or substituted by one or more substituent groups R¹⁰ selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

Where the substituent group R¹⁰ comprises or includes a carbocyclic or heterocyclic group, the said carbocyclic or heterocyclic group may be unsubstituted or may itself be substituted with one or more further substituent groups R¹⁰. In one sub-group of compounds of the formula (I), such further substituent groups R¹⁰ may include carbocyclic or heterocyclic groups, which are typically not themselves further substituted. In another sub-group of compounds of the formula (I), the said further substituents do not include carbocyclic or heterocyclic groups but are otherwise selected from the groups listed above in the definition of R¹⁰.

The substituents R¹⁰ may be selected such that they contain no more than 20 non-hydrogen atoms, for example, no more than 15 non-hydrogen atoms, e.g. no more than 12, or 10, or 9, or 8, or 7, or 6, or 5 non-hydrogen atoms.

One sub-group of substituents R¹⁰ is represented by R^{10a} which consists of substituents selected from halogen, hydroxy, trifluoromethyl, cyano, nitro, carboxy, amino, mono- or di-C₁₋₄ hydrocarbylamino, carbocyclic and heterocyclic groups having from 3 to 7 ring members; a group R^{a}-R^{b} wherein R^{a} is a bond, O, CO, OC(O), NR^{c}C(O), OC(NR^{c}), C(O)O, C(O)NR^{c}, OC(O)O, NR^{c}C(O)O, OC(O)NR^{c}, NR^{c}C(O)NR^{c}, S, SO, SO₂, NR^{c}, SO₂NR^{c} or NR^{c}SO₂; and R^{b} is selected from hydrogen, carbocyclic and heterocyclic groups having from 3 to 7 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 7 ring members and wherein one or more carbon atoms of the C₁₋₈ hydrocarbyl group may optionally be replaced by O, S, SO, SO₂, NR^{c}, OC(O), NR^{c}C(O), OC(NR^{c}), C(O)O, C(O)NR^{c}, OC(O)O, NR^{c}C(O)O, OC(O)NR^{c} or NR^{c}C(O)NR^{c};

R^{c} is selected from hydrogen and C₁₋₄ hydrocarbyl.

Another sub-group of substituents R¹⁰ is represented by R^{10b} which consists of substituents selected from halogen, hydroxy, trifluoromethyl, cyano, amino, mono- or di-C₁₋₄ alkylamino, cyclopropylamino, carbocyclic and heterocyclic groups having from 3 to 7 ring members; a group R^{a}-R^{b} wherein R^{a} is a bond, O, CO, OC(O), NR^{c}C(O), OC(NR^{c}), C(O)O, C(O)NR^{c}, S, SO, SO₂, NR^{c}, SO₂NR^{c} or NR^{c}SO₂; and R^{b} is selected from hydrogen, carbocyclic and heterocyclic groups having from 3 to 7 ring members, and a C₁₋₈ hydrocarbyl group optionally substituted by one or more substituents selected from hydroxy, oxo, halogen, cyano, amino, mono- or di-C₁₋₄ alkylamino, carbocyclic and heterocyclic groups having from 3 to 7 ring members and wherein one or more carbon atoms of the C₁₋₈ hydrocarbyl group may optionally be replaced by O, S, SO, SO₂ or NR^{c};

provided that R^{a} is not a bond when R^{b} is hydrogen; and
R^{c} is selected from hydrogen and C₁₋₄ alkyl.

A further sub-group of substituents R¹⁰ is represented by R^{10c} which consists of substituents selected from:

halogen,
hydroxy,
trifluoromethyl,
cyano,
amino, mono- or di-C₁₋₄ alkylamino,
cyclopropylamino,
monocyclic carbocyclic and heterocyclic groups having from 3 to 7 ring members of which 0, 1 or 2 are selected from O, N and S and the remainder are carbon atoms, wherein the monocyclic carbocyclic and heterocyclic groups are optionally substituted by one or more substituents selected from halogen, hydroxy, trifluoromethyl, cyano and methoxy;
a group R^{a}-R^{b};
R^{a} is a bond, O, CO, OC(O), NR^{c}C(O), OC(NR^{c}), C(O)O, C(O)NR^{c}, S, SO, SO₂, NR^{c}, SO₂NR^{c} or NR^{c}SO₂;
R^{b} is selected from hydrogen, monocyclic carbocyclic and heterocyclic groups having from 3 to 7 ring members of which 0, 1 or 2 are selected from O, N and S and the remainder are carbon atoms, wherein the monocyclic carbocyclic and heterocyclic groups are optionally substituted by one or more substituents selected from halogen, hydroxy, trifluoromethyl, cyano and methoxy;
and R^{b} is further selected from a C₁₋₈ hydrocarbyl group optionally substituted by one or more substituents selected from hydroxy, oxo, halogen, cyano, amino, mono- or di-C₁₋₄ alkylamino, monocyclic carbocyclic and heterocyclic groups having from 3 to 7 ring members of which 0, 1 or 2 are selected from O, N and S and the remainder are carbon atoms, wherein the monocyclic carbocyclic and heterocyclic groups are optionally substituted by one or more substituents selected from halogen, hydroxy, trifluoromethyl, cyano and methoxy, and wherein one or two carbon atoms of the C₁₋₈ hydrocarbyl group may optionally be replaced by O, S or NR^{c}; provided that R^{a} is not a bond when R^{b} is hydrogen; and
R^{c} is selected from hydrogen and C₁₋₄ alkyl.

Where the carbocyclic and heterocyclic groups have a pair of substituents on adjacent ring atoms, the two substituents may be linked so as to form a cyclic group. For example, an adjacent pair of substituents on adjacent carbon atoms of a ring may be linked via one or more heteroatoms and optionally substituted alkylene groups to form a fused oxa-, dioxa-, aza-, diaza- or oxa-aza-cycloalkyl group. Examples of such linked substituent groups include:

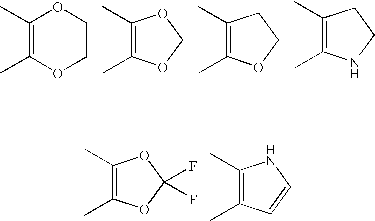

Examples of halogen substituents include fluorine, chlorine, bromine and iodine. Fluorine and chlorine are particularly preferred.

### SPECIFIC EMBODIMENTS OF AND PREFERENCES FOR GP1, GP2, HET, Q^{2a}, G^{a}, V, T, J¹, J² AND R¹ to R¹¹

In formula (I), R⁴ is selected from hydrogen, halogen, C₁₋₅ saturated hydrocarbyl, cyano, CONH₂, CF₃ and NH₂. In one general embodiment, R⁴ is hydrogen.

### GP1

In one embodiment, GP is a group GP1:

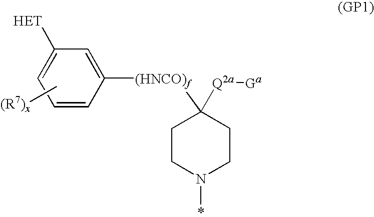

wherein f is 0 or 1, x is 0, 1, 2 or 3 and HET is a monocyclic or bicyclic heterocyclic group containing up to 4 heteroatom ring members and being optionally substituted by one or more substituents R¹¹ selected from halogen, hydroxy, trifluoromethyl, cyano, nitro, carboxy, amino, mono- or di-C₁₋₅ hydrocarbylamino, a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen and C₁₋₅ hydrocarbyl optionally substituted by one or more substituents selected from hydroxy, oxo, halogen, cyano, nitro, carboxy, amino and mono- or di-C₁₋₄ hydrocarbylamino, and wherein one or more carbon atoms of the C₁₋₅ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₅ hydrocarbyl;
X¹ is O, S or NR^{c} and X² is ═O, ═S or ═NR^{c};
T is CH or N;
J¹-J² represents a group selected from N═CH, (R^{q})C═N, HN—C(O), H₂C—C(O), N═N and (R^{q})C═CH;
R^{q} is selected from hydrogen, methyl, chlorine and bromine;
Q^{2a} is a bond or a saturated acyclic hydrocarbon linker group containing from 1 to 3 carbon atoms;
G^{a} is C(O)NR²R³, CN, NR²R³ or OH;
R² and R³ are independently selected from hydrogen; C₁₋₅ alkyl and C₁₋₅ alkanoyl wherein the alkyl and alkanoyl groups are optionally substituted by one or more substituents selected from fluorine, hydroxy, cyano, amino, methylamino, dimethylamino and methoxy; or NR²R³ forms a saturated 4 to 7 membered heterocyclic ring optionally containing, in addition to the nitrogen atom of NR²R³ a further heteroatom selected from O, N and S, the heterocyclic ring being optionally substituted by one or more C₁₋₄ alkyl groups; and
R⁷ is selected from hydrogen, fluorine, chlorine, trifluoromethyl, methoxy, trifluoromethoxy, difluoromethoxy and cyano.

In one group of compounds, f is 0.

In another group of compounds, f is 1.

In one sub-group of compounds, J¹-J² represents a group selected from N═CH, HC═N, HN—C(O), H₂C—C(O), N═N and HC═CH.

In GP1, T can be nitrogen or CH and J¹-J² can represent a group selected from N═CH, N═N, HC═N, HN—C(O), H₂C—C(O) and HC═CH. Thus the bicyclic group can take the form of, for example:
a purine (T is N, J¹-J² is N═CH);
a 3H-imidazo[4,5-b]pyridine (T is CH, J¹-J² is N═CH);
a 7H-pyrrolo[2,3-d]pyrimidine (T is N, J¹-J² is HC═CH);
a 1H-pyrrolo[2,3-b]pyridine (T is CH, J¹-J² is HC═CH);
a 5,7-dihydro-pyrrolo[2,3-d]pyrimidin-6-one (T is N, J¹-J² is H₂C—C(O));
a 3H-[1,2,3]triazolo[4,5-d]pyrimidine (T is N, J¹-J² is N═N);
a 3H-[1,2,3]triazolo[4,5-b]pyridine (T is CH, J¹-J² is N═N);
a 7,9-dihydro-purin-8-one (T is N, J¹-J² is HN—C(O));
a 1H-pyrazolo[3,4-d]pyrimidine (T is N, J¹-J² is HC═N); or
a pyrazolo[3,4-b]pyridine (T is CR⁵, J¹-J² is HC═N).

In one sub-group of compounds, T is N and J¹-J² is HNC(CO).

In another sub-group of compounds, T is N and J¹-J² is N═CH.

In a further sub-group of compounds, T is and J¹-J² is HC═N.

In another sub-group of compounds, T is N and J¹-J² is HC═CH.

In another sub-group of compounds, J¹-J² represents a group selected from HC═N, HC═CH, (Br)C═N, (Cl)C═N, (Me)C═N, (Br)C═CH, (Cl)C═CH and (Me)C═CH.

Q^{2a} is a bond or a saturated acyclic hydrocarbon linker group containing from 1 to 3 carbon atoms. The saturated acyclic hydrocarbon linker group can be a straight or branched chain alkylene group of 1 to 3 carbon atoms. In one sub-group of compounds, Q^{2a} is a bond or a group (CH₂)ₐ where a is 1, 2 or 3. Preferably, Q^{2a} is a bond or a group (CH₂)ₐ where a is 1 or 2, and more preferably is 1. Q^{2a} is a bond or a group (CH₂)ₐ where a is 1, 2

G^{a} is C(O)NR²R³, CN, NR²R³ or OH. Preferably G^{a} is NR²R³.

In one group of compounds, R² and R³ are independently selected from hydrogen; C₁₋₅ alkyl and C₁₋₅ alkanoyl wherein the alkyl and alkanoyl groups are optionally substituted by one or more substituents selected from fluorine, hydroxy, cyano, amino, methylamino, dimethylamino and methoxy.

In one preferred group of compounds, R² and R³ are independently selected from hydrogen and C₁₋₄ alkyl wherein the alkyl groups are optionally substituted by one or more substituents selected from fluorine, hydroxy, amino, methylamino, dimethylamino and methoxy.

More preferably, the optionally substituted alkyl group forming part of NR²R³ is a C₁, C₂ or C₃ alkyl group, for example a methyl group.

In a particular sub-group of compounds, R² and R³ are independently selected from hydrogen and methyl and hence NR²R³ can be an amino, methylamino or dimethylamino group.

In one embodiment, NR²R³ is an amino group. In another particular embodiment, NR²R³ is a methylamino group. In a further particular embodiment, NR²R³ is a dimethylamino group.

In another embodiment, NR²R³ forms a saturated 4 to 7 membered heterocyclic ring optionally containing, in addition to the nitrogen atom of NR²R³ a further heteroatom selected from O, N and S, the heterocyclic ring being optionally substituted by one or more C₁₋₄ alkyl groups. Preferably the heterocyclic ring is a 4 to 6 membered ring and more preferably the heterocyclic ring is a 5 to 6 membered ring. Examples of such heterocyclic rings include azetidine, pyrrolidine, piperidine, piperazine, N-methylpiperazine, morpholine, thiomorpholine and the S-oxide and S,S-dioxide thereof. One particular heterocyclic ring is pyrrolidine.

The benzene ring to which the group HET is attached can have attached thereto 0, 1, 2 or 3 substituents R⁷ in addition to the group HET. Preferably there are 0, 1 or 2 such substituents, and more preferably 0 or 1. In one embodiment, x is 0. In another embodiment x is 1.

The group HET is a monocyclic or bicyclic heterocyclic group containing up to 4 heteroatom ring members and being optionally substituted by one or more substituents R¹¹. The heterocyclic group can be aromatic (heteroaryl) or non-aromatic.

In one embodiment, the group HET is a monocyclic or bicyclic heteroaryl group containing up to 3 heteroatom ring members and being optionally substituted by one or more substituents R¹¹. As such, it may contain 5 to 12 ring members, more usually 5 to 10 ring members. Examples of monocyclic groups are groups containing 5 and 6 ring members. Examples of bicyclic groups are those containing 9 and 10 ring members.

The term “heteroaryl” is used herein to denote a heterocyclic group having aromatic character and includes bicyclic groups wherein one ring is non-aromatic, provided that the other ring is aromatic. Such groups may be attached by the aromatic ring, or by a non-aromatic ring.

Examples of heteroaryl groups are five membered or six membered monocyclic ring or a bicyclic structure formed from fused five and six membered rings or two fused six membered rings. Each ring may contain up to about four heteroatoms typically selected from nitrogen, sulphur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of five membered heteroaryl groups include but are not limited to pyrrole, furan, thiophene, imidazole, furazan, oxazole, oxadiazole, oxatriazole, isoxazole, thiazole, isothiazole, pyrazole, triazole and tetrazole groups.

Examples of six membered heteroaryl groups include but are not limited to pyridine, pyrazine, pyridazine, pyrimidine and triazine.

A bicyclic heteroaryl group may be, for example, a group selected from:
a) a benzene ring fused to a 5- or 6-membered ring containing 1, 2 or 3 ring heteroatoms;
b) a pyridine ring fused to a 5- or 6-membered ring containing 1, 2 or 3 ring heteroatoms;
c) a pyrimidine ring fused to a 5- or 6-membered ring containing 1 or 2 ring heteroatoms;
d) a pyrrole ring fused to a 5- or 6-membered ring containing 1, 2 or 3 ring heteroatoms;
e) a pyrazole ring fused to a 5- or 6-membered ring containing 1 or 2 ring heteroatoms;
f) a pyrazine ring fused to a 5- or 6-membered ring containing 1 or 2 ring heteroatoms;
g) an imidazole ring fused to a 5- or 6-membered ring containing 1 or 2 ring heteroatoms;
h) an oxazole ring fused to a 5- or 6-membered ring containing 1 or 2 ring heteroatoms;
i) an isoxazole ring fused to a 5- or 6-membered ring containing 1 or 2 ring heteroatoms;
j) a thiazole ring fused to a 5- or 6-membered ring containing 1 or 2 ring heteroatoms;
k) an isothiazole ring fused to a 5- or 6-membered ring containing 1 or 2 ring heteroatoms;
l) a thiophene ring fused to a 5- or 6-membered ring containing 1, 2 or 3 ring heteroatoms;
m) a furan ring fused to a 5- or 6-membered ring containing 1, 2 or 3 ring heteroatoms;
n) a cyclohexyl ring fused to a 5- or 6-membered ring containing 1, 2 or 3 ring heteroatoms; and
o) a cyclopentyl ring fused to a 5- or 6-membered ring containing 1, 2 or 3 ring heteroatoms.

Particular examples of bicyclic heteroaryl groups containing a six membered ring fused to a five membered ring include but are not limited to benzfuran, benzthiophene, benzimidazole, benzoxazole, benzisoxazole, benzthiazole, benzisothiazole, isobenzofuran, indole, isoindole, indolizine, indoline, isoindoline, purine (e.g., adenine, guanine), indazole, benzodioxole and pyrazolopyridine groups.

Particular examples of bicyclic heteroaryl groups containing two fused six membered rings include but are not limited to quinoline, isoquinoline, chroman, thiochroman, chromene, isochromene, chroman, isochroman, benzodioxan, quinolizine, benzoxazine, benzodiazine, pyridopyridine, quinoxaline, quinazoline, cinnoline, phthalazine, naphthyridine and pteridine groups.

Examples of heteroaryl groups containing an aromatic ring and a non-aromatic ring include tetrahydroisoquinoline, tetrahydroquinoline, dihydrobenzthiene, dihydrobenzfuran, 2,3-dihydro-benzo[1,4]dioxine, benzo[1,3]dioxole, 4,5,6,7-tetrahydrobenzofuran and indoline.

One subset of heteroaryl groups consists of benzoxazole, pyridine, pyrazole and thiophene, each optionally substituted by one or more substituents R¹¹ or subsets of subgroups thereof as defined herein. In one embodiment, the heteroaryl groups are unsubstituted. In another embodiment, the heteroaryl groups are substituted.

Another subset of heteroaryl groups consists of benzoxazole, pyridine, pyrimidine, pyrazole and thiophene, each optionally substituted by one or more substituents R¹¹.

The group HET is optionally substituted by one or more substituents R¹¹ or subsets of subgroups thereof as defined herein.

When HET is a heteroaryl group, more typically the substituents are selected from a group R^{11a} consisting of fluorine, chlorine, bromine, hydroxy, trifluoromethyl, cyano, carboxy, amino, mono- or di-C₁₋₄ hydrocarbylamino, a group R^{aa}-R^{bb} wherein R^{aa} is a bond, O, CO, OC(O), NR^{cc}C(O), OC(NR^{cc}), C(O)O, C(O)NR^{cc}, OC(O)O, NR^{cc}C(O)O, OC(O)NR^{cc}, NR^{cc}C(O)NR^{cc}, S, SO, SO₂, NR^{cc}, SO₂NR^{cc} and NR^{cc}SO₂; and R^{bb} is selected from hydrogen and C₁₋₄ hydrocarbyl optionally substituted by one or more substituents selected from hydroxy, oxo, fluorine, chlorine, bromine, cyano, carboxy, amino and mono- or di-C₁₋₂ hydrocarbylamino, and wherein one carbon atom of the C₁₋₄ hydrocarbyl group may optionally be replaced by O or S; and R^{cc} is selected from hydrogen and C₁₋₃ alkyl.

When HET is a heteroaryl group, more preferably, the substituents are selected from a group R^{11b} consisting of fluorine, chlorine, bromine, hydroxy, trifluoromethyl, cyano, amino, mono- or di-C₁₋₂ alkylamino, a group R^{aaa}-R^{bbb} wherein R^{aaa} is a bond, O, CO, OC(O), NR^{cc}C(O), OC(NR^{cc}), C(O)O, C(O)NR^{cc}, S, SO, SO₂, NR^{cc}, SO₂NR^{cc} and NR^{cc}SO₂; R^{bbb} is selected from hydrogen and C₁₋₄ alkyl optionally substituted by one or more substituents selected from hydroxy, C₁₋₂ alkoxy, oxo, fluorine, chlorine, bromine, cyano, amino and mono- or di-C₁₋₂ alkylamino; and R^{cc} is hydrogen or C₁₋₃ alkyl.

Particular substituents R¹¹ are selected from fluorine; chlorine; C₁₋₄ alkoxy; trifluoromethyl; trifluoromethoxy; difluoromethoxy; and C₁₋₄ alkyl.

Preferably, there are 0, 1 or 2 substituents and more particularly 0 or 1 substituents.

Preferred substituents R¹¹ are fluorine; chlorine; methoxy; trifluoromethyl; trifluoromethoxy; difluoromethoxy; and methyl.

More preferred substituents are (i) fluorine, chlorine and methyl or (ii) chlorine and methyl.

The group HET may alternatively be a non-aromatic heterocyclic group. Examples of non-aromatic groups are monocyclic and bicyclic heterocyclic groups containing from 1 to 10 ring members and up to three heteroatoms selected from O, N and S. More particularly, the group HET can be a monocyclic heterocyclic group of 4 to 7 ring members of which up to 2 are heteroatoms selected from O, N and S. For example, the monocyclic heterocyclic group can contain a nitrogen heteroatom ring member and optionally one further heteroatom ring member selected from 0 N and S. Particular examples of such heterocyclic groups include azetidine, pyrrolidine, piperidine, azepine, piperazine, morpholine and thiomorpholine. One preferred heterocyclic group is piperidine. In each of the foregoing definitions of the non-aromatic heterocyclic group HET, the heterocyclic ring is optionally substituted by one or more substituents which may be selected from groups R¹¹, R^{11a} and R^{11b} and subsets thereof as defined herein. Particular substituents include C₁₋₄ alkyl, such as methyl. One specific example of a non-aromatic group HET is 4,4-dimethylpiperidine.

### GP2

In another embodiment, GP is a group GP2:

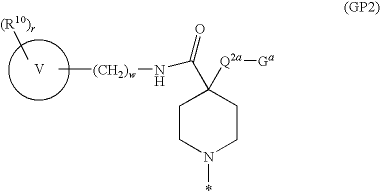

wherein
the ring V is a monocyclic or bicyclic heteroaryl group of 5 to 10 ring members containing up to 4 heteroatom ring members selected from O, N and S;
r is 0, 1, 2, 3 or 4 (e.g. r is 0, 1 or 2);
w is 0 or 1;
T is CH or N;
J¹-J² represents a group selected from N═CH, (R^{q})C═N, HN—C(O), H₂C—C(O), N═N and (R^{q})C═CH;
R^{q} is selected from hydrogen, methyl, chlorine and bromine;
Q^{2a} is a bond or a saturated acyclic hydrocarbon linker group containing from 1 to 3 carbon atoms;
G^{a} is C(O)NR²R³, CN, NR²R³ or OH;
R² and R³ are independently selected from hydrogen; C₁₋₅ alkyl and C₁₋₅ alkanoyl wherein the alkyl and alkanoyl groups are optionally substituted by one or more substituents selected from fluorine, hydroxy, cyano, amino, methylamino, dimethylamino and methoxy; and
   R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

In one embodiment, the moiety r is 0, 1 or 2; i.e. there are 0, 1 or 2 substituents R¹⁰ attached to the ring V.

In one sub-group of compounds, J¹-J² represents a group selected from N═CH, HC═N, HN—C(O), H₂C—C(O), N═N and HC═CH.

In another sub-group of compounds, J¹-J² represents a group selected from HC═N, HC═CH, (Br)C═N, (Cl)C═N, (Me)C═N, (Br)C═CH, (Cl)C═CH and (Me)C═CH.

In GP2, T can be nitrogen or CH and J¹-J² can represent a group selected from N═CH, N═N, HC═N, HN—C(O), H₂C—C(O) and HC═C(R⁶). Thus the bicyclic group can take the form of, for example:
a purine (T is N, J¹-J² is N═CH);
a 3H-imidazo[4,5-b]pyridine (T is CH, J¹-J² is N═CH);
a 7H-pyrrolo[2,3-d]pyrimidine (T is N, J¹-J² is HC═CH);
a 1H-pyrrolo[2,3-b]pyridine (T is CH, J¹-J² is HC═CH);
a 5,7-dihydro-pyrrolo[2,3-d]pyrimidin-6-one (T is N, J¹-J² is H₂C—C(O));
a 3H-[1,2,3]triazolo[4,5-d]pyrimidine (T is N, J¹-J² is N═N);
a 3H-[1,2,3]triazolo[4,5-b]pyridine (T is CH, J¹-J² is N═N);
a 7,9-dihydro-purin-8-one (T is N, J¹-J² is HN—C(O));
a 1H-pyrazolo[3,4-d]pyrimidine (T is N, J¹-J² is HC═N); or
a pyrazolo[3,4-b]pyridine (T is CR⁵, J¹-J² is HC═N).

Particular bicyclic groups are 7H-pyrrolo[2,3-d]pyrimidine and 1H-pyrrolo[2,3-b]pyridine.

In one sub-group of compounds, the bicyclic group is 7H-pyrrolo[2,3-d]pyrimidine.

In another sub-group of compounds, the bicyclic group is 1H-pyrrolo[2,3-b]pyridine.

In a further sub-group of compounds, the bicyclic group is purine (T is N, J¹-J² is N═CH).

Q^{2a} is a bond or a saturated acyclic hydrocarbon linker group containing from 1 to 3 carbon atoms. The saturated acyclic hydrocarbon linker group can be a straight or branched chain alkylene group of 1 to 3 carbon atoms. In one sub-group of compounds, Q^{2a} is a bond or a group (CH₂)ₐ where a is 1 or 2 and preferably is 1.

G^{a} is C(O)NR²R³, CN, NR²R³ or OH. Preferably G^{a} is NR²R³.

In one group of compounds, R² and R³ are independently selected from hydrogen and C₁₋₄ alkyl wherein the alkyl groups are optionally substituted by one or more substituents selected from fluorine, hydroxy, amino, methylamino, dimethylamino and methoxy.

More preferably, the optionally substituted alkyl group forming part of NR²R³ is a C₁, C₂ or C₃ alkyl group, for example a methyl group.

In a particular sub-group of compounds, R² and R³ are independently selected from hydrogen and methyl and hence NR²R³ can be an amino, methylamino or dimethylamino group.

In one embodiment, NR²R³ is an amino group. In another particular embodiment, NR²R³ is a methylamino group. In a further embodiment, NR²R³ is a dimethylamino group.

The heteroaryl ring V can be any of the monocyclic and bicyclic heteroaryl groups listed in the General Preferences and Definitions section above.

Thus, for example, the ring V can be a 5- or 6-membered heteroaryl group containing 1, 2 or 3 (more preferably 1 or 2) heteroatom ring members selected from O, N and S or a 5.6. fused bicyclic heteroaryl group containing 1, 2, 3 or 4 (more preferably 1, 2 or 3 and most preferably 1 or 2) heteroatoms selected from O, N and S.

In one embodiment, the ring V is monocyclic. The ring V preferably contains 1 or 2 heteroatom ring members selected from O, N and S.

In one embodiment, the ring V is a pyridine, pyrazine, pyrimidine, pyridazine, oxazole, imidazole, thiazole, isoxazole, isothiazole, pyrazole or thiophene ring.

Particular monocyclic rings are pyridine (e.g. 2, 3 or 4-pyridyl), pyrazine, pyrimidine, pyridazine, oxazole, imidazole, thiazole, isoxazole, isothiazole, and pyrazole.

In another embodiment, the ring V is bicyclic.

One subset of bicyclic rings consists of benzoimidazole, benzoxazole, benzothiazole, benzofuran, benzothiophene, indole and quinoline.

Particular bicyclic rings are benzoimidazole, benzoxazole, benzothiazole, benzofuran and benzothiophene.

In one preferred embodiment, the monocyclic and bicyclic rings each contain at least one nitrogen ring member.

One preferred group of monocyclic and bicyclic rings V consists of pyridine, pyrazine, isoxazole, pyrazole and benzothiazole.

One particularly preferred monocyclic ring is a 3-pyridyl ring.

Each of the heterocyclic rings V may be unsubstituted or substituted by 1 or 2 substituent groups R¹⁰.

Particular substituents R¹⁰ are the groups of substituents R^{10a}, R^{10b} and R^{10c} as set out in the General Preferences and Definitions section above.

In one embodiment, each substituent R¹⁰ is selected from a group R¹¹ consisting of halogen, hydroxy, trifluoromethyl, cyano, nitro, carboxy, amino, mono- or di-C₁₋₅ hydrocarbylamino, a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen and C₁₋₅ hydrocarbyl optionally substituted by one or more substituents selected from hydroxy, oxo, halogen, cyano, nitro, carboxy, amino and mono- or di-C₁₋₄ hydrocarbylamino, and wherein one or more carbon atoms of the C₁₋₅ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₅ hydrocarbyl;
X¹ is O, S or NR^{c} and X² is ═O, ═S or ═NR^{c}.

More particularly, each substituent may be selected from the groups of substituents R^{11a} and R^{11b} as defined herein.

For example, each substituent R¹⁰ may be selected from a group R²⁴ consisting of fluorine; chlorine; C₁₋₄ alkoxy; trifluoromethyl; trifluoromethoxy; difluoromethoxy; and C₁₋₄ alkyl.

More preferably, R²⁴ is selected from fluorine; chlorine; methoxy; trifluoromethyl; trifluoromethoxy; difluoromethoxy; and methyl.

One sub-group of compounds of the formula (I) wherein GP is a group GP2 is represented by the formula (II):

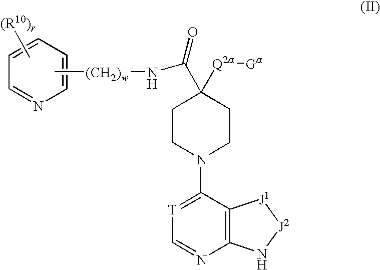

wherein
r is 0, 1 or 2;
w is 0 or 1;

### T is CH or N;

J¹-J² represents a group selected from N═CH, HC═N, HN—C(O), H₂C—C(O), N═N and HC═CH;
Q^{2a} is a bond or a saturated acyclic hydrocarbon linker group containing from 1 to 3 carbon atoms;
G^{a} is C(O)NR²R³, CN, NR²R³ or OH;
R² and R³ are independently selected from hydrogen; C₁₋₅ alkyl and C₁₋₅ alkanoyl wherein the alkyl and alkanoyl groups are optionally substituted by one or more substituents selected from fluorine, hydroxy, cyano, amino, methylamino, dimethylamino and methoxy; and
   R¹⁰ is as defined herein.

Another sub-group of compound of the invention is represented by the formula (III):

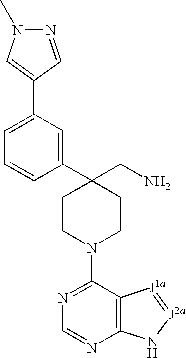

or salts, solvates, N-oxides or tautomers thereof, wherein J^{1a} is selected from CH, C-Me, C—Cl and C—Br; and J^{2a} is selected from N and CH.

A further sub-group of compounds of the invention is represented by the formula (IV):

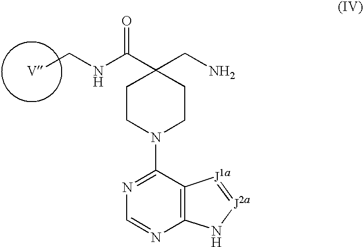

or salts, solvates, N-oxides or tautomers thereof, wherein J^{1a} is selected from N, CH, C-Me, C—Cl and C—Br; J^{2a} is selected from N and CH; and the ring V″ is (i) an optionally substituted heteroaryl ring selected from thienyl, isoxazolyl, indolyl and pyridyl; or (ii) an optionally substituted heteroaryl ring selected from thienyl, isoxazolyl, indolyl, isothiazolyl and pyridyl; wherein in each of (i) and (ii) the optional substituents for the heteroaryl ring are selected from methyl, chlorine, bromine and trifluoromethyl.

Within formula (IV), one sub-set of compounds consists of compounds wherein J^{1a} is selected from CH, C-Me, C—Cl and C—Br and J^{2a} is selected from N and CH.

Another sub-group of compounds of the invention is represented by the formula (IVa):

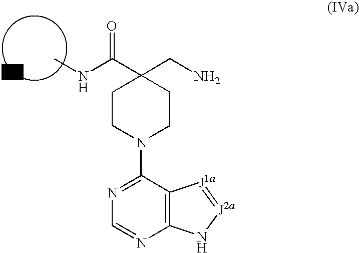

or salts, solvates, N-oxides or tautomers thereof, wherein J^{1a} is selected from N, CH, C-Me, C—Cl and C—Br; J^{2a} is selected from N and CH; and the ring V″ is (i) an optionally substituted heteroaryl ring selected from thienyl, isoxazolyl, indolyl and pyridyl; or (ii) an optionally substituted heteroaryl ring selected from thienyl, isoxazolyl, indolyl, isothiazolyl and pyridyl; wherein in each of (i) and (ii) the optional substituents for the heteroaryl ring are selected from methyl, chlorine, bromine and trifluoromethyl.

Within formula (IVa), one subset of compounds consists of compounds wherein J^{1a} is selected from CH, C-Me, C—Cl and C—Br and J^{2a} is selected from N and CH.

In each of formulae (IV) and (Iva), preferably the optionally substituted heteroaryl ring is selected from 2-thienyl, 5-isoxazolyl, 2-indolyl and 3-pyridyl. For example, the optionally substituted heteroaryl ring can be 2-thienyl substituted by chlorine, methyl and bromine. Alternatively, the heteroaryl ring may be unsubstituted 2-indolyl. In a further alternative, the heteroaryl ring may be 2-isothiazolyl substituted by methyl, e.g. 4-methylthiazol-2-yl and 5-methylthiazol-2-yl.

The various functional groups and substituents making up the compounds of the formula (I) are typically chosen such that the molecular weight of the compound of the formula (I) does not exceed 1000. More usually, the molecular weight of the compound will be less than 750, for example less than 700, or less than 650, or less than 600, or less than 550. More preferably, the molecular weight is less than 525 and, for example, is 500 or less.

Particular compounds of the invention are as illustrated in the examples and as listed below.
4-aminomethyl-1-(7H-pyrrolo[2,3-d]pyrimidin-4-yl)-piperidine-4-carboxylic acid (6-chloro-pyridin-3-ylmethyl)-amide;
4-amino-1-(8-oxo-8,9-dihydro-7H-purin-6-yl)-piperidine-4-carboxylic acid (3-benzooxazol-2-yl-phenyl)-amide;
4-amino-1-(8-oxo-8,9-dihydro-7H-purin-6-yl)-piperidine-4-carboxylic acid [3-(4-methyl-pyridin-2-yl)-phenyl]-amide;
4-amino-1-(7H-pyrrolo[2,3-d]pyrimidin-4-yl)-piperidine-4-carboxylic acid (6-chloro-pyridin-3-ylmethyl)amide;
4-amino-1-(1H-pyrrolo[2,3-b]pyridin-4-yl)-piperidine-4-carboxylic acid (6-chloro-pyridin-3-ylmethyl)-amide;
C-[4-[3-(1-methyl-1H-pyrazol-4-yl)-phenyl]-1-(9H-purin-6-yl)-piperidin-4-yl]-methylamine;
C-[4-[3-(2-methyl-thiophen-3-yl)-phenyl]-1-(9H-purin-6-yl)-piperidin-4-yl]-methylamine;
C-[4-[3-(5-fluoro-pyridin-3-yl)-phenyl]-1-(9H-purin-6-yl)-piperidin-4-yl]-methylamine (; methyl-[4-[3-(1-methyl-1H-pyrazol-4-yl)-phenyl]-1-(9H-purin-6-yl)-piperidin-4-ylmethyl]-amine;
[4-[3-(1-methyl-1H-pyrazol-4-yl)-phenyl]-1-(9H-purin-6-yl)-piperidin-4-yl]-methanol;
4-[3-(1-methyl-1H-pyrazol-4-yl)-phenyl]-1-(9H-purin-6-yl)-piperidine-4-carboxylic acid (2-hydroxy-ethyl)-amide;
6-{4-aminomethyl-4-[3-(1-methyl-1H-pyrazol-4-yl)-phenyl]piperidin-1-yl}-7,9-dihydro-purin-8-one;
C-[4-[3-(1-methyl-1H-pyrazol-4-yl)-phenyl]-1-(1H-pyrazolo[3,4-d]pyrimidin-4-yl)-piperidin-4-yl]-methylamine;
4-amino-1-(7H-pyrrolo[2,3-d]pyrimidin-4-yl)-piperidine-4-carboxylic acid (6-trifluoromethyl-pyridin-3-ylmethyl)-amide hydrochloride;
4-amino-1-(7H-pyrrolo[2,3-d]pyrimidin-4-yl)-piperidine-4-carboxylic acid (5-methyl-pyrazin-2-ylmethyl)-amide hydrochloride;
4-amino-1-(7H-pyrrolo[2,3-d]pyrimidin-4-yl)-piperidine-4-carboxylic acid (5-methyl-isoxazol-3-ylmethyl)-amide hydrochloride;
4-amino-1-(7H-pyrrolo[2,3-d]pyrimidin-4-yl)-piperidine-4-carboxylic acid (1,5-dimethyl-1H-pyrazol-3-ylmethyl)-amide hydrochloride;
4-amino-1-(7H-pyrrolo[2,3-d]pyrimidin-4-yl)-piperidine-4-carboxylic acid (benzothiazol,2-ylmethyl)-amide hydrochloride;
4-amino-1-(7H-pyrrolo[2,3-d]pyrimidin-4-yl)-piperidine-4-carboxylic acid (5-chloro-pyridin-2-ylmethyl)-amide hydrochloride;
4-amino-1-(7H-pyrrolo[2,3-d]pyrimidin-4-yl)-piperidine-4-carboxylic acid (pyridin-2-ylmethyl)-amide hydrochloride;
4-(aminomethyl)-N-((5-bromothiophen-2-yl)methyl)-1-(7H-pyrrolo[2,3-d]pyrimidin-4-yl)piperidine-4-carboxamide;
4-(aminomethyl)-N-((5-chlorothiophen-2-yl)methyl)-1-(7H-pyrrolo[2,3-d]pyrimidin-4-yl)piperidine-4-carboxamide;
4-(aminomethyl)-N-((5-methylthiophen-2-yl)methyl)-1-(7H-pyrrolo[2,3-d]pyrimidin-4-yl)piperidine-4-carboxamide;
4-(aminomethyl)-N-((3-bromoisoxazol-5-yl)methyl)-1-(7H-pyrrolo[2,3-d]pyrimidin-4-yl)piperidine-4-carboxamide;
N-((1H-indol-2-yl)methyl)-4-(aminomethyl)-1-(3-bromo-1H-pyrazolo[3,4-d]pyrimidin-4-yl)piperidine-4-carboxamide;
N-((1H-indol-2-yl)methyl)-4-(aminomethyl)-1-(7H-pyrrolo[2,3-d]pyrimidin-4-yl)piperidine-4-carboxamide;
4-(aminomethyl)-1-(7H-pyrrolo[2,3-d]pyrimidin-4-yl)-N-((6-(trifluoromethyl)pyridin-3-yl)methyl)piperidine-4-carboxamide;
(1-(5-bromo-7H-pyrrolo[2,3-d]pyrimidin-4-yl)-4-(3-(1-methyl-1H-pyrazol-4-yl)phenyl)piperidin-4-yl)methanamine;
(1-(5-chloro-7H-pyrrolo[2,3-d]pyrimidin-4-yl)-4-(3-(1-methyl-1H-pyrazol-4-yl)phenyl)piperidin-4-yl)methanamine;
(4-(3-(1-methyl-1H-pyrazol-4-yl)phenyl)-1-(5-methyl-7H-pyrrolo[2,3-d]pyrimidin-4-yl)piperidin-4-yl)methanamine;
(1-(3-bromo-1H-pyrazolo[3,4-d]pyrimidin-4-yl)-4-(3-(1-methyl-1H-pyrazol-4-yl)phenyl)piperidin-4-yl)methanamine;
N,N-dimethyl-1-(4-(3-(1-methyl-1H-pyrazol-4-yl)phenyl)-1-(9H-purin-6-yl)piperidin-4-yl)methanamine;
6-(4-(3-(1-methyl-1H-pyrazol-4-yl)phenyl)-4-(pyrrolidin-1-ylmethyl)piperidin-1-yl)-9H-purine;
3-(4-(3-(1-methyl-1H-pyrazol-4-yl)phenyl)-1-(9H-purin-6-yl)piperidin-4-yl)propan-1-amine;
2-amino-N-((4-(3-(1-methyl-1H-pyrazol-4-yl)phenyl)-1-(9H-purin-6-yl)piperidin-4-yl)methyl)acetamide;
2-(dimethylamino)-N-((4-(3-(1-methyl-1H-pyrazol-4-yl)phenyl)-1-(9H-purin-6-yl)piperidin-4-yl)methyl)acetamide;
4-[3-(1-methylpyrazol-4-yl)phenyl]-1-(9H-purin-6-yl)piperidine-4-carboxamide;
4-(aminomethyl)-N-[(6-chloropyridin-3-yl)methyl]-1-(7H-pyrrolo[2,3-d]pyrimidin-4-yl)piperidine-4-carboxamide;
4-amino-N-[(5-chlorothiophen-2-yl)methyl]-1-(7H-pyrrolo[2,3-d]pyrimidin-4-yl)piperidine-4-carboxamide;
4-amino-N-[(4-methyl-1,3-thiazol-2-yl)methyl]-1-(7H-pyrrolo[2,3-d]pyrimidin-4-yl)piperidine-4-carboxamide;
4-amino-1-(7H-pyrrolo[2,3-d]pyrimidin-4-yl)-N-(quinolin-3-ylmethyl)piperidine-4-carboxamide;
4-amino-N-[(2-phenyl-1,3-thiazol-4-yl)methyl]-1-(7H-pyrrolo[2,3-d]pyrimidin-4-yl)piperidine-4-carboxamide;
4-amino-N-(pyridin-4-ylmethyl)-1-(7H-pyrrolo[2,3-d]pyrimidin-4-yl)piperidine-4-carboxamide;
4-amino-N-[[3-(4-chlorophenyl)-1,2-oxazol-5-yl]methyl]-1-(7H-pyrrolo[2,3-d]pyrimidin-4-yl)piperidine-4-carboxamide;
4-amino-N-[(1-methylpyrazol-4-yl)methyl]-1-(7H-pyrrolo[2,3-d]pyrimidin-4-yl)piperidine-4-carboxamide;
4-amino-1-(7H-pyrrolo[2,3-d]pyrimidin-4-yl)-piperidine-4-carboxylic acid (2-phenyl-thiazol-5-ylmethyl)-amide;
4-amino-1-(7H-pyrrolo[2,3-d]pyrimidin-4-yl)-piperidine-4-carboxylic acid (3-methyl-thiophen-2-ylmethyl)-amide;
4-amino-1-(7H-pyrrolo[2,3-d]pyrimidin-4-yl)-piperidine-4-carboxylic acid (3-bromo-isoxazol-5-ylmethyl)-amide;
4-amino-1-(7H-pyrrolo[2,3-d]pyrimidin-4-yl)-piperidine-4-carboxylic acid (3-methyl-isoxazol-5-ylmethyl)-amide;
4-amino-1-(7H-pyrrolo[2,3-d]pyrimidin-4-yl)-piperidine-4-carboxylic acid (1H-indol-2-ylmethyl)-amide;
(4-(3-(1-methyl-1H-pyrazol-4-yl)phenyl)-1-(7H-pyrrolo[2,3-d]pyrimidin-4-yl)piperidin-4-yl)methanamine;
4-amino-1-(7H-pyrrolo[2,3-d]pyrimidin-4-yl)-piperidine-4-carboxylic acid (3-benzooxazol-2-yl-phenyl)-amide;
4-amino-1-(7H-pyrrolo[2,3-d]pyrimidin-4-yl)-piperidine-4-carboxylic acid [3-(5-fluoro-pyrimidin-2-yl)-phenyl]-amide;
4-amino-1-(7H-pyrrolo[2,3-d]pyrimidin-4-yl)-piperidine-4-carboxylic acid [3-(4-methyl-pyridin-2-yl)-phenyl]-amide;
4-amino-1-(7H-pyrrolo[2,3-d]pyrimidin-4-yl)-piperidine-4-carboxylic acid [3-(4,4-dimethyl-piperidin-1-yl)-phenyl]-amide;
4-amino-1-(9H-purin-6-yl)-piperidine-4-carboxylic acid [3-(4,4-dimethyl-piperidin-1-yl)-phenyl]-amide;
4-amino-1-(9H-purin-6-yl)-piperidine-4-carboxylic acid (3-benzooxazol-2-yl-phenyl)-amide;
4-(aminomethyl)-N-(4-methylthiazol-2-yl)-1-(7H-pyrrolo[2,3-d]pyrimidin-4-yl)piperidine-4-carboxamide;
4-(aminomethyl)-N-(5-methylthiazol-2-yl)-1-(7H-pyrrolo[2,3-d]pyrimidin-4-yl)piperidine-4-carboxamide;
4-(aminomethyl)-N-(5-fluoropyridin-2-yl)-1-(7H-pyrrolo[2,3-d]pyrimidin-4-yl)piperidine-4-carboxamide;
4-(aminomethyl)-1-(7H-pyrrolo[2,3-d]pyrimidin-4-yl)-N-(5-(trifluoromethyl)pyridin-2-yl)piperidine-4-carboxamide;
4-(aminomethyl)-N-(benzo[d]thiazol-6-yl)-1-(7H-pyrrolo[2,3-d]pyrimidin-4-yl)piperidine-4-carboxamide;
4-(aminomethyl)-N-(benzo[d]thiazol-2-yl)-1-(7H-pyrrolo[2,3-d]pyrimidin-4-yl)piperidine-4-carboxamide;
4-(aminomethyl)-N-(3-methyl-1,2,4-thiadiazol-5-yl)-1-(7H-pyrrolo[2,3-d]pyrimidin-4-yl)piperidine-4-carboxamide;
4-(aminomethyl)-N-(6-(methylsulfonyl)benzo[d]thiazol-2-yl)-1-(7H-pyrrolo[2,3-d]pyrimidin-4-yl)piperidine-4-carboxamide;
4-(aminomethyl)-N-(4-(pyridin-3-yl)thiazol-2-yl)-1-(7H-pyrrolo[2,3-d]pyrimidin-4-yl)piperidine-4-carboxamide;
4-(aminomethyl)-N-(pyridin-2-yl)-1-(7H-pyrrolo[2,3-d]pyrimidin-4-yl)piperidine-4-carboxamide;
4-(aminomethyl)-1-(7H-pyrrolo[2,3-d]pyrimidin-4-yl)-N-(4-(trifluoromethyl)pyridin-2-yl)piperidine-4-carboxamide;
4-(aminomethyl)-N-(5-methylpyridin-2-yl)-1-(7H-pyrrolo[2,3-d]pyrimidin-4-yl)piperidine-4-carboxamide;
(4-(3-fluoro-5-(1-methyl-1H-pyrazol-4-yl)phenyl)-1-(7H-pyrrolo[2,3-d]pyrimidin-4-yl)piperidin-4-yl)methanamine;
(4-(3-fluoro-5-(5-fluoropyridin-3-yl)phenyl)-1-(7H-pyrrolo[2,3-d]pyrimidin-4-yl)piperidin-4-yl)methanamine;
4-(aminomethyl)-N-(5-methylthiazol-2-yl)-1-(9H-purin-6-yl)piperidine-4-carboxamide [4-[3-(1H-pyrazol-4-yl)phenyl]-1-(7H-pyrrolo[3,2-e]pyrimidin-4-yl)piperidin-4-yl]methanamine;
[4-[3-(4-methylpyridin-3-yl)phenyl]-1-(7H-pyrrolo[3,2-e]pyrimidin-4-yl)piperidin-4-yl]methanamine;
[4-(3-pyridin-4-ylphenyl)-1-(7H-pyrrolo[3,2-e]pyrimidin-4-yl)piperidin-4-yl]methanamine;
[4-(3-pyridin-3-ylphenyl)-1-(7H-pyrrolo[3,2-e]pyrimidin-4-yl)piperidin-4-yl]methanamine;
[4-(3-pyrimidin-5-ylphenyl)-1-(7H-pyrrolo[3,2-e]pyrimidin-4-yl)piperidin-4-yl]methanamine;
[4-[3-(furan-2-yl)phenyl]-1-(7H-pyrrolo[3,2-e]pyrimidin-4-yl)piperidin-4-yl]methanamine;
[4-(3-furan-3-ylphenyl)-1-(7H-pyrrolo[3,2-e]pyrimidin-4-yl)piperidin-4-yl]methanamine;
[4-[3-(1,2-oxazol-4-yl)phenyl]-1-(7H-pyrrolo[3,2-e]pyrimidin-4-yl)piperidin-4-yl]methanamine;
4-(aminomethyl)-N-(5-chloropyridin-2-yl)-1-(7H-pyrrolo[2,3-d]pyrimidin-4-yl)piperidine-4-carboxamide;
4-(aminomethyl)-1-(7H-pyrrolo[2,3-d]pyrimidin-4-yl)-N-(6-(trifluoromethyl)pyridin-3-yl)piperidine-4-carboxamide;
   and salts, solvates, tautomers and N-oxides thereof.

### Salts, Solvates, Tautomers, Isomers, N-Oxides, Esters, Prodrugs and Isotopes

Unless otherwise specified, a reference to a particular compound also includes ionic, salt, solvate, and protected forms thereof, for example, as discussed below.

Many compounds of the formula (I) can exist in the form of salts, for example acid addition salts or, in certain cases salts of organic and inorganic bases such as carboxylate, sulphonate and phosphate salts. All such salts are within the scope of this invention, and references to compounds of the formula (I) include the salt forms of the compounds. As in the preceding sections of this application, all references to formula (I) should be taken to refer also to all sub-groups thereof unless the context indicates otherwise.

Salt forms may be selected and prepared according to methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. For example, acid addition salts may be prepared by dissolving the free base in an organic solvent in which a given salt form is insoluble or poorly soluble and then adding the required acid in an appropriate solvent so that the salt precipitates out of solution.

Acid addition salts may be formed with a wide variety of acids, both inorganic and organic. Examples of acid addition salts include salts formed with an acid selected from the group consisting of acetic, 2,2-dichloroacetic, adipic, alginic, ascorbic (e.g. L-ascorbic), L-aspartic, benzenesulphonic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic, (−)-L-malic, malonic, (±)-DL-mandelic, methanesulphonic, naphthalenesulphonic (e.g. naphthalene-2-sulphonic), naphthalene-1,5-disulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, (+)-L-tartaric, thiocyanic, toluenesulphonic (e.g. p-toluenesulphonic), undecylenic and valeric acids, as well as acylated amino acids and cation exchange resins.

One particular group of acid addition salts includes salts formed with hydrochloric, hydriodic, phosphoric, nitric, sulphuric, citric, lactic, succinic, maleic, malic, isethionic, fumaric, benzenesulphonic, toluenesulphonic, methanesulphonic, ethanesulphonic, naphthalenesulphonic, valeric, acetic, propanoic, butanoic, malonic, glucuronic and lactobionic acids. Within this group of salts, a sub-set of salts consists of salts formed with hydrochloric acid or acetic acid.

Another group of acid addition salts includes salts formed from acetic, adipic, ascorbic, aspartic, citric, DL-Lactic, fumaric, gluconic, glucuronic, hippuric, hydrochloric, glutamic, DL-malic, methanesulphonic, sebacic, stearic, succinic and tartaric acids.

The compounds of the invention may exist as mono- or di-salts depending upon the pKa of the acid from which the salt is formed. In stronger acids, the basic pyrazole nitrogen, as well as the nitrogen atom in the group NR²R³, may take part in salt formation. For example, where the acid has a pKa of less than about 3 (e.g. an acid such as hydrochloric acid, sulphuric acid or trifluoroacetic acid), the compounds of the invention will typically form salts with 2 molar equivalents of the acid.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds of the formula (I) contain an amine function, these may form quaternary ammonium salts, for example by reaction with an alkylating agent according to methods well known to the skilled person. Such quaternary ammonium compounds are within the scope of formula (I).

The salt forms of the compounds of the invention are typically pharmaceutically acceptable salts, and examples of pharmaceutically acceptable salts are discussed in Berge et al., 1977, “Pharmaceutically Acceptable Salts,” J. Pharm. Sci., Vol. 66, pp. 1-19. However, salts that are not pharmaceutically acceptable may also be prepared as intermediate forms which may then be converted into pharmaceutically acceptable salts. Such non-pharmaceutically acceptable salts forms, which may be useful, for example, in the purification or separation of the compounds of the invention, also form part of the invention.

Compounds of the formula (I) containing an amine function may also form N-oxides. A reference herein to a compound of the formula (I) that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Compounds of the formula (I) may exist in a number of different geometric isomeric, and tautomeric forms and references to compounds of the formula (I) include all such forms. For the avoidance of doubt, where a compound can exist in one of several geometric isomeric or tautomeric forms and only one is specifically described or shown, all others are nevertheless embraced by formula (I).

For example, when J¹-J² is N═CR⁶, the tautomeric forms A and B are possible for the bicyclic group.

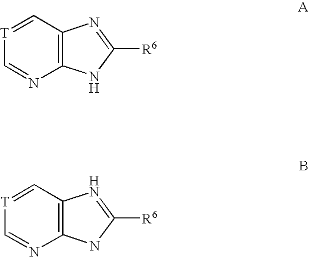

When J¹-J² is HN—CO, the tautomeric forms C, D and E are possible for the bicyclic group.

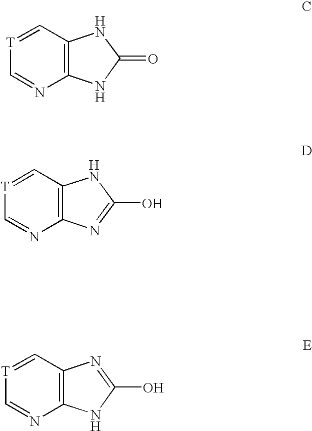

All such tautomers are embraced by formula (I).

Other examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro

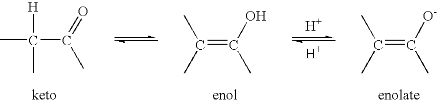

Where compounds of the formula (I) contain one or more chiral centres, and can exist in the form of two or more optical isomers, references to compounds of the formula (I) include all optical isomeric forms thereof (e.g. enantiomers, epimers and diastereoisomers), either as individual optical isomers, or mixtures (e.g. racemic or scalemic mixtures) or two or more optical isomers, unless the context requires otherwise.

The optical isomers may be characterised and identified by their optical activity (i.e. as + and − isomers, or d and l isomers) or they may be characterised in terms of their absolute stereochemistry using the “R and S” nomenclature developed by Cahn, Ingold and Prelog, see Advanced Organic Chemistry by Jerry March, 4^{th} Edition, John Wiley & Sons, New York, 1992, pages 109-114, and see also Cahn, Ingold & Prelog, Angew. Chem. Int. Ed. Engl., 1966, 5, 385-415.

Optical isomers can be separated by a number of techniques including chiral chromatography (chromatography on a chiral support) and such techniques are well known to the person skilled in the art.

As an alternative to chiral chromatography, optical isomers can be separated by forming diastereoisomeric salts with chiral acids such as (+)-tartaric acid, (−)-pyroglutamic acid, (−)-di-toluloyl-L-tartaric acid, (+)-mandelic acid, (−)-malic acid, and (−)-camphorsulphonic, separating the diastereoisomers by preferential crystallisation, and then dissociating the salts to give the individual enantiomer of the free base.

Where compounds of the formula (I) exist as two or more optical isomeric forms, one enantiomer in a pair of enantiomers may exhibit advantages over the other enantiomer, for example, in terms of biological activity. Thus, in certain circumstances, it may be desirable to use as a therapeutic agent only one of a pair of enantiomers, or only one of a plurality of diastereoisomers. Accordingly, the invention provides compositions containing a compound of the formula (I) having one or more chiral centres, wherein at least 55% (e.g. at least 60%, 65%, 70%, 75%, 80%, 85%, 90% or 95%) of the compound of the formula (I) is present as a single optical isomer (e.g. enantiomer or diastereoisomer). In one general embodiment, 99% or more (e.g. substantially all) of the total amount of the compound of the formula (I) may be present as a single optical isomer (e.g. enantiomer or diastereoisomer).

The compounds of the invention include compounds with one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O.

The isotopes may be radioactive or non-radioactive. In one embodiment of the invention, the compounds contain no radioactive isotopes. Such compounds are preferred for therapeutic use. In another embodiment, however, the compound may contain one or more radioisotopes. Compounds containing such radioisotopes may be useful in a diagnostic context.

Esters such as carboxylic acid esters of the compounds of formula (I) bearing a hydroxyl group are also embraced by Formula (I). In one embodiment of the invention, formula (I) includes within its scope esters of compounds of the formula (I) bearing a hydroxyl group. In another embodiment of the invention, formula (I) does not include within its scope esters of compounds of the formula (I) bearing a hydroxyl group. Examples of esters are compounds containing the group —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Particular examples of ester groups include, but are not limited to, —C(═O)OCH₃, —C(═O)OCH₂CH₃, —C(═O)OC(CH₃)₃, and —C(═O)OPh. Examples of acyloxy (reverse ester) groups are represented by —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Particular examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Also encompassed by formula (I) are any polymorphic forms of the compounds, solvates (e.g. hydrates), complexes (e.g. inclusion complexes or clathrates with compounds such as cyclodextrins, or complexes with metals) of the compounds, and pro-drugs of the compounds. By “prodrugs” is meant for example any compound that is converted in vivo into a biologically active compound of the formula (I).

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the hydroxyl groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is:

C₁₋₇alkyl
(e.g., -Me, -Et, -nPr, -iPr, -nBu, -sBu, -iBu, -tBu);
C₁₋₇-aminoalkyl
(e.g., aminoethyl; 2-(N,N-diethylamino)ethyl; 2-(4-morpholino)ethyl); and
acyloxy-C₁₋₇alkyl
(e.g., acyloxymethyl;
acyloxyethyl;
pivaloyloxymethyl;
acetoxymethyl;
1-acetoxyethyl;
1-(1-methoxy-1-methyl)ethyl-carbonxyloxyethyl;
1-(benzoyloxy)ethyl; isopropoxy-carbonyloxymethyl;
1-isopropoxy-carbonyloxyethyl; cyclohexyl-carbonyloxymethyl;
1-cyclohexyl-carbonyloxyethyl;
cyclohexyloxy-carbonyloxymethyl;
1-cyclohexyloxy-carbonyloxyethyl;
(4-tetrahydropyranyloxy) carbonyloxymethyl;
1-(4-tetrahydropyranyloxy)carbonyloxyethyl;
(4-tetrahydropyranyl)carbonyloxymethyl; and
1-(4-tetrahydropyranyl)carbonyloxyethyl).

Also, some prodrugs are activated enzymatically to yield the active compound, or a compound which, upon further chemical reaction, yields the active compound (for example, as in Antibody-directed Enzyme Prodrug Therapy (ADEPT), Gene-directed Enzyme Prodrug Therapy (GDEPT), Polymer-directed Enzyme Prodrug Therapy (PDEPT), Ligand-directed Enzyme Prodrug Therapy (LIDEPT), etc.). For example, the prodrug may be a sugar derivative or other glycoside conjugate, or may be an amino acid ester derivative.

### Methods for the Preparation of Compounds of the Formula (I)

In this section, references to compounds of the formula (I) include each of the sub-groups thereof as defined herein unless the context requires otherwise.

In a further aspect, the invention provides a process for the preparation of a compound of the formula (I) as defined herein.

Compounds of the formula (I) can be prepared by the reaction of a compound of the formula (XVI) where T is N and Hal is chlorine or fluorine (more usually chlorine), with a compound of the formula (XVII) or a protected derivative thereof, where R′ and R″ represent the residues of the group GP.

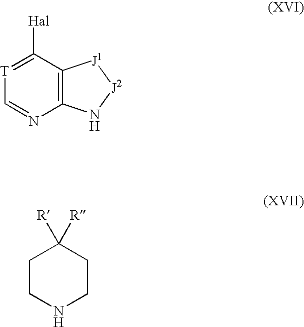

The reaction is typically carried out in a polar solvent such as an alcohol (e.g. ethanol, propanol or n-butanol) at an elevated temperature, for example a temperature in the region from 90° C. to 160° C., optionally in the presence of a non-interfering amine such as triethylamine. The reaction may be carried out in a sealed tube, particularly where the desired reaction temperature exceeds the boiling point of the solvent. When T is N, the reaction is typically carried out at a temperature in the range from about 100° C. to 130° C. but, when T is CH, higher temperatures may be required, for example up to about 160° C., and hence higher boiling solvents such as dimethylformamide or N-methylpyrrolidinone may be used. In general, an excess of the nucleophilic amine will be used and/or an additional non-reacting base such as triethylamine will be included in the reaction mixture. Heating of the reaction mixture may be accomplished by normal means or by the use of a microwave heater.

In order to prepare compounds of the formula (I) wherein T is CH, the hydrogen atom of the group CH may be replaced by an activating group in order to facilitate nucleophilic displacement of the chlorine atom by the amine (XVII). The activating group is typically one which can be removed subsequent to the nucleophilic displacement reaction. One such activating group is an ester group such as ethoxycarbonyl or methoxycarbonyl which can be removed by hydrolysis and decarboxylation. Hydrolysis of the ethoxycarbonyl or methoxycarbonyl group to the carboxylic acid is typically carried out using an aqueous alkali such as sodium hydroxide, and the decarboxylation step is typically conducted by heating to an elevated temperature (e.g. 150° C. to 190° C.).

Compounds of the formula (XVI) are commercially available or can be prepared according to methods well known to the skilled person.

Commercially available compounds of the formula (XVI) include 6-chloro-9H-purine, 2-amino-6-chloropurine, 2-methylthio-6-chloropurine, 4-chloropyrrolo[2,3-d]pyrimidine, 4-chloro-1 h-pyrazolo[3,4-d]pyrimidine, 6-chloro-2-methoxy-7-deazapurine, 6-chloro-7-deazaguanine, 4-chloro-1 h-pyrazolo[3,4-d]pyrimidin-6-ylamine, 7-chloro-3h-[1,2,3]triazolo[4,5-d]pyrimidine, 4-fluoro-7-azaindole, 4-chloro-7-azaindole, 3-bromo-4-chloro-1 h-pyrazolo[3,4-d]pyrimidine, 6-bromo-4-chloro-7h-pyrrolo[2,3-d]pyrimidine and 6-chloro-2-(trifluoromethyl)-9h-purine.

Compounds of the formula (XVI) where T is N and J¹-J² is (Br)C═CH or (Cl)C═CH can be prepared from the corresponding compound wherein J¹-J² is HC═CH by reaction with N-bromosuccinimide (NBS) or N-clorosuccinimide (NCS) respectively. The reaction is typically carried out in a non-protic solvent such as dichloromethane and preferably under nitrogen. Compounds wherein J¹-J² is (Br)C═CH can be converted to the corresponding compound wherein J¹-J² is (R⁷)C═CH where R⁷ is an alkyl group such as methyl by lithiation with an alkyl lithium compound followed by reaction with an alkyl halide such as methyl iodide.

Compounds of the formula (XVI) where T is N and J¹-J² is CH═N, can be prepared from the corresponding hydroxy compounds by reaction with a chlorinating agent such as POCl₃. Compounds of the formula (XVI) where J¹-J² is HN—C(O) can be prepared by the reaction of an ortho-diamino compound of the formula (XVIII) with carbonyl di-imidazole in the presence of a non-interfering base such as triethylamine.

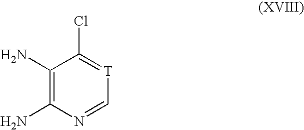

Compounds of the formula (XVI) where T is CH and J¹-J² is H₂C═CH₂) can be prepared from the corresponding N-oxide of the formula (XIX) by reaction with phosphorus oxychloride at an elevated temperature, for example the reflux temperature of POCl₃.

Compounds of the formula ((XVII) in which R′ is a substituted phenyl group (e.g. a phenyl group bearing a substituent HET and R″ is a CH₂NH₂ group (i.e. as in compounds of the formula (I) GP is GP1) can be prepared using the sequence of steps shown in Scheme 3.

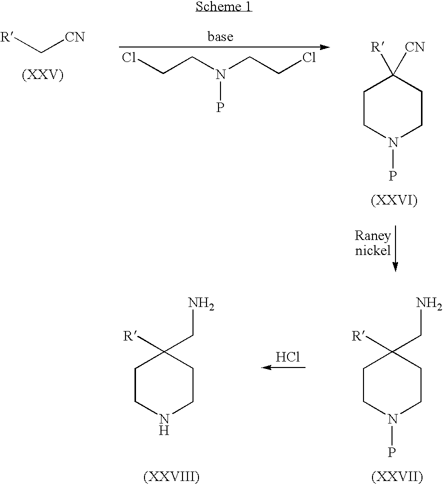

As shown in Scheme 1, the nitrile (XXV) in which R′ is a substituted phenyl group is reacted with a base and N-protected (P=protecting group) bis-(2-chloroethyl)amine to give the piperidine nitrile (XXVI) which can then be reduced to give the amine (XXVII) using Raney nickel and then deprotected (e.g. using HCl when the protecting group is acid labile) to give amine (XXVIII).

Compounds of the formula (I) in which R′ is a substituted phenyl group and R″ is a NH₂ group can also be prepared by the reaction sequence shown in Scheme 2.

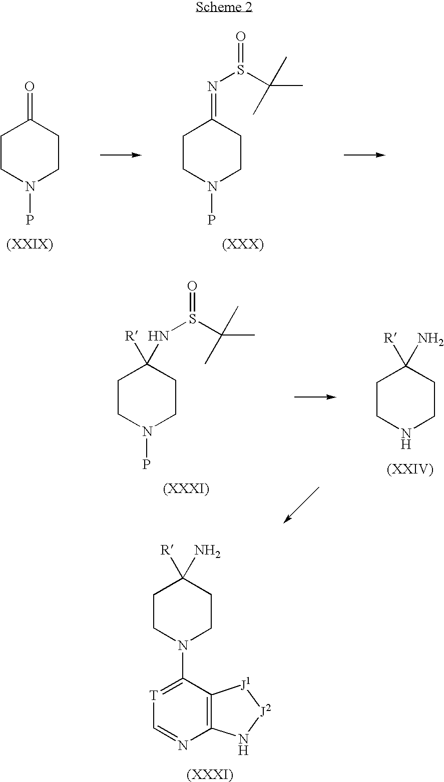

As shown in Scheme 2, a protected 4-piperidone (XXIX), in which P is a protecting group such as Boc, is reacted with tert-butylsulphinimide in the presence of titanium tetraethoxide in a dry polar solvent such as THF to give the sulphinimine (XXX). The reaction is typically carried out with heating, for example to the reflux temperature of the solvent. The sulphinimine (XXX) is then reacted with an organometallic reagent, for example a Grignard reagent such as a substituted phenylmagnesium bromide, suitable for introducing the moiety R′, to give the sulphinamide (XXXI). The tert-butylsulphinyl group can then be removed by hydrolysis in a hydrochloric acid/dioxane/methanol mixture to give the amine (XXIV). The amine (XXIV) can then be reacted with a chloro-heterocycle (XVI) under the conditions described above to give the product (XXXI.

The formation of compounds of the formula (I) wherein GP is GP2 is illustrated by the sequence of reactions set out in Scheme 3.

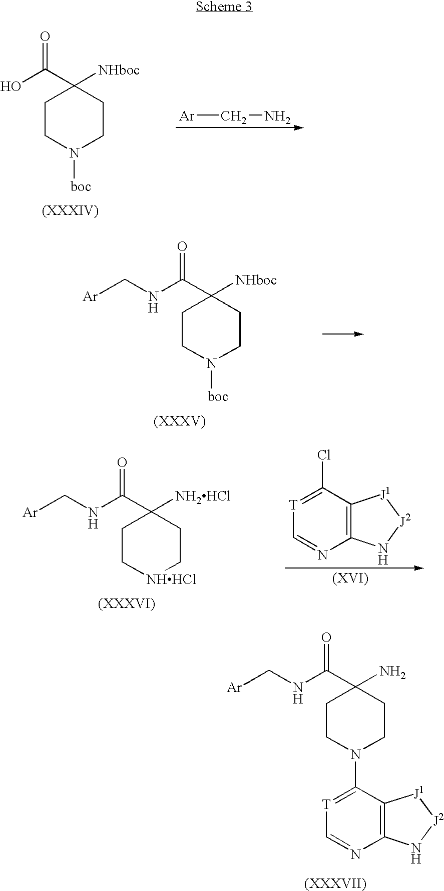

In Scheme 3, the boc-protected piperidine amino acid (XXXIV) is reacted with the heteroarylamine ArCH₂—NH₂ (wherein “Ar is an optionally substituted pyridyl group) using standard amide forming conditions. Thus, for example, the reaction is preferably carried out in the presence of a reagent of the type commonly used in the formation of peptide linkages. Examples of such reagents include 1,3-dicyclohexylcarbodiimide (DCC) (Sheehan et al, J. Amer. Chem Soc. 1955, 77, 1067), 1-ethyl-3-(3′-dimethylaminopropyl)-carbodiimide (referred to herein either as EDC or EDAC) (Sheehan et al, J. Org. Chem., 1961, 26, 2525), uronium-based coupling agents such as O-(7-azabenzotriazol-1-yl)-N,N,N′,N′-tetramethyluronium hexafluorophosphate (HATU) and phosphonium-based coupling agents such as 1-benzo-triazolyloxytris-(pyrrolidino)phosphonium hexafluorophosphate (PyBOP) (Castro et al, Tetrahedron Letters, 1990, 31, 205). Carbodiimide-based coupling agents are advantageously used in combination with 1-hydroxy-7-azabenzotriazole (HOAt) (L. A. Carpino, J. Amer. Chem. Soc., 1993, 115, 4397) or 1-hydroxybenzotriazole (HOBt) (Konig et al, Chem. Ber., 103, 708, 2024-2034). Preferred coupling reagents include EDC (EDAC) and DCC in combination with HOAt or HOBt.

The coupling reaction is typically carried out in a non-aqueous, non-protic solvent such as acetonitrile, dioxan, dimethylsulphoxide, dichloromethane, dimethylformamide or N-methylpyrrolidinone, or in an aqueous solvent optionally together with one or more miscible co-solvents. The reaction can be carried out at room temperature or, where the reactants are less reactive (for example in the case of electron-poor anilines bearing electron withdrawing groups such as sulphonamide groups) at an appropriately elevated temperature. The reaction may be carried out in the presence of a non-interfering base, for example a tertiary amine such as triethylamine or N,N-diisopropylethylamine.

As an alternative, a reactive derivative of the carboxylic acid, e.g. an anhydride or acid chloride, may be used. Reaction with a reactive derivative such an anhydride is typically accomplished by stirring the amine and anhydride at room temperature in the presence of a base such as pyridine.

Compounds of the formula (I) wherein T is CH and J¹-J² is CH═N or CH═CH can be prepared according to the procedure illustrated in Scheme 4.

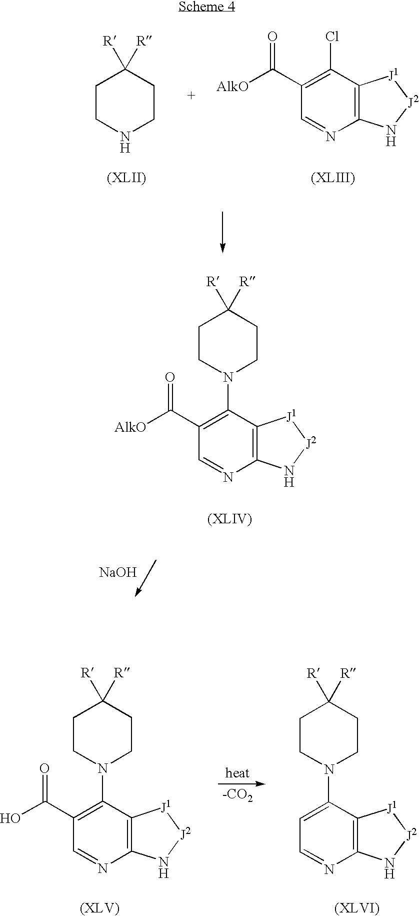

In the sequence of reactions shown in Scheme 4, the starting material is the chlorinated carboxy ester compound (XLIII) which can be prepared by methods generally analogous to methods described in J. Heterocycl. Chem. 1972, 235 and Bioorg. Med. Chem. Lett. 2003, 2405 followed by removal of any unwanted protecting groups where necessary. In formula (XLIII), AlkO is an alkoxy group, e.g. a C₁₋₃ alkoxy group such as methoxy or ethoxy (particularly ethoxy).

The substituted piperidine compound (XLII), suitably protected where necessary, is reacted with the chlorinated carboxy ester compound (XLIII), to give an ester intermediate of the formula (XLIV). The reaction may be carried out in a polar solvent such as a higher boiling alcohol (e.g. n-butanol) in the presence of a non-interfering base such as triethylamine at an elevated temperature (e.g. 90° C. to 130° C., more typically 100° C. to 120° C.). Heating can be effected by means of a microwave heater.

The carboxy ester group in the chlorinated carboxy ester compound (XLIII) functions as an activating group, rendering the chlorine atom more susceptible to nucleophilic displacement. Once the nucleophilic displacement reaction has taken place, the carboxy ester group has served its purpose and can be removed. Accordingly, hydrolysis of the ester intermediate (XLIV) to the carboxylic acid (XLV) is carried out using an aqueous alkali metal hydroxide such as potassium hydroxide or sodium hydroxide with heating where necessary. The carboxylic acid (XLV) is then decarboxylated to give the product (XLVI) by heating to an elevated temperature in excess of 100° C., for example a temperature in the range from about 120° C. to about 180° C.).

Compounds wherein GP is a group GP1 can also be prepared by the reaction of a compound of the formula (XLVII):

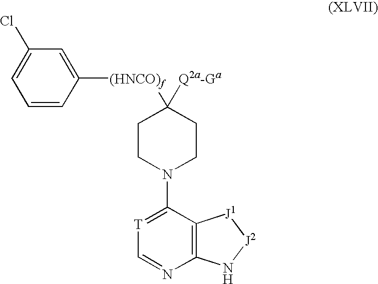

with a boronic acid or boronate suitable for introducing the group HET. The reaction is carried out under Suzuki coupling conditions in the presence of a palladium catalyst and base. Many boronates suitable for use in preparing compounds of the invention are commercially available, for example from Boron Molecular Limited of Noble Park, Australia, or from Combi-Blocks Inc, of San Diego, USA. Where the boronates are not commercially available, they can be prepared by methods known in the art, for example as described in the review article by N. Miyaura and A. Suzuki, Chem. Rev. 1995, 95, 2457. Thus, boronates can be prepared by reacting the corresponding bromo-compound with an alkyl lithium such as butyl lithium and then reacting with a borate ester. The resulting boronate ester derivative can, if desired, be hydrolysed to give the corresponding boronic acid.

Alternatively, compounds wherein GP is a group GP1 can be prepared by the nucleophilic substitution reaction of a compound of the formula (XLVIII):

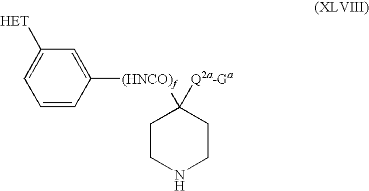

with a compound of the formula (XVI) where T is N and Hal is chlorine or fluorine (more usually chlorine), as described above.

Compounds of the formula (XLVIII) can be prepared by reaction of a compound of the formula (XLIX):

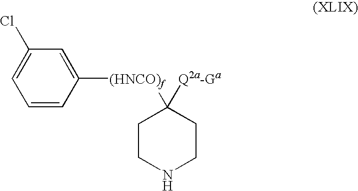

or a protected form thereof, with a boronic acid or boronate suitable for introducing the group HET under the Suzuki coupling conditions described above.

Once formed, many compounds of the formula (I) can be converted into other compounds of the formula (I) using standard functional group interconversions.

Examples of functional group interconversions and reagents and conditions for carrying out such conversions can be found in, for example, Advanced Organic Chemistry, by Jerry March, 4^{th} edition, 119, Wiley Interscience, New York, Fiesers' Reagents for Organic Synthesis, Volumes 1-17, John Wiley, edited by Mary Fieser (ISBN: 0-471-58283-2), and Organic Syntheses, Volumes 1-8, John Wiley, edited by Jeremiah P. Freeman (ISBN: 0-471-31192-8).

### Protecting Groups

In many of the reactions described above, it may be necessary to protect one or more groups to prevent reaction from taking place at an undesirable location on the molecule. Examples of protecting groups, and methods of protecting and deprotecting functional groups, can be found in Protective Groups in Organic Synthesis (T. Green and P. Wuts; 3rd Edition; John Wiley and Sons, 1999).

A hydroxy group may be protected, for example, as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc). An aldehyde or ketone group may be protected, for example, as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid. An amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), or as a 2-(phenylsulphonyl)ethyloxy amide (—NH-Psec). Other protecting groups for amines, such as cyclic amines and heterocyclic N—H groups, include toluenesulphonyl (tosyl) and methanesulphonyl (mesyl) groups and benzyl groups such as a para-methoxybenzyl (PMB) group. A carboxylic acid group may be protected as an ester for example, as: an C₁₋₇ alkyl ester (e.g., a methyl ester; a t-butyl ester); a C₁₋₇ haloalkyl ester (e.g., a C₁₋₇ trihaloalkyl ester); a triC₁₋₇ alkylsilyl-C₁₋₇alkyl ester; or a C₅₋₂₀ aryl-C₁₋₇ alkyl ester (e.g., a benzyl ester; a nitrobenzyl ester); or as an amide, for example, as a methyl amide. A thiol group may be protected, for example, as a thioether (—SR), for example, as: a benzyl thioether; an acetamidomethyl ether (—S—CH₂NHC(═O)CH₃).

### Isolation and Purification of the Compounds of the Invention

The compounds of the invention can be isolated and purified according to standard techniques well known to the person skilled in the art. One technique of particular usefulness in purifying the compounds is preparative liquid chromatography using mass spectrometry as a means of detecting the purified compounds emerging from the chromatography column.

Preparative LC-MS is a standard and effective method used for the purification of small organic molecules such as the compounds described herein. The methods for the liquid chromatography (LC) and mass spectrometry (MS) can be varied to provide better separation of the crude materials and improved detection of the samples by MS. Optimisation of the preparative gradient LC method will involve varying columns, volatile eluents and modifiers, and gradients. Methods are well known in the art for optimising preparative LC-MS methods and then using them to purify compounds. Such methods are described in Rosentreter U, Huber U.; Optimal fraction collecting in preparative LC/MS; J Comb Chem.; 2004; 6(2), 159-64 and Leister W, Strauss K, Wisnoski D, Zhao Z, Lindsley C., Development of a custom high-throughput preparative liquid chromatography/mass spectrometer platform for the preparative purification and analytical analysis of compound libraries; J Comb Chem.; 2003; 5(3); 322-9.

### Chemical Intermediates

Many of the chemical intermediates described above are novel per se and such novel intermediates form a further aspect of the invention.

Examples of such intermediates include, but are not limited to, protected forms of compounds of the formula (I) and sub-groups thereof, such as protected forms of compounds of the formulae (I′), (XXXI), (XXXVII), and (XLVI), as well as compounds of the formulae (XLIV) and (XLV) and protected forms thereof.

### Pharmaceutical Formulations

While it is possible for the active compound to be administered alone, it is preferable to present it as a pharmaceutical composition (e.g. formulation) comprising at least one active compound of the invention together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilizers, or other materials, as described herein.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Pharmaceutical compositions containing compounds of the formula (I) can be formulated in accordance with known techniques, see for example, Remington's Pharmaceutical Sciences, Mack Publishing Company, Easton, Pa., USA.

Accordingly, in a further aspect, the invention provides compounds of the formula (I) and sub-groups thereof as defined herein in the form of pharmaceutical compositions.

The pharmaceutical compositions can be in any form suitable for oral, parenteral, topical, intranasal, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery. The delivery can be by bolus injection, short term infusion or longer term infusion and can be via passive delivery or through the utilisation of a suitable infusion pump.

Pharmaceutical formulations adapted for parenteral administration include aqueous and non-aqueous sterile injection solutions which may contain anti-oxidants, buffers, bacteriostats, co-solvents, organic solvent mixtures, cyclodextrin complexation agents, emulsifying agents (for forming and stabilizing emulsion formulations), liposome components for forming liposomes, gellable polymers for forming polymeric gels, lyophilisation protectants and combinations of agents for, inter alia, stabilising the active ingredient in a soluble form and rendering the formulation isotonic with the blood of the intended recipient. Pharmaceutical formulations for parenteral administration may also take the form of aqueous and non-aqueous sterile suspensions which may include suspending agents and thickening agents (R. G. Strickly, Solubilizing Excipients in oral and injectable formulations, Pharmaceutical Research, Vol 21(2) 2004, p 201-230).

Liposomes are closed spherical vesicles composed of outer lipid bilayer membranes and an inner aqueous core and with an overall diameter of <100 μm. Depending on the level of hydrophobicity, moderately hydrophobic drugs can be solubilized by liposomes if the drug becomes encapsulated or intercalated within the liposome. Hydrophobic drugs can also be solubilized by liposomes if the drug molecule becomes an integral part of the lipid bilayer membrane, and in this case, the hydrophobic drug is dissolved in the lipid portion of the lipid bilayer.

The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use.

The pharmaceutical formulation can be prepared by lyophilising a compound of formula (I), or sub-groups thereof. Lyophilisation refers to the procedure of freeze-drying a composition. Freeze-drying and lyophilisation are therefore used herein as synonyms.

Extemporaneous injection solutions and suspensions may be prepared from sterile powders, granules and tablets.

Pharmaceutical compositions of the present invention for parenteral injection can also comprise pharmaceutically acceptable sterile aqueous or non-aqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

The compositions of the present invention may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

In one preferred embodiment of the invention, the pharmaceutical composition is in a form suitable for i.v. administration, for example by injection or infusion. For intravenous administration, the solution can be dosed as is, or can be injected into an infusion bag (containing a pharmaceutically acceptable excipient, such as 0.9% saline or 5% dextrose), before administration.

In another preferred embodiment, the pharmaceutical composition is in a form suitable for sub-cutaneous (s.c.) administration.

Pharmaceutical dosage forms suitable for oral administration include tablets, capsules, caplets, pills, lozenges, syrups, solutions, powders, granules, elixirs and suspensions, sublingual tablets, wafers or patches and buccal patches.

Thus, tablet compositions can contain a unit dosage of active compound together with an inert diluent or carrier such as a sugar or sugar alcohol, e.g.; lactose, sucrose, sorbitol or mannitol; and/or a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (e.g.; tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. As a further alternative, the active compound can be formulated in a delivery system that provides osmotic control of the release of the compound. Osmotic release and other delayed release or sustained release formulations may be prepared in accordance with methods well known to those skilled in the art.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragées, tablets or capsules.

Pharmaceutical compositions for oral administration can be obtained by combining the active ingredient with solid carriers, if desired granulating a resulting mixture, and processing the mixture, if desired or necessary, after the addition of appropriate excipients, into tablets, dragee cores or capsules. It is also possible for them to be incorporated into plastics carriers that allow the active ingredients to diffuse or be released in measured amounts.

The compounds of the invention can also be formulated as solid dispersions. Solid dispersions are homogeneous extremely fine disperse phases of two or more solids. Solid solutions (molecularly disperse systems), one type of solid dispersion, are well known for use in pharmaceutical technology (see (Chiou and Riegelman, J. Pharm. Sci., 60, 1281-1300 (1971)) and are useful in increasing dissolution rates and increasing the bioavailability of poorly water-soluble drugs.

This invention also provides solid dosage forms comprising the solid solution described above. Solid dosage forms include tablets, capsules and chewable tablets. Known excipients can be blended with the solid solution to provide the desired dosage form. For example, a capsule can contain the solid solution blended with (a) a disintegrant and a lubricant, or (b) a disintegrant, a lubricant and a surfactant. A tablet can contain the solid solution blended with at least one disintegrant, a lubricant, a surfactant, and a glidant. The chewable tablet can contain the solid solution blended with a bulking agent, a lubricant, and if desired an additional sweetening agent (such as an artificial sweetener), and suitable flavours.

The pharmaceutical formulations may be presented to a patient in “patient packs” containing an entire course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

Compositions for topical use include ointments, creams, sprays, patches, gels, liquid drops and inserts (for example intraocular inserts). Such compositions can be formulated in accordance with known methods.

Examples of formulations for rectal or intra-vaginal administration include pessaries and suppositories which may be, for example, formed from a shaped moldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compounds of the formula (I) will generally be presented in unit dosage form and, as such, will typically contain sufficient compound to provide a desired level of biological activity. For example, a formulation may contain from 1 nanogram to 2 grams of active ingredient, e.g. from 1 nanogram to 2 milligrams of active ingredient. Within this range, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 miligrams to 1 gram, of active compound.

The active compound will be administered to a patient in need thereof (for example a human or animal patient) in an amount sufficient to achieve the desired therapeutic effect.

### Protein Kinase Inhibitory Activity

The activity of the compounds of the invention as inhibitors of protein kinase A and protein kinase B can be measured using the assays set forth in the examples below and the level of activity exhibited by a given compound can be defined in terms of the IC50 value. Preferred compounds of the present invention are compounds having an IC₅₀ value of less than 1 μM, more preferably less than 0.1 μM, against protein kinase B.

Some of the compounds of the formula (I) are selective inhibitors of PKB relative to PKA, i.e. the IC₅₀ values against PKB are from 5 to 10 times lower, and more preferably greater than 10 times lower, than the IC₅₀ values against PKA.

### Therapeutic Uses

### Prevention or Treatment of Proliferative Disorders

The compounds of the formula (I) are inhibitors of protein kinase A and protein kinase B. As such, they are expected to be useful in providing a means of preventing the growth of or inducing apoptosis of neoplasias. It is therefore anticipated that the compounds will prove useful in treating or preventing proliferative disorders such as cancers. In particular tumours with deletions or inactivating mutations in PTEN or loss of PTEN expression or rearrangements in the (T-cell lymphocyte) TCL-1 gene may be particularly sensitive to PKB inhibitors. Tumours which have other abnormalities leading to an upregulated PKB pathway signal may also be particularly sensitive to inhibitors of PKB. Examples of such abnormalities include but are not limited to overexpression of one or more PI3K subunits, over-expression of one or more PKB iso forms, or mutations in PI3K, PDK1, or PKB which lead to an increase in the basal activity of the enzyme in question, or upregulation or overexpression or mutational activation of a growth factor receptor such as a growth factor selected from the epidermal growth factor receptor (EGFR), fibroblast growth factor receptor (FGFR), platelet derived growth factor receptor (PDGFR), insulin-like growth factor 1 receptor (IGF-1R) and vascular endothelial growth factor receptor (VEGFR) families.

It is also envisaged that the compounds of the invention will be useful in treating other conditions which result from disorders in proliferation or survival such as viral infections, and neurodegenerative diseases for example. PKB plays an important role in maintaining the survival of immune cells during an immune response and therefore PKB inhibitors could be particularly beneficial in immune disorders including autoimmune conditions.

Therefore, PKB inhibitors could be useful in the treatment of diseases in which there is a disorder of proliferation, apoptosis or differentiation.

PKB inhibitors may also be useful in diseases resulting from insulin resistance and insensitivity, and the disruption of glucose, energy and fat storage such as metabolic disease and obesity.

Examples of cancers which may be inhibited include, but are not limited to, a carcinoma, for example a carcinoma of the bladder, breast, colon (e.g. colorectal carcinomas such as colon adenocarcinoma and colon adenoma), kidney, epidermal, liver, lung, for example adenocarcinoma, small cell lung cancer and non-small cell lung carcinomas, oesophagus, gall bladder, ovary, pancreas e.g. exocrine pancreatic carcinoma, stomach, cervix, endometrium, thyroid, prostate, or skin, for example squamous cell carcinoma; a hematopoetic malignancy for example acute myeloid leukaemia, acute promyelocytic leukaemia, acute lymphoblastic leukaemia, chronic myeloid leukaemia, chronic lymphocytic leukaemia and other B-cell lymphoproliferative diseases, myelodysplastic syndrome, T-cell lymphoproliferative diseases including those derived from Natural Killer cells, Non-Hodgkin's lymphoma and Hodgkin's disease; Bortezomib sensitive and refractory multiple myeloma; hematopoetic diseases of abnormal cell proliferation whether pre malignant or stable such as myeloproliferative diseases including polycythemia vera, essential thrombocythemia and primary myelofibrosis; hairy cell lymphoma, or Burkett's lymphoma; a hematopoietic tumour of myeloid lineage, for example acute and chronic myelogenous leukaemias, myelodysplastic syndrome, or promyelocytic leukaemia; thyroid follicular cancer; a tumour of mesenchymal origin, for example fibrosarcoma or habdomyosarcoma; a tumour of the central or peripheral nervous system, for example astrocytoma, neuroblastoma, glioma or schwannoma; melanoma; seminoma; teratocarcinoma; osteosarcoma; xenoderoma pigmentosum; keratoctanthoma; thyroid follicular cancer; or Kaposi's sarcoma.

One subset of cancers which may be inhibited include, but are not limited to, a carcinoma, for example a carcinoma of the bladder, breast, colon (e.g. colorectal carcinomas such as colon adenocarcinoma and colon adenoma), kidney, epidermal, liver, lung, for example adenocarcinoma, small cell lung cancer and non-small cell lung carcinomas, oesophagus, gall bladder, ovary, pancreas e.g. exocrine pancreatic carcinoma, stomach, cervix, endometrium, thyroid, prostate, or skin, for example squamous cell carcinoma; a hematopoietic tumour of lymphoid lineage, for example leukaemia, acute lymphocytic leukaemia, B-cell lymphoma, T-cell lymphoma, Hodgkin's lymphoma, non-Hodgkin's lymphoma, hairy cell lymphoma, or Burkett's lymphoma; a hematopoietic tumour of myeloid lineage, for example acute and chronic myelogenous leukaemias, myelodysplastic syndrome, or promyelocytic leukaemia; thyroid follicular cancer; a tumour of mesenchymal origin, for example fibrosarcoma or habdomyosarcoma; a tumour of the central or peripheral nervous system, for example astrocytoma, neuroblastoma, glioma or schwannoma; melanoma; seminoma; teratocarcinoma; osteosarcoma; xenoderoma pigmentosum; keratoctanthoma; thyroid follicular cancer; or Kaposi's sarcoma.

Thus, in the pharmaceutical compositions, uses or methods of this invention for treating a disease or condition comprising abnormal cell growth, the disease or condition comprising abnormal cell growth in one embodiment is a cancer.

Particular subsets of cancers include breast cancer, ovarian cancer, colon cancer, prostate cancer, oesophageal cancer, squamous cancer and non-small cell lung carcinomas.

A further subset of cancers includes breast cancer, ovarian cancer, prostate cancer, endometrial cancer and glioma.

It is also possible that some protein kinase B inhibitors can be used in combination with other anticancer agents. For example, it may be beneficial to combine of an inhibitor that induces apoptosis with another agent which acts via a different mechanism to regulate cell growth thus treating two of the characteristic features of cancer development. Examples of such combinations are set out below.

### Immune Disorders

Immune disorders for which PKA and PKB inhibitors may be beneficial include but are not limited to autoimmune conditions and chronic inflammatory diseases, for example systemic lupus erythematosus, autoimmune mediated glomerulonephritis, rheumatoid arthritis, psoriasis, inflammatory bowel disease, and autoimmune diabetes mellitus, Eczema hypersensitivity reactions, asthma, COPD, rhinitis, and upper respiratory tract disease.

### Other Therapeutic Uses

PKB plays a role in apoptosis, proliferation, differentiation and therefore PKB inhibitors could also be useful in the treatment of the following diseases other than cancer and those associated with immune dysfunction; viral infections, for example herpes virus, pox virus, Epstein-Barr virus, Sindbis virus, adenovirus, HIV, HPV, HCV and HCMV; prevention of AIDS development in HIV-infected individuals; cardiovascular diseases for example cardiac hypertrophy, restenosis, atherosclerosis; neurodegenerative disorders, for example Alzheimer's disease, AIDS-related dementia, Parkinson's disease, amyotropic lateral sclerosis, retinitis pigmentosa, spinal muscular atropy and cerebellar degeneration; glomerulonephritis; myelodysplastic syndromes, ischemic injury associated myocardial infarctions, stroke and reperfusion injury, degenerative diseases of the musculoskeletal system, for example, osteoporosis and arthritis, aspirin-sensitive rhinosinusitis, cystic fibrosis, multiple sclerosis, kidney diseases.

### Advantages of Compounds of the Invention

Compounds of the formula (I) and sub-groups thereof as defined herein will have advantages over prior art compounds.

In particular, the compounds of formulae (II), (II), (III) and (IV) have advantages over prior art compounds.

Potentially the compounds of the invention have physiochemical properties suitable for oral exposure.

Compounds of the formula (I) should exhibit improved oral bioavailability relative to prior art compounds. Oral bioavailability can be defined as the ratio (F) of the plasma exposure of a compound when dosed by the oral route to the plasma exposure of the compound when dosed by the intravenous (i.v.) route, expressed as a percentage.

Compounds having an oral bioavailability (F value) of greater than 30%, more preferably greater than 40%, are particularly advantageous in that they may be administered orally rather than, or as well as, by parenteral administration.

Furthermore, compounds of the invention are both more potent and more selective in their activities against different kinases, and demonstrate enhanced selectivity for and potency against PKB in particular.

Compounds of the invention are advantageous over prior art compounds in that they have different susceptibilities to P450 enzymes and in that they exhibit improvements with regard to drug metabolism and pharmacokinetic properties.

Furthermore compounds of the invention should exhibit reduced dosage requirements.

Compounds of the invention are advantageous in that they have improved thermodynamic solubilities, thereby leading potentially to an improved dose:solubility ratio and reduced development risk.

Compounds of the invention also demonstrate improved cell activity in proliferation and clonogenic assays thereby indicating improved anti-cancer activity.

Compounds of the invention are potentially less toxic than prior art compounds.

hERG

In the late 1990s a number of drugs, approved by the US FDA, had to be withdrawn from sale in the US when it was discovered they were implicated in deaths caused by heart malfunction. It was subsequently found that a side effect of these drugs was the development of arrhythmias caused by the blocking of hERG channels in heart cells. The hERG channel is one of a family of potassium ion channels the first member of which was identified in the late 1980s in a mutant Drosophila melanogaster fruitfly (see Jan, L. Y. and Jan, Y. N. (1990). A Superfamily of Ion Channels. Nature, 345(6277):672). The biophysical properties of the hERG potassium ion channel are described in Sanguinetti, M. C., Jiang, C., Curran, M. E., and Keating, M. T. (1995). A Mechanistic Link Between an Inherited and an Acquired Cardiac Arrhythmia: HERG encodes the Ikr potassium channel. Cell, 81:299-307, and Trudeau, M. C., Warmke, J. W., Ganetzky, B., and Robertson, G. A. (1995). HERG, a Human Inward Rectifier in the Voltage-Gated Potassium Channel Family. Science, 269:92-95.

The elimination of hERG blocking activity remains an important consideration in the development of any new drug.

Compounds of formula (I) have reduced, negligible or no hERG ion channel blocking activity.

### Methods of Treatment

It is envisaged that the compounds of the formula (I) and sub-groups thereof as defined herein will be useful in the prophylaxis or treatment of a range of disease states or conditions mediated by protein kinase A and/or protein kinase B. Examples of such disease states and conditions are set out above.

The compounds are generally administered to a subject in need of such administration, for example a human or animal patient, preferably a human.

The compounds will typically be administered in amounts that are therapeutically or prophylactically useful and which generally are non-toxic. However, in certain situations (for example in the case of life threatening diseases), the benefits of administering a compound of the formula (I) may outweigh the disadvantages of any toxic effects or side effects, in which case it may be considered desirable to administer compounds in amounts that are associated with a degree of toxicity.

The compounds may be administered over a prolonged term to maintain beneficial therapeutic effects or may be administered for a short period only. Alternatively they may be administered in a pulsatile or continuous manner.

A typical daily dose of the compound of formula (I) can be in the range from 100 picograms to 100 milligrams per kilogram of body weight, more typically 5 nanograms to 25 milligrams per kilogram of bodyweight, and more usually 10 nanograms to 15 milligrams per kilogram (e.g. 10 nanograms to 10 milligrams, and more typically 1 microgram per kilogram to 20 milligrams per kilogram, for example 1 microgram to 10 milligrams per kilogram) per kilogram of bodyweight although higher or lower doses may be administered where required. The compound of the formula (I) can be administered on a daily basis or on a repeat basis every 2, or 3, or 4, or 5, or 6, or 7, or 10 or 14, or 21, or 28 days for example.

The compounds of the invention may be administered orally in a range of doses, for example 1 to 1500 mg, 2 to 800 mg, or 5 to 500 mg, e.g. 2 to 200 mg or 10 to 1000 mg, particular examples of doses including 10, 20, 50 and 80 mg. The compound may be administered once or more than once each day. The compound can be administered continuously (i.e. taken every day without a break for the duration of the treatment regimen). Alternatively, the compound can be administered intermittently, i.e. taken continuously for a given period such as a week, then discontinued for a period such as a week and then taken continuously for another period such as a week and so on throughout the duration of the treatment regimen. Examples of treatment regimens involving intermittent administration include regimens wherein administration is in cycles of one week on, one week off; or two weeks on, one week off; or three weeks on, one week off; or two weeks on, two weeks off; or four weeks on two weeks off; or one week on three weeks off—for one or more cycles, e.g. 2, 3, 4, 5, 6, 7, 8, 9 or 10 or more cycles.

In one particular dosing schedule, a patient will be given an infusion of a compound of the formula (I) for periods of one hour daily for up to ten days in particular up to five days for one week, and the treatment repeated at a desired interval such as two to four weeks, in particular every three weeks.

More particularly, a patient may be given an infusion of a compound of the formula (I) for periods of one hour daily for 5 days and the treatment repeated every three weeks.

In another particular dosing schedule, a patient is given an infusion over 30 minutes to 1 hour followed by maintenance infusions of variable duration, for example 1 to 5 hours, e.g. 3 hours.

In a further particular dosing schedule, a patient is given a continuous infusion for a period of 12 hours to 5 days, an in particular a continuous infusion of 24 hours to 72 hours.

Ultimately, however, the quantity of compound administered and the type of composition used will be commensurate with the nature of the disease or physiological condition being treated and will be at the discretion of the physician.

The compounds as defined herein can be administered as the sole therapeutic agent or they can be administered in combination therapy with one of more other compounds for treatment of a particular disease state, for example a neoplastic disease such as a cancer as hereinbefore defined. In one embodiment, examples of other therapeutic agents or treatments that may be administered together (whether concurrently or at different time intervals) with the compounds of the formula (I) include but are not limited to:
Topoisomerase I inhibitors
Antimetabolites
Tubulin targeting agents
DNA binder and topoisomerase II inhibitors
Alkylating Agents
Monoclonal Antibodies.
Anti-Hormones
Signal Transduction Inhibitors
Proteasome Inhibitors
DNA methyl transferases
Cytokines and retinoids
Chromatin targeted therapies
Radiotherapy, and,
Other therapeutic or prophylactic agents; for example agents that reduce or alleviate some of the side effects associated with chemotherapy. Particular examples of such agents include anti-emetic agents and agents that prevent or decrease the duration of chemotherapy-associated neutropenia and prevent complications that arise from reduced levels of red blood cells or white blood cells, for example erythropoietin (EPO), granulocyte macrophage-colony stimulating factor (GM-CSF), and granulocyte-colony stimulating factor (G-CSF). Also included are agents that inhibit bone resorption such as bisphosphonate agents e.g. zoledronate, pamidronate and ibandronate, agents that suppress inflammatory responses (such as dexamethazone, prednisone, and prednisolone) and agents used to reduce blood levels of growth hormone and IGF-I in acromegaly patients such as synthetic forms of the brain hormone somatostatin, which includes octreotide acetate which is a long-acting octapeptide with pharmacologic properties mimicking those of the natural hormone somatostatin. Further included are agents such as leucovorin, which is used as an antidote to drugs that decrease levels of folic acid, or folinic acid it self and agents such as megestrol acetate which can be used for the treatment of side-effects including oedema and thromoembolic episodes.

Thus, as described above, the anti-cancer treatment defined hereinbefore may be applied as a sole therapy or may involve, in addition to the compound of the invention, radiotherapy or chemotherapy. The anti-cancer treatment may also involve conventional surgery.

In another embodiment, the chemotherapy may include one or more of the following categories of anti-tumour agents:—

(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661) and bosutinib (SKI-606), and metalloproteinase inhibitors like marimastat, inhibitors of urokinase plasminogen activator receptor function or antibodies to Heparanase];
(iv) inhibitors of growth factor function and cell signalling: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (R115777) and lonafarnib (SCH66336)), inhibitors of cell signalling through MEK (for example AZD6244) and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), compounds such as those disclosed in International Patent Applications WO97/22596, WO 97/30035, WO 97/32856 and WO 98/13354 and compounds that work by other mechanisms (for example linomide, inhibitors of integrin ανβ3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) an endothelin receptor antagonist, for example zibotentan (ZD4054) or atrasentan;
(viii) antisense therapies, for example those which are directed to the targets listed above, such as ISIS 2503, an anti-ras antisense;
(ix) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(x) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Each of the compounds present in the combinations of the invention may be given in individually varying dose schedules and via different routes.

Where the compound of the formula (I) is administered in combination therapy with one, two, three, four or more other therapeutic agents (preferably one or two, more preferably one), the compounds can be administered simultaneously or sequentially. When administered sequentially, they can be administered at closely spaced intervals (for example over a period of 5-10 minutes) or at longer intervals (for example 1, 2, 3, 4 or more hours apart, or even longer periods apart where required), the precise dosage regimen being commensurate with the properties of the therapeutic agent(s).

The compounds of the invention may also be administered in conjunction with non-chemotherapeutic treatments such as radiotherapy, photodynamic therapy, gene therapy; surgery and controlled diets.

For use in combination therapy with another chemotherapeutic agent, the compound of the formula (I) and one, two, three, four or more other therapeutic agents can be, for example, formulated together in a dosage form containing two, three, four or more therapeutic agents. In an alternative, the individual therapeutic agents may be formulated separately and presented together in the form of a kit, optionally with instructions for their use.

A person skilled in the art would know through his or her common general knowledge the dosing regimes and combination therapies to use.

### Methods of Diagnosis

Prior to administration of a compound of the formula (I), a patient may be screened to determine whether a disease or condition from which the patient is or may be suffering is one which would be susceptible to treatment with a compound having activity against protein kinase A and/or protein kinase B.

For example, a biological sample taken from a patient may be analysed to determine whether a condition or disease, such as cancer, that the patient is or may be suffering from is one which is characterised by a genetic abnormality or abnormal protein expression which leads to up-regulation of PKA and/or PKB or to sensitisation of a pathway to normal PKA and/or PKB activity, or to upregulation of a signal transduction component upstream of PKA and/or PKB such as, in the case of PKB, P13K, GF receptor and PDK 1 & 2.

Alternatively, a biological sample taken from a patient may be analysed for loss of a negative regulator or suppressor of the PKB pathway such as PTEN. In the present context, the term “loss” embraces the deletion of a gene encoding the regulator or suppressor, the truncation of the gene (for example by mutation), the truncation of the transcribed product of the gene, or the inactivation of the transcribed product (e.g. by point mutation) or sequestration by another gene product.

The term up-regulation includes elevated expression or over-expression, including gene amplification (i.e. multiple gene copies) and increased expression by a transcriptional effect, and hyperactivity and activation, including activation by mutations. Thus, the patient may be subjected to a diagnostic test to detect a marker characteristic of up-regulation of PKA and/or PKB. The term diagnosis includes screening. By marker we include genetic markers including, for example, the measurement of DNA composition to identify mutations of PKA and/or PKB. The term marker also includes markers which are characteristic of up regulation of PKA and/or PKB and/or other factors which lead to an upregulation of the relevant pathways, including enzyme activity, enzyme levels, enzyme state (e.g. phosphorylated or not) and mRNA levels of the aforementioned proteins.

The above diagnostic tests and screens are typically conducted on a biological sample selected from tumour biopsy samples, blood samples (isolation and enrichment of shed tumour cells), stool biopsies, sputum, chromosome analysis, pleural fluid, peritoneal fluid, bone marrow or urine.

Identification of an individual carrying a mutation in PKA and/or PKB or a rearrangement of TCL-1 or loss of PTEN expression may mean that the patient would be particularly suitable for treatment with a PKA and/or PKB inhibitor. Tumours may preferentially be screened for presence of a PKA and/or PKB variant prior to treatment. The screening process will typically involve direct sequencing, oligonucleotide microarray analysis, or a mutant specific antibody.

Methods of identification and analysis of mutations and up-regulation of proteins are known to a person skilled in the art. Screening methods could include, but are not limited to, standard methods such as reverse-transcriptase polymerase chain reaction (RT-PCR) or in-situ hybridisation.

In screening by RT-PCR, the level of mRNA in the tumour is assessed by creating a cDNA copy of the mRNA followed by amplification of the cDNA by PCR. Methods of PCR amplification, the selection of primers, and conditions for amplification, are known to a person skilled in the art. Nucleic acid manipulations and PCR are carried out by standard methods, as described for example in Ausubel, F. M. et al., eds. Current Protocols in Molecular Biology, 2004, John Wiley & Sons Inc., or Innis, M. A. et-al., eds. PCR Protocols: a guide to methods and applications, 1990, Academic Press, San Diego. Reactions and manipulations involving nucleic acid techniques are also described in Sambrook et al., 2001, 3^{rd} Ed, Molecular Cloning: A Laboratory Manual, Cold Spring Harbor Laboratory Press. Alternatively a commercially available kit for RT-PCR (for example Roche Molecular Biochemicals) may be used, or methodology as set forth in U.S. Pat. Nos. 4,666,828; 4,683,202; 4,801,531; 5,192,659, 5,272,057, 5,882,864, and 6,218,529 and incorporated herein by reference.

An example of an in-situ hybridisation technique for assessing mRNA expression would be fluorescence in-situ hybridisation (FISH) (see Angerer, 1987 Meth. Enzymol., 152: 649).

Generally, in situ hybridization comprises the following major steps: (1) fixation of tissue to be analyzed; (2) prehybridization treatment of the sample to increase accessibility of target nucleic acid, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions. Standard methods for carrying out FISH are described in Ausubel, F. M. et al., eds. Current Protocols in Molecular Biology, 2004, John Wiley & Sons Inc and Fluorescence In Situ Hybridization: Technical Overview by John M. S. Bartlett in Molecular Diagnosis of Cancer, Methods and Protocols, 2nd ed.; ISBN: 1-59259-760-2; March 2004, pps. 077-088; Series: Methods in Molecular Medicine.

Alternatively, the protein products expressed from the mRNAs may be assayed by immunohistochemistry of tumour samples, solid phase immunoassay with microtitre plates, Western blotting, 2-dimensional SDS-polyacrylamide gel electrophoresis, ELISA, flow cytometry and other methods known in the art for detection of specific proteins. Detection methods would include the use of site specific antibodies. The skilled person will recognize that all such well-known techniques for detection of upregulation of PKB, or detection of PKB variants could be applicable in the present case.

Therefore all of these techniques could also be used to identify tumours particularly suitable for treatment with PKA and/or PKB inhibitors.

For example, as stated above, PKB beta has been found to be upregulated in 10-40% of ovarian and pancreatic cancers (Bellacosa et al 1995, Int. J. Cancer 64, 280-285; Cheng et al 1996, PNAS 93, 3636-3641; Yuan et al 2000, Oncogene 19, 2324-2330). Therefore it is envisaged that PKB inhibitors, and in particular inhibitors of PKB beta, may be used to treat ovarian and pancreatic cancers.

PKB alpha is amplified in human gastric, prostate and breast cancer (Staal 1987, PNAS 84, 5034-5037; Sun et al 2001, Am. J. Pathol. 159, 431-437). Therefore it is envisaged that PKB inhibitors, and in particular inhibitors of PKB alpha, may be used to treat human gastric, prostate and breast cancer.

Increased PKB gamma activity has been observed in steroid independent breast and prostate cell lines (Nakatani et al 1999, J. Biol. Chem. 274, 21528-21532). Therefore it is envisaged that PKB inhibitors, and in particular inhibitors of PKB gamma, may be used to treat steroid independent breast and prostate cancers.

### EXPERIMENTAL

The invention will now be illustrated, but not limited, by reference to the specific embodiments described in the following procedures and examples.

In the examples, the following abbreviations may be used.
AcOH acetic acid
BOC/Boc tert-butyloxycarbonyl
BuOH butanol
DIPEA N,N′-diisopropylethylamine
DMA dimethylacetamide
DMAW90 Solvent mixture: DCM: MeOH, AcOH, H₂O (90:18:3:2)
DMAW120 Solvent mixture: DCM: MeOH, AcOH, H₂O (120:18:3:2)
DMAW240 Solvent mixture: DCM: MeOH, AcOH, H₂O (240:20:3:2)
DCM dichloromethane
DMF dimethylformamide
DMSO dimethyl sulphoxide
EDC 1-ethyl-3-(3′-dimethylaminopropyl)-carbodiimide
Et₃N triethylamine
EtOAc ethyl acetate
Et₂O diethyl ether
FMOC/Fmoc fluorenylmethoxycarbonyl
FMOC-PIP(FMOC)OH 1-Fmoc-4-(Fmoc-amino)-piperidine-4-carboxylic acid
h hour(s)
HATU O-(7-azabenzotriazol-1-yl)-N,N,N′,N′-tetramethyluronium hexafluorophosphate
HOAt 1-hydroxyazabenzotriazole
HOBt 1-hydroxybenzotriazole
IPA isopropanol
MeCN acetonitrile
MeOH methanol
min. minutes
Ms mesyl
MsO mesylate
PG protecting group
r.t. room temperature
SiO₂ silica
TBTU N,N,N′,N′-tetramethyl-O-(benzotriazol-1-yl)uronium tetrafluoroborate
TFA trifluoroacetic acid
THF tetrahydrofuran

The starting materials for each of the procedures described below are commercially available unless otherwise specified.

Proton magnetic resonance ('H NMR) spectra were recorded on a Bruker AV400 instrument operating at 400.13 MHz, in Me-d₃-OD at 27° C., unless otherwise stated and are reported as follows: chemical shift 6/ppm (number of protons, multiplicity where s=singlet, d=doublet, t=triplet, q=quartet, m=multiplet, br=broad). The residual protic solvent MeOH (δ_{H}=3.31 ppm) was used as the internal reference.

In the examples, the compounds prepared were characterised by liquid chromatography and mass spectroscopy using the systems and operating conditions set out below. Where chlorine is present, the mass quoted for the compound is for ³⁵Cl. The operating conditions used are described below.

The chemical structure drawings in the Examples below were prepared using ISIS Draw.

In some cases, the hydrogen atoms are not shown, e.g. as in the structure:

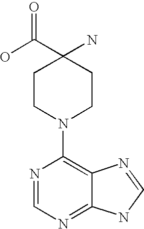

where the hydrogen atoms on the carboxylic acid group, amino group and purine 9H-position are not shown.

### LCT System 1

HPLC System: Waters Alliance 2795 Separations Module

Mass Spec Detector: Waters/Micromass LCT

UV Detector: Waters 2487 Dual λ Absorbance Detector

### Polar Analytical Conditions:

| Eluent A: | Methanol | | |
|---|---|---|---|
| Eluent B: | 0.1% Formic Acid in Water | | |
| Gradient: | Time (mins) | A | B |
| | 0 | 10 | 90 |
| | 0.5 | 10 | 90 |
| | 6.5 | 90 | 10 |
| | 10 | 90 | 10 |
| | 10.5 | 10 | 90 |
| | 15 | 10 | 90 |

| | |
|---|---|
| Flow: | 1.0 ml/min |
| Column: | Supelco DISCOVERY C₁₈ 5 cm × 4.6 mm i.d., 5 μm |

### MS Conditions:

Capillary voltage: 3500v (+ve ESI), 3000v (−ve ESI)

Cone voltage: 40v (+ve ESI), 50v (−ve ESI)

Source Temperature: 100° C.

Scan Range: 50-1000 amu

Ionisation Mode: +ve/−ve electrospray ESI (Lockspray™)

### LCT System 2

HPLC System: Waters Alliance 2795 Separations Module

Mass Spec Detector: Waters/Micromass LCT

UV Detector: Waters 2487 Dual λ Absorbance Detector

### Analytical Conditions:

| Eluent A: | Methanol | | |
|---|---|---|---|
| Eluent B: | 0.1% Formic Acid in Water | | |
| Gradient: | Time (mins) | A | B |
| | 0 | 10 | 90 |
| | 0.6 | 10 | 90 |
| | 1.0 | 20 | 80 |
| | 7.5 | 90 | 10 |
| | 9 | 90 | 10 |
| | 9.5 | 10 | 90 |
| | 10 | 10 | 90 |

| | |
|---|---|
| Flow: | 1 ml/min |
| Column: | Supelco DISCOVERY C₁₈ 5 cm × 4.6 mm i.d., 5 μm |

MS conditions:

Capillary voltage: 3500v (+ve ESI), 3000v (−ve ESI)

Cone voltage: 40v (+ve ESI), 50v (−ve ESI)

Source Temperature: 100° C.

Scan Range: 50-1000 amu

Ionisation Mode: +ve/−ve electrospray ESI (Lockspray™)

### LCT System 3

HPLC system: Waters alliance 2795 Separations Module

Mass Spec Detector: Waters/Micromass LCT

UV Detector: Waters 2478 Dual γ Absorbance Detector

### Analytical Conditions

| Eluent A: | Methanol | | |
|---|---|---|---|
| Eluent B: | 0.1% Formic Acid in Water | | |
| Gradient | Time (mins) | A | B |
| | 0 | 10 | 90 |
| | 0.3 | 10 | 90 |
| | 0.6 | 20 | 80 |
| | 4.5 | 90 | 10 |
| | 5.4 | 90 | 10 |
| | 5.7 | 10 | 90 |
| | 6.0 | 10 | 90 |

| | |
|---|---|
| Flow: | 1 mL/min |
| Column: | Supelco DISCOVERY C₁₈ 3 cm × 4.6 mm i.d., 3 m |

(MS conditions as before)

In the examples below, the following key is used to identify the LCMS conditions

LCT1 LCT System 1—polar analytical conditions

LCT2 LCT System 2—polar analytical conditions

LCT3 LCT System 3—polar analytical conditions

### General Methods

### Method A

### Boc Protection

To a solution of protected amine in a suitable organic solvent (e.g. dichloromethane, DMF, THF) was added a base (e.g. triethylamine, aqueous sodium hydroxide or aqueous sodium bicarbonate, 1 to excess equivalents) and di-tent-butyl dicarbonate (1 to excess equivalents). This mixture was stirred at room temperature for 30 minutes to 18 hours before aqueous workup. The crude product was optionally purified by silica column chromatography eluting with ethyl acetate/petroleum ether to furnish the desired compound.

### Method B

### Boronate Ester Formation

A mixture of a protected aryl halide (preferably an iodide or bromide, 1 equivalent), bis(pinacolato)diboron (1 equivalent), potassium acetate (3 equivalents) and [1,1′-bis(diphenylphosphino)ferrocene]dichloro palladium(II) (0.05 equivalents) in dimethylsulfoxide was heated to 80 deg C. under nitrogen for 2-18 hours. The reaction was then allowed to cool, diluted with ethyl acetate then filtered under suction. The resultant crude material was purified by tituration or silica column chromatography (typically with mixture of ethyl acetate/petrol) to furnish the desired compounds as solids.

### Method C1

Suzuki Coupling—with Microwave Irradiation

A mixture of aryl chloride, bromide or iodide (1 equivalent), inorganic base (typically potassium carbonate or potassium phosphate, 2-6 equivalents), catalyst (bis(tri-t-butylphosphine)palladium (0) for coupling of aryl chlorides; tetrakis(triphenylphosphine)palladium (0) for coupling of aryl bromides or iodides) and 4-(4,4,5,5-tetramethyl-1,3,2-dioxaborolan-2-yl)-1H-pyrazole (1.1-1.5 equivalents) in ethanol/methanol/toluene/water (ca. equal proportions) was irradiated in a CEM Explorer™ microwave to 80-145° C. for 15-90 minutes using ≦100 watts power. The reaction was either concentrated in vacuo or directly partitioned between ethyl acetate and 2N NaOH or water. The aqueous layer was extracted with ethyl acetate and the combined organic layers were on occasions washed with brine, dried (MgSO₄) and concentrated under reduced pressure. In some cases the product precipitated out during work up, this was collected by filtration. If as this stage there was a significant amount of residual starting material, fresh reactants and reagents would be added and the reaction irradiated then worked up for a second time. The crude product was purified by column chromatography (SiO₂), eluting with a mixture of dichloromethane/methanol or dichloromethane/methanol/ammonia or dichloromethane/methanol/acetic acid/H₂O and/or via preparative HPLC to afford the desired compounds.

### Method C2

### Suzuki Coupling—Under Thermal Heating

Under this method, the Suzuki coupling exemplified in Method C1 was conducted as described in C1, but instead the reaction mixture was heated thermally from 50° C. to reflux for a period of 30 minutes to 16 hours.

### Method C3

### Suzuki Coupling—Microwave Irradiation II

A mixture of 6-chloro-7,9-dihydro-purin-8-one (Preparation A, 1-1.3 equivalent), inorganic base (typically potassium carbonate or potassium phosphate, 2-6 equivalents), catalyst (bis(tri-t-butylphosphine)palladium (0) and protected aryl halide (1 equivalents) in ethanol/methanol/toluene/water (ca. equal proportions) was irradiated in a CEM Explorer™ microwave to 80-145° C. for 15-30 minutes using ≦100 watts power. The reaction was either concentrated in vacuo or directly partitioned between ethyl acetate and 2N NaOH or water. The aqueous layer was extracted with ethyl acetate and the combined organic layers were on occasions washed with brine, dried (MgSO₄) and concentrated under reduced pressure. In some cases the product precipitated out during work up, this was collected by filtration. If as this stage there was a significant amount of residual starting material, fresh reactants and reagents would be added and the reaction irradiated then worked up for a second time. The crude product was purified by column chromatography (SiO₂), eluting with a mixture of dichloromethane/methanol or dichloromethane/methanol/ammonia or dichloromethane/methanol/acetic acid/H₂O or petrol/ethyl acetate and/or via preparative HPLC to afford the desired compounds.

### Method D

### Boc Deprotection

To the protected amine, optionally dissolved in a suitable organic solvent (typically dichloromethane), was added a strong organic (e.g. trifluoroacetic acid) or inorganic (e.g. hydrochloric acid in 1,4-dioxane) acid. This mixture was stirred at room temperature for between 10 minutes and 18 hours to furnish the crude amine as a salt. If necessary, purification could be achieved via silica column chromatography using a mixture of dichloromethane, methanol, acetic acid and H₂O or dichloromethane, methanol and ammonia, and/or via ion exchange chromatography and/or by preparative HPLC.

### Method E

### Acetonitrile Addition

To n-BuLi (2.5M in hexanes) (1.25 equivalents), in THF at −78° C., was added MeCN (1.25 equivalents). The mixture was stirred for 30 min at −78° C. followed by addition of a solution of the requisite benzophenone (1.0 equivalent) in THF. The mixture was then allowed to warm to r.t. over 30 min. after which saturated aqueous NH₄Cl was added. The organic layer was separated, washed with brine, dried (Na₂SO₄) and then concentrated in vacuo to furnish the desired compound, which was used in the next step without further purification.

### Method F1

### Nitrile Reduction Using Lithium Aluminium Hydride—I

To LiAlH₄ (2.0 equivalents), in THF at −10° C., was added the nitrile (1.0 equivalent). The mixture was stirred at −10° C. for 30 min. then 0° C. for 30 min. and r.t for 1 hr. The mixture was then cooled to 0° C. and quenched by successive, careful, addition of H₂O (3 equivalents) and 10% aq. NaOH (2 equivalents). After stirring for a further 10 min. the mixture was diluted with THF and filtered. The filtrate was then concentrated in vacuo. and the residue was purified by flash column chromatography on silica gel, eluting with DMAW 90 to afford the desired compound.

### Method F2

### Nitrile Reduction Using Lithium Aluminium Hydride—II

To a solution of the nitrile in organic solvent (typically tetrahydrofuran) at room temperature was added a solution of lithium aluminium hydride in tetrahydrofuran (2 equivalents). The mixture was stirred at room temperature for 1-16 hours then quenched by cautious addition of small amounts of water and sodium hydroxide solution. The reaction was filtered under suction, washing with tetrahydrofuran and methanol then concentrated in vacuo furnishing a crude product that was purified on a silica Biotage column eluting with dichloromethane/methanol or dichloromethane/acetic acid/methanol/water mixtures.

### Method F3

### Nitrile Reduction Using Raney Nickel

A mixture of protected amine and Raney Nickel (typically used was as a suspension in water) in organic solvent (e.g. N,N-dimethylformamide, ethanol and/or tetrahydrofuran), optionally with added base (e.g. aqueous sodium hydroxide solution or methanolic ammonia), was hydrogenated at atmospheric pressure and at room temperature for 18-96 hours. To achieve full reduction, it was occasionally required to refresh the catalyst during this time. When the requisite volume of hydrogen had been consumed, the reaction was filtered under suction using either a celite pad or glass fibre filter paper before concentrating to furnish the desired deprotected amine. This material was ether used crude, or purified by silica column chromatography eluting with mixtures of dichloromethane, methanol, acetic acid and water.

### Method G

### Amide Coupling (EDC, HOBt Method)

To a stirred solution of the acid or sodium salt (1 equivalent) in DMF (10 ml) was added 1-hydroxybenzotriazole (1.2 equivalents), the amine (1-1.2 equivalents) and either diisopropylethylamine or triethylamine (1.2-2.2 eq) followed by N-ethyl-N′-(3-dimethylaminopropyl) carbodiimide hydrochloride (1.2 equivalents). The reaction mixture was either stirred at room temperature or heated at 50-60° C. overnight. The mixture was diluted with ethyl acetate and washed with excess water/aqueous saturated sodium bicarbonate solution, the organic layer was separated and the solvent removed in vacuo to afford the product. The product was either taken on crude or purified by column chromatography on silica (eluting with mixtures of ethyl acetate in petroleum ether).

### Method H1

### Removal of a Carboxybenzyl (Z) Protecting Group by Hydrogenation

A mixture of protected amine and Palladium on carbon (typically 10%, wet) in organic solvent (e.g. ethanol), was hydrogenated at atmospheric pressure and at room temperature for 18-96 hours. To achieve full reduction, it was occasionally required to refresh the catalyst during this time. When the requisite volume of hydrogen had been consumed, the reaction was filtered under suction using either a celite pad or glass fibre filter paper before concentrating to furnish the desired deprotected amine. This material was ether used crude, or purified by silica column chromatography eluting with mixtures of dichloromethane, methanol, acetic acid and water.

### Method H2

Removal of a Carboxybenzyl (Z) Protecting Group by Hydrogenation with In-Situ BOC Protection

The reaction was conducted as described in H1 above, with an excess of di-tent-butyl dicarbonate. Upon work-up, the BOC protected amine was isolated and was optionally purified on silica Biotage column eluting with ethyl acetate/petrol mixtures.

### Method H3

### Removal of a Carboxybenzyl (Z) Protecting Group Under Acidic Conditions

The protected amine was dissolved in hydrobromic acid in acetic acid (40%) and stirred thus for 1-16 hours. The acids were then removed in vacuo and the residue was optionally re-concentrated from methanol. The crude material was purified on a silica Biotage column eluting with mixtures of dichloromethane, methanol, acetic acid and water.

### Method I

### Alkylation of Amine

To a solution of amine or Z-protected amine in N,N-dimethylformamide cooled to 0° C. was added portionwise sodium hydride (1.5 equivalents). After stirring for 10 minutes, a solution of alkylamine (e.g. iodomethane in tert-butyldimethylether, 1-5 equivalents) was added and the mixture was allowed to warm to room temperature. The crude product was isolated by aqueous extraction and optionally purified on a silica Biotage column.

### Method J

### Nucleophilic Substitution of Halo-Bicyclic Compound by Piperidine Compound Under Microwave Irradiation

A mixture of the piperidine, halobicycle (e.g. 6-chloro-9H-purine), triethylamine (2-10 equivalents) and organic solvent (typically n-butanol or N-methylpyrrolidin-2-one) was irradiated in a sealed microwave vessel to 100-200° C. for 1-5 hours. The reaction was typically filtered under suction washing with suitable organic solvents (e.g. methanol, dichloromethane) then concentrated. Optional aqueous work-up was undertaken followed by purification by silica Biotage column eluting with ethyl acetate/petrol, dichloromethane/acetic acid/methanol/water, or dichloromethane/methanolic ammonia to furnish the pure product.

### Method K

### Carboxybenzyl (Z) Protection

To a solution of the amine in tetrahydrofuran was added an aqueous base in water (e.g. sodium carbonate). The reaction was cooled to 0° C. then benzyl chloroformate was added dropwise. The reaction was left stirring for 6-24 hours, warming slowly to room temperature. The reaction was quenched by addition of water then was extracted with ethyl acetate. The combined organic liquors were washed with brine, dried (MgSO₄) and concentrated to furnish a colourless oil. This crude material was purified on a silica Biotage column eluting with ethyl acetate/petrol mixtures.

### Method L

### General Method for HATU Couplings to Give Amides

A solution of HATU (230 mg, 0.605 mmol) in DMA (2 mL) was added to a mixture of the substrate (0.55 mmol), DIPEA (0.287 mL, 1.65 mmol) and the amine in DMA (3 mL). The resulting mixture was stirred at room temperature overnight, after which time LCMS analysis showed complete conversion to the desired products in all cases. The reaction mixtures were then worked up by SCX and concentrated in vacuo. The dry mixture was then dissolved in a 10% solution of TFA in DCM, and stirred at room temperature for 2 hours. The products were concentrated in vacuo, then purified by prep LCMS (purification service).

### Method YY1

### Amide Coupling

To a mixture of carboxylic acid (1 equivalent), amine (1.1 equivalents), 1-hydroxybenzotriazole (1.1 equivalents) and triethylamine (2.2 equivalents (or 3.3 equivalents if hydrochloride of amine was used)) in N-methylpyrrolidinone was added (N-ethyl-N′-(3-dimethylaminopropyl)carbodiimide hydrochloride (1.1 equivalents). The mixture was then heated at 60° C. with stirring for 16 hours. Upon cooling the reaction mixture was diluted with ethyl acetate and the organic layer was washed with 2M aqueous sodium hydroxide, followed by brine. The organic layer was separated, dried (MgSO₄) and the solvent was removed in vacuo to afford the crude amide intermediate. The product was then either triturated from diethyl ether or was purified by flash column chromatography on silica gel, typically using dichloromethane/methanol as eluent.

### Method YY2

### Boc Deprotection

To the protected amine, optionally dissolved in a suitable organic solvent (typically dichloromethane), was added a strong organic (e.g. trifluoroacetic acid) or inorganic (e.g. hydrochloric acid in 1,4-dioxane) acid. This mixture was stirred at room temperature for between 10 minutes and 18 hours to furnish the crude amine as a salt. If necessary, purification could be achieved via silica column chromatography using a mixture of dichloromethane, methanol, acetic acid and H₂O or dichloromethane, methanol and ammonia, and/or via ion exchange chromatography and/or by preparative HPLC.

### Method YY3

### HCl Salt Formation

The amine (1 equivalent) was dissolved or suspended in methanol and 4M HCl in 1,4-dioxane was added (1 equivalent). The mixture was stoppered and stirred for 2 hours and was then concentrated in vacuo. The residue was triturated using diethyl ether and the solid was filtered off in vacuo, washing with diethyl ether. The solid was then dried in the vacuum oven.

### Method YY4

### Nitrile Reduction

A solution of 1M lithium aluminium hydride in tetrahydrofuran (2 equivalents) was further diluted with anhydrous tetrahydrofuran and the solution was cooled to 0° C. under nitrogen. The nitrile (1 equivalent) was dissolved in anhydrous tetrahydrofuran and this solution was added dropwise to the solution of lithium aluminium hydride under nitrogen. The resulting reaction mixture was stirred for 30 minutes at 0° C. and then typically 1 hour at room temperature. The reaction mixture was then cooled to 0° C. and was quenched by cautious addition of water, followed by 10% aqueous sodium hydroxide solution, followed by water. The mixture was stirred for 1 hour and was then filtered in vacuo. The filtrate was concentrated in vacuo and was then purified by ion-exchange chromatography followed by silica column chromatography using a dichloromethane/methanol mixture as eluent.

### PREPARATION OF INTERMEDIATE COMPOUNDS A-G

### Preparation A

### 5-Bromo-4′-cyano-3′,4′,5′,6′-tetrahydro-2′H-[3,4]bipyridinyl-1′-carboxylic acid tert-butyl ester

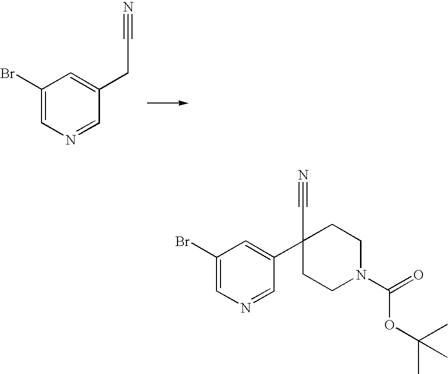

To a solution of (5-bromo-pyridin-3-yl)-acetonitrile (3.62 g, 18.4 mmol) and bis-(2-chloro-ethyl)-carbamic acid tert-butyl ester (made using a method described in J. Chem. Soc., Perkin Trans 1, 2000, p 3444-3450. 4.05 g, 16.7 mmol) in dry N,N-dimethylformamide (15 ml) at room temperature was added sodium hydride (1.53 g, 38.4 mmol). The mixture was heated to 60° C. under nitrogen. After 3 hours an additional 8 ml N,N-dimethylformamide was added. After a further 3 hours the reaction was allowed to cool then water was added and the reaction was extracted with ethyl acetate (×3). The organic liquors were combined and washed with brine, dried (MgSO₄) and concentrated in vacuo. The crude product was purified on a silica Biotage column, eluting 40-65% diethyl ether/petrol furnishing the title compound as a yellow oil (3.55 g, 53%).

### Preparation B

### 4-(Benzyloxycarbonylamino-methyl)-4-[3-(1-methyl-1H-pyrazol-4-yl)-phenyl]-piperidine-1-carboxylic acid tert-butyl ester

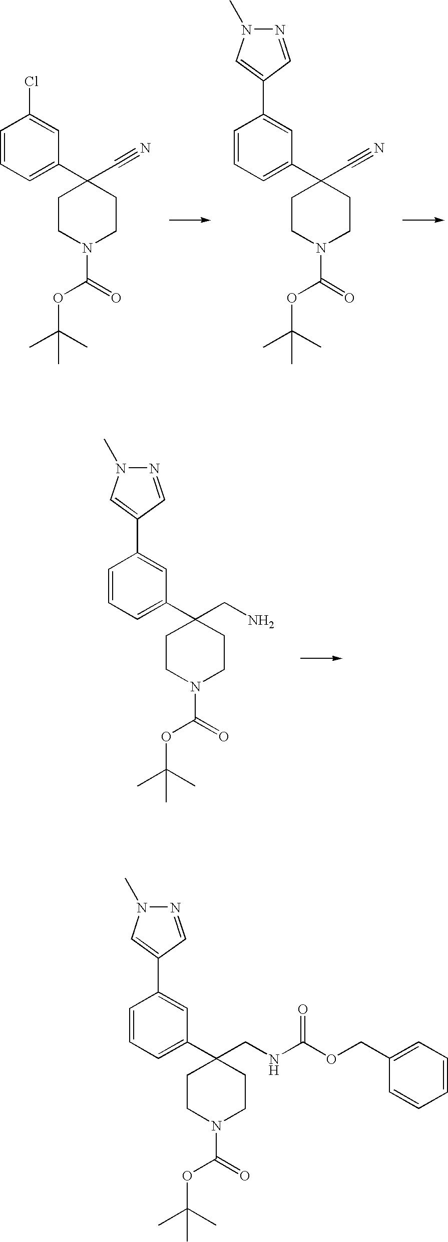

### B1. 4-Cyano-4-[3-(1-methyl-1H-pyrazol-4-yl)-phenyl]-piperidine-1-carboxylic acid tert-butyl ester

A mixture of 4-(3-chloro-phenyl)-4-cyano-piperidine-1-carboxylic acid tert-butyl ester (1.0 g, 3 mmol), 1-methyl-4-(4,4,5,5-tetramethyl-[1,3,2]dioxaborolan-2-yl)-1H-pyrazole (904 mg, 4 mmol), potassium phosphate (2.3 g, 11 mmol), bis(tri-t-butylphosphine)-palladium (0) (96 mg, 0.18 mmol), ethanol (5 ml), methanol (5 ml), toluene (5 ml) and water (5 ml) was heated to 95° C. under nitrogen overnight. The reaction was allowed to cool, water was added and the reaction extracted into ethyl acetate (×2). The organic liquors were washed with brine, dried (MgSO₄) and concentrated in vacuo. The crude product was purified on a silica Biotage column eluting 30-60% ethyl acetate/petrol. The product was obtained as an oil (1.1 g, 96%).

### B2. 4-Aminomethyl-4-[3-(1-methyl-1H-pyrazol-4-yl)-phenyl]-piperidine-1-carboxylic acid tert-butyl ester

To a solution of 4-cyano-4-[3-(1-methyl-1H-pyrazol-4-yl)-phenyl]-piperidine-1-carboxylic acid tert-butyl ester (5.31 g, 14.5 mmol) in ethanol (280 ml) and tetrahydrofuran (70 ml) was added a slurry of Raney Nickel in water (˜7 g) and sodium hydroxide solution (35 ml). This mixture was hydrogenated at room temperature and pressure for 40 hours. The reaction was filtered through celite then concentrated. The residue was wet with water and then extracted into ethyl acetate (×3). The organic liquors were combined and dried (MgSO₄) before concentrating in vacuo (5.4 g, 100%).

### B3. C-4-(Benzyloxycarbonylamino-methyl)-4-[3-(1-methyl-1H-pyrazol-4-yl)-phenyl]-piperidine-1-carboxylic acid tert-butyl ester

To a solution of 4-aminomethyl-4-[3-(1-methyl-1H-pyrazol-4-yl)-phenyl]-piperidine-1-carboxylic acid tert-butyl ester (5.4 g, 14.5 mmol) in tetrahydrofuran (24 ml) was added sodium carbonate in water (2.8 g, 36 mmol in 24 ml). The reaction was cooled to 0° C. then benzyl chloroformate (2.5 ml, 17.4 mmol) was added dropwise. The reaction was left stirring overnight, warming slowly to room temperature. The reaction was quenched by addition of water then was extracted with ethyl acetate (×2). The combined organic liquors were washed with brine, dried (MgSO₄) and concentrated to furnish a colourless oil. This crude material was purified on a silica Biotage column eluting 45-60% ethyl acetate petrol furnishing the product as a white foam (6.7 g, 91%).

### Preparation C

### 4-[3-(1-Methyl-1H-pyrazol-4-yl)-phenyl]-piperidine-1,4-dicarboxylic acid mono-tert-butyl ester

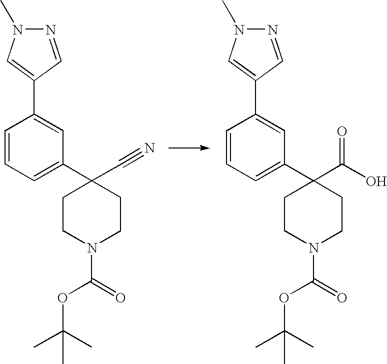

To a suspension of 4-cyano-4-[3-(1-methyl-1H-pyrazol-4-yl)-phenyl]-piperidine-1-carboxylic acid tert-butyl ester (synthesised for Preparation B, 900 mg, 2.5 mmol) in water (2 ml) was added concentrated sulphuric acid (2 ml). This mixture was heated to 100° C. overnight then allowed to cool to room temperature. The reaction was diluted with water (36 ml) then the pH changed to 13 with solid sodium hydroxide whilst cooling the reaction on ice. To the reaction was then added tetrahydrofuran (40 ml) and di-tert-butyl dicarbonate (1.5 g) and the mixture was stirred rapidly for 5 hours at room temperature. The pH was changed to 5 with 2N hydrochloric acid then the mixture was extracted with ethyl acetate (×2). The combined organic liquors were washed with brine, dried (MgSO₄) and concentrated in vacuo to furnish a white solid (1.0 g, 100%).

### Preparation D

### {4-[3-(1-Methyl-1H-pyrazol-4-yl)-phenyl]-piperidin-4-yl}-methanol

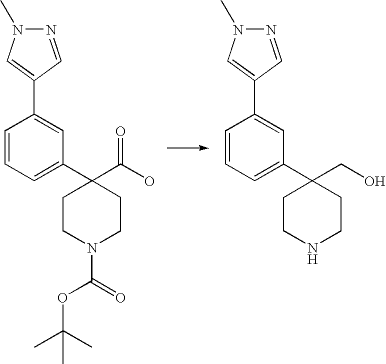

To a solution of 4-[3-(1-methyl-1H-pyrazol-4-yl)-phenyl]-piperidine-1,4-dicarboxylic acid mono-tert-butyl ester (140 mg, 0.36 mmol) in tetrahydrofuran (2 ml) at room temperature and under a nitrogen atmosphere was added a solution of lithium aluminium hydride in tetrahydrofuran (1M solution, 727 ul, 0.727 mmol). The mixture was heated to 50° C. for 3.5 hours then allowed to cool. The reaction was quenched by the sequential addition of water (33 ul), sodium hydroxide solution (15% aqueous, 33 ul), then water (99 ul). The mixture was concentrated and wet with methanol before filtering under suction. The solid was washed with tetrahydrofuran and methanol then concentrated in vacuo. The residue was deprotected by stirring in trifluoroacetic acid (1 ml) and dichloromethane (3 ml) for 30 minutes before concentrating in vacuo and re-concentrating from methanol. The residue was purified on a silica Biotage column eluting DMAW120 to DMAW90 furnishing the title compound (60 mg, 61%).

### Preparation E

### 4-Fluoro-1,3-dihydro-pyrrolo[2,3-b]pyridin-2-one

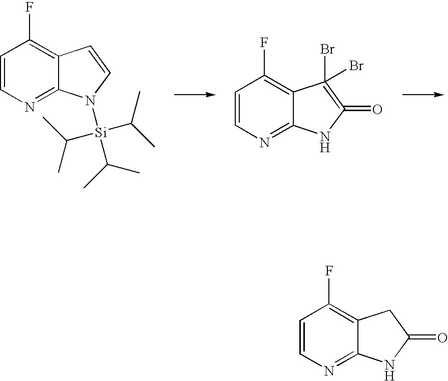

### E1. 3,3-Dibromo-4-fluoro-1,3-dihydro-pyrrolo[2,3-b]pyridin-2-one

To a solution of 4-fluoro-1-triisopropylsilanyl-1H-pyrrolo[2,3-b]pyridine (Org Lett 2003, 5, 5023-5026, 1.0 g, 3.4 mmol) in tert-butanol (25 ml) was added portionwise pyridine tribromide (3.8 g, 11.97 mmol) and this mixture was stirred at room temperature for 3 days. The solvent was removed in vacuo, water and ethyl acetate was added. the mixture was filtered under suction then the organic layer separated. The aqueous fraction was extracted twice with ethyl acetate then the organic liquors were combined and concentrated. The crude product was purified on a silica Biotage column, eluting with petrol/ethyl acetate to furnish the clean product (312 mg, 29%).

### E2. 4-Fluoro-1,3-dihydro-pyrrolo[2,3-b]pyridin-2-one

A mixture of 3,3-dibromo-4-fluoro-1,3-dihydro-pyrrolo[2,3-b]pyridin-2-one (312 mg, 1.0 mmol), acetic acid (4.5 ml), zinc dust (658 mg, 10 mmol) and methanol (4.5 ml) was stirred at room temperature for 3 hours. Brine was added and the reaction was extracted with ethyl acetate. The organic liquors were washed with brine, dried (MgSO₄) and concentrated to furnish the title compound (184 mg, contains ˜40% des-fluorinated product). Used thus in further reactions.

### Preparation F

### 4-Chloro-5,7-dihydro-pyrrolo[2,3-d]pyrimidin-6-one

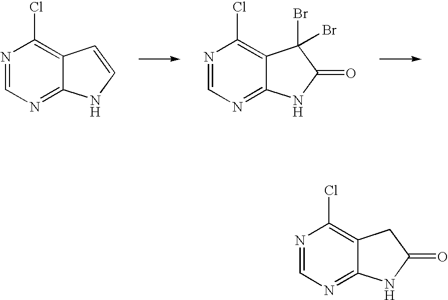

Prepared according to the protocols in Preparation E using 4-chloro-7H-pyrrolo[2,3-d]pyrimidine.

### Preparation G

### C-[4-(3-Chloro-phenyl)-1-(9H-purin-6-yl)-piperidin-4-yl]-methylamine

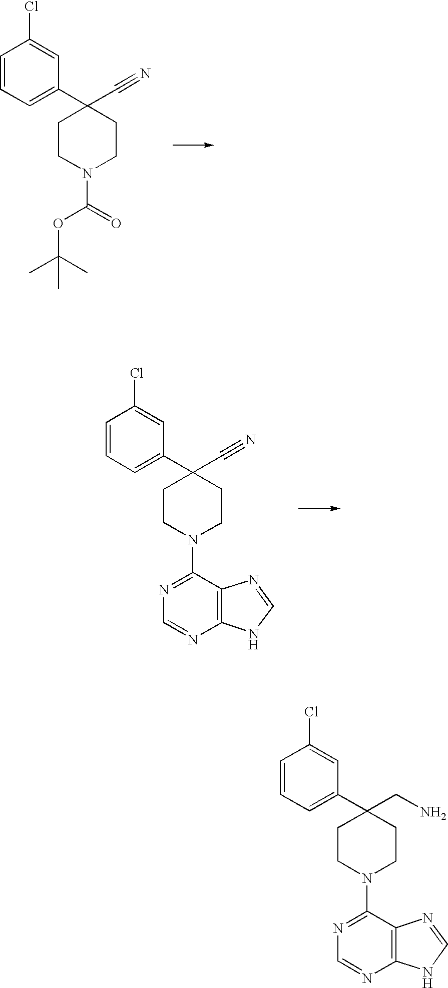

### G1. 4-(3-Chloro-phenyl)-1-(9H-purin-6-yl)-piperidine-4-carbonitrile

To a solution of 4-(3-chloro-phenyl)-4-cyano-piperidine-1-carboxylic acid tert-butyl ester (965 mg, 3.0 mmol) in dichloromethane (10 ml) was added trifluoroacetic acid (4 ml). This mixture was stirred at room temperature for 30 minutes then concentrated in vacuo and re-concentrated from methanol (×2). To this oil was added 6-chloro-9H-purine (464 mg, 3.0 mmol), triethylamine (1.0 ml) and n-butanol (5 ml) then the mixture was heated to 160° C. in a sealed tube in the microwave for 3 hours. The reaction was concentrated in vacuo, triturated with methanol and the solid was dried in a vacuum oven (672 mg, 66%).

### G2. C-[4-(3-Chloro-phenyl)-1-(9H-purin-6-yl)-piperidin-4-yl]-methylamine

To a solution of 4-(3-chloro-phenyl)-1-(9H-purin-6-yl)-piperidine-4-carbonitrile (672 mg, 1.98 mmol) in tetrahydrofuran (20 ml) at room temperature under nitrogen was added lithium aluminium hydride (1M in tetrahydrofuran, 3.97 ml, 4 mmol). A precipitate formed so an additional 20 ml solvent was added. After stirring thus overnight, the reaction was quenched with water (200 μl), sodium hydroxide solution (15%, 200 μl) and then water (600 μl). This mixture was stirred for 30 minutes then the reaction was concentrated in vacuo. The residue was wet with methanol and was filtered under suction. The organic liquors were purified on silica Biotage column eluting DMAW120 to DMAW90. This material was purified by preparative HPLC then re-purified on a second Biotage column to furnish a white solid (131 mg, 19%).

### Preparation H

### 5-bromo-4-chloro-7H-pyrrolo[2,3-d]pyrimidine

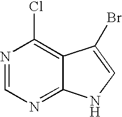

N-Bromosuccinimide (6.84 g, 38.42 mmol) was added portionwise to 4-chloro-7H-pyrrolo[2,3-d]pyrimidine (5 g, 32.56 mmol) in dichloromethane, dry (125 ml) at 20° C. under nitrogen. The resulting suspension was stirred at 20° C. for 1 hour. The reaction mixture was evaporated and the resulting brown solid was triturated with water to give a purple solid which was collected by filtration. The crude solid was triturated with hot MeOH to give a solid which was collected by filtration. The hot trituration was repeated to give 5-bromo-4-chloro-7H-pyrrolo[2,3-d]pyrimidine (5.23 g, 69.1%) as a cream solid.

1H NMR (400.13 MHz, DMSO-d6) δ 7.94 (1H, s), 8.63 (1H, s), 12.95 (1H, s)

MS m/e MH⁺ 232

### Preparation I

### 4,5-dichloro-7H-pyrrolo[2,3-d]pyrimidine

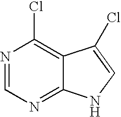

N-Chlorosuccinimide (4.78 g, 35.81 mmol) was added portionwise to a stirred suspension of 4-chloro-7H-pyrrolo[2,3-d]pyrimidine (5 g, 32.56 mmol) in DCM, dry (125 ml) at room temperature. The resulting suspension was stirred for 1 hour then heated to reflux for 5 hours, then allowed to cool down and left to stir at room temperature overnight. The reaction mixture was evaporated and suspended in water (50 mL). The suspension was filtered giving crude product as a grey solid. The solid was suspended in hot methnol and filtered. The solid was then suspended in hot ethyl acetate and filtered to give 4,5-dichloro-7H-pyrrolo[2,3-d]pyrimidine (4.87 g, 80%) as a grey solid.

1H NMR (400.13 MHz, DMSO-d6) δ 7.91 (1H, s), 8.64 (1H, s), 12.87 (1H, s)

MS m/e MH⁺ 188

### Preparation J

### 4-chloro-5-methyl-7H-pyrrolo[2,3-d]pyrimidine

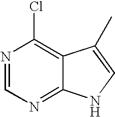

n-Butyllithium (4.08 ml, 6.52 mmol) was added dropwise to 5-bromo-4-chloro-7H-pyrrolo[2,3-d]pyrimidine (689 mg, 2.96 mmol) in tetrahydrofuran (40 ml) at −78° C. over a period of 5 minutes under nitrogen. The resulting suspension was stirred at −78° C. for 30 minutes. Methyl iodide (0.295 ml, 4.74 mmol) was added and the reaction was allowed to warm to ambient temperature. The reaction mixture was diluted with water (25 mL), and extracted with ethyl acetate (50 mL). The organic layer was washed with brine (25 mL) then dried over MgSO4, filtered and evaporated to afford crude product. The crude product was purified by flash silica chromatography, elution gradient 20 to 50% EtOAc in isohexane. Pure fractions were evaporated to dryness to afford 4-chloro-5-methyl-7H-pyrrolo[2,3-d]pyrimidine (244 mg, 49.1%) as a white solid.

1H NMR (400.13 MHz, DMSO-d6) δ 2.42 (3H, d), 7.43 (1H, d), 8.51 (1H, s), 12.22 (1H, s)

MS m/e MH⁺ 168

### Preparation P

### 4-tent-butoxycarbonylamino-1-(7H-pyrrolo[2,3-d]pyrimidin-4-yl)-piperidine-4-carboxylic acid

### P1. 4-tent-butoxycarbonylamino-1-(7H-pyrrolo[2,3-d]pyrimidin-4-yl)-piperidine-4-carboxylic acid ethyl ester

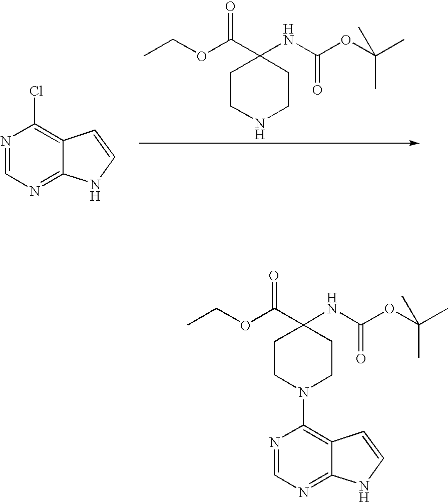

4-tert-butoxycarbonylamino-piperidine-4-carboxylic acid ethyl ester (5 g, 19.4 mmol*) was dissolved in N-methylpyrrolidinone (41 mL) and triethylamine (2.9 mL, 21.3 mmol) was added followed by 4-chloro-7H-pyrrolo[2,3-d]pyrimidine (3.27 g, 21.3 mmol). The resulting mixture was heated at 110° C. under nitrogen for 4 hours. The reaction mixture was allowed to stand for 64 hours. The reaction mixture was diluted with ethyl acetate and the organic was washed three times with water. The organic was separated off, dried (MgSO₄) and concentrated in vacuo. The residue was purified by flash column chromatography on silica gel, eluting with 50/50 ethyl acetate/petroleum ether to afford the title compound as a yellow oil (9.70 g, >100%).

* Commercially available from Astatech (catalogue number: 55743)

### P2. 4-tent-butoxycarbonylamino-1-(7H-pyrrolo[2,3-d]pyrimidin-4-yl)-piperidine-4-carboxylic acid

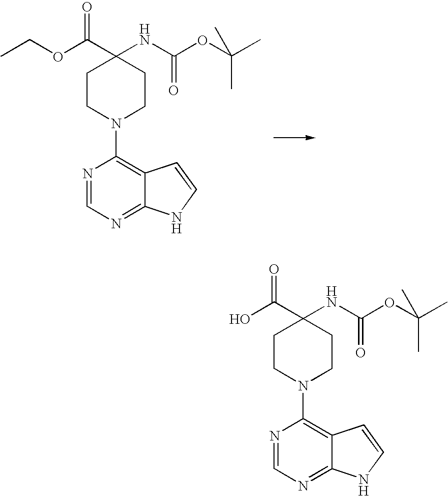

4-tent-butoxycarbonylamino-1-(7H-pyrrolo[2,3-d]pyrimidin-4-yl)-piperidine-4-carboxylic acid ethyl ester (7.28 g, 19.4 mmol) was dissolved in a 1:1 mixture of ethanol and tetrahydrofuran (100 mL in total). A solution of sodium hydroxide (3.88 g, 97 mmol) in water (50 mL) was made up and this was added to the solution of 4-tert-butoxycarbonylamino-1-(7H-pyrrolo[2,3-d]pyrimidin-4-yl)-piperidine-4-carboxylic acid ethyl ester. The resulting mixture was stirred at 60° C. for 18 hours. The reaction was allowed to cool and was concentrated in vacuo. The residue was dissolved in water (100 mL) and was acidified cautiously with conc. HCl to pH 4-5 with ice-cooling. The aqueous was extracted four times with ethyl acetate, each time ensuring an aqueous pH of 4-5. The organics were combined, dried (MgSO₄) and concentrated in vacuo to afford the title compound as a yellow gum (7.3 g, >100%). The product was used without further purification.

### Preparation Q

### 3-(5-Fluoro-pyrimidin-2-yl)-phenylamine

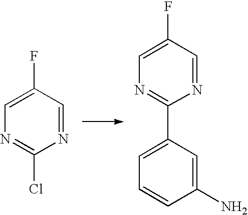

A mixture of 2-chloro-5-fluoro-pyrimidine (900 mg, 6.8 mmol), 3-(4,4,5,5-Tetramethyl-[1,3,2]dioxaborolan-2-yl)-phenylamine (1.63 g, 7.0 mmol), potassium phosphate (3.6 g, 16.9 mmol) and (bis(tri-t-butylphosphine)palladium (0) (175 mg, 0.34 mmol) in ethanol/methanol/toluene/water (2 ml of each) was heated at 80° C. for 2 hours. The reaction was then concentrated in vacuo and partitioned between ethyl acetate and water. The aqueous layer was extracted with ethyl acetate and the combined organic layers were washed with brine, dried (MgSO₄) and concentrated under reduced pressure. The crude product was purified by column chromatography (SiO₂), eluting with a mixture of ethyl acetate/petroleum ether to afford the desired product (1.3 g, 100%)

### Preparation R

4-Amino-piperidine-4-carboxylic acid [3-(4,4-dimethyl-piperidin-1-yl)-phenyl]-amide

### R1. 4,4-Dimethyl-1-(3-nitro-phenyl)-piperidine

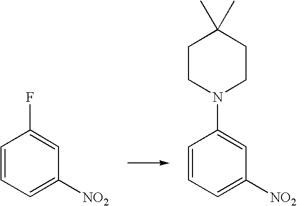

1-Fluoro-3-nitrobenzene (1.49 g, 10.61 mmol), 4,4-dimethylpiperidine (1.20 g, 10.61 mmol) and potassium carbonate (2.20 g, 15.92 mmol) were dissolved in DMF (5 ml) and heated at 90° C. for 18 hours. The reaction mixture was then poured in to water and extracted with ethyl acetate. The organic extracts were combined, washed with brine, dried (MgSO₄) and concentrated under reduced pressure. The crude product was further purified by column chromatography (SiO₂), eluting with a mixture of ethyl acetate/hexane to afford the desired product as a 1:1 mixture with unreacted 1-fluoro-3-nitrobenzene (1.9 g). This was used in the next step without additional purification.

### R2. 3-(4,4-Dimethyl-piperidin-1-yl)-phenylamine

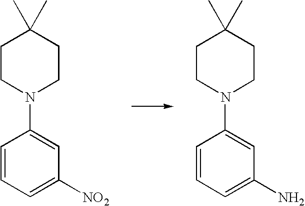

4,4-Dimethyl-1-(3-nitro-phenyl)-piperidine (2.5 g of a 1:1 mixture with 1-fluoro-3-nitrobenzene) was dissolved in ethanol (20 ml) and 10% Palladium on carbon (100 mg) was added. The reaction mixture was then hydrogenated at atmospheric pressure and at room temperature for 4 hours. The reaction mixture was then filtered through a pad aof celite, washed with ethanol and concentrated under reduced pressure. The crude product was further purified by column chromatography (SiO₂), eluting with a mixture of ethyl acetate/hexane to afford the desired product as an oil, which crystallized on standing (0.75 g).

### R3. 4-[3-(4,4-Dimethyl-piperidin-1-yl)-phenylcarbamoyl]-4-(9H-fluoren-9-ylmethoxycarbonylamino)-piperidine-1-carboxylic acid 9H-fluoren-9-ylmethyl ester

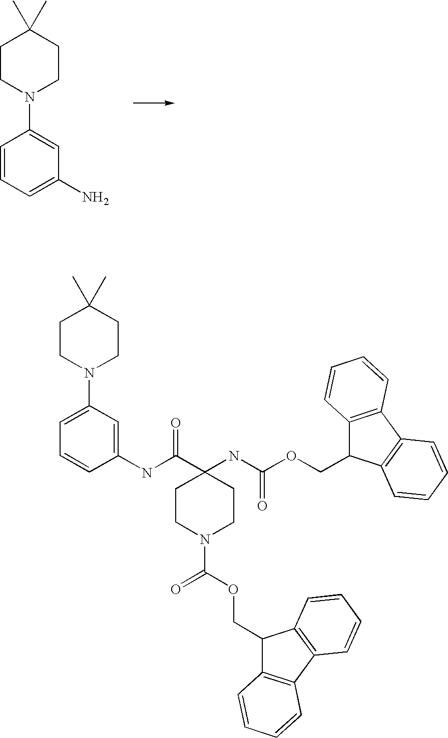

3-(4,4-Dimethyl-piperidin-1-yl)-phenylamine (0.7 g, 3.42 mmol), FMOC-PIP(FMOC)OH (commercially available from BAChem (B-3195.0005), 2.0 g, 3.42 mmol), EDC (0.78 g, 4.1 mmol) and HOBT (0.63 g, 4.1 mmol) were dissolved in DMF (20 ml) and stirred at room temperature for 18 hours. The reaction mixture was then poured in to water and extracted with diethyl ether. The organic extracts were combined, washed with brine, dried (MgSO₄) and concentrated under reduced pressure. The crude product was then purified by column chromatography (SiO₂), eluting with a mixture of ethyl acetate/hexane (1:1) to afford the desired product (2.2 g, 83%).

### R4. 4-Amino-piperidine-4-carboxylic acid [3-(4,4-dimethyl-piperidin-1-yl)-phenyl]-amide

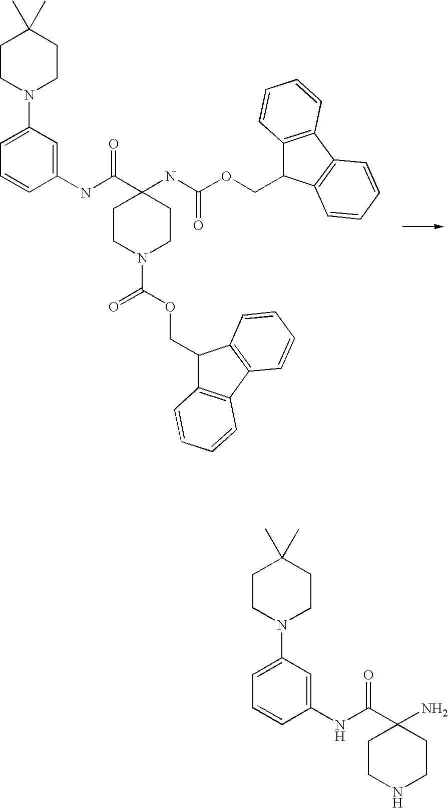

4-[3-(4,4-Dimethyl-piperidin-1-yl)-phenylcarbamoyl]-4-(9H-fluoren-9-ylmethoxycarbonylamino)-piperidine-1-carboxylic acid 9H-fluoren-9-ylmethyl ester (2.2 g, 2.8 mmol) and diisopropylethylamine (5 ml) were dissolved in DMF (20 ml) and stirred at 50° C. for 4 hours. Upon cooling, the reaction mixture was poured in to water and extracted with ethyl acetate. The organic extracts were combined, washed with brine, dried (MgSO₄) and concentrated under reduced pressure. The crude product was then purified by column chromatography (SiO₂), eluting with a mixture of ethyl acetate/hexane to afford the desired product (0.5 g, 53%).

### Example 1

### 4-Aminomethyl-1-(7H-pyrrolo[2,3-d]pyrimidin-4-yl)-piperidine-4-carboxylic acid (6-chloro-pyridin-3-ylmethyl)-amide

### 1A. 4-(6-Chloro-pyridin-3-ylmethylcarbamoyl)-4-cyano-piperidine-1-carboxylic acid tert-butyl ester

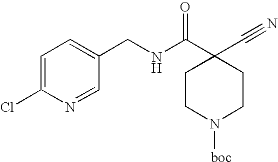

5-Aminomethyl-2-chloropyridine (1.28 mmol) is added to a solution of 4-cyano-piperidine-1,4-dicarboxylic acid mono-tent-butyl ester (250 mg, 0.98 mmol), HATU (486 mg, 1.28 mmol) and Hünig's base (0.86 ml, 4.92 mmol) in DMF (2.5 ml) and stirred at room temperature under an atmosphere of argon. After stirring for 17 h, the reaction mixture is partitioned between dichloromethane and water. The organic layers are then dried, filtered and evaporated. The crude material can be purified by flash silica column chromatography, eluting with 25% ethyl acetate-petrol, to afford the title compound.

### 1B. 4-Cyano-piperidine-4-carboxylic acid (6-chloro-pyridin-3-ylmethyl)-amide

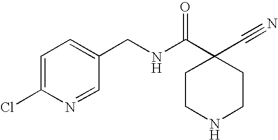

To a solution of the product of Example 1A (0.83 mmol) in methanol (30 ml) at rt is added 4M HCl in dioxane (30 ml). After stirring for 20 h the solution is concentrated to give the deprotected amine as the hydrochloride salt. The crude product can be further purified on SCX-II acidic resin, eluting with methanol then 2M ammonia-methanol, to give the title compound.

### 1C. 4-Cyano-1-(7H-pyrrolo[2,3-d]pyrimidin-4-yl)-piperidine-4-carboxylic acid (6-chloro-pyridin-3-ylmethyl)-amide

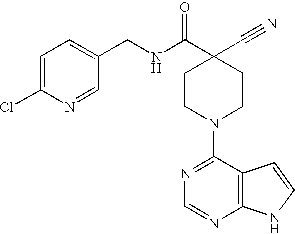

A degassed mixture of 4-cyano-piperidine-4-carboxylic acid (6-chloro-pyridin-3-ylmethyl)-amide (0.80 mmol), 4-chloro-7H-pyrrolo[2,3-d]pyrimidine (122 mg, 0.80 mmol), triethylamine (777 μL, 5.57 mmol) and n-butanol (1.5 mL) is heated to 100° C. for 1.5 h in a microwave. The reaction mixture is then partitioned between ethyl acetate and saturated aqueous ammonium chloride solution. The organic layer is dried, filtered and concentrated. The crude mixture can be purified by flash silica column chromatography, eluting with 10% methanol-dichloromethane to afford the title compound.

### 1D. 4-Aminomethyl-1-(7H-pyrrolo[2,3-d]pyrimidin-4-yl)-piperidine-4-carboxylic acid (6-chloro-pyridin-3-ylmethyl)-amide

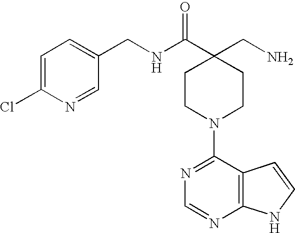

Sodium borohydride (141 mg, 3.73 mmol) is added portionwise, slowly, to a stirred solution of 4-cyano-1-(7H-pyrrolo[2,3-d]pyrimidin-4-yl)-piperidine-4-carboxylic acid (6-chloro-pyridin-3-ylmethyl)-amide (0.37 mmol) and NiCl₂.6H₂O (177 mg, 0.75 mmol) in methanol (3 ml) at 0° C. After 15 minutes, the reaction mixture is allowed to warm to room temperature and stirred for a further 17 hours. The reaction mixture is diluted with methanol and concentrated HCl added (37.3 mmol). The mixture is then heated to reflux for 1 hour. Upon cooling, the solvent is removed in vacuo and the residue purified by flash silica column chromatography, eluting with 10% 2M ammonia in methanol-dichloromethane to afford the title compound.

LC-MS m/z 400/402. ¹H NMR (400 MHz, DMSO-d6): 11.65 (1H, s), 8.64 (1H, t), 8.36 (1H, d), 8.14 (1H, s), 7.79 (1H, dd), 7.47 (1H, d), 7.15 (1H, d), 6.55 (1H, d), 4.35 (2H, d), 4.30-4.23 (2H, m), 3.45-3.35 (2H, m), 2.69 (2H, s), 2.12-2.05 (2H, m), 1.53-1.43 (2H, m).

### Example 2

### 4-Amino-1-(8-oxo-8,9-dihydro-7H-purin-6-yl)-piperidine-4-carboxylic acid (3-benzooxazol-2-yl-phenyl)-amide

### 2A. 4-tert-Butoxycarbonylamino-4-(3-benzooxazol-2-yl-phenylcarbamoyl)-piperidine-1-carboxylic acid tert-butyl ester

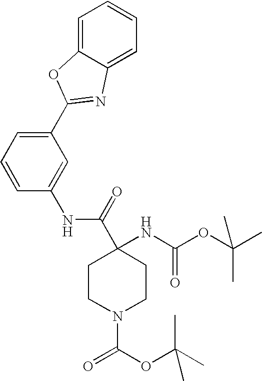

3-Benzooxazol-2-yl-phenylamine (1.74 mmol) is added to a stirred solution of 4-tert-butoxycarbonylamino-piperidine-1,4-dicarboxylic acid mono-tert-butyl ester (600 mg, 1.74 mmol), HATU (861 mg, 2.26 mmol) and Hunig's base (1.52 ml, 8.71 mmol) in DMF (5 ml) and stirred at room temperature under an atmosphere of argon. After stirring for 17 h, the reaction mixture is partitioned between dichloromethane and water. The organic layers are then dried, filtered and evaporated. The crude material can be purified by flash silica column chromatography, eluting with 25% ethyl acetate-petrol, to afford the title compound.

### 2B. 4-Amino-piperidine-4-carboxylic acid (3-benzooxazol-2-yl-phenyl)-amide

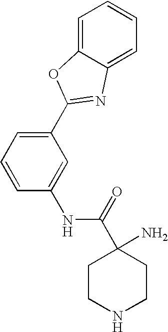

Trifluoroacetic acid (0.5 ml, 6.7 mmol) is added dropwise to a solution of 4-tert-butoxycarbonylamino-4-(3-benzooxazol-2-yl-phenylcarbamoyl)-piperidine-1-carboxylic acid tert-butyl ester in dichloromethane (1 mL). The solution is stirred at rt for 45 min. The solvents are concentrated and the crude mixture is purified on SCX-II acidic resin, eluting with methanol then 2M ammonia-methanol, to give the title compound.

### 2C. 5,6-Diamino-4-chloropyrimidine

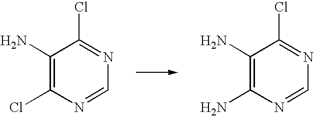

A mixture of 4,6-dichloro-5-aminopyrimidine (Aldrich Chemical Co.) (2.0 g, 12.2 mmol) and concentrated aqueous ammonia (20 ml) was heated to 100° C. in a sealed glass tube with vigorous stirring for 18 hours. The cooled tube was recharged with concentrated aqueous ammonia (8 ml), aggregates were broken up, and the mixture was reheated at 100° C. for a further 28 hours. The mixture was evaporated to dryness and the solids were washed with water (20 ml) and dried to give the product as yellow crystals (1.71 g, 97%). LC/MS (LCT1): Rₜ 1.59 [M+H]⁺ 147, 145.

### 2D. 6-Chloro-7,9-dihydropurin-8-one

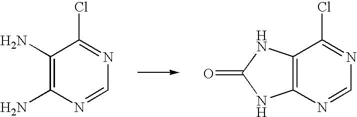

A mixture of the 5,6-diamino-4-chloropyrimidine of Example 6A (1.0 g, 6.92 mmol) and N,N′-carbonyldiimidazole (2.13 g, 13.2 mmol) in 1,4-dioxane (20 ml) was refluxed under argon for 48 hours. The solution was concentrated to a brown oil, which was triturated and washed with dichloromethane to give an off-white solid (1.02 g, 86%) LC/MS (LCT1): Rₜ 2.45 [M+H]⁺173, 171.

### 2E. 4-Amino-1-(8-oxo-8,9-dihydro-7H-purin-6-yl)-piperidine-4-carboxylic acid (3-benzooxazol-2-yl-phenyl)-amide

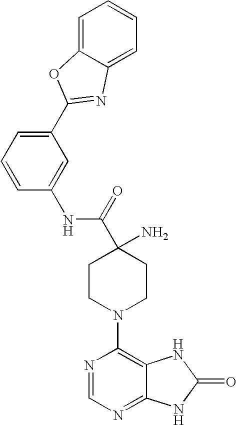

A degassed mixture of 4-amino-piperidine-4-carboxylic acid (3-benzooxazol-2-yl-phenyl)-amide (0.32 mmol), 6-chloro-7,9-dihydro-purin-8-one (50.5 mg, 0.30 mmol), triethylamine (0.3 mL, 2.14 mmol) and n-butanol (3 mL) is stirred at 100° C. for 18 h. The solvents are removed by evaporation and the crude material is purified on SCX-II acidic resin, eluting with methanol then 2M ammonia-methanol to give the title compound. LC-MS m/z 471.

¹H NMR (400 MHz, DMSO-d6): 8.71 (1H, s), 8.14-8.07 (1H, m), 7.95-7.76 (4H, m), 7.56 (1H, t), 7.51-7.38 (2H, m), 4.04 (2H, d), 3.48-3.39 (2H, m), 2.15-2.01 (2H, m), 1.55 (2H, d).

### Example 3

### 4-Amino-1-(8-oxo-8,9-dihydro-7H-purin-6-yl)-piperidine-4-carboxylic acid [3-(4-methyl-pyridin-2-yl)-phenyl]-amide

### 3A. 4-Amino-piperidine-4-carboxylic acid [3-(4-methyl-pyridin-2-yl)-phenyl]-amide

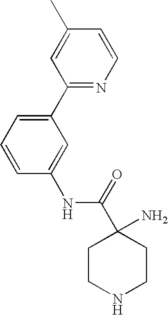

The title compound is prepared according to the methods described in Examples 2A and 2B, using 3-(4-methyl-pyridin-2-yl)-phenylamine instead of 3-benzooxazol-2-yl-phenylamine.

### 3B. 4-Amino-1-(8-oxo-8,9-dihydro-7H-purin-6-yl)-piperidine-4-carboxylic acid [3-(4-methyl-pyridin-2-yl)-phenyl]-amide

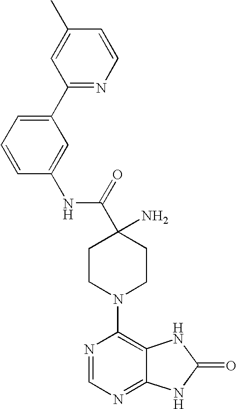

The title compound is prepared according to the methods described in Examples 2C to 2E. LC-MS m/z 445. ¹H NMR (400 MHz, Me-d3-OD): 8.47 (1H, d), 8.21-8.13 (2H, m), 7.75-7.66 (3H, m), 7.48 (1H, t), 7.25 (1H, d), 4.26-4.15 (2H, m), 3.59-3.47 (2H, m), 2.68 (2H, s), 2.48 (3H, s), 2.42-2.31 (2H, m), 1.74 (2H, d).

### Example 4

### 4-Amino-1-(7H-pyrrolo[2,3-d]pyrimidin-4-yl)-piperidine-4-carboxylic acid (6-chloro-pyridin-3-ylmethyl)amide

### 4A 4-tert-Butoxycarbonylamino-4-(6-chloropyridin-3-ylmethylcarbamoyl)-piperidine-1-carboxylic acid tert-butyl ester

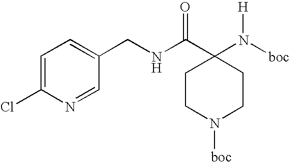

5-Aminomethyl-2-chloropyridine (4 mmol) 4-tert-butoxycarbonylamino-piperidine-1,4-dicarboxylic acid mono-tent-butyl ester compound (1.38 g, 4 mmol), HOBT (0.648 g 4.8 mmol) and EDC (0.92 g, 4.8 mmol) in DMF (20 mls) are stirred at room temperature for 18 h. The reaction mixture is partitioned between dichloromethane and water. The organic layers are then dried, filtered and evaporated. The crude material is purified by flash silica column chromatography, eluting with petroleum ether/ethyl acetate gradient, to afford the title compound.

### 4B 4-Amino-piperidine-4-carboxylic acid 6-chloropyridin-3-ylmethylamide

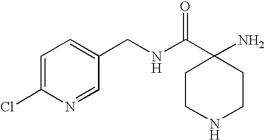

4-tert-Butoxycarbonylamino-4-(6-chloropyridin-3-ylmethylcarbamoyl)-piperidine-1-carboxylic acid tert-butyl ester (3 mM) is dissolved in dichloromethane (30 mls) and trifluoroacetic acid (15 ml). The reaction mixture is stirred at room temperature for 2 h. The solvent is evaporated and the residue loaded onto a 10 g SCX cartridge. The cartridge is eluted with methanol then 2M ammonia in methanol. The methanolic ammonia solution is evaporated under reduced pressure to give the title compound.

### 4C. 4-Amino-1-(7H-pyrrolo[2,3-d]pyrimidin-4-yl)-piperidine-4-carboxylic acid (6-chloro-pyridin-3-ylmethyl)amide

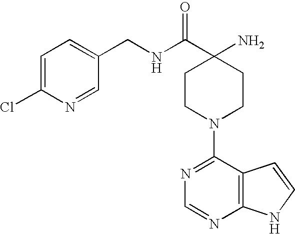

4-Amino-piperidine-4-carboxylic acid 6-chloropyridin-3-ylmethylamide (0.5 mmol) and 6-chlorodeazapurine (76 mgs, 0.5 mmol) in n-butanol (10 mls) with triethylamine (0.28 mls 4 eq) are heated at 120° C. for 66 h. The solvent is evaporated and the residue loaded onto a 10 g SCX cartridge. The cartridge is eluted with methanol then 2M ammonia in methanol. The methanolic ammonia solution is evaporated under reduced pressure to give an oil. The oil is triturated with acetonitrile, the solid obtained collected by filtration to give the title compound. LC-MS m/z 385. ¹H NMR (400 MHz, DMSO-d6): 11.65 (1H, s), 8.67 (1H, s), 8.30 (1H, d), 8.13 (1H, s), 7.72 (1H, dd), 7.46 (1H, d), 7.16 (1H, t), 6.58 (1H, dd), 4.40 (2H, d), 4.29 (2H, d), 3.54 (2H, t), 2.21 (2H, s), 2.03-1.89 (2H, m), 1.45 (2H, d).

### Example 5

### 4-Amino-1-(1H-pyrrolo[2,3-b]pyridin-4-yl)-piperidine-4-carboxylic acid (6-chloro-pyridin-3-ylmethyl)-amide

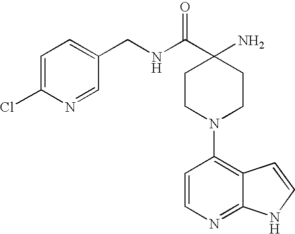

The title compound is prepared using the method described in Example 4 but using 4-fluoro-1-triisopropylsilanyl-1H-pyrrolo[2,3-b]pyridine (Organic Letters (2003), Vol. 5, No. 26, 5023-5025) instead of 4-chloro-7H-pyrrolo[2,3-d]pyrimidine. LC-MS m/z 385. ¹H NMR (Me-d3-OD): 8.33 (1H, d), 7.93 (1H, d), 7.78 (1H, dd), 7.43 (1H, d), 7.18 (1H, d), 6.57-6.47 (2H, m), 4.44 (2H, s), 3.93-3.80 (2H, m), 3.49-3.36 (2H, m), 2.41-2.26 (2H, m), 1.69-1.58 (2H, m).

### Examples 6 to 13

By using the methods and the intermediates described above, the compounds of Examples 6 to 13 were prepared.

| Example | | Chemical | | | |
|---|---|---|---|---|---|
| Number | Compound | Name | Method | N.M.R. Data | M.S. |
| 6 | 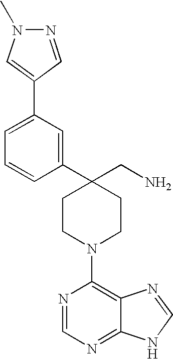 | C-[4-[3-(1- Methyl-1H- pyrazol-4-yl)- phenyl]-1-(9H- purin-6-yl)- piperidin-4- yl]- methylamine | 1. Method J using Preparation B and 6-chloro- 9H-purine 2. Method H3 | ¹H NMR (DMSO- d₆) 8.19 (2H, s), 8.10 (1H, s), 7.90 (1H, s), 7.60 (1H, s), 7.41 (1H, d), 7.32 (1H, t), 7.22 (1H, d), 4.70 (2H, m), 3.38 (3H, s), 3.65 (2H, m), 2.72 (2H, s), 2.25 (2H, m), 1.85 (2H, m) | M/z: 389 |
| 7 | 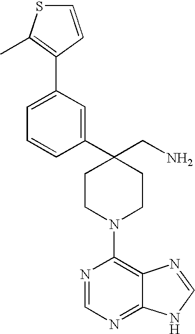 | C-[4-[3-(2- Methyl- thiophen-3-yl)- phenyl]-1-(9H- purin-6-yl)- piperidin-4- yl]- methylamine | Method Cl using Preparation G & 2-methyl- thiophene boronic acid | ¹H NMR (Me-d₃- OD) 8.20 (1H, s), 8.01 (1H, s), 7.58 (1H, s), 7.52 (2H, m), 7.41 (1H, d), 7.30 (1H, d), 6.98 (1H, d), 4.92 (2H, m), 3.65 (2H, m), 2.91 (2H, s), 2.46 (2H, m), 2.33 (3H, s), 1.98 (2H, m) | M/z: 405 |
| 8 | 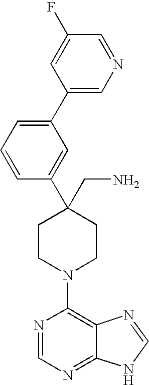 | C-[4-[3-(5- Fluoro- pyridin-3-yl)- phenyl]-1-(9H- purin-6-yl)- piperidin-4- yl]- methylamine (formate salt) | Method Cl using Preparation G & 3- fluoropyridine- 5-boronic acid | ¹H NMR (400 MHz, DMSO-d₆): 8.85 (1H, s), 8.59 (1H, d), 8.31 (1H, s), 8.19 (1H, s), 8.14 (1H, d), 8.10 (1H, s), 7.80 (1H, s), 7.72-7.64 (1H, m), 7.55 (2H, d), 3.68 (2H, s), 3.18 (2H, s), 2.88 (2H, s), 2.33 (2H, d), 2.00-1.86 (2H, m). | M/z: 404 |
| 9 | 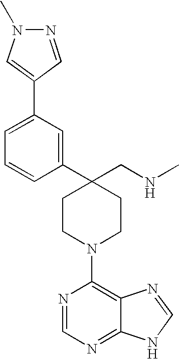 | Methyl-[4-[3- (1-methyl-1H- pyrazol-4-yl)- phenyl]-1-(9H- purin-6-yl)- piperidin-4- ylmethyl]- amine (acetic acid salt) | 1. Method I using Prep- aration B & iodomethane 2. Method J using 6- chloro-9H- purine 3. Method H2 4. Method D | ¹H NMR (Me-d₃- OD) 8.23 (1H, s), 8.07 (1H, s), 8.05 (1H, s), 7.91 (1H, s), 7.72 (1H, s), 7.60 (1H, d), 7.52 (1H, t), 7.42 (1H, d), 5.00 (2H, m), 3.98 (3H, s), 3.63 (2H, m), 2.61 (3H, s), 2.55 (2H, m), 2.00 (5H, m) | M/z: 403 |
| 10 | 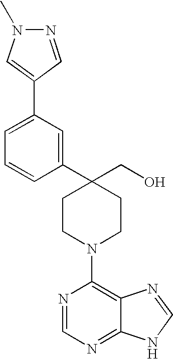 | [4-[3-(1- Methyl-1H- pyrazol-4-yl)- phenyl]-1-(9H- purin-6-yl)- piperidin-4- yl]-methanol | Method J using Preparation D & 6-chloro- 9H-purine | ¹H NMR (400 MHz, DMSO-d₆): 8.19 (1H, s), 8.01 (1H, s), 7.99 (1H, s), 7.85 (1H, s), 7.66 (1H, s), 7.49- 7.32 (3H, m), 4.97 (2H, s), 3.95 (3H, s), 3.66-3.47 (4H, m), 2.39 (2H, d), 2.09-1.95 (2H, m). | M/z: 390 |
| 11 | 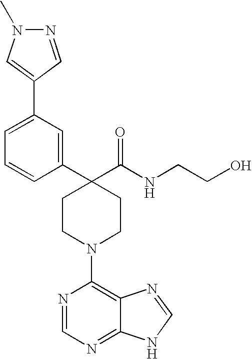 | 4-[3-(1- Methyl-1H- pyrazol-4-yl)- phenyl]-1-(9H- purin-6-yl)- piperidine-4- carboxylic acid (2- hydroxy- ethyl)-amide (acetic acid salt) | 1. Method G using Preparation C & 2- hydroxyethyl- amine 2. Method D 3. Method J using using 6- chloro-9H- purine | ¹H NMR (400 MHz, Me-d₃-OD): 8.22 (1H, s), 8.03 (1H, s), 7.98 (1H, s), 7.84 (1H, s), 7.68 (1H, t), 7.62 (1H, s), 7.45 (1H, d), 7.37 (1H, t), 7.32 (1H, d), 5.03- 4.93 (2H, m), 3.94 (3H, s), 3.81 (2H, t), 3.57 (2H, t), 3.36 (2H, d), 2.66 (2H, d), 2.22-2.10 (2H, m), 2.01 (5H, s). | M/z: 477 |
| 12 | 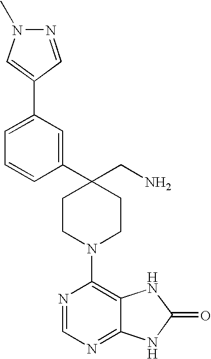 | 6-{4- Aminomethyl- 4-[3-(1- methyl-1H- pyrazol-4-yl)- phenyl]piperidin- 1-yl}-7,9- dihydro-purin- 8-one | 1. Method J using Preparation B and 6-chloro- 7,9-dihydro- purin-8-one 2. Method H3 | ¹H NMR (400 MHz, Me-d3-OD): 8.10 (1H, s), 8.01 (1H, s), 7.87 (1H, s), 7.62 (1H, s), 7.52-7.41 (2H, m), 7.34 (1H, d), 4.09- 3.99 (2H, m), 3.95 (3H, s), 3.40-3.35 (2H, m), 2.84 (2H, s), 2.44 (2H, d), 1.98-1.86 (2H, m). | M/z: 405 |
| 13 | 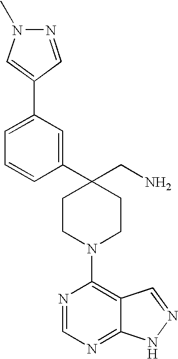 | C-[4-[3-(1- Methyl-1H- pyrazol-4-yl)- phenyl]-1-(1H- pyrazolo[3,4- d]pyrimidin-4- yl)-piperidin- 4-yl]- methylamine | 1. Method J using Preparation B and 4-chloro- 1H- pyrazolo[3,4- d]pyrimidine 2. Method H3 | ¹H NMR (400 MHz, Me-d3-OD): 8.24 (2H, d), 8.03 (1H, s), 7.89 (1H, s), 7.66 (1H, s), 7.54-7.42 (2H, m), 7.38 (1H, d), 4.49 (2H, s), 3.96 (3H, s), 3.63-3.51 (2H, m), 2.86 (2H, s), 2.52 (2H, d), 2.02- 1.89 (2H, m). | M/z: 389 |
| 14 | 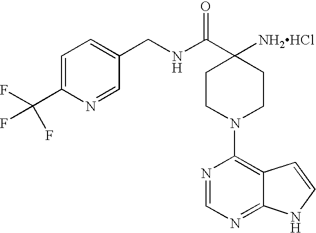 | 4-amino-1- (7H- pyrrolo[2,3- d]pyrimidin- 4-yl)-piperidine-4- carboxylic acid (6- trifluoromethyl- pyridin-3- ylmethyl)- amide hydrochloride | 1. Method YY1 using the product from preparation P and C-(6- trifluoromethyl- pyridin-3-yl)- methylamine as coupling partners 2. Method YY2 3. Method YY3 | ¹H NMR (DMSO- d6): 11.74 (1H, br s), 9.20 (1H, br s), 8.68 (1H, br s), 8.36-7.80 (5H, m), 7.21 (1H, br s), 6.65 (1H, br s), 4.59-4.29 (4H, m), 3.90-3.66 (2H, m), 2.38-2.16 (2H, m), 2.01-1.78 (2H, m). | M/z: 420 |
| 15 | 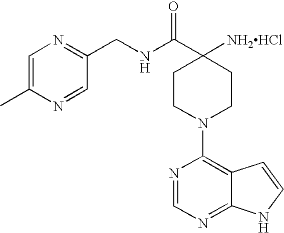 | 4-amino-1- (7H- pyrrolo[2,3- d]pyrimidin- 4-yl)- piperidine-4- carboxylic acid (5- methyl- pyrazin-2- ylmethyl)- amide hydrochloride | 1. Method YY1 using the product from preparation P and C-(5- methyl- pyrazin-2-yl)- methylamine as coupling partners 2. Method YY2 3. Method YY3 | ¹H NMR (Me-d3- OD): 8.46 (2H, d), 8.21 (1H, s), 7.20 (1H, d), 6.69 (1H, d), 4.66-4.51 (4H, m), 3.83-3.70 (2H, m), 2.54 (3H, s), 2.50-2.37 (2H, m), 2.08-1.96 (2H, m). | M/z: 367 |
| 16 | 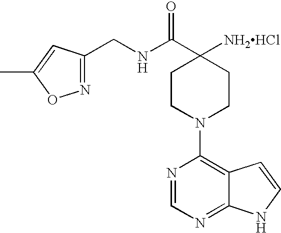 | 4-amino-1- (7H- pyrrolo[2,3- d]pyrimidin-4- yl)-piperidine- 4-carboxylic acid (5- methyl- isoxazol-3- ylmethyl)- amide hydrochloride | 1. Method YY1 using the product from preparation P and C-(5- methyl- isoxazol-3-yl)- methylamine as coupling partners 2. Method YY2 3. Method YY3 | ¹H NMR (DMSO- d6): 11.76 (1H, s), 9.09 (1H, t), 8.65 (2H, s), 8.18 (1H, s), 7.26-7.19 (1H, m), 6.67 (1H, d), 6.12 (1H, s), 4.51- 4.38 (2H, m), 4.32 (2H, d), 3.84-3.66 (2H, m), 2.37 (3H, s), 2.34-2.20 (2H, m), 1.99-1.84 (2H, m). | M/z: 356 |
| 17 | 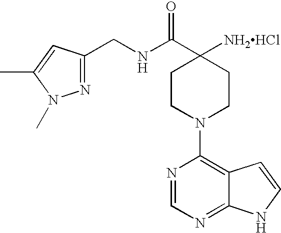 | 4-amino-1- (7H- pyrrolo[2,3- d]pyrimidin-4- yl)-piperidine- 4-carboxylic acid (1,5- dimethyl-1H- pyrazol-3- ylmethyl)- amide hydrochloride | 1. Method YY1 using the product from preparation P and C-(1,5- dimethyl-1H- pyrazol-3-yl)- methylamine as coupling partners 2. Method YY2 3. Method YY3 | ¹H NMR (DMSO- d6): 11.73 (1H, s), 8.80 (1H, t), 8.21 (2H, br s), 8.17 (1H, s), 7.23-7.17 (1H, m), 6.68 (1H, d), 5.87 (1H, s), 4.53-4.38 (2H, m), 4.17 (2H, d), 3.77- 3.59 (5H, m), 2.32-2.14 (5H, m), 1.90-1.78 (2H, m). | M/z: 369 |
| 18 | 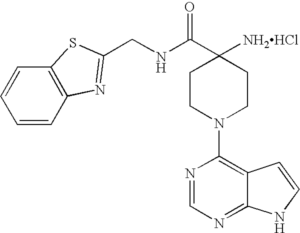 | 4-amino-1- (7H-pyrrolo[2,3- d]pyrimidin-4- yl)-piperidine- 4-carboxylic acid (benzothiazol, 2-ylmethyl)- amide hydrochloride | 1. Method YY1 using the product from preparation P and C- benzothiazol- 2-yl- methylamine hydrochloride as coupling partners 2. Method YY2 3. Method YY3 | ¹H NMR (DMSO- d6): 11.74 (1H, s), 9.48 (1H, br s), 8.18 (1H, s), 8.07 (1H, d), 7.94 (1H, d), 7.56-7.38 (2H, m), 7.26-7.18 (1H, m), 6.73-6.63 (1H, m), 4.82-4.68 (2H, m), 4.58-4.42 (2H, m), 3.83-3.67 (2H, m), 2.39-2.23 (2H, m), 1.98-1.82 (2H, m). | M/z: 408 |
| 19 | 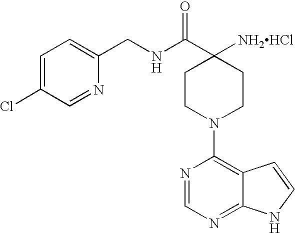 | 4-amino-1- (7H- pyrrolo[2,3- d]pyrimidine-4- yl)-piperidine- 4-carboxylic acid (5-chloro- pyridin-2- ylmethyl)- amide hydrochloride | 1. Method YY4 using 5- chloro pyridine-2- carbonitrile as starting material 2. Method YY1 using the product from preparation P and C-(5- chloro-pyridin- 2-yl)- methylamine (from step 1) as coupling partners 3. Method YY2 4. Method YY3 | ¹H NMR (DMSO- d6): 11.74 (1H, br s), 9.19-9.01 (1H, m), 8.64-8.47 (1H, m), 8.17 (1H, s), 7.96-7.85 (1H, m), 7.33 (1H, d), 7.27- 7.13 (1H, m), 6.75- 6.58 (1H, m), 4.59- 4.32 (4H, m), 3.82- 3.60 (2H,m), 2.38- 2.18 (2H, m), 1.97- 1.76 (2H, m). | M/z: 386 |
| 20 | 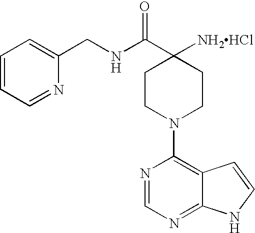 | 4-amino-1- (7H- pyrrolo[2,3- d]pyrimidin-4- yl)-piperidine- 4-carboxylic acid (pyridin- 2-ylmethyl)- amide hydrochloride | 1. Method YY4 using 5- chloropyridine-2- carbonitrile as starting material (this generated the over-reduced side product: C-pyridin-2- yl- methylamine) 2. Method YY1 using the product from preparation P and C-pyridin- 2-yl- methylamine (from step 1) as coupling partners 3. Method YY2 4. Method YY3 | ¹H NMR (DMSO- d6): 11.73 (1H, br s), 9.12-8.94 (1H, m), 8.50 (1H, d), 8.17 (1H, s), 7.84- 7.68 (1H, m), 7.37- 7.13 (3H, m), 6.74- 6.59 (1H, m), 4.54- 4.36 (4H, m), 3.81- 3.62 (2H, m), 2.35- 2.18 (2H, m), 1.94- 1.73 (2H, m). | M/z: 352 |

### Example 21

### 4-(aminomethyl)-N-((5-bromothiophen-2-yl)methyl)-1-(7H-pyrrolo[2,3-d]pyrimidin-4-yl)piperidine-4-carboxamide

### 21A. tert-Butyl 4-cyanopiperidine-1-carboxylate

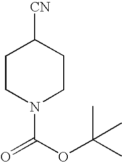

Piperidine-4-carbonitrile (98 g, 889.64 mmol) was dissolved in DCM (1200 ml), to this was added di-tert-butyl dicarbonate (204 g, 934.12 mmol) portionwise. The reaction was stirred at 25° C. for one hour before being evaporated to dryness. The crude gum was dissolved in isohexane (300 ml), cooled in an ice bath and stirred to give a solid which was collected by filtration and dried under vacuum to give tert-butyl 4-cyanopiperidine-1-carboxylate (155 g, 83%) as a white solid.

1H NMR (400.13 MHz, DMSO-d6) δ 1.40 (9H, s), 1.59-1.65 (2H, m), 1.80-1.85 (2H, m), 3.03 (1H, q), 3.14-3.19 (2H, m), 3.51-3.57 (2H, m)

### 21B. 1-tert-Butyl 4-ethyl 4-cyanopiperidine-1,4-dicarboxylate

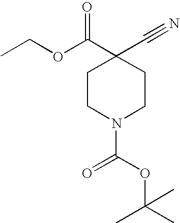

A solution of LDA (107 ml, 214.01 mmol) was added to a stirred solution of tert-butyl 4-cyanopiperidine-1-carboxylate (30 g, 142.67 mmol) in THF (250 ml) at −78° C., under nitrogen. The resulting solution was stirred at −78° C. for 30 minutes. Ethyl chloroformate (16.37 ml, 171.21 mmol) was added. The resulting solution was stirred and allowed to warm to room temperature. The reaction mixture was quenched with saturated NaHCO3 (250 ml), extracted with DCM, and the organic layer was washed with saturated brine (100 ml) then dried over MgSO4, filtered and evaporated to afford the crude material as a orange oil. This material was purified by flash silica chromatography, elution gradient 10% EtOAc in isohexane. Pure fractions were evaporated to dryness to afford 1-tert-butyl 4-ethyl 4-cyanopiperidine-1,4-dicarboxylate (20.80 g, 51.6%) as a yellow oil.

1H NMR (400.13 MHz, CDCl₃) δ 1.33 (3H, t), 1.46 (9H, s), 1.96-2.00 (2H, m), 2.04-2.08 (2H, m), 3.12 (2H, s), 4.09-4.14 (2H, m), 4.29 (2H, q).

### 21C. 1-tert-Butyl 4-ethyl 4-(aminomethyl)piperidine-1,4-dicarboxylate

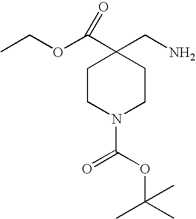

Platinum(IV) oxide (0.724 g, 3.19 mmol) and 1-tert-butyl 4-ethyl 4-cyanopiperidine-1,4-dicarboxylate (9 g, 31.88 mmol) in acetic acid (100 ml) were stirred under an atmosphere of hydrogen at 5 bar and 25° C. for 1 day. The crude product was filtered through celite and the filtrate purified by ion exchange chromatography, using an SCX column. The desired product was eluted from the column using 7M NH3/MeOH and pure fractions were evaporated to dryness to afford 1-tert-butyl 4-ethyl 4-(aminomethyl)piperidine-1,4-dicarboxylate (7.59 g, 83%) as a colourless oil.

1H NMR (400.13 MHz, CDCl₃) δ 1.27-1.28 (3H, m), 1.30-1.37 (2H, m), 1.41 (2H, s), 1.45 (9H, s), 2.10 (2H, d), 2.78 (2H, s), 2.91-2.97 (2H, m), 3.89 (2H, s), 4.21 (2H, q).

### 21D. 1-tert-Butyl 4-ethyl 4-((diphenylmethyleneamino)methyl)piperidine-1,4-dicarboxylate

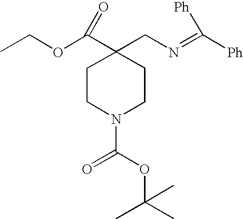

1-tert-Butyl 4-ethyl 4-(aminomethyl)piperidine-1,4-dicarboxylate (7 g, 24.44 mmol), benzophenone imine (4.10 mL, 24.44 mmol) and p-toluenesulfonic acid (1.263 g, 7.33 mmol) were added to DCM (200 mL) and stirred at 25° C. for 3 days. The reaction mixture was quenched with saturated NaHCO3 (100 mL), extracted with DCM (3×100 mL), the organic layer was dried over MgSO4, filtered and evaporated to afford yellow gum. The crude product was purified by flash silica chromatography, elution 0 to 10% EtOAc in isohexane. Pure fractions were evaporated to dryness to afford 1-tert-butyl 4-ethyl 4-((diphenylmethyleneamino)methyl)piperidine-1,4-dicarboxylate (5.86 g, 53.2%) as a colourless gum.

1H NMR (400.13 MHz, CDCl₃) δ 1.25 (3H, t), 1.44 (9H, s), 2.11-2.14 (2H, m), 3.00 (2H, t), 3.46 (2H, s), 3.79-3.81 (2H, m), 4.20 (2H, q), 7.10-7.12 (2H, m), 7.27-7.32 (2H, m), 7.34-7.38 (1H, m), 7.44-7.50 (3H, m), 7.56-7.58 (2H, m)

MS m/e MH⁺ 451

### 21E. Ethyl 4-((diphenylmethyleneamino)methyl)piperidine-4-carboxylate

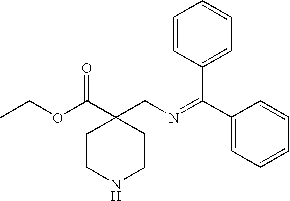

Hydrogen chloride 4M in dioxane (10.52 ml, 42.08 mmol) was added to 1-tert-butyl 4-ethyl 4-((diphenylmethyleneamino)methyl)piperidine-1,4-dicarboxylate (2.37 g, 5.26 mmol) in dioxane (10 ml). The resulting solution was stirred at ambient temperature for 2 hours. The reaction mixture was purified by ion exchange chromatography, using an SCX column. The desired product was eluted from the column using 7M NH3/MeOH and pure fractions were evaporated to dryness to afford ethyl 4-((diphenylmethylene-amino)methyl)piperidine-4-carboxylate (1.530 g, 83%) as a yellow gum.

1H NMR (400.13 MHz, CDCl₃) δ 1.26 (3H, t), 1.45 (2H, d), 2.12-2.15 (2H, m), 2.71-2.77 (2H, m), 2.86-2.91 (2H, m), 3.48 (2H, s), 4.20 (2H, q), 7.10-7.13 (2H, m), 7.27-7.38 (3H, m), 7.40-7.48 (3H, m), 7.57-7.60 (2H, m)

MS m/e MH⁺ 351

### 21F. Ethyl 4-((diphenylmethyleneamino)methyl)-1-(7H-pyrrolo[2,3-d]pyrimidin-4-yl)piperidine-4-carboxylate

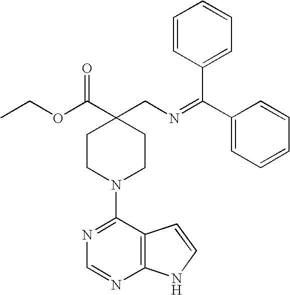

N-Ethyldiisopropylamine (0.982 mL, 5.68 mmol) was added to ethyl 4-((diphenylmethyleneamino)methyl)piperidine-4-carboxylate (1.53 g, 4.37 mmol) and 4-chloro-7H-pyrrolo[2,3-d]pyrimidine (0.670 g, 4.37 mmol) in BuOH (20 mL). The resulting solution was stirred at 60° C. for 24 hours. The reaction mixture was diluted with EtOAc (50 mL), and washed sequentially with water (50 mL) and saturated brine (25 mL). The organic layer was dried over MgSO4, filtered and evaporated to afford crude product. The crude solid was triturated with MeOH to give a solid which was collected by filtration and dried under vacuum to give ethyl 4-((diphenylmethyleneamino)methyl)-1-(7H-pyrrolo[2,3-d]pyrimidin-4-yl)piperidine-4-carboxylate (1.440 g, 70.5%) as a yellow gum.

1H NMR (400.13 MHz, DMSO-d6) δ 1.18 (3H, t), 1.56-1.63 (2H, m), 2.16 (2H, d), 3.33-3.43 (2H, m), 3.46 (2H, s), 4.15 (2H, q), 4.30-4.34 (2H, m), 6.58 (1H, d), 7.15-7.18 (3H, m), 7.35-7.54 (8H, m), 8.12 (1H, s), 11.63 (1H, s)

MS m/e MH⁺ 468

### 21G. 4-((diphenylmethyleneamino)methyl)-1-(7H-pyrrolo[2,3-d]pyrimidin-4-yl)piperidine-4-carboxylic acid

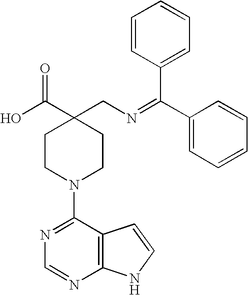

Lithium hydroxide monohydrate (0.646 g, 15.40 mmol) was added to ethyl 4-((diphenylmethyleneamino)methyl)-1-(7H-pyrrolo[2,3-d]pyrimidin-4-yl)piperidine-4-carboxylate (1.44 g, 3.08 mmol) in water (7.50 mL), THF (30 mL) and ethanol (30.0 mL). The resulting solution was stirred at ambient temperature for 7 days. The crude product was purified by ion exchange chromatography, using an SCX column. The desired product was eluted from the column using 7M NH3/MeOH and pure fractions were evaporated to dryness to afford 4-((diphenylmethyleneamino)methyl)-1-(7H-pyrrolo[2,3-d]pyrimidin-4-yl)piperidine-4-carboxylic acid (1.320 g, 98%) as a yellow gum.

MS m/e MH⁺ 440

### 21H. 4-(Aminomethyl)-N-((5-bromothiophen-2-yl)methyl)-1-(7H-pyrrolo[2,3-d]pyrimidin-4-yl)piperidine-4-carboxamide

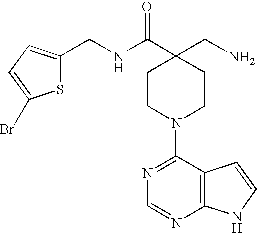

HATU (419 mg, 1.10 mmol) was added in one portion to 4-((diphenylmethylene-amino)methyl)-1-(7H-pyrrolo[2,3-d]pyrimidin-4-yl)piperidine-4-carboxylic acid (440 mg, 1.00 mmol), (5-bromothiophen-2-yl)methanamine hydrochloride (229 mg, 1.00 mmol) and DIPEA (0.699 mL, 4.00 mmol) in DMA (5 ml) at 20° C. under nitrogen. The resulting solution was stirred at 20° C. for 24 hours. The reaction mixture was diluted with EtOAc (100 mL), and washed sequentially with water (2×100 mL) and saturated brine (50 mL). The organic layer was dried over MgSO4, filtered and evaporated to afford crude product. The crude product was purified by flash silica chromatography, elution gradient 0 to 10% MeOH in DCM. Pure fractions were evaporated to dryness to afford N-((5-bromothiophen-2-yl)methyl)-4-((diphenylmethyleneamino)methyl)-1-(7H-pyrrolo[2,3-d]pyrimidin-4-yl)piperidine-4-carboxamide. The product was dissolved in IPA (5.00 ml), water (1 ml) and hydrogen chloride 6N in isopropanol (1.669 ml, 10.01 mmol). The solution was stirred at 20° C. for 24 hours. The crude product was purified by ion exchange chromatography, using an SCX column. The desired product was eluted from the column using 7M NH3/MeOH and pure fractions were evaporated to dryness. The crude gum was triturated with Et2O to give a solid which was collected by filtration and dried under vacuum to give 4-(aminomethyl)-N-((5-bromothiophen-2-yl)methyl)-1-(7H-pyrrolo[2,3-d]pyrimidin-4-yl)piperidine-4-carboxamide (134 mg, 29.8%) as a white solid.

1H NMR (400.13 MHz, DMSO-d6) δ 1.44-1.51 (2H, m), 1.79 (2H, s), 2.07-2.10 (2H, m), 2.66 (2H, s), 3.40 (2H, d), 4.28 (2H, q), 4.42 (2H, d), 6.56 (1H, d), 6.82 (1H, d), 7.03 (1H, d), 7.15 (1H, d), 8.12 (1H, s), 8.71 (1H, t), 11.62 (1H, s)

MS m/e MH⁺ 451

### Example 22

### 4-(Aminomethyl)-N-((5-chlorothiophen-2-yl)methyl)-1-(7H-pyrrolo[2,3-]pyrimidin-4-yl)piperidine-4-carboxamide

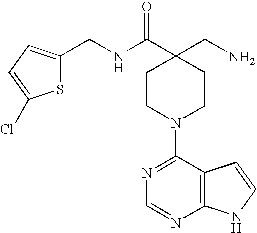

HATU (419 mg, 1.10 mmol) was added in one portion to 4-((diphenylmethyleneamino)methyl)-1-(7H-pyrrolo[2,3-d]pyrimidin-4-yl)piperidine-4-carboxylic acid (440 mg, 1.00 mmol) (Example 21G), (5-chlorothiophen-2-yl)methanamine hydrochloride (184 mg, 1.00 mmol) and DIPEA (0.699 ml, 4.00 mmol) in DMA (5 ml) at 20° C. under nitrogen. The resulting solution was stirred at 20° C. for 24 hours. The reaction mixture was diluted with EtOAc (100 mL), and washed sequentially with water (2×100 mL) and saturated brine (50 mL). The organic layer was dried over MgSO4, filtered and evaporated to afford crude product. The crude product was purified by flash silica chromatography, elution gradient 0 to 5% MeOH in DCM. Pure fractions were evaporated to dryness to afford N-((5-chlorothiophen-2-yl)methyl)-4-((diphenylmethyleneamino)methyl)-1-(7H-pyrrolo[2,3-d]pyrimidin-4-yl)piperidine-4-carboxamide. The product was dissolved in IPA (5.00 ml), water (1 ml) and hydrogen chloride 6N in isopropanol (1.669 ml, 10.01 mmol) added. The solution was stirred at 20° C. for 24 hours. The crude product was purified by ion exchange chromatography, using an SCX column. The desired product was eluted from the column using 7M NH3/MeOH and pure fractions were evaporated to dryness. The gum was triturated with Et2O to give a solid which was collected by filtration and dried under vacuum to give 4-(aminomethyl)-N-((5-chlorothiophen-2-yl)methyl)-1-(7H-pyrrolo[2,3-d]pyrimidin-4-yl)piperidine-4-carboxamide (117 mg, 28.9%) as a white solid.

1H NMR (400.13 MHz, DMSO-d6) δ 1.44-1.51 (2H, m), 2.08 (2H, d), 2.66 (2H, s), 3.40 (2H, s), 4.28 (2H, d), 4.40-4.41 (2H, m), 6.56 (1H, d), 6.84 (1H, d), 6.92 (1H, d), 7.15 (1H, d), 8.12 (1H, s), 8.71 (1H, s), 11.62 (1H, s)

MS m/e MH⁺ 405

### Example 23

### 4-(Aminomethyl)-N-((5-methylthiophen-2-yl)methyl)-1-(7H-pyrrolo[2,3-d]pyrimidin-4-yl)piperidine-4-carboxamide

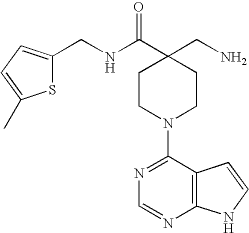

HATU (419 mg, 1.10 mmol) was added in one portion to 4-((diphenylmethylene-amino)methyl)-1-(7H-pyrrolo[2,3-d]pyrimidin-4-yl)piperidine-4-carboxylic acid (440 mg, 1.00 mmol) (Example 21G), (5-methylthiophen-2-yl)methanamine (127 mg, 1.00 mmol) and DIPEA (0.525 ml, 3.00 mmol) in DMA (5 ml) at 20° C. under nitrogen. The resulting solution was stirred at 20° C. for 24 hours. The reaction mixture was diluted with EtOAc (100 mL), and washed sequentially with water (2×100 mL) and saturated brine (50 mL). The organic layer was dried over MgSO4, filtered and evaporated to afford crude product. The crude product was purified by flash silica chromatography, elution gradient 0 to 10% MeOH in DCM. Pure fractions were evaporated to dryness to afford 4-((diphenylmethyleneamino)methyl)-N-((5-methylthiophen-2-yl)methyl)-1-(7H-pyrrolo[2,3-d]pyrimidin-4-yl)piperidine-4-carboxamide. The product was dissolved in IPA (5.00 ml), water (1 ml) and hydrogen chloride 6N in isopropanol (1.669 ml, 10.01 mmol) added. The solution was stirred at 20° C. for 24 hours. The crude product was purified by ion exchange chromatography, using an SCX column. The desired product was eluted from the column using 7M NH3/MeOH and pure fractions were evaporated to dryness. The gum was triturated with Et2O to give a solid which was collected by filtration and dried under vacuum to give 4-(aminomethyl)-N-((5-methylthiophen-2-yl)methyl)-1-(7H-pyrrolo[2,3-d]pyrimidin-4-yl)piperidine-4-carboxamide (80 mg, 20.78%) as a white solid.

1H NMR (400.13 MHz, DMSO-d6) δ 1.42-1.49 (2H, m), 1.66 (2H, s), 2.07-2.11 (2H, m), 2.37 (3H, s), 2.65 (2H, s), 3.38-3.45 (2H, m), 4.25-4.30 (2H, m), 4.41 (2H, d), 6.55 (1H, d), 6.59-6.60 (1H, m), 6.73 (1H, d), 7.14-7.16 (1H, m), 8.12 (1H, s), 8.61 (1H, t), 11.62 (1H, s)

MS m/e MH⁺ 385

### Example 24

### 4-(Aminomethyl)-N-((3-bromoisoxazol-5-yl)methyl)-1-(7H-pyrrolo[2,3-d]pyrimidin-4-yl)piperidine-4-carboxamide

### 24A. 1-tert-Butyl 4-ethyl 4-((tert-butoxycarbonylamino)methyl)piperidine-1,4-dicarboxylate

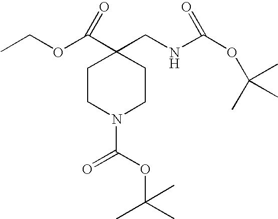

1-tert-Butyl 4-ethyl 4-(aminomethyl)piperidine-1,4-dicarboxylate (7 g, 24.44 mmol) and di-tert-butyl dicarbonate (5.90 ml, 25.67 mmol) were added to DCM (200 ml) and stirred at 25° C. for 1 day. The reaction mixture was quenched with saturated NaHCO3 (100 ml), extracted with DCM (2×100 mL), the organic layer was dried over MgSO4, filtered and evaporated to afford yellow gum. The crude product was purified by flash silica chromatography, elution 15% EtOAc in isohexane. Pure fractions were evaporated to dryness to afford 1-tert-butyl 4-ethyl 4-((tert-butoxycarbonylamino)methyl)piperidine-1,4-dicarboxylate (8.54 g, 90%) as a colourless gum.

1H NMR (400.13 MHz, CDCl₃) δ 1.28 (3H, t), 1.44 (18H, d), 2.01-2.06 (2H, m), 3.08 (2H, d), 3.30 (2H, d), 3.75 (2H, d), 4.19 (4H, m), 4.74 (1H, s)

### 24B. 1-(tert-Butoxycarbonyl)-4-((tert-butoxycarbonylamino)methyl)piperidine-4-carboxylic acid

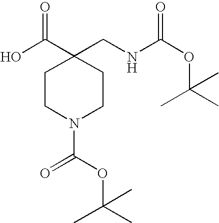

Lithium hydroxide monohydrate (4.61 g, 109.97 mmol) was added to 1-tert-butyl 4-ethyl 4-((tert-butoxycarbonylamino)methyl)piperidine-1,4-dicarboxylate (8.5 g, 21.99 mmol) in water (20.00 ml), methanol (80 ml) and THF (80 ml). The resulting solution was stirred at ambient temperature for 3 hours. The reaction mixture was evaporated to dryness and redissolved in EtOAc (100 mL), and washed with water (50 mL). The aqueous was acidified with 1M citric acid and extracted with ethyl acetate (2×100 ml). The organics were dried (MgSO4), filtered and evaporated to afford crude product 1-(tert-butoxycarbonyl)-4-((tert-butoxycarbonylamino)-methyl)piperidine-4-carboxylic acid (4.65 g, 59.0%) as a yellow gum which was used in the next step without purification.

### 24C. tert-Butyl 4-((3-bromoisoxazol-5-yl)methylcarbamoyl)-4-((tert-butoxycarbonylamino)methyl)piperidine-1-carboxylate

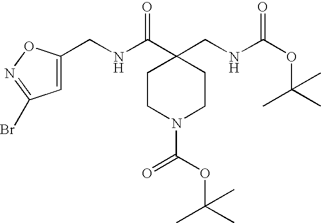

O-(7-Azabenzotriazol-1-yl)-N,N,N′,N′-tetramethyluronium hexafluorophosphate (1.909 g, 5.02 mmol) was added to 1-(tert-butoxycarbonyl)-4-((tert-butoxycarbonylamino)methyl)piperidine-4-carboxylic acid (1.2 g, 3.35 mmol), (3-bromoisoxazol-5-yl)methanamine hydrochloride (0.715 g, 3.35 mmol) and N-Ethyldiisopropylamine (2.317 ml, 13.39 mmol) in DMA (20 ml) at 20° C. over a period of 1 minute under air. The resulting solution was stirred at 20° C. for 1 day. The reaction mixture was diluted with EtOAc (100 mL), and washed sequentially with water (2×100 mL) and saturated brine (50 mL). The organic layer was dried over MgSO4, filtered and evaporated to afford crude product. The crude product was purified by flash silica chromatography, elution gradient 10 to 50% EtOAc in isohexane. Pure fractions were evaporated to dryness to afford tert-butyl 4-((3-bromoisoxazol-5-yl)methylcarbamoyl)-4-((tert-butoxycarbonylamino)methyl)piperidine-1-carboxylate (0.355 g, 20.49%) as a yellow gum.

1H NMR (400.13 MHz, CDCl₃) δ 1.43 (9H, s), 1.45 (9H, s), 1.87-1.93 (2H, m), 3.29-3.34 (4H, m), 3.58-3.64 (2H, m), 4.55 (2H, s), 4.80 (1H, s), 6.26 (1H, s), 6.53 (1H, s)

MS m/e MH⁺ 517

### 24D. 4-(Aminomethyl)-N-((3-bromoisoxazol-5-yl)methyl)piperidine-4-carboxamide

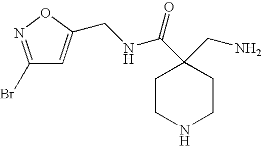

Hydrochloric acid 6N in isopropanol (2.287 ml, 13.72 mmol) was added to tert-butyl 4-((3-bromoisoxazol-5-yl)methylcarbamoyl)-4-((tert-butoxycarbonylamino)-methyl)piperidine-1-carboxylate (355 mg, 0.69 mmol) in IPA (2 ml). The resulting suspension was stirred at 20° C. for 18 hours. The crude product was purified by ion exchange chromatography, using an SCX column. The desired product was eluted from the column using 7M NH3/MeOH and pure fractions were evaporated to dryness to afford 4-(aminomethyl)-N-((3-bromoisoxazol-5-yl)methyl)piperidine-4-carboxamide (196 mg, 90%) as a yellow gum.

1H NMR (400.13 MHz, CDCl₃) δ 1.40-1.47 (2H, m), 1.99-2.04 (2H, m), 2.83 (2H, s), 2.92-2.95 (4H, m), 4.53-4.55 (2H, m), 6.25 (1H, s), 8.72 (1H, s)

MS m/e MH⁺ 317

### 24E. 4-(Aminomethyl)-N-((3-bromoisoxazol-5-yl)methyl)-1-(7H-pyrrolo[2,3-d]pyrimidin-4-yl)piperidine-4-carboxamide

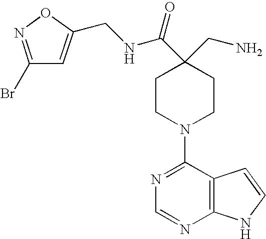

N-Ethyldiisopropylamine (0.129 ml, 0.74 mmol) was added to 4-(aminomethyl)-N-((3-bromoisoxazol-5-yl)methyl)piperidine-4-carboxamide (196 mg, 0.62 mmol) and 4-chloro-7H-pyrrolo[2,3-d]pyrimidine (95 mg, 0.62 mmol) in butan-1-ol (2 ml). The resulting solution was stirred at 60° C. for 3 hours. The reaction mixture was evaporated and the crude product was purified by ion exchange chromatography, using an SCX column. The desired product was eluted from the column using 7M NH3/MeOH and product containing fractions were evaporated to dryness. The product was purified by flash silica chromatography, elution gradient 0 to 15% MeOH in DCM with ammonia. Pure fractions were evaporated to dryness to afford 4-(aminomethyl)-N-((3-bromoisoxazol-5-yl)methyl)-1-(7H-pyrrolo[2,3-d]pyrimidin-4-yl)piperidine-4-carboxamide (117 mg, 43.6%) as a white solid after trituration with diethyl ether.

1H NMR (400.13 MHz, DMSO-d6) δ 1.45-1.52 (4H, m), 2.07-2.10 (2H, m), 2.69 (2H, s), 3.42 (2H, d), 4.24-4.29 (2H, m), 4.48 (2H, s), 6.56 (1H, d), 6.66 (1H, s), 7.15 (1H, d), 8.12 (1H, s), 8.71 (1H, s), 11.62 (1H, s)

MS m/e MH⁺ 434

### Example 25

### N-((1H-Indol-2-yl)methyl)-4-(aminomethyl)-1-(3-bromo-1H-pyrazolo[3,4-d]pyrimidin-4-yl)piperidine-4-carboxamide

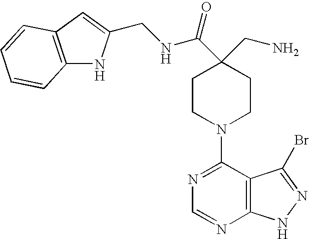

N-Ethyldiisopropylamine (0.066 ml, 0.38 mmol) was added to 3-bromo-4-chloro-1H-pyrazolo[3,4-d]pyrimidine (73.4 mg, 0.31 mmol) and N-((1H-indol-2-yl)methyl)-4-(aminomethyl)piperidine-4-carboxamide (90 mg, 0.31 mmol) in butan-1-ol (2 ml). The resulting solution was stirred at 20° C. for 3 hours. The reaction mixture was evaporated and the crude product was purified by preparative HPLC (Waters XBridge Prep C18 OBD column, 5μ silica, 19 mm diameter, 100 mm length), using decreasingly polar mixtures of water (containing 0.1% TFA) and MeCN as eluents then repeated using decreasingly polar mixtures of water (containing 1% NH3) and MeCN as eluents. Fractions containing the desired compound were evaporated to dryness to afford N-((1H-indol-2-yl)methyl)-4-(aminomethyl)-1-(3-bromo-1H-pyrazolo[3,4-d]pyrimidin-4-yl)piperidine-4-carboxamide (36.0 mg, 23.70%) as a white solid.

1H NMR (400.13 MHz, DMSO-d6) δ 1.57-1.64 (2H, m), 2.22 (2H, d), 2.78 (2H, s), 3.47 (2H, d), 4.15 (2H, d), 4.52 (2H, d), 6.24 (1H, s), 6.91-6.95 (1H, m), 6.99-7.03 (1H, m), 7.32-7.35 (1H, m), 7.43 (1H, d), 8.29 (1H, s), 8.58 (1H, t), 11.15 (1H, s)

MS m/e MH⁺ 483

### Example 26

### N-((1H-Indol-2-yl)methyl)-4-(aminomethyl)-1-(7H-pyrrolo[2,3-d]pyrimidin-4-yl)piperidine-4-carboxamide

### 26A. 1-(tert-Butoxycarbonyl)-4-cyanopiperidine-4-carboxylic acid

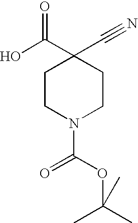

A solution of lithium hydroxide (21.25 ml, 42.50 mmol) in water was added to a stirred solution of 1-tert-butyl 4-ethyl 4-cyanopiperidine-1,4-dicarboxylate (3 g, 10.63 mmol), in THF (42.5 ml) at 25° C. The resulting mixture was stirred at 25° C. for 18 hours. The reaction mixture was diluted with Et₂O (100 mL), and washed with water (50 mL). The aqueous layers were combined and then acidified with citric acid (1N, 50 mL). The product was extracted into DCM. The organic layer was dried over MgSO4, filtered and evaporated to afford crude product 1-(tert-butoxycarbonyl)-4-cyanopiperidine-4-carboxylic acid (2.73 g, 101%) as a yellow liquid.

1H NMR (400.13 MHz, DMSO-d6) δ 1.41 (9H, s), 1.78-1.85 (2H, m), 2.04 (2H, d) 2.95 (2H, t), 3.96 (2H, d), 13.9 (1H, s)

### 26B. tert-Butyl 4-((1H-indol-2-yl)methylcarbamoyl)-4-cyanopiperidine-1-carboxylate

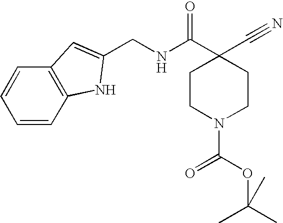

HATU (2224 mg, 5.85 mmol) was added in one portion to 1-(tert-butoxycarbonyl)-4-cyanopiperidine-4-carboxylic acid (1352 mg, 5.32 mmol), (1H-Indol-2-ylmethyl)amine (933 mg, 6.38 mmol) and DIPEA (2.79 ml, 15.96 mmol) in DMA (15 ml) at 20° C. under nitrogen. The resulting solution was stirred at 20° C. for 24 hours. The reaction mixture was diluted with EtOAc (100 mL), and washed sequentially with water (2×100 mL) and saturated brine (50 mL). The organic layer was dried over MgSO4, filtered and evaporated to afford crude product. The crude product was purified by flash silica chromatography, elution gradient 20 to 50% EtOAc in isohexane. Pure fractions were evaporated to dryness to afford tert-butyl 4-((1H-indol-2-yl)methylcarbamoyl)-4-cyanopiperidine-1-carboxylate (930 mg, 45.7%) as a cream solid.

1H NMR (400.13 MHz, CDCl₃) δ 1.47 (9H, s), 1.90 (2H, d), 2.06-2.14 (2H, m), 3.00 (2H, s), 4.22 (2H, s), 4.56 (2H, d), 6.38-6.39 (1H, m), 6.86 (1H, d), 7.09 (1H, t), 7.18 (1H, d), 7.33 (1H, d), 7.56 (1H, d), 8.64 (1H, s)

MS m/e M−H 381

### 26C. tert-Butyl 4-((1H-indol-2-yl)methylcarbamoyl)-4-(aminomethyl)piperidine-1-carboxylate

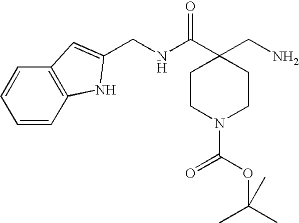

Platinum(IV) oxide (55.2 mg, 0.24 mmol) and tert-butyl 4-((1H-indol-2-yl)methylcarbamoyl)-4-cyanopiperidine-1-carboxylate (930 mg, 2.43 mmol) in acetic acid (30 ml) were stirred under an atmosphere of hydrogen at 1 atmosphere pressure and 25° C. for 1 day. The reaction mixture was filtered and the crude product was purified by ion exchange chromatography, using an SCX column. The desired product was eluted from the column using 7M NH3/MeOH and pure fractions were evaporated to dryness to afford tert-butyl 4-((1H-indol-2-yl)methylcarbamoyl)-4-(aminomethyl)piperidine-1-carboxylate (891 mg, 95%) as a colourless gum.

1H NMR (400.13 MHz, CDCl₃) δ 1.44-1.45 (9H, m), 2.01-2.06 (2H, m), 2.79-2.82 (2H, m), 2.88-2.92 (1H, m), 3.28 (2H, d), 3.66-3.69 (2H, m), 4.52-4.55 (2H, m), 6.30 (1H, s), 7.04-7.08 (1H, m), 7.12-7.16 (1H, m), 7.30-7.32 (1H, m), 7.53 (1H, d), 8.26 (1H, s), 9.24 (1H, s)

MS m/e M−H 385

### 26D. N-((1H-indol-2-yl)methyl)-4-(aminomethyl)piperidine-4-carboxamide

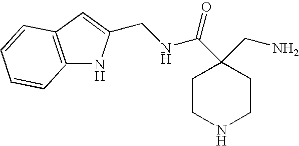

Hydrogen chloride 4M in dioxane (2001 μl, 57.63 mmol) was added to tert-butyl 4-((1H-indol-2-yl)methylcarbamoyl)-4-(aminomethyl)piperidine-1-carboxylate (891 mg, 2.31 mmol). The resulting solution was stirred at ambient temperature for 2 hours. The reaction mixture was dissolved in methanol and purified by ion exchange chromatography, using an SCX column. The desired product was eluted from the column using 7M NH3/MeOH and pure fractions were evaporated to dryness to afford N-((1H-indol-2-yl)methyl)-4-(aminomethyl)piperidine-4-carboxamide (639 mg, 97%) as a yellow gum.

1H NMR (400.13 MHz, CDCl₃) δ 1.38-1.45 (2H, m+H2O), 2.00-2.05 (2H, m), 2.82 (2H, s), 2.86-2.89 (4H, m), 4.54 (2H, s), 6.30 (1H, d), 7.04-7.08 (1H, m), 7.11-7.15 (1H, m), 7.29-7.32 (1H, m), 7.53 (1H, d), 7.95 (1H, s), 9.37 (1H, s)

MS m/e MH⁺ 287

### 26E. N-((1H-Indol-2-yl)methyl)-4-(aminomethyl)-1-(7H-pyrrolo[2,3-d]pyrimidin-4-yl)piperidine-4-carboxamide

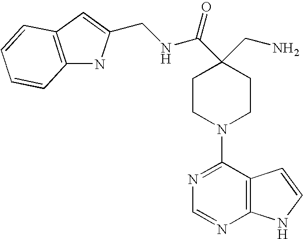

N-Ethyldiisopropylamine (0.109 ml, 0.63 mmol) was added to N-((1H-indol-2-yl)methyl)-4-(aminomethyl)piperidine-4-carboxamide (150 mg, 0.52 mmol) and 4-chloro-7H-pyrrolo[2,3-d]pyrimidine (80 mg, 0.52 mmol) in butan-1-ol (2 ml). The resulting solution was stirred at 60° C. for 18 hours. The reaction mixture was evaporated and the crude product was purified by preparative HPLC (Waters XBridge Prep C18 OBD column, 5μ silica, 19 mm diameter, 100 mm length), using decreasingly polar mixtures of water (containing 1% NH3) and MeCN as eluents. Fractions containing the desired compound were evaporated to dryness to afford N-((1H-indol-2-yl)methyl)-4-(aminomethyl)-1-(7H-pyrrolo[2,3-d]pyrimidin-4-yl)piperidine-4-carboxamide (74.0 mg, 35.0%) as a white solid.

1H NMR (400.13 MHz, DMSO-d6) δ 1.46-1.53 (2H, m), 2.16 (2H, d), 2.73 (2H, s), 3.48 (2H, t), 4.27-4.30 (2H, m), 4.52 (2H, d), 6.23 (1H, s), 6.57 (1H, d), 6.91-6.95 (1H, m), 6.98-7.03 (1H, m), 7.16 (1H, d), 7.32-7.34 (1H, m), 7.43 (1H, d), 8.12 (1H, s), 8.58 (1H, d), 11.17 (1H, s), 11.62 (1H, s)

MS m/e MH⁺ 404

### Example 27

### 4-(Aminomethyl)-1-(7H-pyrrolo[2,3-d]pyrimidin-4-yl)-N-((6-(trifluoromethyl)pyridin-3-yl)methyl)piperidine-4-carboxamide

### 27A. tert-Butyl 4-cyano-4-((6-(trifluoromethyl)pyridin-3-yl)methylcarbamoyl)piperidine-1-carboxylate

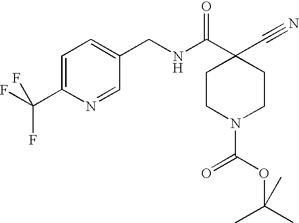

HATU (3.29 g, 8.65 mmol) was added in one portion to 1-(tert-butoxycarbonyl)-4-cyanopiperidine-4-carboxylic acid (2 g, 7.87 mmol), 3-aminomethyl-6-(trifluoromethyl)pyridine (1.385 g, 7.87 mmol) and DIPEA (4.12 ml, 23.60 mmol) in DMA (20 ml) at 20° C. under nitrogen. The resulting solution was stirred at 20° C. for 24 hours. The reaction mixture was diluted with EtOAc (100 mL), and washed sequentially with water (2×100 mL) and saturated brine (50 mL). The organic layer was dried over MgSO4, filtered and evaporated to afford crude product. The crude product was purified by flash silica chromatography, elution gradient 20 to 50% EtOAc in isohexane. Pure fractions were evaporated to dryness to afford tert-butyl 4-cyano-4-((6-(trifluoromethyl)pyridin-3-yl)methylcarbamoyl)piperidine-1-carboxylate (1.760 g, 54.3%) as a white solid.

1H NMR (400.13 MHz, CDCl₃) δ 1.46 (9H, s), 1.92 (2H, d), 2.06-2.14 (2H, m), 3.02 (2H, t), 4.22 (2H, s), 4.57-4.59 (2H, m), 6.79 (1H, s), 7.68 (1H, d), 7.79-7.82 (1H, m), 8.66 (1H, d)

MS m/e M−H 411

### 27B. tert-Butyl 4-(aminomethyl)-4-((6-(trifluoromethyl)pyridin-3-yl)methylcarbamoyl)piperidine-1-carboxylate

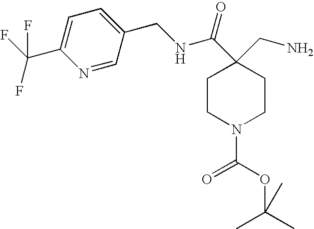

Platinum(IV) oxide (0.097 g, 0.43 mmol) and tert-butyl 4-cyano-4-((6-(trifluoromethyl)pyridin-3-yl)methylcarbamoyl)piperidine-1-carboxylate (1.76 g, 4.27 mmol) in acetic acid (30 ml) were stirred under an atmosphere of hydrogen at 1 atm and 25° C. for 1 day. The reaction mixture was filtered and the crude product was purified by ion exchange chromatography, using an SCX column. The desired product was eluted from the column using 7M NH3/MeOH and pure fractions were evaporated to dryness to afford tert-butyl 4-(aminomethyl)-4-((6-(trifluoromethyl)pyridin-3-yl)methylcarbamoyl)piperidine-1-carboxylate (1.200 g, 67.5%) as a colourless gum.

MS m/e M−H 415

### 27C. 4-(Aminomethyl)-N-((6-(trifluoromethyl)pyridin-3-yl)methyl)piperidine-4-carboxamide

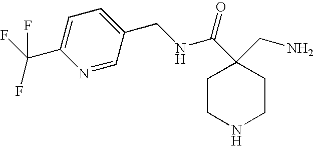

Hydrogen chloride 4M in dioxane (5.76 ml, 23.05 mmol) was added to tert-butyl 4-(aminomethyl)-4-((6-(trifluoromethyl)pyridin-3-yl)methylcarbamoyl)piperidine-1-carboxylate (1.2 g, 2.88 mmol). The resulting solution was stirred at ambient temperature for 2 hours. The reaction mixture was dissolved in methanol and purified by ion exchange chromatography, using an SCX column. The desired product was eluted from the column using 7M NH3/MeOH and pure fractions were evaporated to dryness to afford 4-(aminomethyl)-N-((6-(trifluoromethyl)pyridin-3-yl)methyl)piperidine-4-carboxamide (0.670 g, 73.5%) as a yellow gum.

MS m/e MH⁺ 317

### 27D. 4-((Diphenylmethyleneamino)methyl)-N-((6-(trifluoromethyl)pyridin-3-yl)methyl)piperidine-4-carboxamide

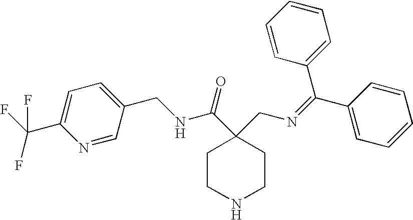

4-(aminomethyl)-N-((6-(trifluoromethyl)pyridin-3-yl)methyl)piperidine-4-carboxamide (500 mg, 1.58 mmol), benzophenone imine (0.265 ml, 1.58 mmol) and p-toluenesulfonic acid (82 mg, 0.47 mmol) were added to DCM (10 ml) and stirred at 25° C. for 1 day. The reaction mixture was quenched with saturated NaHCO3 (20 ml), extracted with DCM (3×20 mL), the organic layer was dried over MgSO4, filtered and evaporated to afford yellow gum. The crude product was purified by flash silica chromatography, elution 0 to 10% MeOH in DCM then 10% methanol in DCM with ammonia. Pure fractions were evaporated to dryness to afford 4-((diphenylmethyleneamino)methyl)-N-((6-(trifluoromethyl)pyridin-3-yl)methyl)piperidine-4-carboxamide (352 mg, 46.3%) as a colourless dry film.

MS m/e MH⁺ 481

### 27E. 4-(Aminomethyl)-1-(7H-pyrrolo[2,3-d]pyrimidin-4-yl)-N-((6-(trifluoromethyl)pyridin-3-yl)methyl)piperidine-4-carboxamide

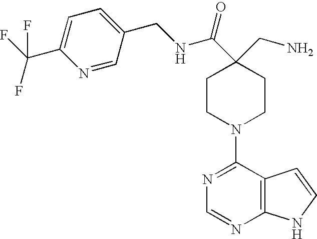

N-Ethyldiisopropylamine (0.153 ml, 0.88 mmol) was added to 4-((diphenylmethyleneamino)methyl)-N-((6-(trifluoromethyl)pyridin-3-yl)methyl)piperidine-4-carboxamide (352 mg, 0.73 mmol) and 4-chloro-7H-pyrrolo[2,3-d]pyrimidine (112 mg, 0.73 mmol) in butan-1-ol (4 ml). The resulting solution was stirred at 80° C. for 18 hours. The reaction mixture was diluted with EtOAc (50 mL), and washed sequentially with water (50 mL) and saturated brine (25 mL). The organic layer was dried over MgSO4, filtered and evaporated to afford crude product. The crude product was purified by flash silica chromatography, elution gradient 2 to 4% MeOH in DCM with ammonia. Pure fractions were evaporated to dryness to afford 4-((diphenylmethyleneamino)methyl)-1-(7H-pyrrolo[2,3-d]pyrimidin-4-yl)-N-((6-(trifluoromethyl)pyridin-3-yl)methyl)piperidine-4-carboxamide as a colourless gum.

4-((Diphenylmethyleneamino)methyl)-1-(7H-pyrrolo[2,3-d]pyrimidin-4-yl)-N-((6-(trifluoromethyl)pyridin-3-yl)methyl)piperidine-4-carboxamide (0.00 μg) was suspended in water (1 ml) and Hydrogen chloride 6N in isopropanol (1.221 ml, 7.33 mmol) and stirred at ambient temperature for 24 hours. The crude product was purified by ion exchange chromatography, using an SCX column. The desired product was eluted from the column using 7M NH3/MeOH and product containing fractions were evaporated to dryness. The crude product was purified by preparative HPLC (Waters XBridge Prep C18 OBD column, 5μ silica, 19 mm diameter, 100 mm length), using decreasingly polar mixtures of water (containing 1% NH3) and MeCN as eluents. Fractions containing the desired compound were evaporated to dryness. The crude product was purified by flash silica chromatography, elution gradient 2 to 10% MeOH in DCM with ammonia. Pure fractions were evaporated to dryness to afford 4-(aminomethyl)-1-(7H-pyrrolo[2,3-d]pyrimidin-4-yl)-N-((6-(trifluoromethyl)pyridin-3-yl)methyl)piperidine-4-carboxamide (87 mg, 27.4%) as a white solid.

1H NMR (400.13 MHz, DMSO-d6) δ 1.47-1.54 (2H, m), 2.08-2.11 (2H, m), 2.71 (2H, s), 3.42 (2H, d), 4.25-4.28 (2H, m), 4.46 (2H, d), 6.56 (1H, d), 7.15 (1H, s), 7.86 (1H, d), 7.98 (1H, d), 8.12 (1H, s), 8.68 (1H, t), 8.71 (1H, s), 11.62 (1H, s)

MS m/e MH⁺ 434

### Example 28

### (1-(5-bromo-7H-pyrrolo[2,3-d]pyrimidin-4-yl)-4-(3-(1-methyl-1H-pyrazol-4-yl)phenyl)piperidin-4-yl)methanamine

### 28A. tert-Butyl 4-cyano-4-(3-(1-methyl-1H-pyrazol-4-yl)phenyl)piperidine-1-carboxylate

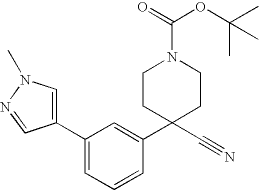

tert-Butyl 4-(3-bromophenyl)-4-cyanopiperidine-1-carboxylate (5.49 g, 15.04 mmol), 1,1′-bis(diphenylphosphino)ferrocenedichloropalladium(II) (0.544 g, 0.75 mmol), 1-methyl-4-(4,4,5,5-tetramethyl-1,3,2-dioxaborolan-2-yl)-1H pyrazole (3.13 g, 15.04 mmol) and potassium phosphate, tribasic (12.77 g, 60.17 mmol) were dissolved in dioxane (40 mL) and heated at 75° C. overnight. The reaction was evaporated to dryness, quenched with water (50 mL), extracted with diethyl ether (3×75 mL), the organic layer was dried over MgSO4, filtered and evaporated to afford black solid. The crude product was purified by flash silica chromatography, elution gradient 0 to 5% MeOH in DCM. Pure fractions were evaporated to dryness to afford tert-butyl 4-cyano-4-(3-(1-methyl-1H-pyrazol-4-yl)phenyl)piperidine-1-carboxylate (4.32 g, 78%) as a orange gum.

1H NMR (400.13 MHz, CDCl₃) δ 1.49 (9H, d), 1.91-2.02 (2H, m), 2.10 (2H, t), 3.19-3.25 (2H, m), 3.95 (3H, s), 4.29 (2H, s), 7.29-7.31 (1H, m), 7.38-7.48 (2H, m), 7.56 (1H, d), 7.64 (1H, s), 7.76-7.76 (1H, m)

MS m/e (M-tBu)⁺ 311

### 28B. tert-Butyl 4-(aminomethyl)-4-(3-(1-methyl-1H-pyrazol-4-yl)phenyl)piperidine-1-carboxylate

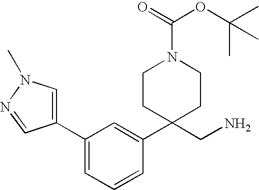

Tert-butyl 4-cyano-4-(3-(1-methyl-1H-pyrazol-4-yl)phenyl)piperidine-1-carboxylate (2.74 g, 7.48 mmol) and platinum(IV) oxide (0.3 g, 1.32 mmol) in acetic acid (50 mL) were stirred under an atmosphere of hydrogen at 5 bar at 25° C. for 16 hours. The reaction mixture was filtered through celite and the crude product was purified by ion exchange chromatography, using an SCX column. The desired product was eluted from the column using 7M NH3/MeOH and pure fractions were evaporated to dryness to afford tert-butyl 4-(aminomethyl)-4-(3-(1-methyl-1H-pyrazol-4-yl)phenyl)piperidine-1-carboxylate (2.350 g, 85%) as a yellow gum.

1H NMR (400.13 MHz, CDCl₃) δ 1.43 (9H, d), 1.69-1.76 (2H, m), 2.20 (2H, d), 2.79 (2H, s), 3.08-3.14 (2H, m), 3.73 (2H, d), 3.95 (3H, s), 7.15-7.18 (1H, m), 7.32-7.38 (3H, m), 7.60 (1H, s), 7.74 (1H, s)

MS m/e (M-tBu)⁺ 315

### 28C. tert-Butyl 4-((diphenylmethyleneamino)methyl)-4-(3-(1-methyl-1H-pyrazol-4-yl)phenyl)piperidine-1-carboxylate

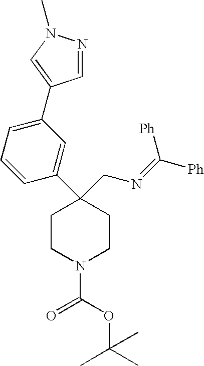

tert-Butyl 4-(aminomethyl)-4-(3-(1-methyl-1H-pyrazol-4-yl)phenyl)piperidine-1-carboxylate (2.34 g, 6.32 mmol), benzophenone imine (1.060 ml, 6.32 mmol) and p-toluenesulfonic acid (0.326 g, 1.89 mmol) were added to DCM (60 ml) and stirred at 25° C. for 1 day. The reaction mixture was quenched with saturated NaHCO3 (20 ml), extracted with DCM (3×20 mL), the organic layer was dried over MgSO4, filtered and evaporated to afford yellow gum. The crude product was purified by flash silica chromatography, elution 0 to 50% EtOAc in isohexane. Pure fractions were evaporated to dryness to afford tert-butyl 4-((diphenylmethyleneamino)methyl)-4-(3-(1-methyl-1H-pyrazol-4-yl)phenyl)piperidine-1-carboxylate (2.450 g, 72.5%) as a colourless gum.

1H NMR (400.13 MHz, CDCl₃) δ 1.44 (9H, s), 1.98-2.05 (2H, m), 2.30 (2H, d), 3.09-3.16 (2H, m), 3.45 (2H, s), 3.75 (2H, d), 3.89 (3H, s), 6.65-6.68 (2H, m), 7.09-7.12 (1H, m), 7.25-7.42 (10H, m), 7.51-7.54 (2H, m), 7.61-7.62 (1H, m)

MS m/e MH⁺ 535

### 28D. N-(Diphenylmethylene)-1-(4-(3-(1-methyl-1H-pyrazol-4-yl)phenyl)piperidin-4-yl)methanamine

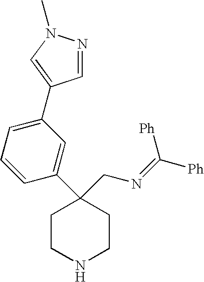

Hydrogen chloride 4M in dioxane (9.16 ml, 36.66 mmol) was added to tert-butyl 4-((diphenylmethyleneamino)methyl)-4-(3-(1-methyl-1H-pyrazol-4-yl)phenyl)piperidine-1-carboxylate (2.45 g, 4.58 mmol) in dioxane (15 ml). The resulting solution was stirred at ambient temperature for 2 hours. The reaction mixture was dissolved in methanol and purified by ion exchange chromatography, using an SCX column. The desired product was eluted from the column using 7M NH3/MeOH and pure fractions were evaporated to dryness to afford N-(diphenylmethylene)-1-(4-(3-(1-methyl-1H-pyrazol-4-yl)phenyl)piperidin-4-yl)methanamine (1.790 g, 90%) as a yellow dry film.

1H NMR (400.13 MHz, CDCl₃) δ 2.03-2.09 (2H, m), 2.30 (2H, d), 2.78-2.84 (2H, m), 2.94-3.00 (2H, m), 3.48 (2H, s), 3.89 (3H, s), 6.65-6.68 (2H, m), 7.11-7.15 (1H, m), 7.27-7.34 (8H, m), 7.24-7.36 (2H, m), 7.51-7.54 (2H, m), 7.62-7.62 (1H, m)

MS m/e MH⁺ 435

### 28E. (1-(5-bromo-7H-pyrrolo[2,3-d]pyrimidin-4-yl)-4-(3-(1-methyl-1H-pyrazol-4-yl)phenyl)piperidin-4-yl)methanamine

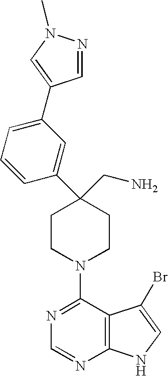

N-Ethyldiisopropylamine (0.125 ml, 0.72 mmol) was added to 5-bromo-4-chloro-7H-pyrrolo[2,3-d]pyrimidine (139 mg, 0.60 mmol) (Preparation H) and N-(diphenylmethylene)-1-(4-(3-(1-methyl-1H-pyrazol-4-yl)phenyl)piperidin-4-yl)methanamine (260 mg, 0.60 mmol) in butan-1-ol (4 ml). The resulting solution was stirred at 20° C. for 4 days. The reaction mixture was diluted with EtOAc (50 mL), and washed sequentially with water (50 mL) and saturated brine (50 mL). The organic layer was dried over MgSO4, filtered and evaporated to afford crude product. The crude product was purified by flash silica chromatography, elution gradient 0 to 5% MeOH in DCM. Pure fractions were evaporated to dryness to afford 1-(1-(5-bromo-7H-pyrrolo[2,3-d]pyrimidin-4-yl)-4-(3-(1-methyl-1H-pyrazol-4-yl)phenyl)piperidin-4-yl)-N-(diphenylmethylene)methanamine as a yellow gum. The material was dissolved in IPA (4.00 ml) and water (1 ml). Hydrogen chloride 6N in isopropanol (0.997 ml, 5.98 mmol) was added and the solution stirred at 20° C. for 18 hours. The crude product was purified by ion exchange chromatography, using an SCX column. The product was eluted from the column using 7M NH3/MeOH and pure fractions were evaporated to dryness. The gum was triturated with Et2O to give a solid which was collected by filtration and dried under vacuum to give (1-(5-bromo-7H-pyrrolo[2,3-d]pyrimidin-4-yl)-4-(3-(1-methyl-1H-pyrazol-4-yl)phenyl)piperidin-4-yl)methanamine (128 mg, 45.9%) as a cream solid.

1H NMR (400.13 MHz, DMSO-d6) δ 2.02 (2H, t), 2.32 (2H, d), 2.76 (2H, s), 3.83 (5, d), 7.24 (1H, d), 7.35 (1H, t), 7.42 (1H, d), 7.51 (1H, s), 7.56 (1H, s), 7.88 (1H, s), 8.17 (1H, s), 8.23 (1H, s)

MS m/e MH⁺ 466

### Example 29

### (1-(5-Chloro-7H-pyrrolo[2,3-d]pyrimidin-4-yl)-4-(3-(1-methyl-1H-pyrazol-4-yl)phenyl)piperidin-4-yl)methanamine

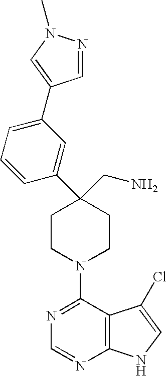

N-Ethyldiisopropylamine (0.120 ml, 0.69 mmol) was added to 4,5-dichloro-7H-pyrrolo[2,3-d]pyrimidine (108 mg, 0.58 mmol) (Preparation I) and N-(diphenylmethylene)-1-(4-(3-(1-methyl-1H-pyrazol-4-yl)phenyl)piperidin-4-yl)methanamine (250 mg, 0.58 mmol) (Example 28D) in butan-1-ol (4 ml). The resulting solution was stirred at 110° C. for 2 hours. Hydrogen chloride 6N in isopropanol (0.959 ml, 5.75 mmol) and water (1 ml) were added and heating continued for 10 minutes. The crude product was purified by ion exchange chromatography, using an SCX column. The product was eluted from the column using 7M NH3/MeOH and product containing fractions were evaporated to dryness. The crude product was purified by preparative HPLC (Waters XBridge Prep C18 OBD column, 5μ silica, 19 mm diameter, 100 mm length), using decreasingly polar mixtures of water (containing 1% NH3) and MeCN as eluents. Fractions containing the desired compound were evaporated to dryness to afford (1-(5-chloro-7H-pyrrolo[2,3-d]pyrimidin-4-yl)-4-(3-(1-methyl-1H-pyrazol-4-yl)phenyl)piperidin-4-yl)methanamine (81 mg, 33.4%) as a white solid.

1H NMR (400.13 MHz, DMSO-d6) δ 1.98-2.01 (2H, m), 2.26 (2H, s), 2.72 (2H, s), 3.87 (5H, s), 7.24 (1H, s), 7.35 (1H, t), 7.40-7.42 (1H, m), 7.45 (1H, s), 7.56 (1H, s), 7.87-7.88 (1H, m), 8.17 (1H, s), 8.21 (1H, s)

MS m/e MH⁺ 422

### Example 30

### (4-(3-(1-methyl-1H-pyrazol-4-yl)phenyl)-1-(5-methyl-7H-pyrrolo[2,3-d]pyrimidin-4-yl)piperidin-4-yl)methanamine

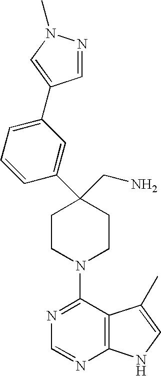

N-Ethyldiisopropylamine (0.120 ml, 0.69 mmol) was added to 4-chloro-5-methyl-7H-pyrrolo[2,3-d]pyrimidine (96 mg, 0.58 mmol) (Preparation J) and N-(diphenylmethylene)-1-(4-(3-(1-methyl-1H-pyrazol-4-yl)phenyl)piperidin-4-yl)methanamine (250 mg, 0.58 mmol) (Example 28D) in butan-1-ol (4 ml). The resulting solution was stirred at 110° C. for 2 hours. Hydrogen chloride 6N in isopropanol (0.959 ml, 5.75 mmol) and water (1 ml) were added and heating continued for 10 minutes. The product was purified by ion exchange chromatography, using an SCX column. The product was eluted from the column using 7M NH3/MeOH and product containing fractions were evaporated to dryness. The crude product was purified by preparative HPLC (Waters XBridge Prep C18 OBD column, 5μ silica, 19 mm diameter, 100 mm length), using decreasingly polar mixtures of water (containing 1% NH3) and MeCN as eluents. Fractions containing the desired compound were evaporated to dryness to afford (4-(3-(1-methyl-1H-pyrazol-4-yl)phenyl)-1-(5-methyl-7H-pyrrolo[2,3-d]pyrimidin-4-yl)piperidin-4-yl)methanamine (41.0 mg, 17.75%) as a white solid.

1H NMR (400.13 MHz, DMSO-d6) δ 1.95-2.01 (2H, m), 2.26-2.30 (2H, m), 2.36-2.37 (3H, m), 2.72 (2H, s), 3.19 (2H, t), 3.67-3.71 (2H, m), 3.87 (3H, s), 7.03 (1H, s), 7.23 (1H, d), 7.35 (1H, t), 7.41 (1H, d), 7.55 (1H, s), 7.87-7.87 (1H, m), 8.16 (2H, d), 11.44 (1H, s)

MS m/e MH⁺ 402

### Example 31

### (1-(3-Bromo-1H-pyrazolo[3,4-d]pyrimidin-4-yl)-4-(3-(1-methyl-1H-pyrazol-4-yl)phenyl)piperidin-4-yl)methanamine

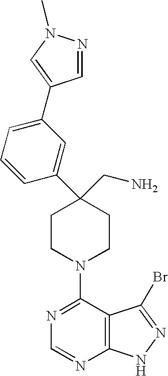

N-Ethyldiisopropylamine (0.120 ml, 0.69 mmol) was added to 3-bromo-4-chloro-1H-pyrazolo[3,4-d]pyrimidine (134 mg, 0.58 mmol) and N-(diphenylmethylene)-1-(4-(3-(1-methyl-1H-pyrazol-4-yl)phenyl)piperidin-4-yl)methanamine (250 mg, 0.58 mmol) (Example 28D) in butan-1-ol (4 ml). The resulting solution was stirred at 20° C. for 4 hours. HCl 6N in isopropanol (1 mL, 0.41 mmol) and water (0.5 ml) were added and the reaction stirred for 18 hours. The crude product was purified by ion exchange chromatography, using an SCX column. The crude product was eluted from the column using 7M NH3/MeOH. The crude product was purified by preparative HPLC (Waters XBridge Prep C18 OBD column, 5μ silica, 19 mm diameter, 100 mm length), using decreasingly polar mixtures of water (containing 1% NH3) and MeCN as eluents. Fractions containing the desired compound were evaporated to dryness to afford impure product as a yellow gum. The crude product was purified by flash silica chromatography, elution gradient 5% MeOH in DCM with ammonia. Pure fractions were evaporated to dryness. Trituration with diethyl ether gave (1-(3-bromo-1H-pyrazolo[3,4-d]pyrimidin-4-yl)-4-(3-(1-methyl-1H-pyrazol-4-yl)phenyl)piperidin-4-yl)methanamine (78 mg, 29.0%) as a white solid.

1H NMR (400.13 MHz, DMSO-d6) δ 1.96-2.02 (2H, m), 2.32 (2H, d), 2.74 (2H, s), 3.44 (2H, d), 3.87 (3H, s), 4.06-4.10 (2H, m), 7.24 (1H, d), 7.36 (1H, t), 7.42 (1H, d), 7.57 (1H, s), 7.88 (1H, s), 8.17 (1H, s), 8.28 (1H, s)

MS m/e MH⁺ 467

### Example 32

### Preparation of N,N-dimethyl-1-(4-(3-(1-methyl-1H-pyrazol-4-yl)phenyl)-1-(9H-purin-6-yl)piperidin-4-yl)methanamine

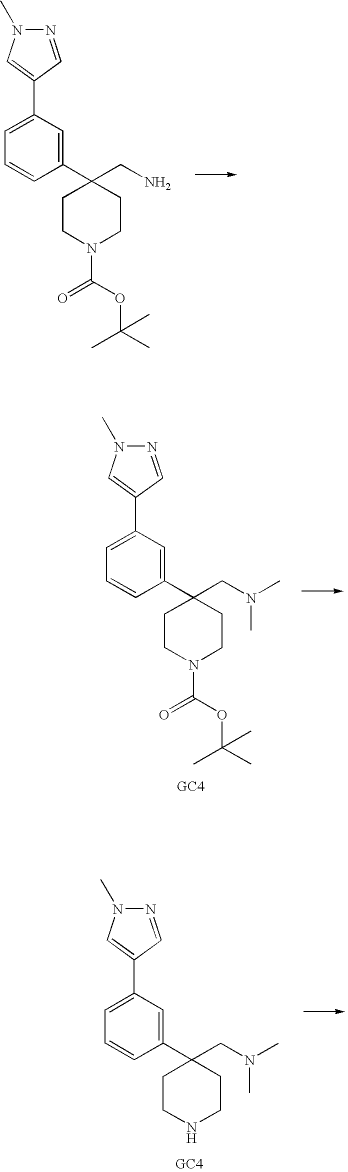

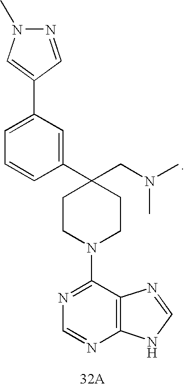

### (GC4). tert-Butyl 4-((dimethylamino)methyl)-4-(3-(1-methyl-1H-pyrazol-4-yl)phenyl)piperidine-1-carboxylate

Formaldehyde (37% aqueous solution) (2.010 mL, 26.99 mmol) was added to tert-butyl 4-(aminomethyl)-4-(3-(1-methyl-1H-pyrazol-4-yl)phenyl)piperidine-1-carboxylate (200 mg, 0.54 mmol (Preparation B2 above) and acetic acid (0.031 mL, 0.54 mmol) in. The resulting solution was stirred at ambient temperature for 10 minutes then sodium triacetoxyborohydride (343 mg, 1.62 mmol) added in one portion and the reaction mixture stirred at ambient temperature for 16 hours. The reaction mixture was concentrated and adjusted to pH 7 with saturated NaHCO3 then extracted with DCM (20 ml) and filtered through a phase transfer cup. Both the aqueous and organic layers were subjected to ion exchange chromatography, using an SCX column. The crude product was eluted from the column using 2M NH3/MeOH then purified by flash silica chromatography, elution gradient 0 to 5% MeOH in DCM. Pure fractions were evaporated to dryness to afford tert-butyl 4-((dimethylamino)methyl)-4-(3-(1-methyl-1H-pyrazol-4-yl)phenyl)piperidine-1-carboxylate (119 mg, 55.3%) as a colourless gum; Mass Spectrum: M+H⁺ 399.

### 32B. (GC5). N,N-dimethyl-1-(4-(3-(1-methyl-1H-pyrazol-4-yl)phenyl)piperidin-4-yl)methanamine

TFA (2 ml) was added to tert-butyl 4-((dimethylamino)methyl)-4-(3-(1-methyl-1H-pyrazol-4-yl)phenyl)piperidine-1-carboxylate (119 mg, 0.30 mmol) in DCM (2 mL). The resulting solution was stirred at ambient temperature for 1 hour. The reaction mixture was purified by ion exchange chromatography, using an SCX column. The desired product was eluted from the column using 2M NH3/MeOH and pure fractions were evaporated to dryness to afford N,N-dimethyl-1-(4-(3-(1-methyl-1H-pyrazol-4-yl)phenyl)piperidin-4-yl)methanamine (83 mg, 93%) as a colourless gum; Mass Spectrum: M+H⁺ 299.

### 32C. N,N-dimethyl-1-(4-(3-(1-methyl-1H-pyrazol-4-yl)phenyl)-1-(9H-purin-6-yl)piperidin-4-yl)methanamine

N,N-Dimethyl-1-(4-(3-(1-methyl-1H-pyrazol-4-yl)phenyl)piperidin-4-yl)methanamine (83 mg, 0.28 mmol), 6-chloropurine (45.1 mg, 0.29 mmol) and triethylamine (0.194 mL, 1.39 mmol) were dissolved in butan-1-ol (2 mL) and heated to 100° C. for 16 hours. The reaction mixture was cooled to ambient temperature and the solvents evaporated. The crude product was purified by preparative HPLC using decreasingly polar mixtures of water (containing 1% NH3) and MeCN as eluents. Fractions containing the desired compound were evaporated to dryness to afford the desired N,N-dimethyl-1-(4-(3-(1-methyl-1H-pyrazol-4-yl)phenyl)-1-(9H-purin-6-yl)piperidin-4-yl)methanamine (69 mg, 59.6%) as a white solid; NMR Spectrum: ¹H NMR (399.902 MHz, CDCl₃) δ 2.03 (8H, m), 2.39 (2H, m), 2.49 (2H, s), 3.68 (2H, m), 3.96 (3H, s), 4.95 (2H, m), 7.32 (3H, m), 7.51 (1H, s), 7.62 (1H, s), 7.76 (1H, m), 7.90 (1H, s), 8.34 (1H, s); Mass Spectrum: M+H⁺ 417.

### Example 33

### Preparation of 6-(4-(3-(1-methyl-1H-pyrazol-4-yl)phenyl)-4-(pyrrolidin-1-ylmethyl)piperidin-1-yl)-9H-purine

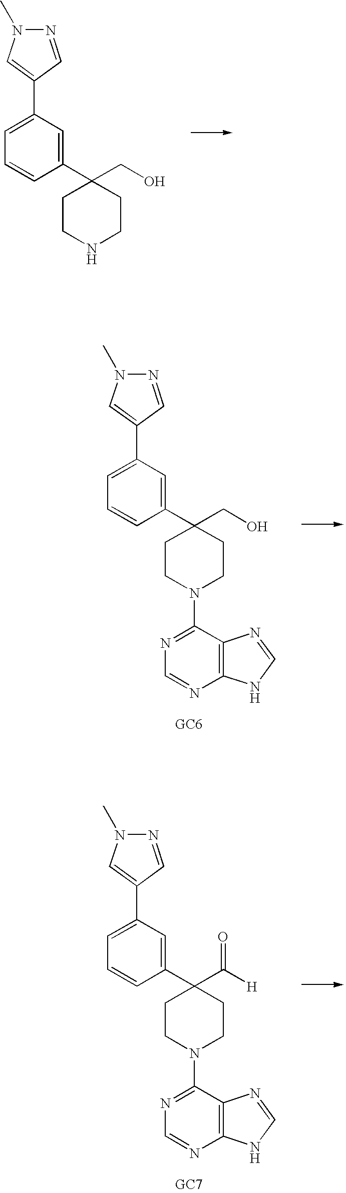

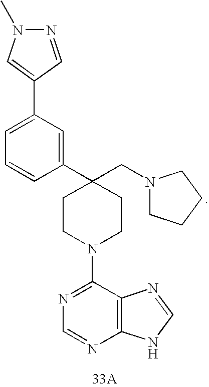

### GC6. (4-(3-(1-methyl-1H-pyrazol-4-yl)phenyl)-1-(9H-purin-6-yl)piperidin-4-yl)methanol

(4-(3-(1-Methyl-1H-pyrazol-4-yl)phenyl)piperidin-4-yl)methanol (454 mg, 1.67 mmol, (described in Preparation D above), 6-chloropurine (272 mg, 1.76 mmol) and triethylamine (1.166 mL, 8.37 mmol) were dissolved in butan-1-ol (15 mL) and heated to 100° C. for 16 hours. The reaction mixture was cooled then evaporated to dryness and redissolved in DCM and MeOH (50 mL), and washed with water (20 mL) and saturated brine (20 mL). The organic layer was dried over MgSO4, filtered and evaporated to the crude product. The crude gum was triturated with Et2O to give a solid which was collected by filtration and dried under high vacuum at 60° C. for 16 hours to give the title compound as a yellow solid (620 mg, 95%); NMR Spectrum: ¹H NMR (399.902 MHz, DMSO) δ 1.86 (2H, m), 2.13 (2H, m), 3.35 (2H, m), 3.45 (2H, m), 3.79 (3H, s), 4.56 (1H, m), 4.78 (2H, m), 7.20 (1H, m), 7.26 (1H, m), 7.33 (1H, m), 7.52 (1H, s), 7.80 (1H, s), 8.02 (1H, s), 8.10 (2H, m), 12.88 (1H, s); Mass Spectrum: M+H⁺ 390.

### 33B. (GC7). 4-(3-(1-methyl-1H-pyrazol-4-yl)phenyl)-1-(9H-purin-6-yl)piperidine-4-carbaldehyde

Dess-Martin Periodinane (692 mg, 1.63 mmol) was added in one portion to (4-(3-(1-methyl-1H-pyrazol-4-yl)phenyl)-1-(9H-purin-6-yl)piperidin-4-yl)methanol (489 mg, 1.26 mmol) in dichloromethane (10 mL) at room temperature. The resulting solution was stirred at room temperature for 16 hours. The reaction mixture was diluted with DCM (10 mL) and washed with 2M NaOH (25 mL). The aqueous layer was acidified with 2M HCl to pH 8 then extracted into DCM (2×50 ml) then the combined organics were dried over MgSO4, filtered and evaporated to afford crude product. Any insoluble material was dissolved in MeOH and added to the crude product then re-evaporated. The crude product was purified by flash silica chromatography, elution gradient 0 to 5% MeOH in DCM. Pure fractions were evaporated to dryness to afford 4-(3-(1-methyl-1H-pyrazol-4-yl)phenyl)-1-(9H-purin-6-yl)piperidine-4-carbaldehyde (144 mg, 29.6%) as a white solid; NMR Spectrum: ¹H NMR (399.902 MHz, DMSO) δ 1.99 (2H, m), 2.48 (2H, m), 3.55 (2H, m), 3.78 (3H, s), 4.89 (2H, m), 7.11 (1H, m), 7.31 (1H, m), 7.43 (1H, m), 7.47 (1H, m), 7.82 (1H, m), 8.05 (1H, s), 8.13 (2H, m), 9.52 (1H, s), 12.94 (1H, s); Mass Spectrum: M+H⁺ 388.

### 33C. 6-(4-(3-(1-methyl-1H-pyrazol-4-yl)phenyl)-4-(pyrrolidin-1-ylmethyl)piperidin-1-yl)-9H-purine

Pyrrolidine (0.415 mL, 4.97 mmol) was added to 4-(3-(1-methyl-1H-pyrazol-4-yl)phenyl)-1-(9H-purin-6-yl)piperidine-4-carbaldehyde (77 mg, 0.20 mmol) and dried 3 A molecular sieves in a mixture of MeOH (3 ml) and EtOH (3 ml). The resulting solution was stirred at ambient temperature for 16 hours. The solvents were evaporated then anhydrous DCM (5 ml) and anhydrous MeOH (5 ml) were added to the residues followed by Sodium Borohydride (22.56 mg, 0.60 mmol) and the mixture was stirred at ambient temperature for 3 hours. The reaction mixture was diluted with DCM (50 mL), and was acidified with acetic acid then added to an SCX column. The desired product was eluted from the column using 2M NH3/MeOH then combined with the evaporated organic layer and solvents re-evaporated to give the crude product. The crude product was purified by preparative HPLC using decreasingly polar mixtures of water (containing 1% NH3) and MeCN as eluents. Fractions containing the desired compound were evaporated to dryness to afford 6-(4-(3-(1-methyl-1H-pyrazol-4-yl)phenyl)-4-(pyrrolidin-1-ylmethyl)piperidin-1-yl)-9H-purine (56.0 mg, 63.7%) as a beige solid; NMR Spectrum: ¹H NMR (399.902 MHz, DMSO) δ 1.43 (4H, m), 1.85 (2H, m), 2.17 (6H, m), 2.63 (2H, s), 3.62 (2H, m), 3.79 (3H, s), 4.65 (2H, m), 7.19 (1H, m), 7.25 (1H, m), 7.32 (1H, m), 7.53 (1H, s), 7.80 (1H, s), 8.02 (1H, s), 8.11 (2H, m), 12.43 (1H, s); Mass Spectrum: M+H⁺ 443.

### Example 34

### Preparation of 3-(4-(3-(1-methyl-1H-pyrazol-4-yl)phenyl)-1-(9H-purin-6-yl)piperidin-4-yl)propan-1-amine

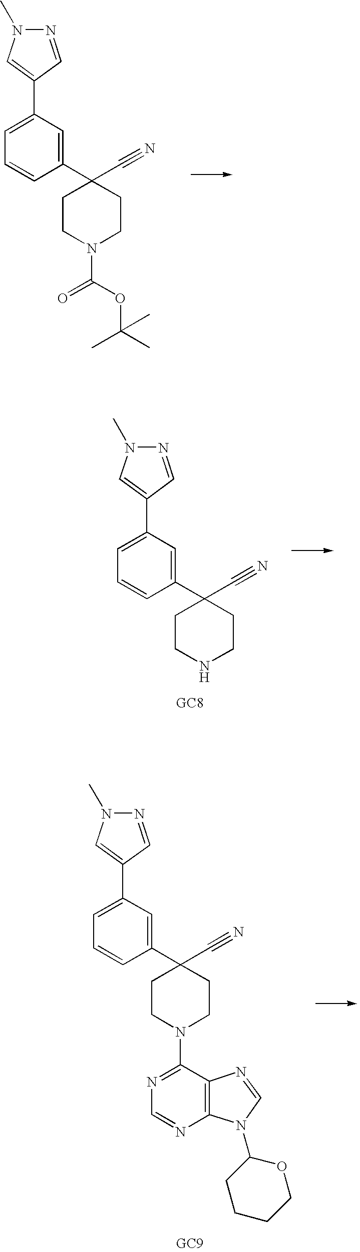

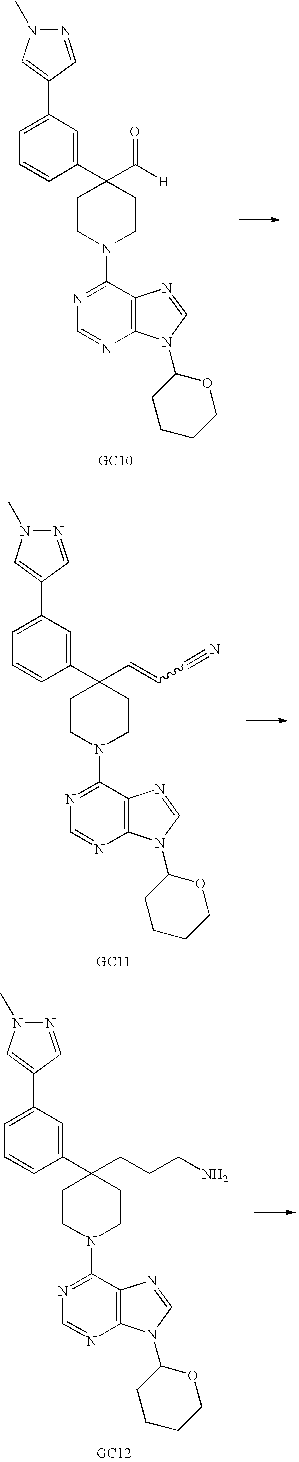

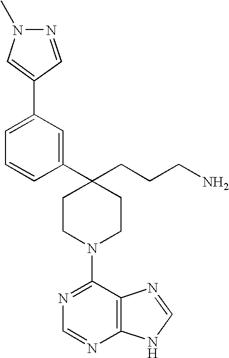

### 34A. GC8. 4-(3-(1-methyl-1H-pyrazol-4-yl)phenyl)piperidine-4-carbonitrile

TFA (5 ml) was added to tert-butyl 4-cyano-4-(3-(1-methyl-1H-pyrazol-4-yl)phenyl)piperidine-1-carboxylate (1 g, 2.73 mmol) (Preparation B1 above) in DCM (5 mL). The resulting solution was stirred at ambient temperature for 1 hour. The reaction mixture was purified by ion exchange chromatography, using an SCX column. The desired product was eluted from the column using 2M NH3/MeOH and pure fractions were evaporated to dryness to afford 4-(3-(1-methyl-1H-pyrazol-4-yl)phenyl)piperidine-4-carbonitrile (0.708 g, 97%) as a yellow gum; Mass Spectrum: M+H⁺ 308 (MeCN adduct).

### 34B. GC9. 4-(3-(1-methyl-1H-pyrazol-4-yl)phenyl)-1-(9-(tetrahydro-2H-pyran-2-yl)-9H-purin-6-yl)piperidine-4-carbonitrile

4-(3-(1-Methyl-1H-pyrazol-4-yl)phenyl)piperidine-4-carbonitrile (708 mg, 2.66 mmol), 6-chloro-9-(tetrahydro-2-pyranyl)purine (666 mg, 2.79 mmol) and triethylamine (1.853 mL, 13.29 mmol) were dissolved in butan-1-ol (10 mL) and heated to 100° C. for 16 hours. The reaction mixture was cooled to ambient temperature then evaporated to dryness and redissolved in DCM (20 mL) and washed with water (10 mL). The organic layer was evaporated to afford the crude product. The crude product was purified by flash silica chromatography on neutral silica, elution gradient 0 to 5% MeOH in DCM. Pure fractions were evaporated to dryness to afford 4-(3-(1-methyl-1H-pyrazol-4-yl)phenyl)-1-(9-(tetrahydro-2H-pyran-2-yl)-9H-purin-6-yl)piperidine-4-carbonitrile (938 mg, 75%) as a pale yellow solid; NMR Spectrum: ¹H NMR (399.902 MHz, DMSO) δ 1.53 (2H, m), 1.68 (1H, m), 1.89 (2H, m), 2.13 (3H, m), 2.24 (2H, m), 3.33 (2H, m), 3.63 (1H, m), 3.78 (3H, s), 3.96 (1H, m), 5.58 (3H, m), 7.34 (2H, m), 7.48 (1H, m), 7.64 (1H, s), 7.84 (1H, s), 8.13 (1H, s), 8.24 (1H, s), 8.35 (1H, s); Mass Spectrum: M+H⁺ 469.

### 34C. GC10. 4-(3-(1-methyl-1H-pyrazol-4-yl)phenyl)-1-(9-(tetrahydro-2H-pyran-2-yl)-9H-purin-6-yl)piperidine-4-carbaldehyde

A solution of diisobutylaluminum hydride (1M in toluene) (15.88 mL, 15.88 mmol) was added dropwise to a stirred solution of 4-(3-(1-methyl-1H-pyrazol-4-yl)phenyl)-1-(9-(tetrahydro-2H-pyran-2-yl)-9H-purin-6-yl)piperidine-4-carbonitrile (3.46 g, 7.38 mmol) in toluene (115 mL) at −78° C., over a period of 15 minutes under nitrogen. The resulting solution was stirred at −78° C. for 3 hours then −40° C. for 2 hrs. The reaction mixture was quenched with MeOH (50 mL) followed by saturated ammonium chloride (50 ml) and allowed to warm to room temperature overnight. The reaction mixture was filtered through celite washing with EtOAc (3×100 mL) and DCM (3×100 mL) then evaporated to dryness and redissolved in DCM (200 mL), then washed with water (200 mL), dried over magnesium sulfate and evaporated to give the crude product. The crude product was purified by flash silica chromatography, elution gradient 0 to 5% MeOH in DCM. Fractions containing product were evaporated to dryness to afford 4-(3-(1-methyl-1H-pyrazol-4-yl)phenyl)-1-(9-(tetrahydro-2H-pyran-2-yl)-9H-purin-6-yl)piperidine-4-carbaldehyde (1.880 g, 54.0%) as a white solid; Mass Spectrum: M+H⁺ 472.

### 34D. GC11. 3-(4-(3-(1-methyl-1H-pyrazol-4-yl)phenyl)-1-(9-(tetrahydro-2H-pyran-2-yl)-9H-purin-6-yl)piperidin-4-yl)acrylonitrile

A solution of cyanomethylphosphonic acid diethyl ester (103 mg, 0.58 mmol) in THF (2 mL) was added dropwise to a stirred suspension of sodium hydride (25.4 mg, 0.58 mmol) in THF (5 mL) at ambient temperature under nitrogen. The resulting solution was stirred at ambient temperature for 10 minutes then a solution of 4-(3-(1-methyl-1H-pyrazol-4-yl)phenyl)-1-(9-(tetrahydro-2H-pyran-2-yl)-9H-purin-6-yl)piperidine-4-carbaldehyde (250 mg, 0.53 mmol) in THF (2 mL) was added and the solution was stirred for 3 hours. The reaction mixture was quenched with MeOH (0.5 mL), evaporated to dryness and redissolved in DCM (20 mL) and washed with water (20 mL). The aqueous layer was re-extracted with DCM (20 mL) then the organic layers were combined and dried over MgSO4, filtered and evaporated to afford crude product. The crude product was purified by flash silica chromatography, elution gradient 40 to 60% EtOAc in DCM. Pure fractions were evaporated to dryness to afford 3-(4-(3-(1-methyl-1H-pyrazol-4-yl)phenyl)-1-(9-(tetrahydro-2H-pyran-2-yl)-9H-purin-6-yl)piperidin-4-yl)acrylonitrile (192 mg, 73.2%) as a white solid; NMR Spectrum: ¹H NMR (399.902 MHz, DMSO) δ 1.52 (2H, m), 1.66 (1H, m), 1.88 (2H, m), 2.15 (5H, m), 3.61 (1H, m), 3.79 (3H, s), 3.94 (1H, m), 4.04 (2H, m), 4.34 (2H, m), 5.59 (1H, m), 5.66 (1H, d), 6.99 (1H, d), 7.15 (1H, m), 7.29 (1H, m), 7.38 (1H, m), 7.50 (1H, m), 7.82 (1H, m), 8.11 (1H, s), 8.18 (1H, s), 8.30 (1H, s); Mass Spectrum: M+H⁺ 495.

### 34E. GC12. 3-(4-(3-(1-methyl-1H-pyrazol-4-yl)phenyl)-1-(9-(tetrahydro-2H-pyran-2-yl)-9H-purin-6-yl)piperidin-4-yl)propan-1-amine

3-(4-(3-(1-Methyl-1H-pyrazol-4-yl)phenyl)-1-(9-(tetrahydro-2H-pyran-2-yl)-9H-purin-6-yl)piperidin-4-yl)acrylonitrile (192 mg, 0.39 mmol) and Raney(R) Nickel 50% slurry in water (4 g, 23.34 mmol) in ethanol (50 mL) were stirred under an atmosphere of hydrogen at 1 bar and 25° C. for 1 hour.

The catalyst was filtered and washed with EtOH (20 mL) and the solvents evaporated to give the crude product. The crude product was purified by flash silica chromatography, elution gradient 0 to 20% 2M ammonia in MeOH in DCM. Fractions were evaporated to dryness to afford 3-(4-(3-(1-methyl-1H-pyrazol-4-yl)phenyl)-1-(9-(tetrahydro-2H-pyran-2-yl)-9H-purin-6-yl)piperidin-4-yl)propan-1-amine (118 mg, 60.7%) as a colourless gum; Mass Spectrum: M+H⁺ 501.

### 34F. 3-(4-(3-(1-methyl-1H-pyrazol-4-yl)phenyl)-1-(9H-purin-6-yl)piperidin-4-yl)propan-1-amine

TFA (1 mL) was added to 3-(4-(3-(1-methyl-1H-pyrazol-4-yl)phenyl)-1-(9-(tetrahydro-2H-pyran-2-yl)-9H-purin-6-yl)piperidin-4-yl)propan-1-amine (118 mg, 0.24 mmol) in DCM (1 mL). The resulting solution was stirred at ambient temperature for 1 hour then added to a SCX column. The crude product was eluted from the column using 2M NH3/MeOH and evaporated to dryness. The crude product was purified by preparative HPLC (Waters XBridge Prep C18 OBD column, 5μ silica, 19 mm diameter, 100 mm length), using decreasingly polar mixtures of water (containing 1% NH3) and MeCN as eluents. Fractions containing the desired compound were evaporated to dryness to afford 3-(4-(3-(1-methyl-1H-pyrazol-4-yl)phenyl)-1-(9H-purin-6-yl)piperidin-4-yl)propan-1-amine (55.0 mg, 56.0%) as a white solid; NMR Spectrum: ¹H NMR (399.902 MHz, DMSO) δ 0.95 (2H, m), 1.56 (2H, m), 1.75 (2H, m), 2.17 (2H, m), 2.29 (2H, m), 3.74 (5H, m), 4.53 (2H, m), 7.15 (1H, d), 7.26 (1H, m), 7.32 (1H, d), 7.48 (1H, s), 7.81 (1H, s), 8.00 (1H, s), 8.10 (2H, m) Purine NH and NH2 missing; Mass Spectrum: M+H⁺ 417.

### Example 35

### Preparation of 2-amino-N-((4-(3-(1-methyl-1H-pyrazol-4-yl)phenyl)-1-(9H-purin-6-yl)piperidin-4-yl)methyl)acetamide

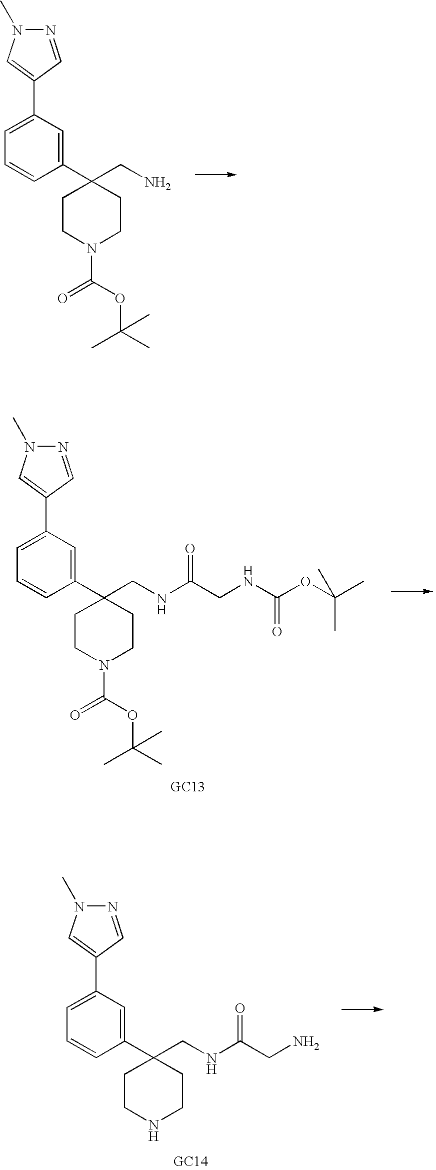

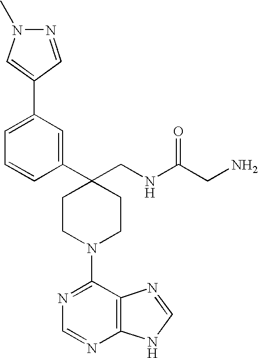

### 35A. GC13. tert-butyl 4-((2-(tert-butoxycarbonylamino)acetamido)methyl)-4-(3-(1-methyl-1H-pyrazol-4-yl)phenyl)piperidine-1-carboxylate

A solution of tert-butyl 4-(aminomethyl)-4-(3-(1-methyl-1H-pyrazol-4-yl)phenyl)piperidine-1-carboxylate (142 mg, 0.38 mmol) (described in Preparation B2 above) in DMF (1 mL) was added to a stirred solution of N-(tert-butoxycarbonyl)glycine (81 mg, 0.46 mmol), O-(7-azabenzotriazol-1-yl)-N,N,N′,N′-tetramethyluronium hexafluoro-phosphate (175 mg, 0.46 mmol) and N,N-diisopropylethylamine (0.200 mL, 1.15 mmol) in DMF (2 mL). The resulting solution was stirred at ambient temperature for 4 hours. The reaction mixture was evaporated to dryness and redissolved in EtOAc (25 mL), and washed sequentially with water (20 mL), saturated NaHCO3 (20 mL), and saturated brine (20 mL). The organic layer was dried over MgSO4, filtered and evaporated to afford tert-butyl 4-((2-(tert-butoxycarbonylamino)acetamido)methyl)-4-(3-(1-methyl-1H-pyrazol-4-yl)phenyl)piperidine-1-carboxylate (200 mg, 99%) as a yellow gum which was used without further purification; Mass Spectrum: M+H⁺ 528.

### 35B. GC14. 2-amino-N-((4-(3-(1-methyl-1H-pyrazol-4-yl)phenyl)piperidin-4-yl)methyl)acetamide

TFA (2 ml) was added to tert-butyl 4-((2-(tert-butoxycarbonylamino)acetamido)methyl)-4-(3-(1-methyl-1H-pyrazol-4-yl)phenyl)piperidine-1-carboxylate (200 mg, 0.38 mmol) in DCM (2 mL). The resulting solution was stirred at ambient temperature for 2 hours. The reaction mixture was purified by ion exchange chromatography, using an SCX column. The desired product was eluted from the column using 2M NH3/MeOH and pure fractions were evaporated to dryness to afford 2-amino-N-((4-(3-(1-methyl-1H-pyrazol-4-yl)phenyl)piperidin-4-yl)methyl)acetamide (92 mg, 74.1%) as a colourless gum; Mass Spectrum: M+H⁺ 328.

### 35C. 2-amino-N-((4-(3-(1-methyl-1H-pyrazol-4-yl)phenyl)-1-(9H-purin-6-yl)piperidin-4-yl)methyl)acetamide

2-Amino-N-((4-(3-(1-methyl-1H-pyrazol-4-yl)phenyl)piperidin-4-yl)methyl)acetamide (92 mg, 0.28 mmol), 6-chloropurine (45.6 mg, 0.30 mmol) and triethylamine (196 μl, 1.41 mmol) were dissolved in butan-1-ol (3 mL) and heated to 60° C. for 90 minutes. The reaction was cooled to ambient temperature and the solvents evaporated. The crude product was purified by preparative HPLC using decreasingly polar mixtures of water (containing 1% NH3) and MeCN as eluents. Fractions containing the desired compound were evaporated to dryness to afford the desired 2-amino-N-((4-(3-(1-methyl-1H-pyrazol-4-yl)phenyl)-1-(9H-purin-6-yl)piperidin-4-yl)methyl)acetamide (69 mg, 55.1%) as a white solid; NMR Spectrum: ¹H NMR (399.902 MHz, DMSO) δ 1.81 (2H, m), 2.16 (2H, m), 3.12 (2H, s), 3.33 (2H, d), 3.66 (2H, m), 3.81 (3H, s), 4.66 (2H, m), 7.21 (1H, m), 7.30 (1H, m), 7.38 (1H, m), 7.55 (1H, s), 7.59 (1H, t), 7.84 (1H, s), 8.03 (1H, s), 8.12 (2H, s) Purine NH and NH2 missing; Mass Spectrum: M+H⁺ 446.

### Example 36

### Preparation of 2-(dimethylamino)-N-((4-(3-(1-methyl-1H-pyrazol-4-yl)phenyl)-1-(9H-purin-6-yl)piperidin-4-yl)methyl)acetamide

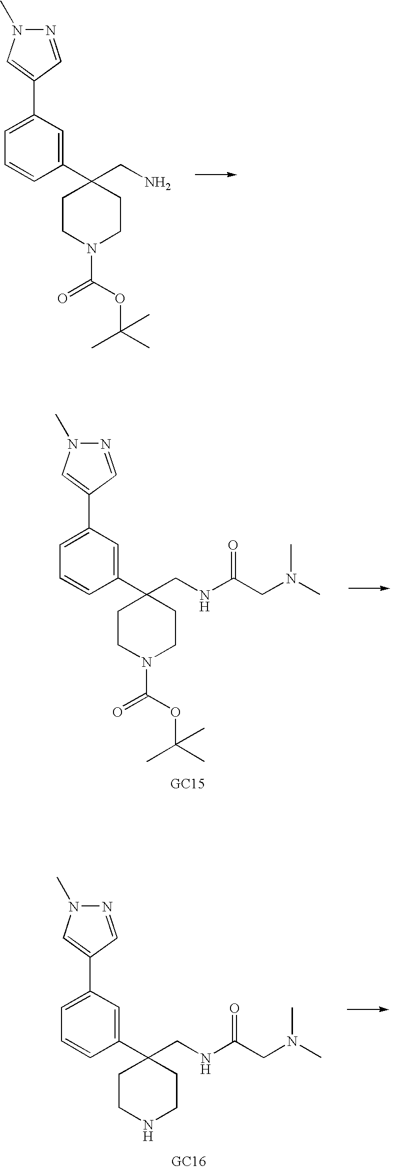

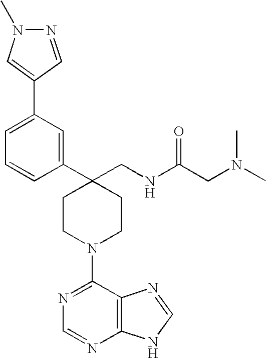

### 36A. GC15. tert-butyl 442-(dimethylamino)acetamido)methyl)-4-(3-(1-methyl-1H-pyrazol-4-yl)phenyl)piperidine-1-carboxylate

A solution of tert-butyl 4-(aminomethyl)-4-(3-(1-methyl-1H-pyrazol-4-yl)phenyl)piperidine-1-carboxylate (142 mg, 0.38 mmol) (described in Preparation B2 above) in DMF (1 mL) was added to a stirred solution of N,N-Dimethylglycine (47.4 Mg, 0.46 Mmol), O-(7-Azabenzotriazol-1-Yl)-N,N,N′,N′-Tetramethyluronium Hexafluoro-Phosphate (175 mg, 0.46 mmol) and N,N-Diisopropylethylamine (0.200 μl, 1.15 μmol) in DMF (2 mL). The resulting solution was stirred at ambient temperature for 4 hours. The reaction mixture was evaporated to dryness and redissolved in EtOAc (25 mL), and washed sequentially with water (20 mL), saturated NaHCO3 (20 mL), and saturated brine (20 mL). The organic layer was dried over MgSO4, filtered and evaporated to afford tert-butyl 4-((2-(dimethylamino)acetamido)methyl)-4-(3-(1-methyl-1H-pyrazol-4-yl)phenyl)piperidine-1-carboxylate (201 mg, 115%) as a yellow gum which was used without further purification; Mass Spectrum: M+H⁺ 456.

### 36B. GC16. 2-(dimethylamino)-N-((4-(3-(1-methyl-1H-pyrazol-4-yl)phenyl)piperidin-4-yl)methyl)acetamide

TFA (2 ml) was added to tert-butyl 4-((2-(dimethylamino)acetamido)methyl)-4-(3-(1-methyl-1H-pyrazol-4-yl)phenyl)piperidine-1-carboxylate (201 mg, 0.44 mmol) in DCM (2 mL). The resulting solution was stirred at ambient temperature for 2 hours. The reaction mixture was purified by ion exchange chromatography, using an SCX column. The desired product was eluted from the column using 2M NH3/MeOH and pure fractions were evaporated to dryness to afford 2-(dimethylamino)-N-((4-(3-(1-methyl-1H-pyrazol-4-yl)phenyl)piperidin-4-yl)methyl)acetamide (121 mg, 77%) as a colourless gum; Mass Spectrum: M+H⁺ 356.

### 36C. 2-(dimethylamino)-N-((4-(3-(1-methyl-1H-pyrazol-4-yl)phenyl)-1-(9H-purin-6-yl)piperidin-4-yl)methyl)acetamide

2-(Dimethylamino)-N-((4-(3-(1-methyl-1H-pyrazol-4-yl)phenyl)piperidin-4-yl)methyl)acetamide (121 mg, 0.34 mmol), 6-chloropurine (55.2 mg, 0.36 mmol) and triethylamine (237 μL, 1.70 mmol) were dissolved in butan-1-ol (3 mL) and heated to 100° C. for 90 minutes. The reaction was cooled to ambient temperature and the solvents evaporated. The crude product was purified by preparative HPLC using decreasingly polar mixtures of water (containing 1% NH3) and MeCN as eluents. Fractions containing the desired compound were evaporated to dryness to afford the desired 2-(dimethylamino)-N-((4-(3-(1-methyl-1H-pyrazol-4-yl)phenyl)-1-(9H-purin-6-yl)piperidin-4-yl)methyl)acetamide (22 mg, 13.65%) as a white solid; NMR Spectrum: ¹H NMR (399.902 MHz, DMSO) δ 1.79 (2H, m), 2.00 (6H, s), 2.14 (2H, m), 2.77 (2H, s), 3.32 (2H, m), 3.65 (2H, m), 3.80 (3H, s), 4.65 (2H, m), 7.19 (2H, m), 7.29 (1H, m), 7.37 (1H, m), 7.53 (1H, s), 7.83 (1H, s), 8.03 (1H, s), 8.12 (2H, m), 12.89 (1H, s); Mass Spectrum: M+H⁺ 474.

### Example 37

### Preparation of 4-[3-(1-methylpyrazol-4-yl)phenyl]-1-(9H-purin-6-yl)piperidine-4-carboxamide

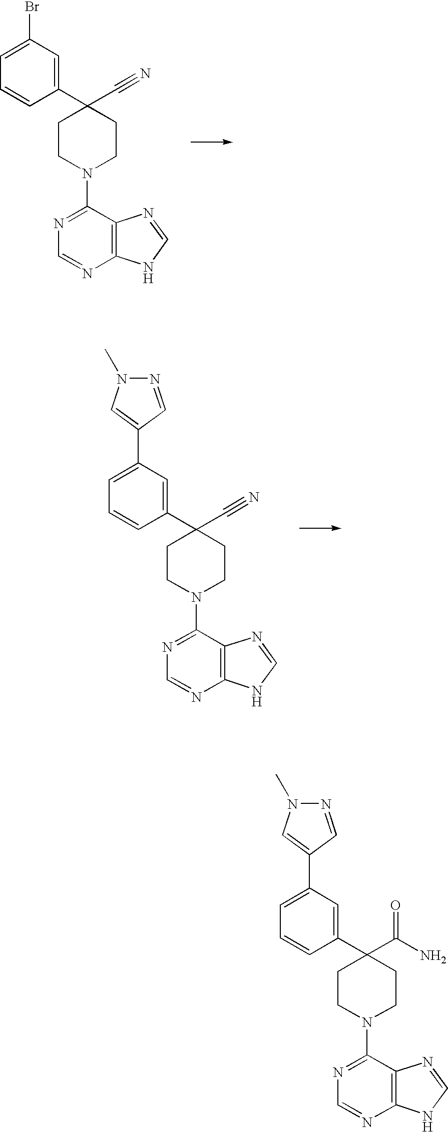

### 37A. GC17. 4-[3-(1-methylpyrazol-4-yl)phenyl]-1-(9H-purin-6-yl)piperidine-4-carbonitrile

4-(3-Bromophenyl)-1-(9H-purin-6-yl)piperidine-4-carbonitrile (300 mg, 0.783 mmol (prepared by a method similar to that described for 4-(3-chlorophenyl)-1-(9H-purin-6-yl)piperidine-4-carbonitrile in Preparation G1 above), 1-Methyl-4-(4,4,5,5-tetramethyl-1,3,2-dioxaborolan-2-yl)-1H-pyrazole (212 mg, 1.018 mmol) and tri-potassium orthophosphate (600 mg, 2.82 mmol) were suspended in a mixture of MeOH/EtOH/toluene/water (1:1:1:1, 8 ml) and bubbled with nitrogen for 5 minutes. Bis(tri-t-butylphosphine)-palladium(0) (24 mg, 0.047 mmol) was added and the mixture heated to 95° C. for 18 hours. The reaction mixture was cooled to room temperature then diluted with water (100 ml) and extracted with EtOAc (100 ml) then the organic layer was dried over MgSO4 and the solvents evaporated. The crude product was pre-absorbed onto silica and purified by flash silica chromatography eluting with a gradient of 0-5% MeOH in DCM. Fractions containing product were combined and evaporated to give 4-[3-(1-methylpyrazol-4-yl)phenyl]-1-(9H-purin-6-yl)piperidine-4-carbonitrile (123 mg, 41%) as a white solid; NMR Spectrum: ¹H NMR (399.902 MHz, DMSO) δ 2.09 (m, 2H), 2.25 (m, 2H), 3.31 (m, 2H), 3.78 (s, 3H), 5.61 (m, 2H), 7.34 (m, 2H), 7.48 (m, 1H), 7.64 (s, 1H), 7.84 (s, 1H), 8.09 (s, 1H), 8.13 (s, 1H), 8.20 (s, 1H), 13.00 (s, 1H); Mass Spectrum: M+H⁺ 385.

### 37B. 4-[3-(1-methylpyrazol-4-yl)phenyl]-1-(9H-purin-6-yl)piperidine-4-carboxamide

4-[3-(1-Methylpyrazol-4-yl)phenyl]-1-(9H-purin-6-yl)piperidine-4-carbonitrile (117 mg, 0.304 mmol) was added to dioxane (15 ml) and heated to 80° C. Water (5 ml) and 2M aqueous sodium hydroxide (900 μl) were added and the mixture heated to 100° C. for 20 hours. Further 2M aq sodium hydroxide (3 ml) was added and the mixture was left to stir at 100° C. for a further 72 hrs. The reaction mixture was cooled to room temperature and the solvents evaporated. The residues were suspended in water (10 ml), 2M aq sodium hydroxide (10 ml) was added and the insoluble material removed by filtration. The filtrate was acidified to pH 6 with 2M aq HCl and the resulting white precipitate was collected by filtration and dried to give the crude product, which was purified by preparative HPLC using decreasingly polar mixtures of water (containing 1% NH3) and MeCN as eluents. Fractions containing the desired compound were evaporated to dryness to afford 4-[3-(1-methylpyrazol-4-yl)phenyl]-1-(9H-purin-6-yl)piperidine-4-carboxamide (45 mg, 37%) as a white solid; NMR Spectrum: ¹H NMR (399.902 MHz, DMSO) δ 1.81 (2H, m), 2.55 (2H, m), 3.44 (2H, m), 3.79 (3H, s), 5.00 (2H, m), 6.99 (1H, s), 7.16 (1H, m), 7.23 (2H, m), 7.34 (1H, m), 7.50 (1H, s), 7.76 (1H, s), 8.02 (1H, s), 8.06 (1H, s), 8.12 (1H, s), 12.91 (1H, s); Mass Spectrum: M+H⁺ 403.

### Example 38

### 4-(aminomethyl)-N-[(6-chloropyridin-3-yl)methyl]-1-(7H-pyrrolo[2,3-d]pyrimidin-4-yl)piperidine-4-carboxamide

### 38A. tert-Butyl 4-((6-chloropyridin-3-yl)methylcarbamoyl)-4-cyanopiperidine-1-carboxylate

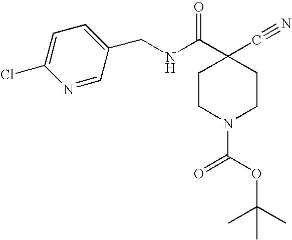

HATU (1.255 g, 3.30 mmol) was added in one portion to 1-(tert-butoxycarbonyl)-4-cyanopiperidine-4-carboxylic acid (0.763 g, 3 mmol), (6-chloropyridin-3-yl)methanamine (0.428 g, 3.00 mmol) and DIPEA (1.572 mL, 9.00 mmol) in DMA (20 mL) at 25° C. under nitrogen. The resulting solution was stirred at 60° C. for 4 hours. The reaction mixture was evaporated to dryness and redissolved in DCM (150 mL), and washed sequentially with citric acid (50 mL), water (50 mL), and saturated NaHCO3 (100 mL). The organic layer was dried over MgSO4, filtered and evaporated to afford crude product. The crude product thus obtained was concentrated, then purified by flash silica chromatography, elution gradient 0 to 100% EtOAc in isohexane. Pure fractions were evaporated to afford tert-butyl 4-((6-chloropyridin-3-yl)methylcarbamoyl)-4-cyanopiperidine-1-carboxylate (0.451 g, 39.7%) as a colourless gum which solidified on drying under high vacuum. Additionally, the deprotected amine, N-((6-chloropyridin-3-yl)methyl)-4-cyanopiperidine-4-carboxamide (0.240 g, 28.7%), was also recovered.

1H NMR (400.13 MHz, DMSO-d6) δ 1.41 (9H, s), 1.81-1.89 (2H, m), 2.05 (2H, d), 2.90-3.00 (2H, m), 3.98 (2H, d), 4.34 (2H, d), 7.49 (1H, d), 7.72-7.74 (1H, m), 8.32 (1H, d), 8.94 (1H, t).

MS m/e MH⁺ 277.

### 38B. N-((6-chloropyridin-3-yl)methyl)-4-cyanopiperidine-4-carboxamide

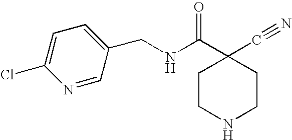

The title compound was isolated from the preparation of the Boc derivative of Example 38A. Deprotection of the Boc derivative can also be effected by reaction with hydrochloric acid.

1H NMR (400.13 MHz, DMSO-d6) δ 1.76-1.83 (2H, m), 1.93 (2H, d), 2.61-2.74 (2H, m), 2.91-2.98 (2H, m), 4.34 (2H, d), 7.49 (1H, d), 7.73 (1H, dd), 8.31 (1H, d), 8.87 (1H, t).

MS m/e MH⁺ 279.

### 38C. N-((6-Chloropyridin-3-yl)methyl)-4-cyano-1-(7H-pyrrolo[2,3-d]pyrimidin-4-yl)piperidine-4-carboxamide

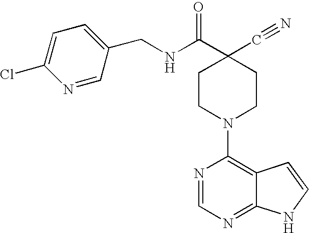

N-ethyldiisopropylamine (0.384 mL, 2.15 mmol) was added to N-((6-chloropyridin-3-yl)methyl)-4-cyanopiperidine-4-carboxamide (240 mg, 0.86 mmol) and 6-chloro-7-deazapurine (132 mg, 0.86 mmol) in DMA (20 mL) at 25° C. The resulting solution was stirred at 90° C. for 3 hours. The crude product was purified by ion exchange chromatography, using an SCX column. The desired product was eluted from the column using 7M NH3/MeOH and pure fractions were evaporated to dryness, to give N-((6-chloropyridin-3-yl)methyl)-4-cyano-1-(7H-pyrrolo[2,3-d]pyrimidin-4-yl)piperidine-4-carboxamide (66.0%). The product was used crude in the next step without further purification.

MS m/e MH⁺ 396.

### 38D. 4-(aminomethyl)-N-[(6-chloropyridin-3-yl)methyl]-1-(7H-pyrrolo[2,3-d]pyrimidin-4-yl)piperidine-4-carboxamide

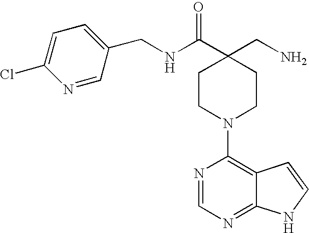

N-((6-chloropyridin-3-yl)methyl)-4-cyano-1-(7H-pyrrolo[2,3-d]pyrimidin-4-yl)piperidine-4-carboxamide (340 mg, 0.86 mmol), Raney nickel (200 mg, 2.33 mmol) and 2.0 N sodium hydroxide (3 mL, 6.00 mmol) were added to ethanol (30 mL). 7N ammonia/ethanol (10 mL) was added. This was placed under a balloon of hydrogen and stirred for 4 hours. The reaction mixture was filtered through celite and the solvent evaporated to dryness. The crude product was purified by flash silica chromatography, elution gradient 0 to 10% ammonia/MeOH in DCM. Pure fractions were evaporated to dryness to afford 4-(aminomethyl)-N-((6-chloropyridin-3-yl)methyl)-1-(7H-pyrrolo[2,3-d]pyrimidin-4-yl)piperidine-4-carboxamide (64.0 mg, 18.61%) as a colourless gum.

1H NMR (400.13 MHz, DMSO-d6) δ 1.45-1.52 (2H, m), 2.06-2.10 (2H, m), 2.68 (2H, s), 3.41 (2H, t), 4.23-4.29 (2H, m), 4.35 (2H, d), 6.55 (1H, d), 7.14-7.16 (1H, m), 7.46 (1H, d), 7.77 (1H, dd), 8.12 (1H, s), 8.35 (1H, d), 8.60 (1H, t), 11.62 (1H, s).

MS m/e MH⁺ 400.

### Examples 39 to 45

By using the HATU amide coupling procedure described in Method L above and the procedures described in Example 38, but with the appropriate heteroarylmethyl amine in place of (6-chloropyridin-3-yl)methanamine, the compounds of Examples 39 to 45 were prepared.

### Example 39

### 4-Amino-N-[(5-chlorothiophen-2-yl)methyl]-1-(7H-pyrrolo[2,3-d]pyrimidin-4-yl)piperidine-4-carboxamide

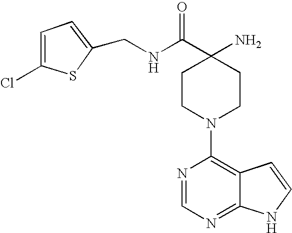

1H NMR (400.13 MHz, DMSO-d₆) δ 1.43 (2H, d), 1.93-2.00 (2H, m), 2.10 (2H, s), 3.51-3.58 (2H, m), 4.35-4.41 (4H, m), 6.58-6.59 (1H, m), 6.81 (1H, d), 6.92 (1H, d), 7.15-7.17 (1H, m), 8.13 (1H, s), 8.66 (1H, s), 11.63 (1H, s).

MS m/e MH⁺ 391.

### Example 40

### 4-Amino-N-[(4-methyl-1,3-thiazol-2-yl)methyl]-1-(7H-pyrrolo[2,3-d]pyrimidin-4-yl)piperidine-4-carboxamide

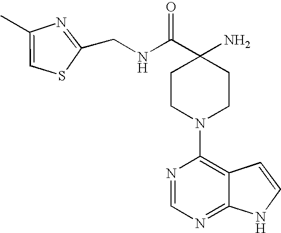

1H NMR (400.13 MHz, DMSO-d₆) δ 1.48 (2H, d), 1.96-2.03 (2H, m), 2.32 (3H, d), 3.55-3.62 (2H, m), 4.38-4.41 (2H, m), 4.50 (2H, s), 6.58-6.60 (1H, m), 7.10 (1H, d), 7.15-7.17 (1H, m), 8.13 (1H, s), 8.86 (1H, s), 11.63 (1H, s).

MS m/e MH⁺ 372.

### Example 41

### 4-Amino-1-(7H-pyrrolo[2,3-d]pyrimidin-4-yl)-N-(quinolin-3-ylmethyl)piperidine-4-carboxamide

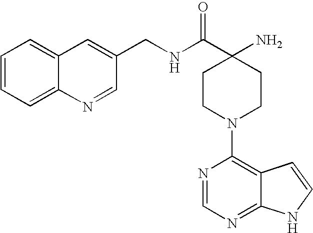

1H NMR (400.13 MHz, DMSO-d₆) δ 1.49 (2H, d), 1.97-2.04 (2H, m), 2.18 (2H, s), 3.53-3.60 (2H, m), 4.40-4.43 (2H, m), 4.50 (2H, d), 6.58-6.59 (1H, m), 7.15-7.16 (1H, m), 7.58-7.62 (1H, m), 7.70-7.75 (1H, m), 7.94-7.96 (1H, m), 8.00 (1H, d), 8.13 (1H, s), 8.14 (1H, d), 8.72 (1H, s), 8.84 (1H, d), 11.63 (1H, s).

MS m/e MH⁺ 402.

### Example 42

### 4-Amino-N-[(2-phenyl-1,3-thiazol-4-yl)methyl]-1-(7H-pyrrolo[2,3-d]pyrimidin-4-yl)piperidine-4-carboxamide

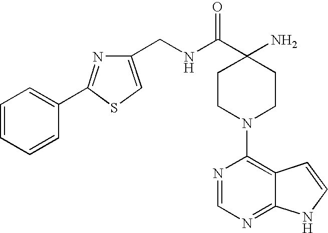

1H NMR (400.13 MHz, DMSO-d₆) δ 1.50 (2H, d), 1.98-2.06 (2H, m), 2.21 (2H, s), 3.54-3.61 (2H, m), 4.40 (1H, d), 4.44 (3H, d), 6.58-6.60 (1H, m), 7.15-7.17 (1H, m), 7.39 (1H, s), 7.48-7.52 (3H, m), 7.92-7.93 (2H, m), 8.13 (1H, s), 8.62 (1H, s), 11.63 (1H, s).

MS m/e MH⁺ 434.

### Example 43

### 4-Amino-N-(pyridin-4-ylmethyl)-1-(7H-pyrrolo[2,3-d]pyrimidin-4-yl)piperidine-4-carboxamide

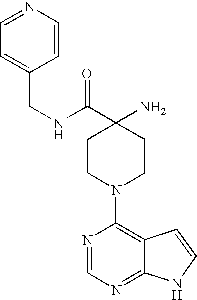

1H NMR (400.13 MHz, DMSO-d₆) δ 1.50 (2H, d), 1.96-2.03 (2H, m), 2.19 (2H, s), 3.53-3.60 (2H, m), 4.31 (2H, d), 4.40-4.43 (2H, m), 6.58-6.60 (1H, m), 7.15-7.17 (1H, m), 7.22-7.24 (2H, m), 8.13 (1H, s), 8.47-8.49 (2H, m), 8.62-8.68 (1H, m) 11.63 (1H, s).

MS m/e MH⁺ 352.

### Example 44

### 4-amino-N-[[3-(4-chlorophenyl)-1,2-oxazol-5-yl]methyl]-1-(7H-pyrrolo[2,3-d]pyrimidin-4-yl)piperidine-4-carboxamide

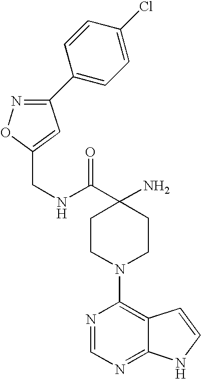

1H NMR (400.13 MHz, DMSO-d₆) δ 1.50 (2H, d), 1.95-2.03 (2H, m), 2.18 (2H, s), 3.54-3.61 (2H, m), 4.39-4.42 (2H, m), 4.47 (2H, s), 6.58-6.60 (1H, m), 6.83 (1H, s), 7.15-7.17 (1H, m), 7.57-7.59 (2H, m), 7.87-7.90 (2H, m), 8.13 (1H, s), 8.71 (1H, s), 11.63 (1H, s).

MS m/e MH⁺ 452.

### Example 45

### 4-amino-N-[(1-methylpyrazol-4-yl)methyl]-1-(7H-pyrrolo[2,3-d]pyrimidin-4-yl)piperidine-4-carboxamide

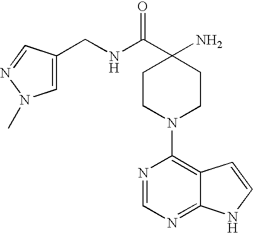

1H NMR (400.13 MHz, DMSO-d₆) δ 1.41 (2H, d), 1.93-2.00 (2H, m), 2.09 (2H, s), 3.49-3.56 (2H, m), 3.77 (3H, s), 4.09 (2H, d), 4.38-4.42 (2H, m), 6.57-6.58 (1H, m), 7.15-7.16 (1H, m), 7.29 (1H, s), 7.52 (1H, s), 8.13 (1H, s), 8.24 (1H, s), 11.62 (1H, s).

MS m/e MH⁺ 355.51.

### Example 46

### 4-Amino-1-(7H-pyrrolo[2,3-d]pyrimidin-4-yl)-piperidine-4-carboxylic acid (2-phenyl-thiazol-5-ylmethyl)-amide

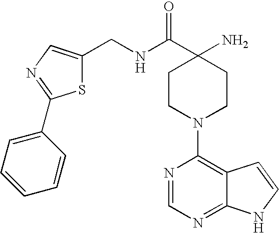

4-tent-Butoxycarbonylamino-1-(7H-pyrrolo[2,3-d]pyrimidin-4-yl)-piperidine-4-carboxylic acid (200 mg, 0.55 mmol) was dissolved in DMA (10 mL) and to the resulting solution were added HATU (228 mg, 0.60 mmol), C-(2-phenyl-thiazol-5-yl)-methylamine (114 mg, 0.60 mmol) and DIPEA (0.30 mL, 1.65 mmol). The reaction mixture was stirred overnight before being evaporated to dryness. The resulting gum was redissolved in acetonitrile (5 mL) and treated with 6.0 N HCl in propan-2-ol (5 mL) and heated at 60° C. for 2 hours. The reaction was quenched with 2.0 N NaOH (30 mL), extracted with DCM (3×30 mL), dried (MgSO4) and solvent removed in vacuo to yield a semi solid. The solid was triturated with hot acetonitrile to afford 4-amino-1-(7H-pyrrolo[2,3-d]pyrimidin-4-yl)-piperidine-4-carboxylic acid (2-phenyl-thiazol-5-ylmethyl)-amide (28 mg, 12%) as an off white solid which was filtered and dried. LC-MS m/z 432/434. 1H NMR (400.132 MHz, DMSO) δ 1.54-1.51 (m, 2H), 2.08-2.01 (m, 2H), 2.55-2.85 (vbrs, 2H), 3.61-3.55 (m, 2H), 4.45-4.41 (m, 4H), 6.60 (s, 1H), 7.16 (s, 1H), 7.39 (s, 1H), 7.53-7.46 (m, 4H), 7.94-7.91 (m, 2H), 8.14 (s, 1H), 8.64 (s, 1H).

### Example 47

### 4-Amino-1-(7H-pyrrolo[2,3-d]pyrimidin-4-yl)-piperidine-4-carboxylic acid (3-methyl-thiophen-2-ylmethyl)-amide

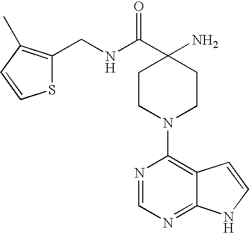

Following the method described in Example 46, but using C-(3-methyl-thiophen-2-yl)-methylamine in place of C-(2-phenyl-thiazol-5-yl)-methylamine, gave the title compound 4-amino-1-(7H-pyrrolo[2,3-d]pyrimidin-4-yl)-piperidine-4-carboxylic acid (3-methyl-thiophen-2-ylmethyl)-amide (65 mg, 32%). LC-MS m/z 369/371. 1H NMR (400.132 MHz, CDCl₃) δ 1.56-1.49 (m, 2H), 1.66 (brs, 2H), 2.22 (s, 3H), 2.41-2.35 (m, 2H), 3.61-3.67 (m, 2H), 4.61-4.46 (m, 4H), 6.51 (s, 1H), 6.81 (d, 1H), 7.12-7.08 (m, 2H), 7.87 (s, 1H), 8.34 (s, 1H), 10.48 (s, 1H).

### Example 48

### 4-Amino-1-(7H-pyrrolo[2,3-d]pyrimidin-4-yl)-piperidine-4-carboxylic acid (3-bromo-isoxazol-5-ylmethyl)-amide

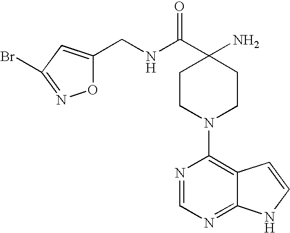

Following the method described in Example 46, but using C-(3-bromo-isoxazol-5-yl)-methylamine in place of C-(2-phenyl-thiazol-5-yl)-methylamine, gave the title compound 4-amino-1-(7H-pyrrolo[2,3-d]pyrimidin-4-yl)-piperidine-4-carboxylic acid (3-bromo-isoxazol-5-ylmethyl)-amide (60 mg, 26%). LC-MS m/z 419/421. 1H NMR (400.132 MHz, DMSO) δ 1.49-1.46 (m, 2H), 1.98-1.93 (m, 2H), 2.21 (brs, 2H), 3.59-3.53 (t, 2H), 4.43-4.37 (m, 4H), 6.59 (s, 2H), 7.16 (s, 1H), 8.13 (s, 1H), 8.70 (vbrs, 1H), 11.62 (s, 1H).

### Example 49

### 4-Amino-1-(7H-pyrrolo[2,3-d]pyrimidin-4-yl)-piperidine-4-carboxylic acid (3-methyl-isoxazol-5-ylmethyl)-amide

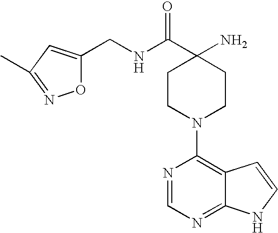

Following the method described in Example 46, but using C-(3-methyl-isoxazol-5-yl)-methylamine in place of C-(2-phenyl-thiazol-5-yl)-methylamine, gave the title compound (155 mg, 44%). LC-MS m/z 354/356. 1H NMR (400.132 MHz, DMSO) δ 1.48-1.44 (m, 2H), 1.96 (ddd, 2H), 2.15 (brs, 2H), 2.19 (s, 3H), 3.59-3.53 (m, 2H), 4.41-4.35 (m, 4H), 6.12 (s, 1H), 6.59-6.58 (m, 1H), 7.17-7.15 (m, 1H), 8.13 (s, 1H), 8.62 (s, 1H), 11.63 (s, 1H).

### Example 50

### 4-Amino-1-(7H-pyrrolo[2,3-d]pyrimidin-4-yl)-piperidine-4-carboxylic acid (1H-indol-2-ylmethyl)-amide

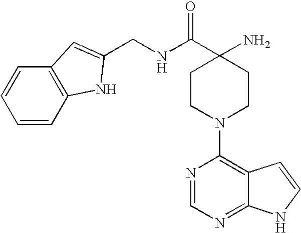

Following the method described in Example 46, but using C-(1H-indol-2-yl)-methylamine in place of C-(2-phenyl-thiazol-5-yl)-methylamine, gave the title compound (56 mg, 14%). LC-MS m/z 388/390. 1H NMR (400.132 MHz, DMSO) δ 1.43-1.40 (m, 2H), 2.02 (ddd, 2H), 2.17 (brs, 2H), 3.54-3.48 (m, 2H), 4.44-4.41 (m, 4H), 6.58-6.58 (m, 1H), 6.98 (t, 1H), 7.08 (t, 1H), 7.17-7.14 (m, 1H), 7.25 (s, 1H), 7.36 (d, 1H), 7.54 (d, 1H), 8.13 (s, 1H), 8.22 (t, 1H), 10.89 (s, 1H), 11.63 (s, 1H).

### Example 51

### (4-(3-(1-Methyl-1H-pyrazol-4-yl)phenyl)-1-(7H-pyrrolo[2,3-d]pyrimidin-4-yl)piperidin-4-yl)methanamine

### 51A. tert-Butyl 4-cyano-4-(3-(1-methyl-1H-pyrazol-4-yl)phenyl)piperidine-1-carboxylate

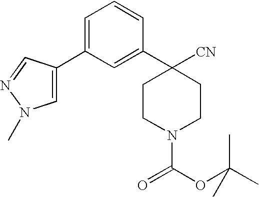

tert-butyl 4-(3-bromophenyl)-4-cyanopiperidine-1-carboxylate (1.000 g, 2.74 mmol), 1,1′ bis(diphenylphosphino)ferrocenedichloropalladium(II) (0.099 g, 0.14 mmol), 1-methyl-4-(4,4,5,5-Tetramethyl-1,3,2-dioxaborolan-2-yl)-1H pyrazole (0.854 g, 4.11 mmol) and Potassium phosphate, tribasic (2.325 g, 10.95 mmol) were dissolved in Dioxane (40 mL) and heated at 75° C. overnight. The reaction was evaporated to dryness, quenched with water (50 mL), extracted with diethyl ether (3×75 mL), the organic layer was dried over MgSO4, filtered and evaporated to afford black solid. The crude product was purified by flash silica chromatography, elution gradient 0 to 5% MeOH in DCM. Pure fractions were evaporated to dryness to afford tert-butyl 4-cyano-4-(3-(1-methyl-1H-pyrazol-4-yl)phenyl)piperidine-1-carboxylate (0.750 g, 74.8%) as a white solid; m/z (ESI+) (M+H)+=367; HPLC tR=2.34 min; 1H NMR (400.132 MHz, CDCl₃) δ 1.51 (9H, s), 2.04-1.98 (2H, m), 2.15-2.12 (2H, m), 3.31-3.17 (2H, m), 3.99 (3H, s), 4.44-4.24 (2H, m), 7.33 (1H, d), 7.42 (1H, t), 7.47 (1H, d), 7.58 (1H, s), 7.68 (1H, s), 7.80 (1H, s).

### 51B. tert-Butyl 4-(aminomethyl)-4-(3-(1-methyl-1H-pyrazol-4-yl)phenyl)piperidine-1-carboxylate

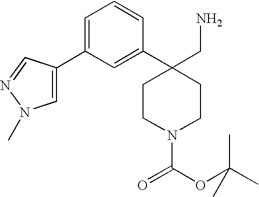

The product of Example 51A was subjected to hydrogenation over a platinum oxide catalyst and the resulting reaction mixture was filtered and the solvent evaporated to dryness to afford an orange gum. The reaction mixture was then quenched with 2M NaOH (15 mL), extracted with diethyl ether (3×100 mL), the organic layer was dried over MgSO4, filtered and evaporated to afford yellow gum. The crude product was purified by flash silica chromatography, elution gradient 0 to 10% MeOH in DCM. Pure fractions were evaporated to dryness to afford tert-butyl 4-(aminomethyl)-4-(3-(1-methyl-1H-pyrazol-4-yl)phenyl)piperidine-1-carboxylate (0.640 g, 84%) as a yellow gum; m/z (ESI+) (M+H)+=371; HPLC tR=1.84 min; 1H NMR (400.132 MHz, CDCl₃) δ 1.02 (2H, s), 1.44 (9H, s), 1.76-1.69 (2H, m), 2.21-2.18 (2H, m), 4.01 (2H, s), 3.14-3.07 (2H, m), 3.73 (2H, d), 3.94 (3H, s), 7.17-7.15 (1H, m), 7.38-7.34 (3H, m), 7.61 (1H, s), 7.73 (1H, s).

### 51C. (4-(3-(1-Methyl-1H-pyrazol-4-yl)phenyl)piperidin-4-yl)methanamine dihydrochloride

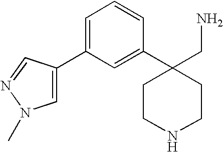

tert-Butyl 4-(aminomethyl)-4-(3-(1-methyl-1H-pyrazol-4-yl)phenyl)piperidine-1-carboxylate (0.640 g, 1.73 mmol) was dissolved in acetonitrile (15 mL). Hydrochloric acid (10 mL, 60.00 mmol) was added to the resulting solution and the reaction mixture was stirred for 1 hour. The precipitate was collected by filtration, washed with MeCN (20 mL) and air dried to afford (4-(3-(1-methyl-1H-pyrazol-4-yl)phenyl)piperidin-4-yl)methanamine dihydrochloride (0.490 g, 83%) as a white solid, which was used without further purification; m/z (ESI+) (M+H)+=271; HPLC tR=2.50 min.

### 51D. (4-(3-(1-Methyl-1H-pyrazol-4-yl)phenyl)-1-(7H-pyrrolo[2,3-d]pyrimidin-4-yl)piperidin-4-yl)methanamine

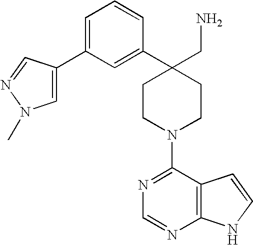

(4-(3-(1-Methyl-1H-pyrazol-4-yl)phenyl)piperidin-4-yl)methanamine dihydrochloride (0.200 g, 0.58 mmol), 6-chloro-7-deazapurine (0.098 g, 0.64 mmol) and N-ethyldiisopropylamine (0.403 mL, 2.33 mmol) were dissolved in DMF (25 mL) and heated at 80° C. overnight. The reaction mixture was evaporated to dryness and the crude product was purified by ion exchange chromatography, using an SCX column. The desired product was eluted from the column using 7M NH3/MeOH to afford a yellow foam. The crude product was purified by preparative HPLC using decreasingly polar mixtures of water (containing 1% AcOH) and MeCN as eluents. Fractions containing the desired compound were evaporated to dryness to afford (4-(3-(1-methyl-1H-pyrazol-4-yl)phenyl)-1-(7H-pyrrolo[2,3-d]pyrimidin-4-yl)piperidin-4-yl)methanamine (0.042 g, 18.61%) as a white foam; m/z (ESI+) (M+H)+=388; HPLC tR=1.53 min; 1H NMR (400.132 MHz, CDCl₃) δ 2.00-1.93 (2H, m), 2.43-2.39 (2H, m), 2.90 (2H, s), 3.66-3.60 (2H, m), 3.98 (3H, s), 4.39-4.35 (2H, m), 6.54 (1H, d), 7.07 (1H, d), 7.28-7.26 (1H, m), 7.43-7.36 (2H, m), 7.48 (1H, s), 7.64 (1H, s), 7.78 (1H, s), 8.33 (1H, s).

### Example 52

### 4-Amino-1-(7H-pyrrolo[2,3-d]pyrimidin-4-yl)-piperidine-4-carboxylic acid (3-benzooxazol-2-yl-phenyl)-amide

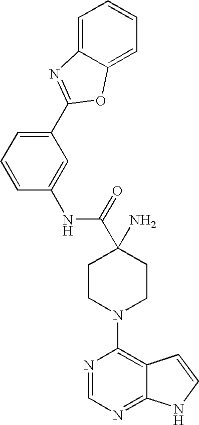

Intermediate 2B and 6-chloro-7-deaza purine were coupled as described in Example 2E. Purification was carried out using a silica Biotage column eluting with 0-15% MeOH/DCM.

1H NMR (400 MHz, Me-d3-OD): 8.61 (1H, t), 8.18 (1H, s), 8.04-8.00 (1H, m), 7.87-7.83 (1H, m), 7.78-7.74 (1H, m), 7.74-7.69 (1H, m), 7.57 (1H, t), 7.47-7.41 (2H, m), 7.16 (1H, d), 6.68 (1H, d), 4.64-4.56 (2H, m), 3.73 (2H, t), 2.39-2.32 (2H, m), 1.73 (2H, d). M/z: 454

### Example 53

### 4-Amino-1-(7H-pyrrolo[2,3-d]pyrimidin-4-yl)-piperidine-4-carboxylic acid [3-(5-fluoro-pyrimidin-2-yl)-phenyl]-amide

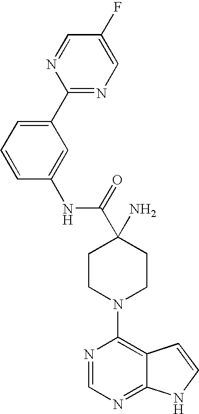

The product of preparation Q was reacted according to Examples 2A, 2B and 2E but replacing 6-chloro-7,9-dihydro-purin-8-one with 6-chloro-7-deaza purine.

1H NMR (400 MHz, DMSO-d6): 11.68 (1H, s), 8.98 (2H, s), 8.70 (1H, s), 8.15 (1H, s), 8.03 (1H, d), 7.45 (1H, t), 7.17 (1H, t), 6.62 (1H, s), 4.45 (2H, d), 3.60 (2H, t), 2.03-2.12 (2H, m), 1.57 (2H, d). M/z: 433

### Example 54

### 4-Amino-1-(7H-pyrrolo[2,3-d]pyrimidin-4-yl)-piperidine-4-carboxylic acid [3-(4-methyl-pyridin-2-yl)-phenyl]-amide

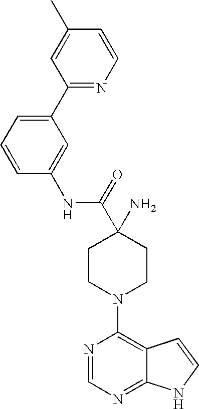

Intermediate 3A and 6-chloro-7-deaza purine were coupled as described in Example 2E. Purification using silica Biotage column eluting with 0-8% MeOH/DCM was required

1H NMR (400 MHz, Me-d3-OD): 8.50-8.42 (1H, m), 8.21-8.14 (2H, m), 7.76-7.64 (3H, m), 7.47 (1H, t), 7.23 (1H, d), 7.16 (1H, d), 6.67 (1H, d), 4.66-4.54 (2H, m), 3.77-3.63 (2H, m), 2.51-2.42 (3H, m), 2.42-2.28 (2H, m), 1.72 (2H, d). M/z: 428

### Example 55

### 4-Amino-1-(7H-pyrrolo[2,3-d]pyrimidin-4-yl)-piperidine-4-carboxylic acid [3-(4,4-dimethyl-piperidin-1-yl)-phenyl]-amide

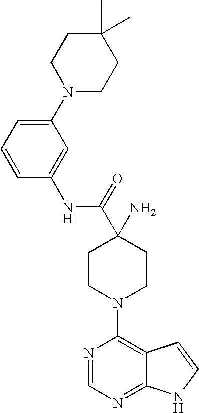

The product of preparation R was reacted according to Example 2E but replacing 6-chloro-7,9-dihydro-purin-8-one with 6-chloro-7-deaza purine.

1H NMR (400 MHz, DMSO-d6): 11.67 (1H, s), 8.15 (1H, s), 7.30 (1H, s), 7.18 (1H, s), 7.14-7.00 (2H, m), 6.62 (2H, d), 4.47 (2H, d), 4.09 (1H, q), 3.56 (2H, t), 3.18 (2H, d), 3.12 (4H, t), 2.17-1.96 (2H, m), 1.53 (2H, d), 1.42 (4H, t), 0.95 (6H, s). M/z: 448

### Example 56

### 4-Amino-1-(9H-purin-6-yl)-piperidine-4-carboxylic acid [3-(4,4-dimethyl-piperidin-1-yl)-phenyl]-amide

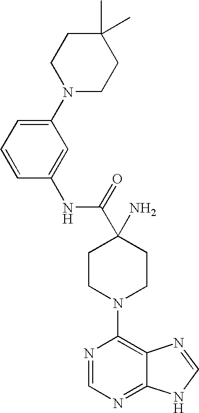

The product of preparation R was reacted according to Example 2E but replacing 6-chloro-7,9-dihydro-purin-8-one with 6-chloropurine.

1H NMR (400 MHz, DMSO-d6): 8.21 (1H, s), 8.11 (1H, s), 7.30 (1H, s), 7.15-7.01 (2H, m), 6.64 (1H, d), 5.30-4.97 (2H, br s), 4.09 (1H, q), 3.60 (2H, br s), 3.18 (2H, d), 3.12 (4H, t), 2.10-1.95 (2H, m), 1.53 (2H, d), 1.42 (4H, t), 0.95 (6H, s). M/z: 449

### Example 57

### 4-Amino-1-(9H-purin-6-yl)-piperidine-4-carboxylic acid (3-benzooxazol-2-yl-phenyl)-amide

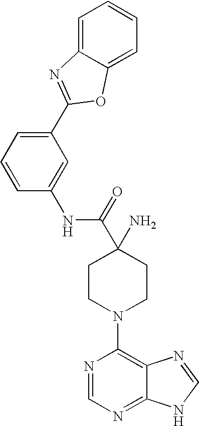

Intermediate 2B and 6-chloropurine were coupled as described in Example 2E. Purification was carried out using a silica Biotage column eluting with DMAW90.

1H NMR (400 MHz, DMSO-d6): 13.06-12.97 (1H, m), 8.71 (1H, t), 8.23 (1H, s), 8.12 (1H, s), 7.95-7.84 (2H, m), 7.84-7.76 (2H, m), 7.56 (1H, t), 7.49-7.39 (2H, m), 3.68 (2H, s), 2.14-2.01 (2H, m), 1.91 (2H, s), 1.61 (2H, d). M/z: 455

### Example 58

### 4-(Aminomethyl)-N-(4-methylthiazol-2-yl)-1-(7H-pyrrolo[2,3-d]pyrimidin-4-yl)piperidine-4-carboxamide

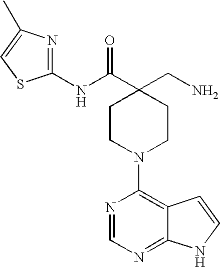

HATU (111 mg, 0.29 mmol) was added in one portion to 4-((tert-butoxycarbonyl-amino)methyl)-1-(7H-pyrrolo[2,3-d]pyrimidin-4-yl)piperidine-4-carboxylic acid (100 mg, 0.27 mmol), 4-methylthiazol-2-amine (30.4 mg, 0.27 mmol) and di-isopropylethylamine (140 μl, 0.80 mmol) in DMA (666 μl) at 25° C. under nitrogen. The resulting solution was stirred at 60° C. for 1 hour. The reaction mixture was diluted with EtOAc (200 mL), and washed sequentially with water (2×100 mL) and saturated brine (75 mL). The organic layer was dried over magnesium sulphate, filtered and evaporated to afford crude product. This was then dissolved in DCM (666 μl) and trifluoroacetic acid (152 mg, 1.33 mmol) added and the reaction stirred at room temperature for 6 hours. The solvent was removed under reduced pressure to give crude product. The crude product was purified by preparative HPLC (Waters XBridge Prep C18 OBD column, 5μ silica, 19 mm diameter, 100 mm length), using decreasingly polar mixtures of water (containing 0.1% TFA) and MeCN as eluents. Fractions containing the desired compound were evaporated to dryness and the gum obtained dissolved in dichloromethane (5 ml) and basified with sodium bicarbonate (5 ml). The organic phase was collected, dried over magnesium sulphate, filtered and concentrated to afford 4-(aminomethyl)-N-(4-methylthiazol-2-yl)-1-(7H-pyrrolo[2,3-d]pyrimidin-4-yl)piperidine-4-carboxamide (7.00 mg, 7.0%) as a gum.

1H NMR (400.132 MHz, DMSO) δ 1.54-1.59 (2H, m), 2.11-2.17 (2H, m), 2.87 (3H, s), 3.17 (2H, d), 3.64-3.69 (2H, m), 4.15-4.19 (2H, m), 6.58-6.59 (1H, m), 6.72 (1H, s), 7.17 (1H, t), 8.13 (1H, s), 11.69 (1H, s)

MS m/e MH⁺ 372

### Example 59

### 4-(aminomethyl)-N-(5-methylthiazol-2-yl)-1-(7H-pyrrolo[2,3-d]pyrimidin-4-yl)piperidine-4-carboxamide

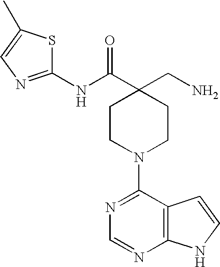

HATU (334 mg, 0.88 mmol) was added in one portion to 4-((tert-butoxycarbonylamino)methyl)-1-(7H-pyrrolo[2,3-d]pyrimidin-4-yl)piperidine-4-carboxylic acid (300 mg, 0.80 mmol) and DIPEA (0.419 ml, 2.40 mmol) in DMA (5 ml) and the reaction mixture was stirred at room temperature for 10 minutes. 5-methylthiazol-2-amine (91 mg, 0.80 mmol) was added and the resulting solution was stirred at 50° C. for 24 hours. The crude product was purified by ion exchange chromatography, using an SCX column. The desired product was eluted from the column using 7M NH3/MeOH and pure fractions were evaporated to dryness to afford tert-butyl (4-(5-methylthiazol-2-ylcarbamoyl)-1-(7H-pyrrolo[2,3-d]pyrimidin-4-yl)piperidin-4-yl)methylcarbamate. This material was then dissolved in DCM (5.00 ml) and trifluoroacetic acid (0.616 ml, 7.99 mmol) added. The reaction was stirred at room temperature for 2 hours. The crude product was purified by ion exchange chromatography, using an SCX column. The desired product was eluted from the column using 7M NH3/MeOH and pure fractions were evaporated to dryness. The crude product was purified by preparative HPLC (Waters XBridge Prep C18 OBD column, 5μ silica, 19 mm diameter, 100 mm length), using decreasingly polar mixtures of water (containing 1% NH3) and MeCN as eluents. Fractions containing the desired compound were evaporated to dryness to afford 4-(aminomethyl)-N-(5-methylthiazol-2-yl)-1-(7H-pyrrolo[2,3-d]pyrimidin-4-yl)piperidine-4-carboxamide (117 mg, 39.4%) as a white solid.

1H NMR (400.13 MHz, DMSO-d6) δ 1.53-1.60 (2H, m), 2.08-2.14 (2H, m), 2.33 (3H, d), 2.87 (2H, s), 3.65-3.71 (2H, m), 4.12-4.18 (2H, m), 5.95 (2H, s), 6.58-6.59 (1H, m), 7.11 (1H, d), 7.16-7.18 (1H, m), 8.13 (1H, s), 11.68 (1H, s)

MS m/e MH⁺ 372

### Example 60

### 4-(aminomethyl)-N-(5-fluoropyridin-2-yl)-1-(7H-pyrrolo[2,3-d]pyrimidin-4-yl)piperidine-4-carboxamide

### 60A. tert-butyl 4-cyano-4-(5-fluoropyridin-2-ylcarbamoyl)piperidine-1-carboxylate

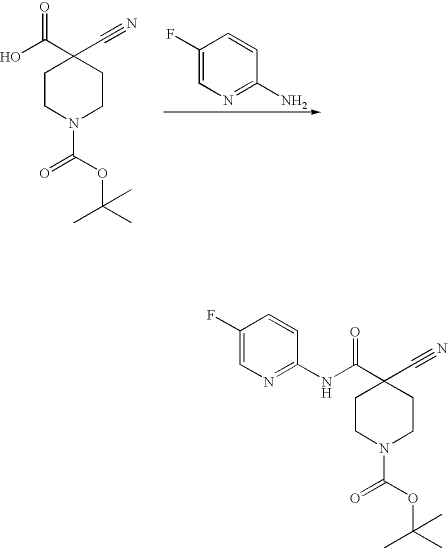

HATU (1121 mg, 2.95 mmol) was added in one portion to 1-(tert-butoxycarbonyl)-4-cyanopiperidine-4-carboxylic acid (Example 26A) (500 mg, 1.97 mmol) and DIPEA (1.030 ml, 5.90 mmol) in DMA (5 ml) at 20° C. under nitrogen. The resulting solution was stirred at 20° C. for 10 minutes then 2-Amino-5-fluoropyridine (265 mg, 2.36 mmol) added. The reaction mixture was stirred at 50° C. for 6 hours then at 20° C. for 18 hours. The reaction mixture was diluted with EtOAc (50 mL), and washed sequentially with water (2×50 mL) and saturated brine (50 mL). The organic layer was dried over MgSO₄, filtered and evaporated to afford crude product. The crude product was purified by flash silica chromatography, elution gradient 10 to 50% EtOAc in isohexane. Pure fractions were evaporated to dryness to afford and triturated with diethyl ether to afford tert-butyl 4-cyano-4-(5-fluoropyridin-2-ylcarbamoyl)piperidine-1-carboxylate (611 mg, 89%) as a white solid.

1H NMR (400.13 MHz, DMSO-d6) δ 1.42 (9H, s), 1.97-2.05 (2H, m), 2.21-2.24 (2H, m), 2.97 (2H, s), 4.01-4.05 (2H, m), 7.78-7.83 (1H, m), 8.00-8.03 (1H, m), 8.41 (1H, d), 10.98 (1H, s)

MS m/e M−H 347

### 60B. tert-Butyl 4-(aminomethyl)-4-(5-fluoropyridin-2-ylcarbamoyl)piperidine-1-carboxylate

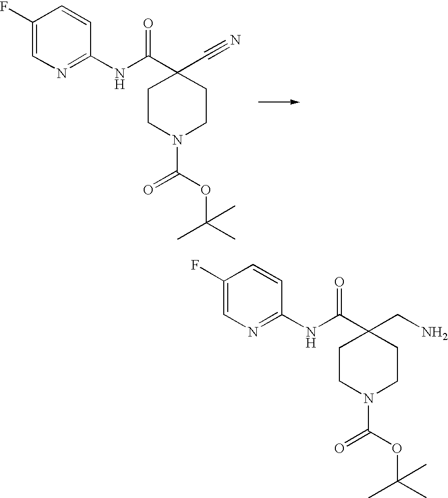

Platinum(iv) oxide (39.8 mg, 0.18 mmol) and tert-butyl 4-cyano-4-(5-fluoropyridin-2-ylcarbamoyl)piperidine-1-carboxylate (611 mg, 1.75 mmol) in acetic Acid (10 ml) were stirred under an atmosphere of hydrogen at 1 atm and 25° C. for 1 day. The reaction mixture was filtered through Celite and the crude product was purified by ion exchange chromatography, using an SCX column. The desired product was eluted from the column using 7M NH3/MeOH and pure fractions were evaporated to dryness to afford tert-butyl 4-(aminomethyl)-4-(5-fluoropyridin-2-ylcarbamoyl)piperidine-1-carboxylate (600 mg, 97%) as a colourless gum.

MS m/e MH⁺ 353

### 60C. 4-(Aminomethyl)-N-(5-fluoropyridin-2-yl)piperidine-4-carboxamide

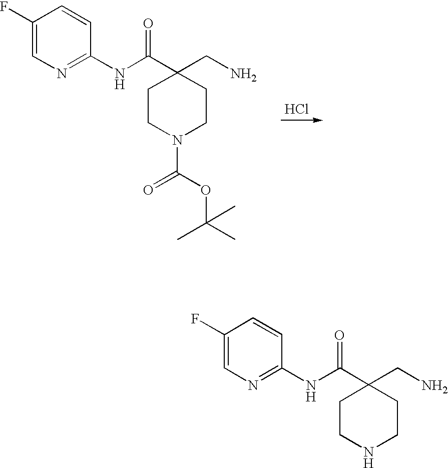

Hydrogen chloride 4M in dioxane (2.128 ml, 8.51 mmol) was added to tert-butyl 4-(aminomethyl)-4-(5-fluoropyridin-2-ylcarbamoyl)piperidine-1-carboxylate (600 mg, 1.70 mmol) in dioxane (5 ml). The resulting solution was stirred at 20° C. for 3 hours. The crude product was purified by ion exchange chromatography, using an SCX column. The desired product was eluted from the column using 7M NH3/MeOH and pure fractions were evaporated to dryness to afford 4-(aminomethyl)-N-(5-fluoropyridin-2-yl)piperidine-4-carboxamide (330 mg, 77%) as a colourless gum.

MS m/e M−H 251

### 60D. 4-(Aminomethyl)-N-(5-fluoropyridin-2-yl)-1-(7H-pyrrolo[2,3-d]pyrimidin-4-yl)piperidine-4-carboxamide

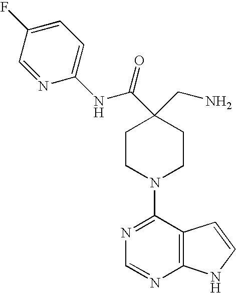

N-Ethyldiisopropylamine (0.684 ml, 3.92 mmol) was added to 4-(aminomethyl)-N-(5-fluoropyridin-2-yl)piperidine-4-carboxamide (330 mg, 1.31 mmol) and 4-chloro-7H-pyrrolo[2,3-d]pyrimidine (241 mg, 1.57 mmol) in DMA (5 ml). The resulting solution was stirred at 60° C. for 18 hours. The crude product was purified by ion exchange chromatography, using an SCX column. The desired product was eluted from the column using 7M NH3/MeOH and product containing fractions were evaporated to dryness. The impure product was dissolved in DMF (2 ml) and a solid precipitated out which was collected by filtration, washed with methanol and vacuum oven dried at 40° C. overnight to give 4-(aminomethyl)-N-(5-fluoropyridin-2-yl)-1-(7H-pyrrolo[2,3-d]pyrimidin-4-yl)piperidine-4-carboxamide (65.0 mg, 13.4%) as a white solid.

1H NMR (400.13 MHz, DMSO-d6) δ 1.51-1.58 (2H, m), 2.08-2.12 (2H, m), 2.87 (2H, s), 3.78-3.80 (2H, m), 4.09-4.11 (2H, m), 6.58-6.59 (1H, m), 7.16-7.18 (1H, m), 7.70-7.75 (1H, m), 8.12-8.15 (2H, m), 8.29 (1H, d), 11.67 (1H, s)

MS m/e MH⁺ 370

### Example 61

### 4-(Aminomethyl)-1-(7H-pyrrolo[2,3-d]pyrimidin-4-yl)-N-(5-(trifluoromethyl)pyridin-2-yl)piperidine-4-carboxamide

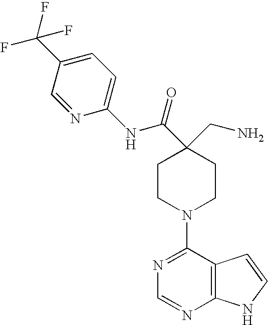

### 61A. tert-Butyl 4-cyano-4-(5-(trifluoromethyl)pyridin-2-ylcarbamoyl)piperidine-1-carboxylate

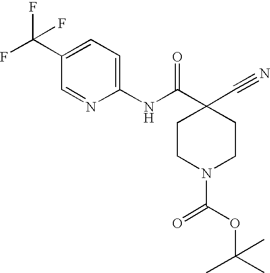

HATU (1121 mg, 2.95 mmol) was added in one portion to 1-(tert-butoxycarbonyl)-4-cyanopiperidine-4-carboxylic acid (Example 26A) (500 mg, 1.97 mmol) and DIPEA (1.030 ml, 5.90 mmol) in DMA (5 ml) at 20° C. under nitrogen. The resulting solution was stirred at 20° C. for 10 minutes then 2-amino-5-(trifluoromethyl)pyridine (383 mg, 2.36 mmol) added. The reaction mixture was stirred at 50° C. for 6 hours then at 20° C. for 18 hours. The reaction mixture was diluted with EtOAc (50 mL), and washed sequentially with water (2×50 mL) and saturated brine (50 mL). The organic layer was dried over MgSO4, filtered and evaporated to afford crude product. The crude product was purified by flash silica chromatography, elution gradient 20 to 40% EtOAc in isohexane. Pure fractions were evaporated to dryness to afford and triturated with diethyl ether to afford tert-butyl 4-cyano-4-(5-(trifluoromethyl)pyridin-2-ylcarbamoyl)piperidine-1-carboxylate (554 mg, 70.7%) as a white solid.

1H NMR (400.13 MHz, DMSO-d6) δ 1.42 (9H, s), 2.00-2.07 (2H, m), 2.25 (2H, d), 2.97 (2H, s), 4.04 (2H, d), 8.19-8.27 (2H, m), 8.80 (1H, t), 11.38 (1H, s)

MS m/e M−H 397

### 61B. tert-Butyl 4-(aminomethyl)-4-(5-(trifluoromethyl)pyridin-2-ylcarbamoyl)piperidine-1-carboxylate

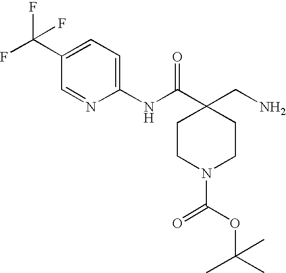

Platinum(IV) oxide (31.6 mg, 0.14 mmol) and tert-butyl 4-cyano-4-(5-(trifluoromethyl)pyridin-2-ylcarbamoyl)piperidine-1-carboxylate (554 mg, 1.39 mmol) in acetic acid (20 ml) were stirred under an atmosphere of hydrogen at 1 atm and 25° C. for 1 day. The reaction mixture was filtered through celite and the crude product was purified by ion exchange chromatography, using an SCX column. The desired product was eluted from the column using 7M NH3/MeOH and pure fractions were evaporated to dryness to afford tert-butyl 4-(aminomethyl)-4-(5-(trifluoromethyl)pyridin-2-ylcarbamoyl)piperidine-1-carboxylate (450 mg, 80%) as a colourless gum.

MS m/e M−H 401

### 61C. 4-(Aminomethyl)-N-(5-(trifluoromethyl)pyridin-2-yl)piperidine-4-carboxamide

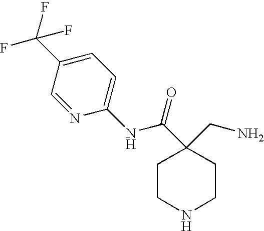

Hydrogen chloride 4M in dioxane (1.367 ml, 5.47 mmol) was added to tert-butyl 4-(aminomethyl)-4-(5-(trifluoromethyl)pyridin-2-ylcarbamoyl)piperidine-1-carboxylate (440 mg, 1.09 mmol) in dioxane (5 ml). The resulting solution was stirred at 20° C. for 3 hours. The crude product was purified by ion exchange chromatography, using an SCX column. The desired product was eluted from the column using 7M NH3/MeOH and pure fractions were evaporated to dryness to afford 4-(aminomethyl)-N-(5-(trifluoromethyl)pyridin-2-yl)piperidine-4-carboxamide (359 mg, 109%) as a colourless gum.

MS m/e MH⁺ 303

### 61D. 4-(Aminomethyl)-1-(7H-pyrrolo[2,3-d]pyrimidin-4-yl)-N-(5-(trifluoromethyl)pyridin-2-yl)piperidine-4-carboxamide

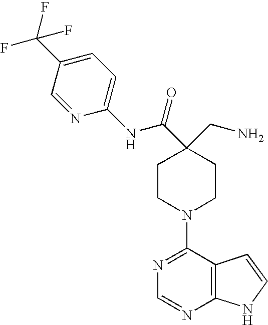

N-Ethyldiisopropylamine (0.621 ml, 3.56 mmol) was added to 4-(aminomethyl)-N-(5-(trifluoromethyl)pyridin-2-yl)piperidine-4-carboxamide (359 mg, 1.19 mmol) and 4-chloro-7H-pyrrolo[2,3-d]pyrimidine (219 mg, 1.43 mmol) in DMA (5 ml). The resulting solution was stirred at 60° C. for 18 hours. The crude product was purified by ion exchange chromatography, using an SCX column. The desired product was eluted from the column using 7M NH3/MeOH and product containing fractions were evaporated to dryness. The crude product was purified by preparative HPLC (Waters XTerra C18 column, 5μ silica, 19 mm diameter, 100 mm length), using decreasingly polar mixtures of water (containing 0.1% TFA) and MeCN as eluents. Fractions containing the desired compound were stirred with MP-carbonate then evaporated to dryness. The crude product was purified by ion exchange chromatography, using an SCX column. The desired product was eluted from the column using 7M NH3/MeOH and pure fractions were evaporated to dryness, triturated with methanol, filtered and washed with diethyl ether to afford 4-(aminomethyl)-1-(7H-pyrrolo[2,3-d]pyrimidin-4-yl)-N-(5-(trifluoromethyl)pyridin-2-yl)piperidine-4-carboxamide (109 mg, 21.9%) as a white solid.

1H NMR (400.13 MHz, DMSO-d6) δ 1.54-1.60 (2H, m), 2.08-2.14 (2H, m), 2.90 (2H, s), 3.76-3.83 (2H, m), 4.08-4.14 (2H, m), 6.59 (1H, d), 7.17-7.18 (1H, m), 8.13-8.18 (2H, m), 8.28 (1H, d), 8.67-8.68 (1H, m), 11.68 (1H, s)

MS m/e MH⁺ 420

### Example 62

### 4-(Aminomethyl)-N-(benzo[d]thiazol-6-yl)-1-(7H-pyrrolo[2,3-d]pyrimidin-4-yl)piperidine-4-carboxamide

### 62A. Ethyl 4-(aminomethyl)piperidine-4-carboxylate

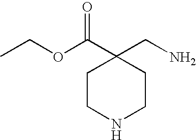

Hydrogen chloride 4M in dioxane (33.2 ml, 132.7 mmol) was added to 1-tent-butyl 4-ethyl 4-(aminomethyl)piperidine-1,4-dicarboxylate (Example 21C) (7.6 g, 26.5 mmol) in dioxane (35 ml). The resulting solution was stirred at 20° C. for 3 hours. The crude product was purified by ion exchange chromatography, using an SCX column. The desired product was eluted from the column using 7M NH3/MeOH and pure fractions were evaporated to dryness to afford ethyl 4-(aminomethyl)piperidine-4-carboxylate (3.34 g, 67.6%) as a yellow liquid.

1H NMR (400.13 MHz, CDCl₃) δ 1.23-1.30 (3H, m), 1.26-1.37 (2H, m), 2.12 (2H, d), 2.65-2.72 (2H, m), 2.77 (2H, s), 2.94-2.99 (2H, m), 4.21 (2H, q).

### 62B. Ethyl 4-(aminomethyl)-1-(7H-pyrrolo[2,3-d]pyrimidin-4-yl)piperidine-4-carboxylate

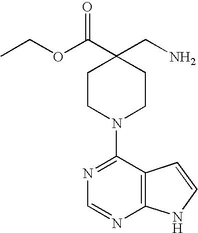

N-Ethyldiisopropylamine (3.70 ml, 21.26 mmol) was added to ethyl 4-(aminomethyl)piperidine-4-carboxylate (Example 62A) (3.3 g, 17.7 mmol) and 4-chloro-7H-pyrrolo[2,3-d]pyrimidine (2.72 g, 17.72 mmol) in DMA (35 ml). The resulting solution was stirred at 60° C. for 18 hours. The crude product was purified by ion exchange chromatography, using an SCX column. The desired product was eluted from the column using 7M NH3/MeOH and pure fractions were evaporated to dryness to afford ethyl 4-(aminomethyl)-1-(7H-pyrrolo[2,3-d]pyrimidin-4-yl)piperidine-4-carboxylate (5.08 g, 95%) as a beige solid.

1H NMR (400.13 MHz, DMSO-d₆) δ 1.22 (3H, t), 1.44-1.51 (2H, m), 2.04-2.07 (2H, m), 2.67 (2H, d), 3.23-3.30 (2H, m), 4.15 (2H, q), 4.39-4.44 (2H, m), 6.59 (1H, t), 7.16-7.17 (1H, m), 8.12 (1H, s), 11.67 (1H, s)

MS m/e MH⁺ 304.

### 62C. Ethyl 4-((tert-butoxycarbonylamino)methyl)-1-(7H-pyrrolo[2,3-d]pyrimidin-4-yl)piperidine-4-carboxylate

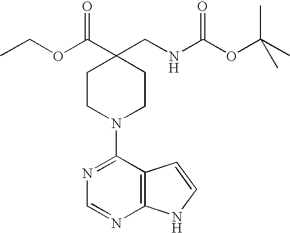

Di-tent-butyl dicarbonate (470 mg, 2.15 mmol) was added to ethyl 4-(aminomethyl)-1-(7H-pyrrolo[2,3-d]pyrimidin-4-yl)piperidine-4-carboxylate (Example 62B) (653 mg, 2.15 mmol) and triethylamine (0.300 ml, 2.15 mmol) in DCM (10 ml). The resulting suspension was stirred at ambient temperature for 2 hours. The reaction mixture was diluted with DCM (50 mL), and washed sequentially with water (50 mL) and saturated brine (50 mL). The organic layer was dried over MgSO₄, filtered and evaporated. The crude product was purified by flash silica chromatography, elution gradient 20 to 100% EtOAc in isohexane. Pure fractions were evaporated to dryness to afford ethyl 4-((tert-butoxycarbonylamino)methyl)-1-(7H-pyrrolo[2,3-d]pyrimidin-4-yl)piperidine-4-carboxylate (468 mg, 53.9%) as a colourless oil which solidified on standing.

1H NMR (400.13 MHz, DMSO-d6) δ 1.22 (3H, t), 1.36-1.38 (9H, m), 1.42-1.49 (2H, m), 2.05 (2H, d), 3.13 (2H, d), 3.20 (2H, t), 4.09-4.14 (2H, m), 4.45 (2H, d), 6.58 (1H, d), 6.94 (1H, t), 7.16 (1H, d), 8.13 (1H, d), 11.65 (1H, s).

MS m/e MH⁺ 404.

### 62D 4-((tert-Butoxycarbonylamino)methyl)-1-(7H-pyrrolo[2,3-d]pyrimidin-4-yl)piperidine-4-carboxylic acid

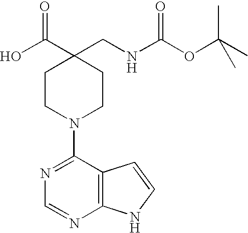

Lithium hydroxide monohydrate (0.556 g, 13.26 mmol) was added to ethyl 4-((tert-butoxycarbonylamino)methyl)-1-(7H-pyrrolo[2,3-d]pyrimidin-4-yl)piperidine-4-carboxylate (Example 62C) (1.07 g, 2.65 mmol) in water (6.25 ml), THF (25 ml) and ethanol (25.00 ml). The resulting solution was stirred at 20° C. for 1 day. The reaction mixture was diluted with EtOAc (20 mL) and washed with water (20 mL). The aqueous was adjusted to pH5 with 1M citric acid solution then extracted with EtOAc (3×50 mL). The organic extracts were washed with saturated brine (25 mL) then dried over MgSO4, filtered and evaporated to afford desired product 4-((tert-butoxycarbonylamino)methyl)-1-(7H-pyrrolo[2,3-d]pyrimidin-4-yl)piperidine-4-carboxylic acid (0.628 g, 63.1%) as a white foam.

1H NMR (400.13 MHz, DMSO-d6) δ 1.36 (9H, s), 1.44-1.51 (2H, m), 1.99-2.04 (2H, m), 3.14 (2H, d), 3.25 (2H, s), 4.43-4.46 (2H, m), 6.64 (1H, s), 6.84 (1H, t), 7.21 (1H, s), 8.16 (1H, s), 11.82 (1H, s)

MS m/e MH⁺ 376.

### 62E. 4-(Aminomethyl)-N-(benzo[d]thiazol-6-yl)-1-(7H-pyrrolo[2,3-d]pyrimidin-4-yl)piperidine-4-carboxamide

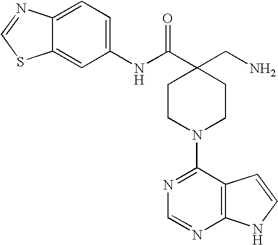

HATU (223 mg, 0.59 mmol) was added in one portion to 4-((tert-butoxycarbonylamino)methyl)-1-(7H-pyrrolo[2,3-d]pyrimidin-4-yl)piperidine-4-carboxylic acid (200 mg, 0.53 mmol) and DIPEA (0.279 ml, 1.60 mmol) in DMA (5 ml) and the reaction stirred at room temperature for 10 minutes. Benzo[d]thiazol-6-amine (80 mg, 0.53 mmol) was added and the resulting solution was stirred at 50° C. for 24 hours. The reaction mixture was diluted with EtOAc (25 mL), and washed sequentially with 2M NaOH (20 mL), water (20 mL), and saturated brine (20 mL). The organic layer was dried over MgSO4, filtered and evaporated to afford crude tert-butyl (4-(benzo[d]thiazol-6-ylcarbamoyl)-1-(7H-pyrrolo[2,3-d]pyrimidin-4-yl)piperidin-4-yl)methylcarbamate. The crude product was dissolved in DCM (5.00 ml) and trifluoroacetic acid (0.410 ml, 5.33 mmol) added. The reaction was stirred at ambient temperature for 2 hours then was purified by ion exchange chromatography, using an SCX column. The desired product was eluted from the column using 7M NH3/MeOH and pure fractions were evaporated to dryness to afford crude product. The crude product was purified by preparative HPLC (Waters XTerra C18 column, 5μ silica, 19 mm diameter, 100 mm length), using decreasingly polar mixtures of water (containing 1% NH3) and MeCN as eluents. Fractions containing the desired compound were evaporated to dryness to afford 4-(aminomethyl)-N-(benzo[d]thiazol-6-yl)-1-(7H-pyrrolo[2,3-d]pyrimidin-4-yl)piperidine-4-carboxamide (61.0 mg, 28.1%) as a white solid.

1H NMR (400.13 MHz, DMSO-d6) δ 1.55-1.62 (2H, m), 2.20 (2H, s), 2.87 (2H, s), 3.64-3.70 (2H, m), 4.22-4.26 (2H, m), 6.59-6.60 (1H, m), 7.16-7.17 (1H, m), 7.61-7.64 (1H, m), 8.01 (1H, d), 8.13 (1H, s), 8.54 (1H, d), 9.25 (1H, s), 11.63 (1H, s)

MS m/e MH⁺ 408

### Example 63

### 4-(Aminomethyl)-N-(benzo[d]thiazol-2-yl)-1-(7H-pyrrolo[2,3-d]pyrimidin-4-yl)piperidine-4-carboxamide

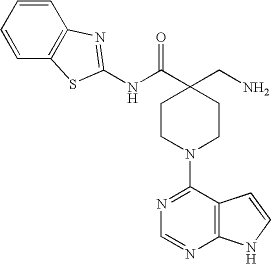

HATU (223 mg, 0.59 mmol) was added in one portion to 4-((tert-butoxycarbonyl-amino)methyl)-1-(7H-pyrrolo[2,3-d]pyrimidin-4-yl)piperidine-4-carboxylic acid (Example 62D) (200 mg, 0.53 mmol) and DIPEA (0.279 ml, 1.60 mmol) in DMA (5 ml) and the reaction stirred at room temperature for 10 minutes. Benzo[d]thiazol-2-amine (80 mg, 0.53 mmol) was added and the resulting solution was stirred at 50° C. for 24 hours. The crude product was purified by ion exchange chromatography, using an SCX column. The desired product was eluted from the column using 7M NH3/MeOH and pure fractions were evaporated to dryness to afford tert-butyl (4-(benzo[d]thiazol-2-ylcarbamoyl)-1-(7H-pyrrolo[2,3-d]pyrimidin-4-yl)piperidin-4-yl)methylcarbamate. This material was then dissolved in DCM (5.00 ml) and trifluoroacetic acid (0.410 ml, 5.33 mmol) added. The reaction was stirred at room temperature for 2 hours. The crude product was purified by ion exchange chromatography, using an SCX column. The desired product was eluted from the column using 7M NH3/MeOH and pure fractions were evaporated to dryness. The crude product was purified by preparative HPLC (Waters XBridge Prep C18 OBD column, 5μ silica, 19 mm diameter, 100 mm length), using decreasingly polar mixtures of water (containing 1% NH3) and MeCN as eluents. Fractions containing the desired compound were evaporated to dryness to afford 4-(aminomethyl)-N-(benzo[d]thiazol-2-yl)-1-(7H-pyrrolo[2,3-d]pyrimidin-4-yl)piperidine-4-carboxamide (54.0 mg, 24.8%) as a white solid.

1H NMR (400.13 MHz, DMSO-d6) δ 1.56-1.63 (2H, m), 2.14-2.19 (2H, m), 2.95 (2H, s), 3.73-3.78 (2H, m), 4.13-4.17 (2H, m), 6.59-6.61 (1H, m), 7.17-7.18 (1H, m), 7.22-7.26 (1H, m), 7.36-7.40 (1H, m), 7.67 (1H, d), 7.89-7.92 (1H, m), 8.14 (1H, s), 11.68 (1H, s)

MS m/e MH⁺ 408

### Example 64

### 4-(Aminomethyl)-N-(3-methyl-1,2,4-thiadiazol-5-yl)-1-(7H-pyrrolo[2,3-d]pyrimidin-4-yl)piperidine-4-carboxamide

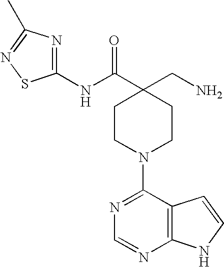

HATU (223 mg, 0.59 mmol) was added in one portion to 4-((tert-butoxycarbonylamino)-methyl)-1-(7H-pyrrolo[2,3-d]pyrimidin-4-yl)piperidine-4-carboxylic acid (Example 62D) (200 mg, 0.53 mmol) and DIPEA (0.279 ml, 1.60 mmol) in DMA (4 ml) and the reaction stirred at room temperature for 10 minutes. 5-Amino-3-methyl-1,2,4-thiadiazole (61.3 mg, 0.53 mmol) was added and the resulting solution was stirred at 50° C. for 24 hours. The crude product was purified by ion exchange chromatography, using an SCX column. The desired product was eluted from the column using 7M NH3/MeOH and pure fractions were evaporated to dryness to afford tert-butyl (4-(3-methyl-1,2,4-thiadiazol-5-ylcarbamoyl)-1-(7H-pyrrolo[2,3-d]pyrimidin-4-yl)piperidin-4-yl)methylcarbamate. This was then dissolved in DCM (4.00 ml) and trifluoroacetic acid (0.410 ml, 5.33 mmol) added. The reaction was stirred at ambient temperature for 2 hours. The crude product was purified by ion exchange chromatography, using an SCX column. The desired product was eluted from the column using 7M NH3/MeOH and pure fractions were evaporated to dryness to afford crude product. The crude product was purified by preparative HPLC (Phenomenex Gemini C18 110A (axia) column, 5μ silica, 19 mm diameter, 100 mm length), using decreasingly polar mixtures of water (containing 1% NH3) and MeCN as eluents. Fractions containing the desired compound were evaporated to dryness to afford crude product. The crude product was purified by ion exchange chromatography, using an SCX column. The desired product was eluted from the column using 7M NH3/MeOH and pure fractions were evaporated to dryness, triturated with diethyl ether to afford 4-(aminomethyl)-N-(3-methyl-1,2,4-thiadiazol-5-yl)-1-(7H-pyrrolo[2,3-d]pyrimidin-4-yl)piperidine-4-carboxamide (20.0 mg, 10.1%) as a white solid.

1H NMR (400.13 MHz, DMSO-d6) δ 1.54-1.61 (2H, m), 2.14-2.18 (2H, m), 2.35 (3H, s), 2.97 (2H, s), 3.69-3.74 (2H, m), 4.13-4.17 (2H, m), 6.58 (1H, d), 7.17 (1H, s), 8.13 (1H, s), 11.64 (1H, s)

MS m/e MH⁺ 373

### Example 65

### 4-(Aminomethyl)-N-(6-(methylsulfonyl)benzo[d]thiazol-2-yl)-1-(7H-pyrrolo[2,3-d]pyrimidin-4-yl)piperidine-4-carboxamide

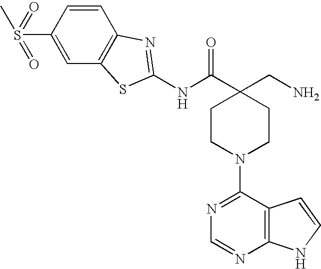

HATU (223 mg, 0.59 mmol) was added in one portion to 4-((tert-butoxycarbonyl-amino)methyl)-1-(7H-pyrrolo[2,3-d]pyrimidin-4-yl)piperidine-4-carboxylic acid (Example 62D) (200 mg, 0.53 mmol) and DIPEA (0.279 ml, 1.60 mmol) in DMA (5 ml) and the reaction stirred at room temperature for 10 minutes. 6-(methylsulfonyl)benzo[d]thiazol-2-amine (122 mg, 0.53 mmol) was added and the resulting solution was stirred at 50° C. for 24 hours. The reaction mixture was diluted with EtOAc (25 mL), and washed sequentially with water (20 mL), and saturated brine (20 mL). The organic layer was dried over MgSO4, filtered and evaporated to afford crude tert-butyl (4-(6-(methylsulfonyl)benzo[d]thiazol-2-ylcarbamoyl)-1-(7H-pyrrolo[2,3-d]pyrimidin-4-yl)piperidin-4-yl)methylcarbamate. The crude product was dissolved in DCM (5.00 ml) and trifluoroacetic acid (0.410 ml, 5.33 mmol) added. The reaction was stirred at ambient temperature for 2 hours. The crude product was purified by ion exchange chromatography, using an SCX column. The desired product was eluted from the column using 7M NH3/MeOH and pure fractions were evaporated to dryness to afford crude product. The crude product was purified by preparative HPLC (Phenomenex Gemini C18 110A (axia) column, 5μ silica, 19 mm diameter, 100 mm length), using decreasingly polar mixtures of water (containing 1% NH3) and MeCN as eluents. Fractions containing the desired compound were evaporated to dryness to afford crude product. The crude product was purified by ion exchange chromatography, using an SCX column. The desired product was eluted from the column using 7M NH3/MeOH and pure fractions were evaporated to dryness to afford 4-(aminomethyl)-N-(6-(methylsulfonyl)benzo[d]thiazol-2-yl)-1-(7H-pyrrolo[2,3-d]pyrimidin-4-yl)piperidine-4-carboxamide (48.0 mg, 18.6%) as a white solid.

1H NMR (400.13 MHz, DMSO-d6) δ 1.58-1.62 (3H, m), 2.17-2.21 (2H, m), 2.45 (1H, s), 3.03 (2H, s), 3.20 (3H, s), 3.80-3.85 (2H, m), 4.11-4.15 (2H, m), 6.60 (1H, d), 7.16-7.18 (1H, m), 7.68 (1H, d), 7.77-7.80 (1H, m), 8.14 (1H, s), 8.34 (1H, d), 11.64 (1H, s)

MS m/e MH⁺ 486

### Example 66

### 4-(aminomethyl)-N-(4-(pyridin-3-yl)thiazol-2-yl)-1-(7H-pyrrolo[2,3-d]pyrimidin-4-yl)piperidine-4-carboxamide

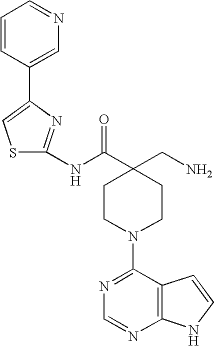

HATU (223 mg, 0.59 mmol) was added in one portion to 4-((tert-butoxycarbonylamino)methyl)-1-(7H-pyrrolo[2,3-d]pyrimidin-4-yl)piperidine-4-carboxylic acid (Example 62D) (200 mg, 0.53 mmol) and DIPEA (0.279 ml, 1.60 mmol) in DMA (5 ml) and the reaction stirred at room temperature for 10 minutes. 4-(Pyridin-3-yl)thiazol-2-amine (94 mg, 0.53 mmol) was added and the resulting solution was stirred at 50° C. for 24 hours. The reaction mixture was diluted with EtOAc (25 mL), and washed sequentially with 2M NaOH (20 mL), water (20 mL), and saturated brine (20 mL). The organic layer was dried over MgSO4, filtered and evaporated to afford crude tert-butyl (4-(2-ethylphenylcarbamoyl)-1-(7H-pyrrolo[2,3-d]pyrimidin-4-yl)piperidin-4-yl)methylcarbamate. The crude product was dissolved in DCM (5.00 ml) and trifluoroacetic acid (0.410 ml, 5.33 mmol) added. The reaction was stirred at ambient temperature for 2 hours. The crude product was purified by ion exchange chromatography, using an SCX column. The desired product was eluted from the column using 7M NH3/MeOH and fractions were evaporated to afford crude product. The crude product was purified by preparative HPLC (Waters XBridge Prep C18 OBD column, 5μ silica, 19 mm diameter, 100 mm length), using decreasingly polar mixtures of water (containing 1% NH3) and MeCN as eluents. Fractions containing the desired compound were evaporated to dryness to afford 4-(aminomethyl)-N-(4-(pyridin-3-yl)thiazol-2-yl)-1-(7H-pyrrolo[2,3-d]pyrimidin-4-yl)piperidine-4-carboxamide (6.00 mg, 2.6%) as a white solid.

1H NMR (400.13 MHz, DMSO-d6) δ 1.58-1.64 (2H, m), 2.17-2.21 (2H, m), 2.94 (2H, s), 3.69-3.74 (2H, m), 4.15-4.22 (2H, m), 6.59-6.60 (1H, m), 7.16-7.18 (1H, m), 7.43-7.47 (1H, m), 7.75 (1H, s), 8.14 (1H, s), 8.22-8.25 (1H, m), 8.51-8.52 (1H, m), 9.12 (1H, d), 11.65 (1H, s)

MS m/e MH⁺ 435

### Example 67

### 4-(Aminomethyl)-N-(pyridin-2-yl)-1-(7H-pyrrolo[2,3-d]pyrimidin-4-yl)piperidine-4-carboxamide

### 67A. tert-Butyl 4-cyano-4-(pyridin-2-ylcarbamoyl)piperidine-1-carboxylate

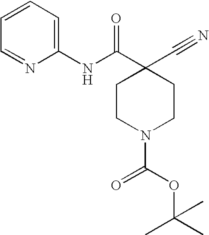

HATU (1121 mg, 2.95 mmol) was added in one portion to 1-(tert-butoxycarbonyl)-4-cyanopiperidine-4-carboxylic acid (Example 26A) (500 mg, 1.97 mmol) and DIPEA (1.030 ml, 5.90 mmol) in DMA (5 ml) at 20° C. under nitrogen. The resulting solution was stirred at 20° C. for 10 minutes then 2-aminopyridine (222 mg, 2.36 mmol) added. The reaction mixture was stirred at 50° C. for 6 hours then at 20° C. for 18 hours. The reaction mixture was diluted with EtOAc (50 mL), and washed sequentially with water (2×50 mL) and saturated brine (50 mL). The organic layer was dried over MgSO4, filtered and evaporated to afford crude product. The crude product was purified by flash silica chromatography, elution gradient 10 to 50% EtOAc in isohexane. Pure fractions were evaporated to dryness and triturated with diethyl ether to afford tert-butyl 4-cyano-4-(pyridin-2-ylcarbamoyl)piperidine-1-carboxylate (535 mg, 82%) as a white solid.

1H NMR (400.13 MHz, DMSO-d6) δ 1.42 (9H, s), 1.98-2.05 (2H, m), 2.22-2.25 (2H, m), 2.97 (2H, s), 4.02-4.05 (2H, m), 7.18-7.21 (1H, m), 7.82-7.86 (1H, m), 7.97-8.00 (1H, m), 8.38-8.40 (1H, m), 10.85 (1H, s)

MS m/e M−H 329

### 67B. Preparation of tert-butyl 4-(aminomethyl)-4-(pyridin-2-ylcarbamoyl)piperidine-1-carboxylate

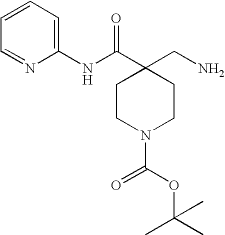

Platinum(IV) oxide (36.8 mg, 0.16 mmol) and tert-butyl 4-cyano-4-(pyridin-2-ylcarbamoyl)piperidine-1-carboxylate (535 mg, 1.62 mmol) in acetic acid (20 ml) were stirred under an atmosphere of hydrogen at 1 atm and 25° C. for 1 day. The reaction mixture was filtered and the crude product was purified by ion exchange chromatography, using an SCX column. The desired product was eluted from the column using 7M NH3/MeOH and pure fractions were evaporated to dryness to afford tert-butyl 4-(aminomethyl)-4-(pyridin-2-ylcarbamoyl)piperidine-1-carboxylate (500 mg, 92%) as a colourless gum.

MS m/e MH⁺ 335

### 67C. Preparation of 4-(aminomethyl)-N-(pyridin-2-yl)piperidine-4-carboxamide

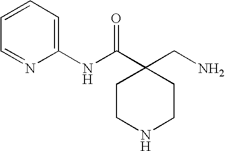

Hydrogen chloride 4M in dioxane (1.869 ml, 7.48 mmol) was added to tert-butyl 4-(aminomethyl)-4-(pyridin-2-ylcarbamoyl)piperidine-1-carboxylate (500 mg, 1.50 mmol) in dioxane (5 ml). The resulting solution was stirred at 20° C. for 3 hours. The crude product was purified by ion exchange chromatography, using an SCX column. The desired product was eluted from the column using 7M NH3/MeOH and pure fractions were evaporated to dryness to afford 4-(aminomethyl)-N-(pyridin-2-yl)piperidine-4-carboxamide (200 mg, 57.1%) as a colourless gum.

MS m/e MH⁺ 235

### 67D. Preparation of 4-(aminomethyl)-N-(pyridin-2-yl)-1-(7H-pyrrolo[2,3-d]pyrimidin-4-yl)piperidine-4-carboxamide

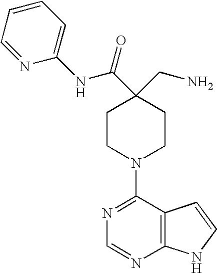

N-Ethyldiisopropylamine (0.446 ml, 2.56 mmol) was added to 4-(aminomethyl)-N-(pyridin-2-yl)piperidine-4-carboxamide (200 mg, 0.85 mmol) and 4-chloro-7H-pyrrolo[2,3-d]pyrimidine (157 mg, 1.02 mmol) in DMA (5 ml). The resulting solution was stirred at 60° C. for 18 hours. The reaction mixture was diluted with water (500 mL) and extracted with EtOAc (3×500 mL). The organic extracts were washed sequentially with water (500 mL) and saturated brine (200 mL). The organic layer was dried over MgSO4, filtered and evaporated to afford crude product. The crude product was purified by preparative HPLC (Phenomenex Gemini C18 110A (axia) column, 5μ silica, 19 mm diameter, 100 mm length), using decreasingly polar mixtures of water (containing 1% NH3) and MeCN as eluents. Fractions containing the desired compound were evaporated to dryness to afford 4-(aminomethyl)-N-(pyridin-2-yl)-1-(7H-pyrrolo[2,3-d]pyrimidin-4-yl)piperidine-4-carboxamide (17.0 mg, 5.7%) as a white solid.

1H NMR (400.13 MHz, DMSO-d6) δ 1.52-1.59 (2H, m), 2.10-2.14 (2H, m), 2.89 (2H, d), 3.75-3.82 (2H, m), 4.09-4.14 (2H, m), 6.59 (1H, d), 7.06-7.09 (1H, m), 7.16 (1H, d), 7.74-7.78 (1H, m), 8.09 (1H, d), 8.13 (1H, s), 8.28-8.30 (1H, m), 11.63 (1H, s)

MS m/e MH⁺ 352

### Example 68

### 4-(aminomethyl)-1-(7H-pyrrolo[2,3-d]pyrimidin-4-yl)-N-(4-(trifluoromethyl)pyridin-2-yl)piperidine-4-carboxamide

### 68A. tert-Butyl 4-cyano-4-(4-(trifluoromethyl)pyridin-2-ylcarbamoyl)piperidine-1-carboxylate

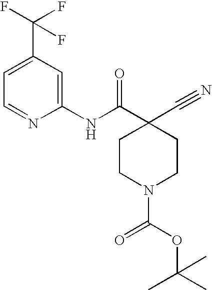

HATU (1121 mg, 2.95 mmol) was added in one portion to 1-(tert-butoxycarbonyl)-4-cyanopiperidine-4-carboxylic acid (Example 26A) (500 mg, 1.97 mmol) and DIPEA (1.030 ml, 5.90 mmol) in DMA (5 ml) at 20° C. under nitrogen. The resulting solution was stirred at 20° C. for 10 minutes then 4-(trifluoromethyl)pyridin-2-amine (319 mg, 1.97 mmol) added. The reaction mixture was stirred at 50° C. for 6 hours then at 20° C. for 18 hours. The reaction mixture was diluted with EtOAc (50 mL), and washed sequentially with water (2×50 mL) and saturated brine (50 mL). The organic layer was dried over MgSO4, filtered and evaporated to afford crude product. The crude product was purified by flash silica chromatography, elution gradient 20 to 40% EtOAc in isohexane. Pure fractions were evaporated to dryness and triturated with diethyl ether to afford tert-butyl 4-cyano-4-(4-(trifluoromethyl)pyridin-2-ylcarbamoyl)piperidine-1-carboxylate (569 mg, 72.6%) as a white solid.

1H NMR (400.13 MHz, DMSO-d6) δ 1.42 (9H, s), 1.99-2.08 (2H, m), 2.23-2.26 (2H, m), 2.98 (2H, s), 4.02-4.06 (2H, m), 7.58-7.59 (1H, m), 8.32 (1H, s), 8.68-8.69 (1H, m), 11.39 (1H, s)

MS m/e M−H 397

### 68B. tert-Butyl 4-(aminomethyl)-4-(4-(trifluoromethyl)pyridin-2-ylcarbamoyl)piperidine-1-carboxylate

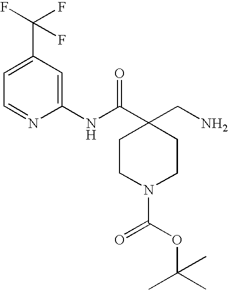

Platinum(IV) oxide (32.4 mg, 0.14 mmol) and tert-butyl 4-cyano-4-(4-(trifluoromethyl)pyridin-2-ylcarbamoyl)piperidine-1-carboxylate (569 mg, 1.43 mmol) in acetic acid (20 ml) were stirred under an atmosphere of hydrogen at 1 atm and 25° C. for 1 day. The reaction mixture was filtered through celite and the crude product was purified by ion exchange chromatography, using an SCX column. The desired product was eluted from the column using 7M NH3/MeOH and pure fractions were evaporated to dryness to afford tert-butyl 4-(aminomethyl)-4-(4-(trifluoromethyl)pyridin-2-ylcarbamoyl)piperidine-1-carboxylate (464 mg, 81%) as a colourless gum.

MS m/e M−H 401

### 68C. tert-Butyl 4-((diphenylmethyleneamino)methyl)-4-(4-(trifluoromethyl)pyridin-2-ylcarbamoyl)piperidine-1-carboxylate

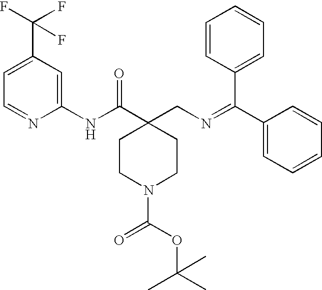

tert-Butyl 4-(aminomethyl)-4-(4-(trifluoromethyl)pyridin-2-ylcarbamoyl)piperidine-1-carboxylate (464 mg, 1.15 mmol), benzophenone imine (0.193 ml, 1.15 mmol) and p-toluenesulfonic acid (59.6 mg, 0.35 mmol) were added to DCM (10 ml) and stirred at 25° C. overnight. The reaction mixture was quenched with saturated NaHCO3 (25 ml), extracted with DCM (3×25 mL), the organic layer was dried over MgSO4, filtered and evaporated to afford yellow gum. The crude product was purified by flash silica chromatography, elution 10-40% EtOAc in isohexane. Pure fractions were evaporated to dryness to afford tert-butyl 4-((diphenylmethyleneamino)methyl)-4-(4-(trifluoromethyl)pyridin-2-ylcarbamoyl)piperidine-1-carboxylate (367 mg, 56.2%) colourless gum.

1H NMR (400.13 MHz, CDCl₃) δ 1.43 (9H, s), 2.13-2.19 (2H, m), 3.41-3.50 (6H, m), 7.15-7.17 (2H, m), 7.22-7.24 (1H, m), 7.39-7.53 (6H, m), 7.79-7.82 (2H, m), 8.48 (1H, d), 8.55 (1H, d), 11.54 (1H, s)

MS m/e MH⁺ 567

### 68D. 4-((Diphenylmethyleneamino)methyl)-N-(4-(trifluoromethyl)pyridin-2-yl)piperidine-4-carboxamide

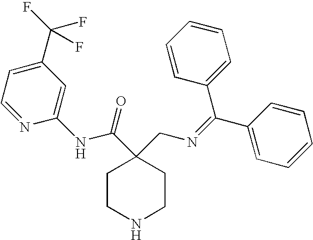

Hydrogen chloride 4M in dioxane (1.232 ml, 4.93 mmol) was added to tert-butyl 4-((diphenylmethyleneamino)methyl)-4-(4-(trifluoromethyl)pyridin-2-ylcarbamoyl)piperidine-1-carboxylate (349 mg, 0.62 mmol) in dioxane (5 ml). The resulting solution was stirred at ambient temperature for 2 hours. The reaction mixture was dissolved in methanol and purified by ion exchange chromatography, using an SCX column. The desired product was eluted from the column using 7M NH3/MeOH and pure fractions were evaporated to dryness to afford 4-((diphenylmethyleneamino)methyl)-N-(4-(trifluoromethyl)pyridin-2-yl)piperidine-4-carboxamide (278 mg, 97%) as a yellow gum.

1H NMR (400.13 MHz, CDCl₃) δ 2.12-2.18 (2H, m), 2.73-2.79 (2H, m), 2.92-2.98 (2H, m), 3.52 (2H, s), 7.15-7.18 (2H, m), 7.20-7.22 (1H, m), 7.37-7.52 (7H, m), 7.78-7.81 (2H, m), 8.47 (1H, d), 8.57 (1H, s), 11.36 (1H, s)

MS m/e MH⁺ 467

### 68E. 4-((diphenylmethyleneamino)methyl)-1-(7H-pyrrolo[2,3-d]pyrimidin-4-yl)-N-(4-(trifluoromethyl)pyridin-2-yl)piperidine-4-carboxamide

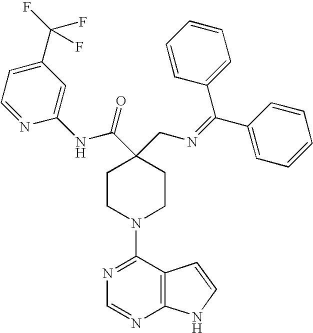

N-Ethyldiisopropylamine (0.311 ml, 1.79 mmol) was added to 4-((diphenylmethylene-amino)methyl)-N-(4-(trifluoromethyl)pyridin-2-yl)piperidine-4-carboxamide (278 mg, 0.60 mmol) and 4-chloro-7H-pyrrolo[2,3-d]pyrimidine (110 mg, 0.72 mmol) in butan-1-ol (3 ml). The resulting solution was stirred at 60° C. for 18 hours. The reaction mixture was diluted with EtOAc (50 mL), and washed sequentially with water (50 mL) and saturated brine (25 mL). The organic layer was dried over MgSO4, filtered and evaporated to afford crude product. The crude product was purified by flash silica chromatography, elution gradient 80 to 100% EtOAc in isohexane. Pure fractions were evaporated to dryness to afford 4-((diphenylmethyleneamino)methyl)-1-(7H-pyrrolo[2,3-d]pyrimidin-4-yl)-N-(4-(trifluoromethyl)pyridin-2-yl)piperidine-4-carboxamide (247 mg, 71.0%) as a white solid.

1H NMR (400.13 MHz, DMSO-d6) δ 1.58-1.64 (2H, m), 2.30-2.34 (2H, m), 3.62-3.67 (4H, m), 4.08 (2H, m), 6.57 (1H, s), 7.14-7.16 (1H, m), 7.19-7.21 (2H, m), 7.37-7.41 (2H, m), 7.43-7.53 (5H, m), 7.55-7.57 (2H, m), 8.11 (1H, s), 8.47 (1H, s), 8.64 (1H, d), 11.09 (1H, s), 11.63 (1H, s)

MS m/e MH⁺ 584

### 68F. 4-(Aminomethyl)-1-(7H-pyrrolo[2,3-d]pyrimidin-4-yl)-N-(4-(trifluoromethyl)pyridin-2-yl)piperidine-4-carboxamide

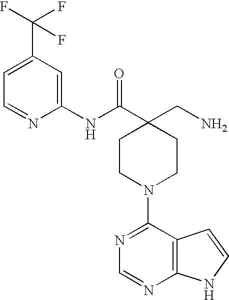

Hydrogen chloride 4M in dioxane (0.846 ml, 3.39 mmol) was added to 4-((diphenylmethyleneamino)methyl)-1-(7H-pyrrolo[2,3-d]pyrimidin-4-yl)-N-(4-(trifluoromethyl)pyridin-2-yl)piperidine-4-carboxamide (247 mg, 0.42 mmol) in dioxane (5 ml) and water (1 ml). The resulting solution was stirred at ambient temperature for 24 hours. The reaction mixture was dissolved in methanol and purified by ion exchange chromatography, using an SCX column. The desired product was eluted from the column using 7M NH3/MeOH and pure fractions were evaporated to dryness and triturated with diethyl ether to afford 4-(aminomethyl)-1-(7H-pyrrolo[2,3-d]pyrimidin-4-yl)-N-(4-(trifluoromethyl)pyridin-2-yl)piperidine-4-carboxamide (118 mg, 66.5%) as a white solid.

1H NMR (400.13 MHz, DMSO-d6) δ 1.55-1.61 (2H, m), 2.10-2.16 (2H, m), 2.90 (2H, s), 3.76-3.83 (2H, m), 4.09-4.15 (2H, m), 6.58 (1H, d), 7.17 (1H, d), 7.44-7.45 (1H, m), 8.13 (1H, s), 8.42 (1H, s), 8.58 (1H, d), 11.64 (1H, s);

MS m/e MH⁺ 420.

### Example 69

### 4-(Aminomethyl)-N-(5-methylpyridin-2-yl)-1-(7H-pyrrolo[2,3-d]pyrimidin-4-yl)piperidine-4-carboxamide

### 69A. tert-butyl 4-cyano-4-(5-methylpyridin-2-ylcarbamoyl)piperidine-1-carboxylate

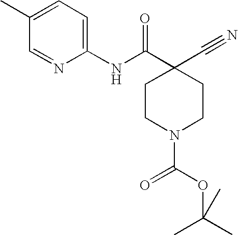

HATU (1121 mg, 2.95 mmol) was added in one portion to 1-(tert-butoxycarbonyl)-4-cyanopiperidine-4-carboxylic acid (500 mg, 1.97 mmol) and DIPEA (1.030 ml, 5.90 mmol) in DMA (5 ml) at 20° C. under nitrogen. The resulting solution was stirred at 20° C. for 10 minutes then 5-methylpyridin-2-amine (213 mg, 1.97 mmol) added. The reaction mixture was stirred at 50° C. for 6 hours then at 20° C. for 18 hours. The reaction mixture was diluted with EtOAc (50 mL), and washed sequentially with water (2×50 mL) and saturated brine (50 mL). The organic layer was dried over MgSO4, filtered and evaporated to afford crude product. The crude product was purified by flash silica chromatography, elution gradient 20 to 40% EtOAc in isohexane. Pure fractions were evaporated to dryness and triturated with diethyl ether to afford tert-butyl 4-cyano-4-(5-methylpyridin-2-ylcarbamoyl)piperidine-1-carboxylate (508 mg, 75%) as a white solid.

1H NMR (400.13 MHz, DMSO-d6) δ 1.38-1.47 (9H, m), 1.96-2.04 (2H, m), 2.20-2.23 (2H, m), 2.27 (3H, s), 2.96-3.00 (2H, s), 4.02 (2H, t), 7.64-7.67 (1H, m), 7.87 (1H, d), 8.22-8.23 (1H, m), 10.75 (1H, s)

MS m/e M−H 343

### 69B. tert-butyl 4-(aminomethyl)-4-(5-methylpyridin-2-ylcarbamoyl)piperidine-1-carboxylate

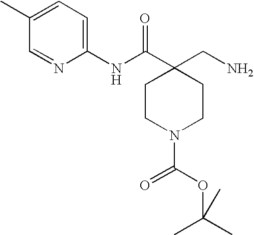

Platinum(IV) oxide (33.5 mg, 0.15 mmol) and tert-butyl 4-cyano-4-(5-methylpyridin-2-ylcarbamoyl)piperidine-1-carboxylate (508 mg, 1.47 mmol) in acetic acid (20 ml) were stirred under an atmosphere of hydrogen at 1 atm and 25° C. for 1 day. The reaction mixture was filtered through celite and the crude product was purified by ion exchange chromatography, using an SCX column. The desired product was eluted from the column using 7M NH3/MeOH and pure fractions were evaporated to dryness to afford tert-butyl 4-(aminomethyl)-4-(5-methylpyridin-2-ylcarbamoyl)piperidine-1-carboxylate (479 mg, 93%) as a colourless gum.

MS m/e MH⁺ 349

### 69C. tert-Butyl 4-((diphenylmethyleneamino)methyl)-4-(5-methylpyridin-2-ylcarbamoyl)piperidine-1-carboxylate

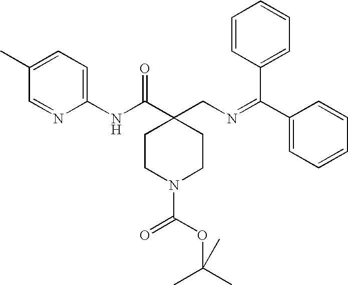

tert-Butyl 4-(aminomethyl)-4-(5-methylpyridin-2-ylcarbamoyl)piperidine-1-carboxylate (479 mg, 1.37 mmol), benzophenone imine (0.231 ml, 1.37 mmol) and p-toluenesulfonic acid (71.0 mg, 0.41 mmol) were added to DCM (10 ml) and stirred at 25° C. overnight. The reaction mixture was quenched with saturated NaHCO3 (25 ml), extracted with DCM (3×25 mL), the organic layer was dried over MgSO4, filtered and evaporated to afford yellow gum. The crude product was purified by flash silica chromatography, elution 10-40% EtOAc in isohexane. Pure fractions were evaporated to dryness to afford tert-butyl 4-((diphenylmethyleneamino)methyl)-4-(5-methylpyridin-2-ylcarbamoyl)piperidine-1-carboxylate (435 mg, 61.7%) as a colourless gum.

1H NMR (400.13 MHz, CDCl₃) δ 1.43 (9H, s), 2.14-2.20 (2H, m), 2.29 (3H, s), 3.47-3.50 (8H, m), 7.13-7.16 (2H, m), 7.36-7.45 (3H, m), 7.46-7.52 (4H, m), 7.77-7.80 (2H, m), 8.10-8.15 (2H, m), 10.80 (1H, s)

MS m/e MH⁺ 513

### 69D. 4-((Diphenylmethyleneamino)methyl)-N-(5-methylpyridin-2-yl)piperidine-4-carboxamide

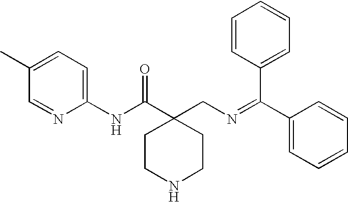

Hydrogen chloride 4M in dioxane (1.697 ml, 6.79 mmol) was added to tert-butyl 4-((diphenylmethyleneamino)methyl)-4-(5-methylpyridin-2-ylcarbamoyl)piperidine-1-carboxylate (435 mg, 0.85 mmol) in dioxane (5 ml). The resulting solution was stirred at ambient temperature for 2 hours. The reaction mixture was dissolved in methanol and purified by ion exchange chromatography, using an SCX column. The desired product was eluted from the column using 7M NH3/MeOH and pure fractions were evaporated to dryness to afford 4-((diphenylmethyleneamino)methyl)-N-(5-methylpyridin-2-yl)piperidine-4-carboxamide (310 mg, 89%) as a yellow gum.

1H NMR (400.13 MHz, CDCl₃) δ 1.46-1.52 (2H, m), 2.10 (1H, s), 2.13-2.19 (2H, m), 2.29 (3H, s), 2.74-2.80 (2H, m), 2.90-2.97 (2H, m), 3.50 (2H, s), 7.13-7.16 (2H, m), 7.35-7.43 (3H, m), 7.43-7.52 (4H, m), 7.77-7.80 (2H, m), 8.13-8.15 (2H, m), 10.59 (1H, s)

MS m/e MH⁺ 413

### 69E. 4-((Diphenylmethyleneamino)methyl)-N-(5-methylpyridin-2-yl)-1-(7H-pyrrolo[2,3-d]pyrimidin-4-yl)piperidine-4-carboxamide

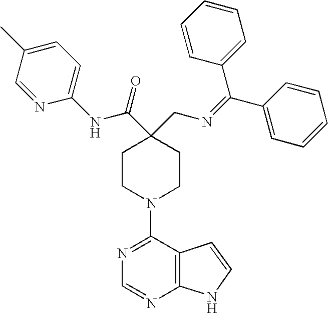

N-Ethyldiisopropylamine (0.393 ml, 2.25 mmol) was added to 4-((diphenylmethyleneamino)methyl)-N-(5-methylpyridin-2-yl)piperidine-4-carboxamide (310 mg, 0.75 mmol) and 4-chloro-7H-pyrrolo[2,3-d]pyrimidine (138 mg, 0.90 mmol) in butan-1-ol (3 ml). The resulting solution was stirred at 60° C. for 18 hours. The reaction mixture was diluted with EtOAc (50 mL), and washed sequentially with water (50 mL) and saturated brine (25 mL). The organic layer was dried over MgSO4, filtered and evaporated to afford crude product. The crude product was purified by flash silica chromatography, elution gradient 80 to 100% EtOAc in isohexane. Pure fractions were evaporated to dryness to afford 4-((diphenylmethyleneamino)methyl)-N-(5-methylpyridin-2-yl)-1-(7H-pyrrolo[2,3-d]pyrimidin-4-yl)piperidine-4-carboxamide (296 mg, 74.4%) as a white solid.

1H NMR (400.13 MHz, DMSO-d6) δ 1.56-1.63 (2H, m), 2.26-2.31 (5H, m), 3.60-3.65 (4H, m), 4.06 (2H, t), 6.57 (1H, d), 7.14-7.15 (1H, m), 7.19-7.21 (2H, m), 7.37-7.53 (6H, m), 7.57-7.63 (3H, m), 8.03 (1H, d), 8.11 (1H, s), 8.18-8.19 (1H, m), 10.50 (1H, s), 11.62 (1H, s)

MS m/e MH⁺ 530

### 69F. 4-(Aminomethyl)-N-(5-methylpyridin-2-yl)-1-(7H-pyrrolo[2,3-d]pyrimidin-4-yl)piperidine-4-carboxamide

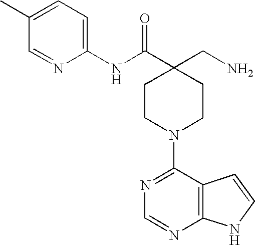

Hydrogen chloride 4M in dioxane (1.118 ml, 4.47 mmol) was added to 4-((diphenylmethyleneamino)methyl)-N-(5-methylpyridin-2-yl)-1-(7H-pyrrolo[2,3-d]pyrimidin-4-yl)piperidine-4-carboxamide (296 mg, 0.56 mmol) in dioxane (5 ml) and water (1 ml). The resulting solution was stirred at ambient temperature for 24 hours. The reaction mixture was dissolved in methanol and purified by ion exchange chromatography, using an SCX column. The desired product was eluted from the column using 7M NH3/MeOH and pure fractions were evaporated to dryness and triturated with diethyl ether to afford 4-(aminomethyl)-N-(5-methylpyridin-2-yl)-1-(7H-pyrrolo[2,3-d]pyrimidin-4-yl)piperidine-4-carboxamide (150 mg, 73.4%) as a white solid.

1H NMR (400.13 MHz, DMSO-d6) δ 1.52-1.59 (2H, m), 2.09-2.15 (2H, m), 2.24 (3H, s), 2.87 (2H, s), 3.74-3.80 (2H, m), 4.09-4.15 (2H, m), 6.58 (1H, d), 7.16 (1H, d), 7.57-7.59 (1H, m), 7.99 (1H, d), 8.12-8.13 (2H, m), 11.63 (1H, s)

MS m/e MH⁺ 366

### Example 70

### (4-(3-fluoro-5-(1-methyl-1H-pyrazol-4-yl)phenyl)-1-(7H-pyrrolo[2,3-d]pyrimidin-4-yl)piperidin-4-yl)methanamine

### 70A. tert-Butyl 4-(3-bromo-5-fluorophenyl)-4-cyanopiperidine-1-carboxylate

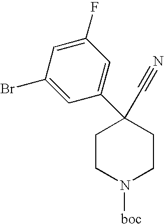

Sodium hydride (60% in mineral oil) (2.78 g, 69.51 mmol) was added portionwise to tert-butyl bis(2-chloroethyl)carbamate (6.38 g, 26.36 mmol) and 2-(3-bromo-5-fluorophenyl)acetonitrile (5.13 g, 23.97 mmol) in DMF (35 mL) under nitrogen. The resulting mixture was stirred at 65-70° C. for 1 hour and then at room temperature overnight. The reaction mixture was poured onto ice/water (250 ml) and extracted with EtOAc (×3). The combined extracts were washed with water followed by saturated brine, dried over magnesium sulphate, filtered and evaporated. The crude product was purified by flash silica chromatography, elution gradient 5 to 15% EtOAc in isohexane. Pure fractions were evaporated in vacuo to afford tert-butyl 4-(3-bromo-5-fluorophenyl)-4-cyanopiperidine-1-carboxylate (3.83 g, 41.7%) as a light brown gum.

1H NMR (399.9 MHz, CDCl₃) δ 1.49 (9H, s), 1.87-1.93 (2H, m), 2.04-2.09 (2H, m), 3.10-3.28 (2H, m), 4.20-4.40 (2H, bs), 7.13-7.16 (1H, m), 7.23-7.26 (1H, m), 7.42 (1H, m)

### 70B. (4-(3-Bromo-5-fluorophenyl)piperidin-4-yl)methanamine

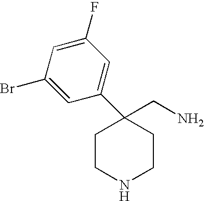

Borane tetrahydrofuran complex (27 mL, 27.4 mmol) was added to tert-butyl 4-(3-bromo-5-fluorophenyl)-4-cyanopiperidine-1-carboxylate (2.100 g, 5.48 mmol) in THF (20 mL) over a period of 5 minutes, under nitrogen, and the resulting solution stirred at ambient temperature for 2 hours. The reaction mixture was quenched with methanol (50 mL), a 4M solution of HCl in dioxane (50 mL) added and the mixture stirred at reflux for 3 hours.

The mixture was cooled and the crude product purified by ion exchange chromatography, using an SCX column. The desired product was eluted from the column using 2M NH3/MeOH and pure fractions were evaporated in vacuo to afford (4-(3-bromo-5-fluorophenyl)piperidin-4-yl)methanamine (1.170 g, 74.3%) as a colourless gum.

1H NMR (399.9 MHz, CDCl₃) δ 1.70-1.77 (2H, m), 1.30 (2H, bs), 2.02-2.08 (2H, m), 2.69-2.75 (2H, m), 2.77 (2H, s), 2.90-2.96 (2H, m), 6.95-6.99 (1H, m), 7.11-7.14 (1H, m), 7.24 (1H, m).

### 70C. (4-(3-Bromo-5-fluorophenyl)-1-(7H-pyrrolo[2,3-d]pyrimidin-4-yl)piperidin-4-yl)methanamine

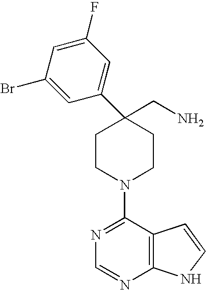

DIPEA (0.937 mL, 5.36 mmol) was added to 4-chloro-7H-pyrrolo[2,3-d]pyrimidine (0.412 g, 2.68 mmol) and (4-(3-bromo-5-fluorophenyl)piperidin-4-yl)methanamine (0.77 g, 2.68 mmol) in n-BuOH (10 mL) and the resulting mixture stirred at 80° C. for 6 hours. The mixture was evaporated to dryness and the residue triturated with methanol to afford (4-(3-bromo-5-fluorophenyl)-1-(7H-pyrrolo[2,3-d]pyrimidin-4-yl)piperidin-4-yl)methanamine (467 mg, 1.16 mmol, 43.1%) as a white solid. The methanol solvent was evaporated in vacuo and the residue triturated with acetone to give a second crop of (4-(3-bromo-5-fluorophenyl)-1-(7H-pyrrolo[2,3-d]pyrimidin-4-yl)piperidin-4-yl)methanamine (170 mg, 0.42 mmol, 15.7%) as a white solid.

1H NMR (399.9 MHz, DMSO-d6) δ 1.93-1.98 (2H, m), 2.27-2.30 (2H, m), 3.12 (2H, s), 3.42-3.48 (2H, m), 4.23-4.26 (2H, m), 6.60 (1H, s), 7.19 (1H, m), 7.41 (1H, d), 7.50-7.54 (2H, m), 7.70-7.73 (2H, m), 8.15 (1H, s), 11.69 (1H, s)

MS m/e MH⁺ 404, 406

### 70D. (4-(3-Fluoro-5-(1-methyl-1H-pyrazol-4-yl)phenyl)-1-(7H-pyrrolo[2,3-d]pyrimidin-4-yl)piperidin-4-yl)methanamine

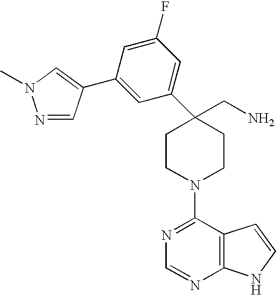

Bis(tri-t-butylphosphine)palladium(0) (7.66 mg, 0.01 mmol) was added in one portion to a carefully degassed solution of (4-(3-bromo-5-fluorophenyl)-1-(7H-pyrrolo[2,3-d]pyrimidin-4-yl)piperidin-4-yl)methanamine (0.101 g, 0.25 mmol), 1-methyl-4-(4,4,5,5-tetramethyl-1,3,2-dioxaborolan-2-yl)-1H-pyrazole (0.104 g, 0.50 mmol) and potassium phosphate tribasic (0.186 g, 0.87 mmol) in a mixture of toluene (3 mL), ethanol (6.00 mL) and water (3.00 mL), at 25°, under nitrogen. The resulting solution was stirred at 80° C. for 2 hours. The crude product was purified by ion exchange chromatography, using an SCX column. The desired product was eluted from the column using 2M NH₃/MeOH and the fractions evaporated to dryness. The crude product was purified by preparative HPLC (Waters XTerra C18 column, 5μ silica, 19 mm diameter, 100 mm length), using decreasingly polar mixtures of water (containing 1% NH3) and MeCN as eluents. Fractions containing the desired compound were evaporated to dryness to afford (4-(3-fluoro-5-(1-methyl-1H-pyrazol-4-yl)phenyl)-1-(7H-pyrrolo[2,3-d]pyrimidin-4-yl)piperidin-4-yl)methanamine (0.076 g, 75%) as a colourless gum.

1H NMR (500.13 MHz, DMSO-d6) δ 1.04 (2H, bs), 1.86-1.92 (2H, m), 2.19-2.24 (2H, m), 2.73 (2H, s), 3.45-3.51 (2H, m), 3.87 (3H, s), 4.21-4.24 (2H, m), 6.58 (1H, d), 7.03-7.05 (1H, m), 7.16 (1H, d), 7.26-7.29 (1H, m), 7.42 (1H, s), 7.95 (1H, m), 8.13 (1H, s), 8.25 (1H, s), 11.62 (1H, bs)

MS m/e MH⁺ 406

### Example 71

### (4-(3-Fluoro-5-(5-fluoropyridin-3-yl)phenyl)-1-(7H-pyrrolo[2,3-d]pyrimidin-4-yl)piperidin-4-yl)methanamine

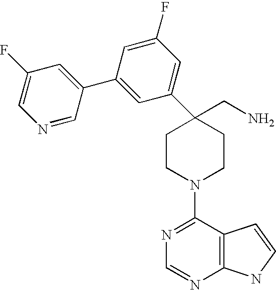

Bis(tri-t-butylphosphine)palladium(0) (7.66 mg, 0.01 mmol) was added in one portion to a carefully degassed solution of (4-(3-bromo-5-fluorophenyl)-1-(7H-pyrrolo[2,3-d]pyrimidin-4-yl)piperidin-4-yl)methanamine (0.101 g, 0.25 mmol), 3-fluoro-5-(4,4,5,5-tetramethyl-1,3,2-dioxaborolan-2-yl)pyridine (0.111 g, 0.50 mmol) and Potassium phosphate tribasic (0.186 g, 0.87 mmol) in a mixture of toluene (3 mL), ethanol (6.00 mL) and water (3.00 mL), at 25°, under nitrogen. The resulting solution was stirred at 80° C. for 6 hours. The reaction was incomplete and further 3-fluoro-5-(4,4,5,5-tetramethyl-1,3,2-dioxaborolan-2-yl)pyridine (0.111 g, 0.50 mmol), Potassium phosphate tribasic (0.186 g, 0.87 mmol) and BIS(TRI-T-BUTYLPHOSPHINE)PALLADIUM(0) (7.66 mg, 0.01 mmol) were added and the mixture was stirred at 80° C. for a further 7 hours. The crude product was purified by ion exchange chromatography, using an SCX column. The desired product was eluted from the column using 2M NH₃/MeOH and fractions evaporated to dryness. The residue was purified by preparative HPLC (Waters XBridge Prep C18 OBD column, 5μ silica, 21 mm diameter, 100 mm length), using decreasingly polar mixtures of water (containing 1% NH3) and MeCN as eluents. Fractions containing the desired compound were evaporated to dryness to afford (4-(3-fluoro-5-(5-fluoropyridin-3-yl)phenyl)-1-(7H-pyrrolo[2,3-d]pyrimidin-4-yl)piperidin-4-yl)methanamine (0.022 g, 20.6%) as a white solid.

1H NMR (700.03 MHz, DMSO-d6) δ 1.15 (2H, s), 1.90-1.94 (2H, m), 2.27-2.30 (2H, m), 2.75 (2H, s), 3.47-3.51 (2H, m), 4.22-4.26 (2H, m), 6.59 (1H, d), 7.16 (1H, d), 7.33-7.35 (1H, m), 7.54-7.56 (1H, m), 7.62 (1H, s), 8.12 (1H, s), 8.16-8.18 (1H, m), 8.61 (1H, d), 8.88 (1H, t), 11.61 (1H, s)

MS m/e MH+ 421

### Example 72

### 4-(Aminomethyl)-N-(5-methylthiazol-2-yl)-1-(9H-purin-6-yl)piperidine-4-carboxamide

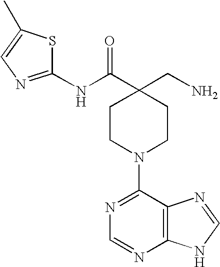

HATU (222 mg, 0.58 mmol) was added in one portion to 4-((tert-butoxycarbonyl-amino)methyl)-1-(9H-purin-6-yl)piperidine-4-carboxylic acid* (200 mg, 0.53 mmol), 5-methylthiazol-2-amine (60.7 mg, 0.53 mmol) and DIPEA (278 μl, 1.59 mmol) in DMA (2657 μl) at 25° C., under nitrogen. The resulting solution was stirred at 60° C. for 3 hours. The crude reaction mixture was then purified using ion exchange chromatography (5 g SCX-2 column, 20% 7M NH₃ in MeOH/DCM eluent). The NH₃ fraction was then evaporated to dryness. The BOC-group was then removed by reaction with trifluoroacetic acid (123 μl, 1.59 mmol) in DCM (5 ml), the reaction mixture being stirred at room temperature for 16 hrs. The crude product was purified by ion exchange chromatography, again using a 5 g SCX-2 column and 20% 7M NH₃ in MeOH/DCM eluent. The NH₃ fraction was evaporated to dryness, as before, to afford 4-(aminomethyl)-N-(5-methylthiazol-2-yl)-1-(9H-purin-6-yl)piperidine-4-carboxamide (161 mg, 81%) as a fine white solid.

1H NMR (400.13 MHz, CDCl₃) 1.59-1.66 (2H, td), 2.29 (2H, d), 2.32-2.33 (1H, d), 2.40 (3H, s), 2.99 (2H, s), 3.49 (1H, s), 4.00 (2H, broad), 4.99 (2H, broad), 7.09 (1H, s), 7.27 (1H, s), 7.95 (1H, s), 8.37 (1H, s)

MS m/e MH⁺ 373.37

*4-((tert-Butoxycarbonylamino)methyl)-1-(9H-purin-6-yl)piperidine-4-carboxylic acid was synthesised following the procedure for 4-((tert-butoxycarbonylamino)methyl)-1-(7H-pyrrolo[2,3-d]pyrimidin-4-yl)piperidine-4-carboxylic acid (Example 62D) but substituting 4-chloro-7H-pyrazolo[2,3-c]pyrimidine for 4-chloro-7H-pyrrolo[2,3-d]pyrimidine.

### Example 73

### [4-[3-(1H-Pyrazol-4-yl)phenyl]-1-(7H-pyrrolo[3,2-e]pyrimidin-4-yl)piperidin-4-yl]methanamine

### 73A. (4-(3-bromophenyl)piperidin-4-yl)methanamine dihydrochloride

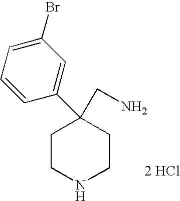

A solution of borane-tetrahydrofuran complex (233 ml, 232.71 mmol) was added to a stirred solution of tert-butyl 4-(3-bromophenyl)-4-cyanopiperidine-1-carboxylate (17 g, 46.54 mmol) (commercially available from Syntech) in THF (170 ml) at 25° C., over a period of 10 minutes under nitrogen. The resulting solution was stirred at 25° C. for 3 hours. The reaction mixture was quenched by the careful dropwise addition of methanol (250 mL), followed by 4M HCl in dioxane (250 mL). This mixture was heated at reflux for 6 hours, then stirred at 25° C. overnight. The product was concentrated in vacuo, then suspended in dichloromethane (4-(3-bromophenyl)piperidin-4-yl)methanamine dihydrochloride (14.75 g, 93%) was recovered by filtration as a white solid.

M/z: [M+H]⁺ 269; ¹H NMR (400.13 MHz, DMSO-d₆) δ 2.09-2.14 (2H, m), 2.33-2.40 (2H, m), 2.74-2.78 (2H, m), 3.07-3.14 (2H, m), 3.21 (2H, s), 7.39 (1H, t), 7.46 (1H, d), (1H, d), 7.64 (1H, s), 8.00 (2H, s), 9.25-9.32 (2H, m).

### 73B. (4-(3-bromophenyl)-1-(7H-pyrrolo[2,3-d]pyrimidin-4-yl)piperidin-4-yl)methanamine

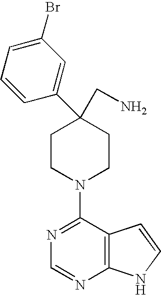

N-Ethyldiisopropylamine (3.46 ml, 20.00 mmol) was added to 4-chloro-7H-pyrrolo[2,3-d]pyrimidine (0.768 g, 5.00 mmol) and (4-(3-bromophenyl)piperidin-4-yl)methanamine dihydrochloride (1.711 g, 5 mmol) in BuOH (25.00 ml). The resulting solution was stirred at 80° C. for 1 hour. The mixture was evaporated to dryness and the crude product was purified by ion exchange chromatography, using an SCX column. The desired product was eluted from the column using 7M NH3/MeOH and pure fractions were evaporated to dryness. The crude product was purified by flash silica chromatography, elution gradient 0 to 10% 7N ammonia/methanol in DCM. Pure fractions were evaporated to dryness to afford (4-(3-bromophenyl)-1-(7H-pyrrolo[2,3-d]pyrimidin-4-yl)piperidin-4-yl)methanamine (1.010 g, 52.3%) as a foam, which solidified under vacuum to give a white solid.

M/z: [M+H]⁺ 386; ¹H NMR (400.13 MHz, CDCl₃) δ 0.78 (2H, s), 1.89-1.96 (2H, m), 2.29-2.33 (2H, m), 2.85 (2H, s), 3.54-3.60 (2H, m), 4.31-4.37 (2H, m), 6.52 (1H, d), 7.08 (1H, d), 7.29 (1H, d), 7.31-7.34 (1H, m), 7.39-7.42 (1H, m), 7.52 (1H, t), 8.33 (1H, s), 10.66 (1H, s).

### 73C. [4-[3-(1H-pyrazol-4-yl)phenyl]-1-(7H-pyrrolo[3,2-e]pyrimidin-4-yl)piperidin-4-yl]methanamine

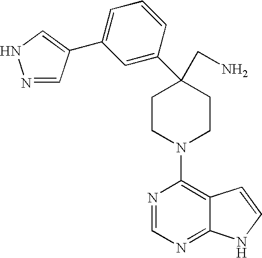

[4-(3-bromophenyl)-1-(7H-pyrrolo[3,2-e]pyrimidin-4-yl)piperidin-4-yl]methanamine (193 mg, 0.5 mmol), bis-(tri-t-butyl)phosphine palladium (13 mg, 0.05 mmol), potassium phosphate (318 mg, 1.5 mmol) and pyrazole-4-boronic acid were combined in a glass tube. The tube was evacuated and backfilled with nitrogen several times. Toluene (1.5 mL), ethanol (3 mL) and water (1.5 mL) were added, and the mixture was stirred at room temperature, and again degassed by repeated evacuation and backfilling with nitrogen. The mixture was then heated at 80° C. for 2 hours, then cooled to room temperature, and partitioned between water and DCM. The DCM layer was separated, and further purified using an SCX column. The product was recovered from the SCX column using 7N methanolic ammonia, which was then concentrated in vacuo. The residue was purified by reverse phase HPLC (Xbridge column, 19×10 mm, 5 micron C18. Modifier=1% 0.880 ammonia in water/acetonitrile) to give the title compound (84 mg, 45%) as a solid.

M/z: [M+H]⁺ 375; ¹H NMR (400.13 MHz, DMSO-d₆) δ 1.84-1.92 (2H, m), 2.21-2.27 (2H, m), 2.70 (2H, s), 3.42-3.49 (2H, m), 4.21-4.28 (2H, m), 6.58 (1H, d), 7.15-7.17 (1H, m), 7.22-7.24 (1H, m), 7.35 (1H, t), 7.46 (1H, d), 7.61 (1H, s), 7.96 (1H, s), 8.11 (1H, s), 8.23 (1H, s), 11.65 (1H, s), 12.93 (1H, s).

### Examples 74 to 80

[4-(3-bromophenyl)-1-(7H-pyrrolo[3,2-e]pyrimidin-4-yl)piperidin-4-yl]methanamine (Example 73B) was reacted with the appropriate boronic acid or boronic acid pinacol ester, using the procedure described for Example 73C to give the compounds of Examples 74 to 80.

| Example | | | Retention | |
|---|---|---|---|---|
| Number | Compound | Chemical Name | time | M.S. |
| 74 | 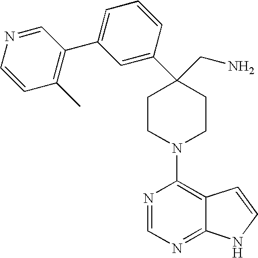 | [4-[3-(4-methylpyridin-3- yl)phenyl]-1-(7H- pyrrolo[3,2-e]pyrimidin-4- yl)piperidin-4- yl]methanamine | 1.78 min | [M + H]⁺ 399 |
| 75 | 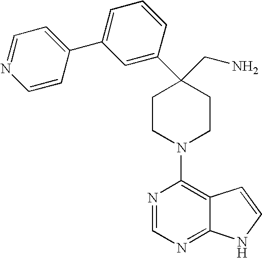 | [4-(3-pyridin-4-ylphenyl)- 1-(7H-pyrrolo[3,2- e]pyrimidin-4-yl)piperidin- 4-yl]methanamine | 1.66 min | [M + H]⁺ 385 |
| 76 | 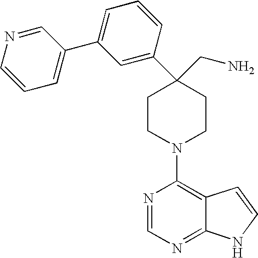 | [4-(3-pyridin-3-ylphenyl)- 1-(7H-pyrrolo[3,2- e]pyrimidin-4-yl)piperidin- 4-yl]methanamine | 1.69 min | [M + H]⁺ 385 |
| 77 | 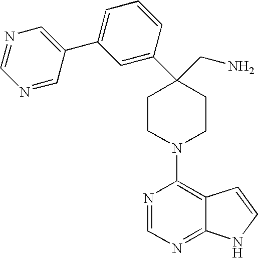 | [4-(3-pyrimidin-5- ylphenyl)-1-(7H- pyrrolo[3,2-e]pyrimidin-4- yl)piperidin-4- yl]methanamine | 1.5 min | [M + H]⁺ 386 |
| 78 | 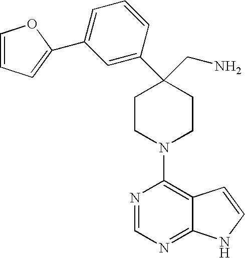 | [4-[3-(furan-2-yl)phenyl]-1- (7H-pyrrolo[3,2- e]pyrimindin-4-yl)piperidin- 4-yl]methanamine | 2.03 min | [M + H]⁺ 374 |
| 79 | 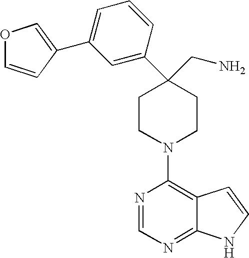 | [4-(3-furan-3-ylphenyl)-1- (7H-pyrrolo[3,2- e]pyrimidin-4-yl)piperidin- 4-yl]methanamine | 1.89 min | [M + H]⁺ 374 |
| 80 | 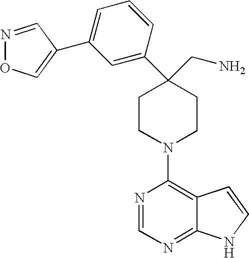 | [4-[3-(1,2-oxazol-4- yl)phenyl]-1-(7H- pyrrolo[3,2-e]pyrimidin-4- yl)piperidin-4- yl]methanamine | 1.65 min | [M + H]⁺ 375 |

The LCMS retention time data for Examples 74 to 80 were generated using a Waters 2790 or 2795 LCMS system with a Phenomenex Gemini C18, 5 micron column (50×2 mm at a flow rate of 1.1 mL/min), under a 5 minute gradient of 2.5 to 97.5% acetonitrile in water, containing 0.05% ammonium hydroxide as polar modifier.

### Example 81

### 4-(Aminomethyl)-N-(5-chloropyridin-2-yl)-1-(7H-pyrrolo[2,3-d]pyrimidin-4-yl)piperidine-4-carboxamide

### 81A. tert-Butyl 4-(5-chloropyridin-2-ylcarbamoyl)-4-cyanopiperidine-1-carboxylate

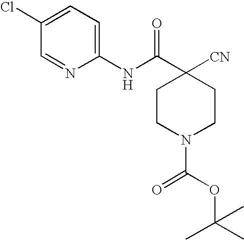

HATU (822 mg, 2.16 mmol) was added in one portion to 1-(tert-butoxycarbonyl)-4-cyanopiperidine-4-carboxylic acid (500 mg, 1.97 mmol), 5-chloropyridin-2-amine (253 mg, 1.97 mmol) and DIPEA (1.030 ml, 5.90 mmol) in DMA (20 ml) at 20° C. under nitrogen. The resulting solution was stirred at 20° C. for 24 hours. The reaction mixture was diluted with EtOAc (100 mL), and washed sequentially with water (2×100 mL) and saturated brine (50 mL). The organic layer was dried over MgSO4, filtered and evaporated to afford crude product. The crude product was purified by flash silica chromatography, elution gradient 10 to 50% EtOAc in isohexane. Pure fractions were evaporated to dryness to afford tert-butyl 4-(5-chloropyridin-2-ylcarbamoyl)-4-cyanopiperidine-1-carboxylate (340 mg, 47.4%) as a white solid.

1H NMR (400.132 MHz, DMSO) δ 1.42 (9H, s), 1.97-2.05 (2H, m), 2.23 (2H, d), 2.96 (2H, s), 4.04 (2H, d), 7.96-8.04 (2H, m), 8.46 (1H, d), 11.07 (1H, s) ¹H NMR (400.132 MHz, DMSO) δ 1.42 (9H, s), 1.97-2.05 (2H, m), 2.23 (2H, d), 2.96 (2H, s), 4.04 (2H, d), 7.96-8.04 (2H, m), 8.46 (1H, d), 11.07 (1H, s)

MS m/e MH⁺ 363

### 81B. tert-Butyl 4-(aminomethyl)-4-(5-chloropyridin-2-ylcarbamoyl)piperidine-1-carboxylate

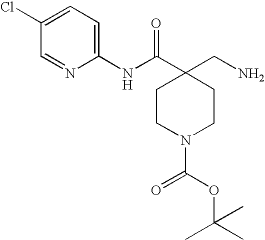

Platinum(IV) oxide (23.65 mg, 0.10 mmol) and tert-butyl 4-(5-chloropyridin-2-ylcarbamoyl)-4-cyanopiperidine-1-carboxylate (380 mg, 1.04 mmol) in acetic acid (5208 μl) were stirred under an atmosphere of hydrogen at 1 atm and 25° C. for 7 hours.

The reaction mixture was filtered through Celite and the crude product was purified by ion exchange chromatography, using an SCX column. The desired product was eluted from the column using 7M NH3/MeOH and pure fractions were evaporated to dryness to afford crude product. The crude product was purified by flash silica chromatography, elution gradient 0 to 20% 3.5M methanolic ammonia in DCM. Pure fractions were evaporated to dryness to afford tert-butyl 4-(aminomethyl)-4-(5-chloropyridin-2-ylcarbamoyl)piperidine-1-carboxylate (50 mg, 13%) as a gum.

MS m/e MH⁺ 367

### 81C. 4-(Aminomethyl)-N-(5-chloropyridin-2-yl)-1-(7H-pyrrolo[2,3-d]pyrimidin-4-yl)piperidine-4-carboxamide

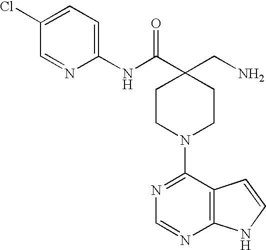

HCl (4M in Dioxane) (1000 μl, 0.14 mmol) was added to a solution of tert-butyl 4-(aminomethyl)-4-(5-chloropyridin-2-ylcarbamoyl)piperidine-1-carboxylate (50 mg, 0.14 mmol) in THF (339 μl) and the reaction heated at 60° C. for 2 hours. The reaction was cooled to room temperature and purified by ion exchange chromatography, using an SCX column. The desired product was eluted from the column using 7M NH3/MeOH and pure fractions were evaporated to dryness to afford a white solid. To this was added butan-1-ol (339 μl), 4-chloro-7H-pyrrolo[2,3-d]pyrimidine (20.82 mg, 0.14 mmol) and N-ethyldiisopropylamine (59.0 μl, 0.34 mmol) and the reaction heated at 80° C. for 5 hours. The crude product was purified by ion exchange chromatography, using an SCX column. The desired product was eluted from the column using 7M NH3/MeOH and pure fractions were evaporated to dryness to afford crude product. The crude product was purified by preparative HPLC (Waters XBridge Prep C18 OBD column, 5μ silica, 21 mm diameter, 100 mm length), using decreasingly polar mixtures of water (containing 5% ammonia) and MeCN as eluents. Fractions containing the desired compound were evaporated to dryness to afford 4-(aminomethyl)-N-(5-chloropyridin-2-yl)-1-(7H-pyrrolo[2,3-d]pyrimidin-4-yl)piperidine-4-carboxamide (4.50 mg, 8.60%) as a white solid.

1H NMR (400.132 MHz, DMSO) δ 1.52-1.60 (2H, m), 2.05-2.13 (2H, m), 2.87 (2H, s), 3.75-3.81 (2H, m), 4.06-4.14 (2H, m), 6.58-6.59 (1H, m), 7.17 (1H, t), 7.89 (1H, dd), 8.13 (2H, m), 8.34 (1H, d), 11.68 (1H, s)

MS m/e MH⁺ 386

### Example 82

### 4-(Aminomethyl)-1-(7H-pyrrolo[2,3-d]pyrimidin-4-yl)-N-(6-(trifluoromethyl)pyridin-3-yl)piperidine-4-carboxamide

### 82A. tert-Butyl 4-cyano-4-(6-(trifluoromethyl)pyridin-3-ylcarbamoyl)piperidine-1-carboxylate

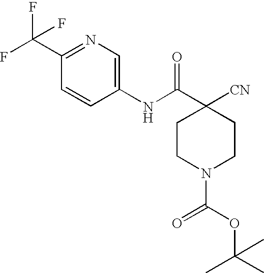

6-(Trifluoromethyl)pyridin-3-amine (319 mg, 1.97 mmol) was added in one portion to 1-(tert-butoxycarbonyl)-4-cyanopiperidine-4-carboxylic acid (500 mg, 1.97 mmol), HATU (822 mg, 2.16 mmol) and DIPEA (1030 μl, 5.90 mmol) in DMA (9832 μl) at 20° C. under nitrogen. The resulting solution was stirred at 20° C. for 24 hours. The reaction mixture was diluted with EtOAc (10 ml), and washed sequentially with water (2×10 ml) and saturated brine (10 ml). The organic layer was dried over MgSO4, filtered and evaporated to afford crude product. The crude product was purified by flash silica chromatography, elution gradient 10 to 50% EtOAc in isohexane. Pure fractions were evaporated to dryness to afford tert-butyl 4-cyano-4-(6-(trifluoromethyl)pyridin-3-ylcarbamoyl)piperidine-1-carboxylate (330 mg, 42.1%) as a white solid.

1H NMR (400.132 MHz, DMSO) δ 1.42 (9H, s), 1.97-2.05 (2H, m), 2.22 (2H, d), 3.00 (2H, s), 4.07 (2H, d), 7.93 (1H, d), 8.34-8.36 (1H, m), 8.97 (1H, d), 10.72 (1H, s)

MS m/e MH⁺ 397

### 82B. tert-Butyl 4-(aminomethyl)-4-(6-(trifluoromethyl)pyridin-3-ylcarbamoyl)piperidine-1-carboxylate

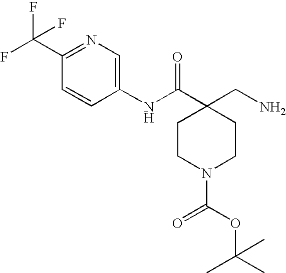

Platinum(IV) oxide (25.08 mg, 0.11 mmol) and tert-butyl 4-cyano-4-(6-(trifluoromethyl)-pyridin-3-ylcarbamoyl)piperidine-1-carboxylate (440 mg, 1.10 mmol) in acetic acid (5522 μl) were stirred under an atmosphere of hydrogen at 1 atm and 25° C. for 7 hours. The reaction mixture was filtered through celite and then the crude product was purified by ion exchange chromatography, using an SCX column. The desired product was eluted from the column using 7M NH3/MeOH and pure fractions were evaporated to dryness to afford crude product. The crude product was purified by flash silica chromatography, elution gradient 0 to 20% 7M methanolic ammonia in DCM. Pure fractions were evaporated to dryness to afford tert-butyl 4-(aminomethyl)-4-(6-(trifluoromethyl)pyridin-3-ylcarbamoyl)piperidine-1-carboxylate (160 mg, 36.0%) as a colourless gum.

MS m/e MH⁺ 401

### 82C. 4-(Aminomethyl)-1-(7H-pyrrolo[2,3-d]pyrimidin-4-yl)-N-(6-(trifluoromethyl)pyridin-3-yl)piperidine-4-carboxamide

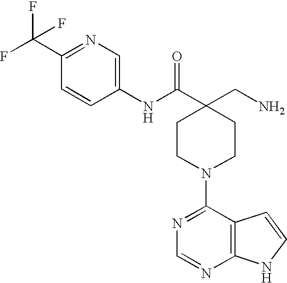

HCl (4M in Dioxane) (1000 μl, 0.40 mmol) was added to a solution of tert-butyl 4-(aminomethyl)-4-(6-(trifluoromethyl)pyridin-3-ylcarbamoyl)piperidine-1-carboxylate (160 mg, 0.40 mmol) in THF (994 μl) and the reaction heated at 60° C. for 2 hours. The reaction was cooled to room temperature and purified by ion exchange chromatography, using an SCX column. The desired product was eluted from the column using 7M NH3/MeOH and pure fractions were evaporated to dryness to afford the intermediate de-protected starting material as a white solid. To this was added butan-1-ol (994 μl), 4-chloro-7H-pyrrolo[2,3-d]pyrimidine (61.1 mg, 0.40 mmol) and N-Ethyldiisopropylamine (173 μl, 0.99 mmol) and the reaction heated at 80° C. for 5 hours. The crude product was purified by ion exchange chromatography, using an SCX column. The desired product was eluted from the column using 7M NH3/MeOH and pure fractions were evaporated to dryness to afford crude product. The crude product was purified by preparative HPLC (Waters XBridge Prep C18 OBD column, 5μ silica, 21 mm diameter, 100 mm length), using decreasingly polar mixtures of water (containing 5% ammonia) and MeCN as eluents. Fractions containing the desired compound were evaporated to dryness to afford 4-(aminomethyl)-1-(7H-pyrrolo[2,3-d]pyrimidin-4-yl)-N-(6-(trifluoromethyl)pyridin-3-yl)piperidine-4-carboxamide (31.0 mg, 18.6%) as a white solid.

1H NMR (400.132 MHz, DMSO) δ 1.55-1.61 (2H, m), 2.16-2.20 (2H, m), 2.86 (2H, s), 3.57-3.64 (2H, m), 4.23-4.27 (2H, m), 6.59-6.60 (1H, m), 7.17-7.18 (1H, m), 7.87 (1H, d), 8.13 (1H, s), 8.37 (1H, dd), 8.93 (1H, d), 11.67 (1H, s)

MS m/e MH⁺ 420

### Biological Activity

### Example 83

### Measurement of PKA Kinase Inhibitory Activity (IC₅₀)

Compounds of the invention can be tested for PK inhibitory activity using the PKA catalytic domain from Upstate Biotechnology (#14-440) and the 9 residue PKA specific peptide (GRTGRRNSI), also from Upstate Biotechnology (#12-257), as the substrate. A final concentration of 1 nM enzyme is used in a buffer that includes 20 mM MOPS pH 7.2, 40 μM ATP/γ³³P-ATP and 50 mM substrate. Compounds are added in dimethylsulphoxide (DMSO) solution to a final DMSO concentration of 2.5%. The reaction is allowed to proceed for 20 minutes before addition of excess orthophosphoric acid to quench activity. Unincorporated γ³³P-ATP is then separated from phosphorylated proteins on a Millipore MAPH filter plate. The plates are washed, scintillant is added and the plates are then subjected to counting on a Packard Topcount.

The % inhibition of the PKA activity is calculated and plotted in order to determine the concentration of test compound required to inhibit 50% of the PKA activity (IC₅₀).

### Example 84

### Measurement of PKB Kinase Inhibitory Activity (IC₅₀)

The inhibition of protein kinase B (PKB) activity by compounds can be determined essentially as described by Andjelkovic et al. (Mol. Cell. Biol. 19, 5061-5072 (1999)) but using a fusion protein described as PKB-PIF and described in full by Yang et al (Nature Structural Biology 9, 940-944 (2002)). The protein is purified and activated with PDK1 as described by Yang et al. The peptide AKTide-2T (H-A-R—K—R-E-R-T-Y—S—F-G-H—H-A-OH) obtained from Calbiochem (#123900) is used as a substrate. A final concentration of 0.6 nM enzyme is used in a buffer that includes 20 mM MOPS pH 7.2, 30 μM ATP/γ³³P-ATP and 25 μM substrate. Compounds are added in DMSO solution to a final DMSO concentration of 2.5%. The reaction is allowed to proceed for 20 minutes before addition of excess orthophosphoric acid to quench activity. The reaction mixture is transferred to a phosphocellulose filter plate where the peptide binds and the unused ATP is washed away. After washing, scintillant is added and the incorporated activity measured by scintillation counting.

The % inhibition of the PKB activity is calculated and plotted in order to determine the concentration of test compound required to inhibit 50% of the PKB activity (IC₅₀).

Following the protocol described above, the IC₅₀ values of the compounds of Examples 1, 2, 3, 4, 5, 8, 9, 10, 13-20 and 52-57 have been found to have IC₅₀ values of less than 1 μM.

### Example 85

### In Vitro MDA-MB-468 human breast adenocarcinoma GSK-3 Phosphorylation Assay

This assay determines the ability of test compounds to inhibit phosphorylation of Serine-9 residue in Glycogen Synthase Kinase-3beta (GSK-3β) as a surrogate marker of cellular PKB (Akt) activity, as assessed using Acumen Explorer Fluorescent Plate-Reader technology. A MDA-MB-468 human breast adenocarcinoma cell line (LGC Promochem, Teddington, Middlesex, UK, Catalogue No. HTB-132) was routinely maintained in Dulbecco's modified Eagle's growth medium (DMEM; Invitrogen Limited, Paisley, UK Catalogue No. 11966-025) containing 10% heat-inactivated foetal calf serum (FCS; Sigma, Poole, Dorset, UK, Catalogue No. F0392) and 1% L-glutamine (Gibco, Catalogue No. 25030-024) at 37° C. with 5% CO₂ up to a confluency of 70-90%.

For the phosphorylation assay, the cells were detached from the culture flask using Trypsin-EDTA (Invitrogen Limited, Catalogue No. 25300-062) and seeded into the wells of a black transparent-bottom Corning Costar Polystyrene 96 well plate (Fisher Scientific UK, Loughborough, Leicestershire, UK; Catalogue No. 3904 and DPS-130-020K) at a density of 5000 cells per well in 100 μl of complete growth media. The cells were incubated overnight at 37° C. with 5% CO₂ to allow them to adhere.

On day 2, the cells were treated with test compounds and incubated for 2 hours at 37° C. with 5% CO₂. Test compounds were prepared as 10 mM stock solutions in DMSO and dosed directly to required concentration into test wells using non-contact (acoustic dispensing of multiple 2.5 nl droplets directly into assay wells) ECHO dosing technology (Labcyte Inc. Sunnyvale, Calif., USA). Each plate contained control wells without test compound.

20 μl of fixing buffer (Phosphate Buffered Saline (PBS) containing 10% formaldehyde; Sigma; Catalogue No. F1635) was then added to each well to give a final well concentration 1.6%. Plates were then incubated for 30 minutes at room temperature prior to the fixative being removed. Each well was washed once with 250 μl of PBS and then 50 μl PBS added to each well. PBS was then aspirated and cells permeabilised and blocked by incubating each well with 50 μl of permeabilisation/blocking buffer (PBS containing 0.5% Tween 20 (Sigma; Catalogue No. P5927) and 5% Marvel Milk Powder (Andrews Pharmacy Ltd, Macclesfield, Cheshire, UK; Catalogue No. APC100199)) for 1 hour at room temperature prior to staining.

Following removal of Perm/Block buffer, 50 μl of primary anti-phospho-GSK-3β antibody (Cell Signalling Technology (New England Biolabs (Uk) Ltd.), Hitchin, Hertfordshire, UK; Catalogue No. 9336 diluted 1:400 in Blocking buffer (PBS containing 5% Marvel and 0.05% Tween/Polysorbate 20) was added to each well and incubated overnight at 4° C.

Each well was washed three times in 250 μl of wash buffer (PBS containing 0.05% polysorbate 20), and cells incubated for 1 hour at room temperature with 50 μl of secondary fluorescently-labelled anti-rabbit Alexa Fluor 488 antibody (Molecular Probes, Invitrogen Limited, Catalogue No. A11008) diluted 1:750 in blocking buffer. Plates were washed three times in 250 μl of wash buffer and stored containing 50 μl of PBS at 4° C. until required.

Plates were analysed using an Acumen Explorer Plate-reader to quantify level of fluorescent signal that represents quantity of phosphorylated-GSK-3β. Active compounds caused a decrease in phospho-GSK-3β phosphorylation relative to the maximum (undosed) control for each assay, which is measured by the number of phosphorylated objects per well, and enabled potency of PKB (Akt) inhibitors to be determined.

IC₅₀ calculation—IC₅₀ is the concentration of compound required to give 50% effect over the range of activity affected by the compound, between maximum (no compound) and minimum (excess level of compound) response control data. IC₅₀ values were determined by fitting background corrected, dose response assay data to a 4 parameter logistic curve fit equation model with the minimum response set to zero. This was done using an in-house developed algorithm within the Origin graphing software package (OriginLab Corporation, Northampton, Mass., USA).

Compounds of the invention were tested in the above assay and the mean IC₅₀ values of the compounds tested are set out in the table below.

| Example Number | IC₅₀ (μM) |
|---|---|
| 4 | 0.88 |
| 6 | 0.031 |
| 7 | <0.008 |
| 8 | <0.013 |
| 9 | 0.4 |
| 10 | 5.1 |
| 14 | 1.4 |
| 21 | <0.0059 |
| 22 | 0.016 |
| 23 | 0.043 |
| 24 | 0.0086 |
| 25 | 0.01 |
| 26 | 0.023 |
| 27 | 0.11 |
| 28 | 0.0072 |
| 29 | 0.011 |
| 30 | 0.027 |
| 31 | 0.022 |
| 32 | 0.29 |
| 33 | 0.74 |
| 34 | >2 |
| 35 | 1.3 |
| 36 | 2.8 |
| 37 | >30 |
| 39 | 0.3 |
| 40 | 3.4 |
| 41 | 4.1 |
| 42 | 7.1 |
| 43 | >19 |
| 44 | 19 |
| 45 | >19 |
| 46 | 14 |
| 47 | 3.9 |
| 48 | 0.38 |
| 49 | 4.2 |
| 50 | 0.7 |
| 51 | <0.017 |
| 52 | 5.1 |
| 59 | 0.2 |
| 1D & 38 | 0.059 |
| 60 | 0.33 |
| 61 | 0.047 |
| 63 | 0.17 |
| 70 | 0.059 |
| 71 | 0.017 |
| 72 | 0.31 |
| 73 | 0.15 |
| 74 | 0.055 |
| 75 | 0.065 |
| 76 | 0.033 |
| 77 | 0.035 |
| 78 | 0.05 |
| 79 | 0.048 |
| 80 | 0.1 |
| 81 | 0.073 |
| 82 | 0.054 |

Individual IC₅₀ values were not included in the calculation of the mean IC₅₀ value if they were seen to be obvious outliers, i.e. not within approximately three-fold of the IC₅₀ of two other sets of data for the same compound.

### Example 86

### Anti-Proliferative Activity

The anti-proliferative activities of compounds of the invention are determined by measuring the ability of the compounds to inhibition of cell growth in a number of cell lines. Inhibition of cell growth is measured using the Alamar Blue assay (Nociari, M. M, Shalev, A., Benias, P., Russo, C. Journal of Immunological Methods 1998, 213, 157-167). The method is based on the ability of viable cells to reduce resazurin to its fluorescent product resorufin. For each proliferation assay cells are plated onto 96 well plates and allowed to recover for 16 hours prior to the addition of inhibitor compounds for a further 72 hours. At the end of the incubation period 10% (v/v) Alamar Blue is added and incubated for a further 6 hours prior to determination of fluorescent product at 535 nM ex/590 nM em. In the case of the non-proliferating cell assay cells are maintained at confluence for 96 hour prior to the addition of inhibitor compounds for a further 72 hours. The number of viable cells is determined by Alamar Blue assay as before. All cell lines are obtained from ECACC (European Collection of cell Cultures) or ATCC.

### Example 87

### hERG Activity

The activity of compounds of the invention against the hERG K⁺ ion channel can be determined using the assay described in the article by M. H. Bridgland-Taylor et al., Journal of Pharmcaological and Toxicological Methods, 54 (2006), 189-199.

Using the above assay, it was found that the compounds of Examples 1, 4 and 6 have ICₛₒ values of greater than 32 μM.

### Pharmaceutical Formulations

### Example 88

### (i) Tablet Formulation

A tablet composition containing a compound of the formula (I) is prepared by mixing 50 mg of the compound with 197 mg of lactose (BP) as diluent, and 3 mg magnesium stearate as a lubricant and compressing to form a tablet in known manner.

### (ii) Capsule Formulation

A capsule formulation is prepared by mixing 100 mg of a compound of the formula (I) with 100 mg lactose and filling the resulting mixture into standard opaque hard gelatin capsules.

### (iii) Injectable Formulation I

A parenteral composition for administration by injection can be prepared by dissolving a compound of the formula (I) (e.g. in a salt form) in water containing 10% propylene glycol to give a concentration of active compound of 1.5% by weight. The solution is then sterilised by filtration, filled into an ampoule and sealed.

### (iv) Injectable Formulation II

A parenteral composition for injection is prepared by dissolving in water a compound of the formula (I) (e.g. in salt form) (2 mg/ml) and mannitol (50 mg/ml), sterile filtering the solution and filling into sealable 1 ml vials or ampoules.

### v) Injectable formulation III

A formulation for i.v. delivery by injection or infusion can be prepared by dissolving the compound of formula (I) (e.g. in a salt form) in water at 20 mg/ml. The vial is then sealed and sterilised by autoclaving.

### vi) Injectable formulation IV

A formulation for i.v. delivery by injection or infusion can be prepared by dissolving the compound of formula (I) (e.g. in a salt form) in water containing a buffer (e.g. 0.2 M acetate pH 4.6) at 20 mg/ml. The vial is then sealed and sterilised by autoclaving.

### (vii) Subcutaneous Injection Formulation

A composition for sub-cutaneous administration is prepared by mixing a compound of the formula (I) with pharmaceutical grade corn oil to give a concentration of 5 mg/ml. The composition is sterilised and filled into a suitable container.

### viii) Lyophilised Formulation

Aliquots of formulated compound of formula (I) are put into 50 ml vials and lyophilized. During lyophilisation, the compositions are frozen using a one-step freezing protocol at (−45° C.). The temperature is raised to −10° C. for annealing, then lowered to freezing at −45° C., followed by primary drying at +25° C. for approximately 3400 minutes, followed by a secondary drying with increased steps if temperature to 50° C. The pressure during primary and secondary drying is set at 80 millitor.

### EQUIVALENTS

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1-112. (canceled)

113. A compound of the formula (I): 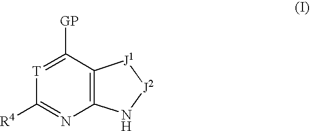
or salts, solvates, tautomers or N-oxides thereof, wherein
(1) GP is a group GP1: 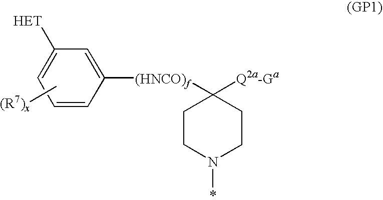
wherein f is 0 or 1, x is 0, 1, 2 or 3 and HET is a monocyclic or bicyclic heterocyclic group containing up to 4 heteroatom ring members and being optionally substituted by one or more substituents R¹¹ selected from halogen, hydroxy, trifluoromethyl, cyano, nitro, carboxy, amino, mono- or di-C₁₋₅ hydrocarbylamino and a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen and C₁₋₅ hydrocarbyl optionally substituted by one or more substituents selected from hydroxy, oxo, halogen, cyano, nitro, carboxy, amino and mono- or di-C₁₋₄ hydrocarbylamino, and wherein one or more carbon atoms of the C₁₋₅ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₅ hydrocarbyl;
X¹ is O, S or NR^{c} and X² is ═O, ═S or ═NR^{c};
T is CH or N;
J¹-J² represents a group selected from N═CH, (R^{q})C═N, HN—C(O), H₂C—C(O), N═N and (R^{q})C═CH;
R^{q} is selected from hydrogen, methyl, chlorine and bromine;
Q^{2a} is a bond or a saturated acyclic hydrocarbon linker group containing from 1 to 3 carbon atoms;
G^{a} is C(O)NR²R³, CN, NR²R³ or OH;
R² and R³ are independently selected from hydrogen; C₁₋₅ alkyl and C₁₋₅ alkanoyl wherein the alkyl and alkanoyl groups are optionally substituted by one or more substituents selected from fluorine, hydroxy, cyano, amino, methylamino, dimethylamino and methoxy; or NR²R³ forms a saturated 4 to 7 membered heterocyclic ring optionally containing, in addition to the nitrogen atom of NR²R³ a further heteroatom selected from O, N and S, the heterocyclic ring being optionally substituted by one or more C₁₋₄ alkyl groups;
R⁴ is selected from hydrogen, halogen, C₁₋₅ saturated hydrocarbyl, cyano, CONH₂, CF₃ and NH₂; and
R⁷ is selected from hydrogen, fluorine, chlorine, trifluoromethyl, methoxy, trifluoromethoxy, difluoromethoxy and cyano; or
(2) GP is a group GP2: 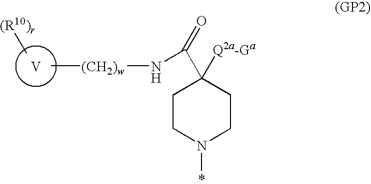
wherein
the ring V is a monocyclic or bicyclic heteroaryl group of 5 to 10 ring members containing up to 4 heteroatom ring members selected from O, N and S;
r is 0, 1, 2, 3 or 4;
w is 0 or 1;
T is CH or N;
J¹-J² represents a group selected from N═CH, (R^{q})C═N, HN—C(O), H₂C—C(O), N═N and (R^{q})C═CH;
R^{q} is selected from hydrogen, methyl, chlorine and bromine;
Q^{2a} is a bond or a saturated acyclic hydrocarbon linker group containing from 1 to 3 carbon atoms;
G^{a} is C(O)NR²R³, CN, NR²R³ or OH;
R² and R³ are independently selected from hydrogen; C₁₋₅ alkyl and C₁₋₅ alkanoyl wherein the alkyl and alkanoyl groups are optionally substituted by one or more substituents selected from fluorine, hydroxy, cyano, amino, methylamino, dimethylamino and methoxy; or NR²R³ forms a saturated 4 to 7 membered heterocyclic ring optionally containing, in addition to the nitrogen atom of NR²R³ a further heteroatom selected from O, N and S, the heterocyclic ring being optionally substituted by one or more C₁₋₄ alkyl groups;
R⁴ is selected from hydrogen, halogen, C₁₋₅ saturated hydrocarbyl, cyano, CONH₂, CF₃ and NH₂; and
R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members, and a group R^{a}-R^{b}; wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

114. A compound according to claim 113 wherein the moiety GP is a group GP2 or a group GP1 in which R² and R³ are independently selected from hydrogen; C₁₋₅ alkyl and C₁₋₅ alkanoyl wherein the alkyl and alkanoyl groups are optionally substituted by one or more substituents selected from fluorine, hydroxy, cyano, amino, methylamino, dimethylamino and methoxy.

115. A compound according to claim 113 wherein the moiety GP is a group GP1 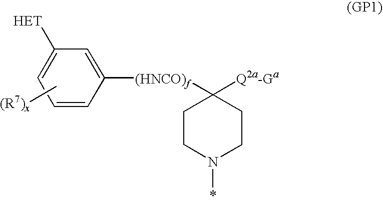
wherein f is 0 or 1, x is 0, 1, 2 or 3 and HET is a monocyclic or bicyclic heterocyclic group containing up to 4 heteroatom ring members and being optionally substituted by one or more substituents R¹¹ selected from halogen, hydroxy, trifluoromethyl, cyano, nitro, carboxy, amino, mono- or di-C₁₋₅ hydrocarbylamino and a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen and C₁₋₅ hydrocarbyl optionally substituted by one or more substituents selected from hydroxy, oxo, halogen, cyano, nitro, carboxy, amino and mono- or di-C₁₋₄ hydrocarbylamino, and wherein one or more carbon atoms of the C₁₋₅ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₅ hydrocarbyl;
X¹ is O, S or NR^{c} and X² is ═O, ═S or ═NR^{c};
T is CH or N;
J¹-J² represents a group selected from N═CH, (R^{q})C═N, HN—C(O), H₂C—C(O), N═N and (R^{q})C═CH;
R^{q} is selected from hydrogen, methyl, chlorine and bromine;
Q^{2a} is a bond or a saturated acyclic hydrocarbon linker group containing from 1 to 3 carbon atoms;
G^{a} is C(O)NR²R³, CN, NR²R³ or OH;
R² and R³ are independently selected from hydrogen; C₁₋₅ alkyl and C₁₋₅ alkanoyl wherein the alkyl and alkanoyl groups are optionally substituted by one or more substituents selected from fluorine, hydroxy, cyano, amino, methylamino, dimethylamino and methoxy; or NR²R³ forms a saturated 4 to 7 membered heterocyclic ring optionally containing, in addition to the nitrogen atom of NR²R³ a further heteroatom selected from O, N and S, the heterocyclic ring being optionally substituted by one or more C₁₋₄ alkyl groups; and
R⁷ is selected from hydrogen, fluorine, chlorine, trifluoromethyl, methoxy, trifluoromethoxy, difluoromethoxy and cyano.

116. A compound according to claim 115 wherein Q^{2a} is a bond or a group (CH₂)ₐ where a is 1, 2 or 3.

117. A compound according to claim 115 wherein G^{a} is NR²R³.

118. A compound according to claim 115 wherein HET is benzoxazole, pyridine, pyrazole or thiophene, each optionally substituted with one or more substituents selected from fluorine; chlorine; C₁₋₄ alkoxy; trifluoromethyl; trifluoromethoxy; difluoromethoxy; and C₁₋₄ alkyl.

119. A compound according to claim 113 wherein GP is a group GP2: 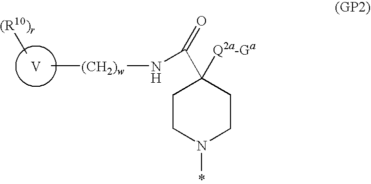
wherein
the ring V is a monocyclic or bicyclic heteroaryl group of 5 to 10 ring members containing up to 4 heteroatom ring members selected from O, N and S;
r is 0, 1, 2, 3 or 4;
w is 0 or 1;
T is CH or N;
J¹-J² represents a group selected from N═CH, (R^{q})C═N, HN—C(O), H₂C—C(O), N═N and (R^{q})C═CH;
R^{q} is selected from hydrogen, methyl, chlorine and bromine;
Q^{2a} is a bond or a saturated acyclic hydrocarbon linker group containing from 1 to 3 carbon atoms;
G^{a} is C(O)NR²R³, CN, NR²R³ or OH;
R² and R³ are independently selected from hydrogen; C₁₋₅ alkyl and C₁₋₅ alkanoyl wherein the alkyl and alkanoyl groups are optionally substituted by one or more substituents selected from fluorine, hydroxy, cyano, amino, methylamino, dimethylamino and methoxy; and
R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹)X or X¹C(X²)X¹;
R^{c} is selected from hydrogen and C₁₋₄ hydrocarbyl; and
X¹ is O, S or NR^{c} and X² is ═O, ═S or ═NR^{c}.

120. A compound according to claim 119 wherein Q^{2a} is a bond or a group (CH₂)ₐ where a is 1, 2 or 3.

121. A compound according to claim 119 wherein G^{a} is NR²R³.

122. A compound according to claim 119 wherein the ring V is a 5- or 6-membered monocyclic heteroaryl group containing 1, 2 or 3 heteroatom ring members selected from O, N and S or a 5.6 fused bicyclic heteroaryl group containing 1, 2, 3 or 4 (more preferably 1, 2 or 3 and most preferably 1 or 2) heteroatoms selected from O, N and S.

123. A compound according to claim 119 wherein the ring V is a pyridine, pyrazine, pyrimidine, pyridazine, oxazole, imidazole, thiazole, isoxazole, isothiazole, pyrazole or thiophene ring.

124. A compound according to claim 119 wherein the ring V is bicyclic and is a benzoimidazole, benzoxazole, benzothiazole, benzofuran, benzothiophene, indole or quinoline ring.

125. A compound of the formula (II): 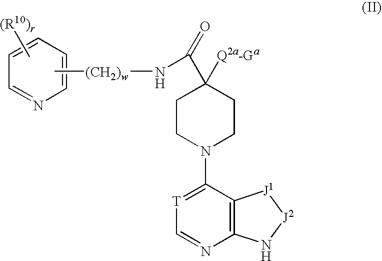
or salts, tautomers or N-oxides thereof, wherein
wherein
r is 0, 1 or 2;
w is 0 or 1;
T is CH or N;
J¹-J² represents a group selected from N═CH, (R^{q})C═N, HN—C(O), H₂C—C(O), N═N and (R^{q})C═CH;
R^{q} is selected from hydrogen, methyl, chlorine and bromine;
Q^{2a} is a bond or a saturated acyclic hydrocarbon linker group containing from 1 to 3 carbon atoms;
G^{a} is C(O)NR²R³, CN, NR²R³ or OH;
R² and R³ are independently selected from hydrogen; C₁₋₅ alkyl and C₁₋₅ alkanoyl wherein the alkyl and alkanoyl groups are optionally substituted by one or more substituents selected from fluorine, hydroxy, cyano, amino, methylamino, dimethylamino and methoxy; and
R¹⁰ is as defined in any one of the preceding claims.

126. A compound according to claim 113 having the formula (III): 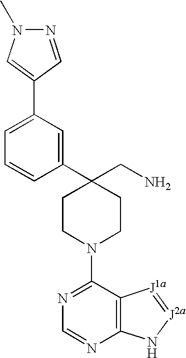
or salts, N-oxides or tautomers thereof, wherein J^{1a} is selected from CH, C-Me, C—Cl and C—Br; and J^{2a} is selected from N and CH.

127. A compound according to claim 113 having the formula (IV): 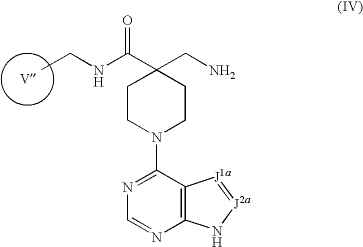
or salts, N-oxides or tautomers thereof, wherein J^{1a} is selected from N, CH, C-Me, C—Cl and C—Br; J^{2a} is selected from N and CH; and the ring V″ is (i) an optionally substituted heteroaryl ring selected from thienyl, isoxazolyl, indolyl and pyridyl; or (ii) an optionally substituted heteroaryl ring selected from thienyl, isoxazolyl, indolyl, isothiazolyl and pyridyl; wherein in each of (i) and (ii) the optional substituents for the heteroaryl ring are selected from methyl, chlorine, bromine and trifluoromethyl.

128. A compound according to claim 113 having the formula (IVa): 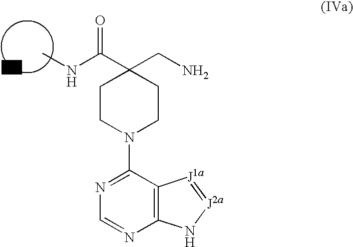
or salts, N-oxides or tautomers thereof, wherein J^{1a} is selected from N, CH, C-Me, C—Cl and C—Br; J^{2a} is selected from N and CH; and the ring V″ is (i) an optionally substituted heteroaryl ring selected from thienyl, isoxazolyl, indolyl and pyridyl; or (ii) an optionally substituted heteroaryl ring selected from thienyl, isoxazolyl, indolyl, isothiazolyl and pyridyl; wherein in each of (i) and (ii) the optional substituents for the heteroaryl ring are selected from methyl, chlorine, bromine and trifluoromethyl.

129. A compound according to claim 113 selected from:
4-aminomethyl-1-(7H-pyrrolo[2,3-d]pyrimidin-4-yl)-piperidine-4-carboxylic acid (6-chloro-pyridin-3-ylmethyl)-amide;
4-amino-1-(8-oxo-8,9-dihydro-7H-purin-6-yl)-piperidine-4-carboxylic acid (3-benzooxazol-2-yl-phenyl)-amide;
4-amino-1-(8-oxo-8,9-dihydro-7H-purin-6-yl)-piperidine-4-carboxylic acid [3-(4-methyl-pyridin-2-yl)-phenyl]-amide;
4-amino-1-(7H-pyrrolo[2,3-d]pyrimidin-4-yl)-piperidine-4-carboxylic acid (6-chloro-pyridin-3-ylmethyl)amide;
4-amino-1-(1H-pyrrolo[2,3-b]pyridin-4-yl)-piperidine-4-carboxylic acid (6-chloro-pyridin-3-ylmethyl)-amide;
C-[4-[3-(1-methyl-1H-pyrazol-4-yl)-phenyl]-1-(9H-purin-6-yl)-piperidin-4-yl]-methylamine;
C-[4-[3-(2-methyl-thiophen-3-yl)-phenyl]-1-(9H-purin-6-yl)-piperidin-4-yl]-methylamine;
C-[4-[3-(5-fluoro-pyridin-3-yl)-phenyl]-1-(9H-purin-6-yl)-piperidin-4-yl]-methylamine (; methyl-[4-[3-(1-methyl-1H-pyrazol-4-yl)-phenyl]-1-(9H-purin-6-yl)-piperidin-4-ylmethyl]-amine;
[4-[3-(1-methyl-1H-pyrazol-4-yl)-phenyl]-1-(9H-purin-6-yl)-piperidin-4-yl]-methanol;
4-[3-(1-methyl-1H-pyrazol-4-yl)-phenyl]-1-(9H-purin-6-yl)-piperidine-4-carboxylic acid (2-hydroxy-ethyl)-amide;
6-{4-aminomethyl-4-[3-(1-methyl-1H-pyrazol-4-yl)-phenyl]piperidin-1-yl}-7,9-dihydro-purin-8-one;
C-[4-[3-(1-methyl-1H-pyrazol-4-yl)-phenyl]-1-(1H-pyrazolo[3,4-d]pyrimidin-4-yl)-piperidin-4-yl]-methylamine;
4-amino-1-(7H-pyrrolo[2,3-d]pyrimidin-4-yl)-piperidine-4-carboxylic acid (6-trifluoromethyl-pyridin-3-ylmethyl)-amide hydrochloride;
4-amino-1-(7H-pyrrolo[2,3-d]pyrimidin-4-yl)-piperidine-4-carboxylic acid (5-methyl-pyrazin-2-ylmethyl)-amide hydrochloride;
4-amino-1-(7H-pyrrolo[2,3-d]pyrimidin-4-yl)-piperidine-4-carboxylic acid (5-methyl-isoxazol-3-ylmethyl)-amide hydrochloride;
4-amino-1-(7H-pyrrolo[2,3-d]pyrimidin-4-yl)-piperidine-4-carboxylic acid (1,5-dimethyl-1H-pyrazol-3-ylmethyl)-amide hydrochloride;
4-amino-1-(7H-pyrrolo[2,3-d]pyrimidin-4-yl)-piperidine-4-carboxylic acid (benzothiazol,2-ylmethyl)-amide hydrochloride;
4-amino-1-(7H-pyrrolo[2,3-d]pyrimidin-4-yl)-piperidine-4-carboxylic acid (5-chloro-pyridin-2-ylmethyl)-amide hydrochloride;
4-amino-1-(7H-pyrrolo[2,3-d]pyrimidin-4-yl)-piperidine-4-carboxylic acid (pyridin-2-ylmethyl)-amide hydrochloride;
4-(aminomethyl)-N-((5-bromothiophen-2-yl)methyl)-1-(7H-pyrrolo[2,3-d]pyrimidin-4-yl)piperidine-4-carboxamide;
4-(aminomethyl)-N-((5-chlorothiophen-2-yl)methyl)-1-(7H-pyrrolo[2,3-]pyrimidin-4-yl)piperidine-4-carboxamide;
4-(aminomethyl)-N-((5-methylthiophen-2-yl)methyl)-1-(7H-pyrrolo[2,3-d]pyrimidin-4-yl)piperidine-4-carboxamide;
4-(aminomethyl)-N-((3-bromoisoxazol-5-yl)methyl)-1-(7H-pyrrolo[2,3-d]pyrimidin-4-yl)piperidine-4-carboxamide;
N-((1H-indol-2-yl)methyl)-4-(aminomethyl)-1-(3-bromo-1H-pyrazolo[3,4-d]pyrimidin-4-yl)piperidine-4-carboxamide;
N-((1H-indol-2-yl)methyl)-4-(aminomethyl)-1-(7H-pyrrolo[2,3-d]pyrimidin-4-yl)piperidine-4-carboxamide;
4-(aminomethyl)-1-(7H-pyrrolo[2,3-d]pyrimidin-4-yl)-N-((6-(trifluoromethyl)pyridin-3-yl)methyl)piperidine-4-carboxamide;
(1-(5-bromo-7H-pyrrolo[2,3-d]pyrimidin-4-yl)-4-(3-(1-methyl-1H-pyrazol-4-yl)phenyl)piperidin-4-yl)methanamine;
(1-(5-chloro-7H-pyrrolo[2,3-d]pyrimidin-4-yl)-4-(3-(1-methyl-1H-pyrazol-4-yl)phenyl)piperidin-4-yl)methanamine;
(4-(3-(1-methyl-1H-pyrazol-4-yl)phenyl)-1-(5-methyl-7H-pyrrolo[2,3-d]pyrimidin-4-yl)piperidin-4-yl)methanamine;
(1-(3-bromo-1H-pyrazolo[3,4-d]pyrimidin-4-yl)-4-(3-(1-methyl-1H-pyrazol-4-yl)phenyl)piperidin-4-yl)methanamine;
N,N-dimethyl-1-(4-(3-(1-methyl-1H-pyrazol-4-yl)phenyl)-1-(9H-purin-6-yl)piperidin-4-yl)methanamine;
6-(4-(3-(1-methyl-1H-pyrazol-4-yl)phenyl)-4-(pyrrolidin-1-ylmethyl)piperidin-1-yl)-9H-purine;
3-(4-(3-(1-methyl-1H-pyrazol-4-yl)phenyl)-1-(9H-purin-6-yl)piperidin-4-yl)propan-1-amine;
2-amino-N-((4-(3-(1-methyl-1H-pyrazol-4-yl)phenyl)-1-(9H-purin-6-yl)piperidin-4-yl)methyl)acetamide;
2-(dimethylamino)-N-((4-(3-(1-methyl-1H-pyrazol-4-yl)phenyl)-1-(9H-purin-6-yl)piperidin-4-yl)methyl)acetamide;
4-[3-(1-methylpyrazol-4-yl)phenyl]-1-(9H-purin-6-yl)piperidine-4-carboxamide;
4-(aminomethyl)-N-[(6-chloropyridin-3-yl)methyl]-1-(7H-pyrrolo[2,3-d]pyrimidin-4-yl)piperidine-4-carboxamide;
4-amino-N-[(5-chlorothiophen-2-yl)methyl]-1-(7H-pyrrolo[2,3-d]pyrimidin-4-yl)piperidine-4-carboxamide;
4-amino-N-[(4-methyl-1,3-thiazol-2-yl)methyl]-1-(7H-pyrrolo[2,3-d]pyrimidin-4-yl)piperidine-4-carboxamide;
4-amino-1-(7H-pyrrolo[2,3-d]pyrimidin-4-yl)-N-(quinolin-3-ylmethyl)piperidine-4-carboxamide;
4-amino-N-[(2-phenyl-1,3-thiazol-4-yl)methyl]-1-(7H-pyrrolo[2,3-d]pyrimidin-4-yl)piperidine-4-carboxamide;
4-amino-N-(pyridin-4-ylmethyl)-1-(7H-pyrrolo[2,3-d]pyrimidin-4-yl)piperidine-4-carboxamide;
4-amino-N-[[3-(4-chlorophenyl)-1,2-oxazol-5-yl]methyl]-1-(7H-pyrrolo[2,3-d]pyrimidin-4-yl)piperidine-4-carboxamide;
4-amino-N-[(1-methylpyrazol-4-yl)methyl]-1-(7H-pyrrolo[2,3-d]pyrimidin-4-yl)piperidine-4-carboxamide;
4-amino-1-(7H-pyrrolo[2,3-d]pyrimidin-4-yl)-piperidine-4-carboxylic acid (2-phenyl-thiazol-5-ylmethyl)-amide;
4-amino-1-(7H-pyrrolo[2,3-d]pyrimidin-4-yl)-piperidine-4-carboxylic acid (3-methyl-thiophen-2-ylmethyl)-amide;
4-amino-1-(7H-pyrrolo[2,3-d]pyrimidin-4-yl)-piperidine-4-carboxylic acid (3-bromo-isoxazol-5-ylmethyl)-amide;
4-amino-1-(7H-pyrrolo[2,3-d]pyrimidin-4-yl)-piperidine-4-carboxylic acid (3-methyl-isoxazol-5-ylmethyl)-amide;
4-amino-1-(7H-pyrrolo[2,3-d]pyrimidin-4-yl)-piperidine-4-carboxylic acid (1H-indol-2-ylmethyl)-amide;
(4-(3-(1-methyl-1H-pyrazol-4-yl)phenyl)-1-(7H-pyrrolo[2,3-d]pyrimidin-4-yl)piperidin-4-yl)methanamine;
4-amino-1-(7H-pyrrolo[2,3-d]pyrimidin-4-yl)-piperidine-4-carboxylic acid (3-benzooxazol-2-yl-phenyl)-amide;
4-amino-1-(7H-pyrrolo[2,3-d]pyrimidin-4-yl)-piperidine-4-carboxylic acid [3-(5-fluoro-pyrimidin-2-yl)-phenyl]-amide;
4-amino-1-(7H-pyrrolo[2,3-d]pyrimidin-4-yl)-piperidine-4-carboxylic acid [3-(4-methyl-pyridin-2-yl)-phenyl]-amide;
4-amino-1-(7H-pyrrolo[2,3-d]pyrimidin-4-yl)-piperidine-4-carboxylic acid [3-(4,4-dimethyl-piperidin-1-yl)-phenyl]-amide;
4-amino-1-(9H-purin-6-yl)-piperidine-4-carboxylic acid [3-(4,4-dimethyl-piperidin-1-yl)-phenyl]-amide;
4-amino-1-(9H-purin-6-yl)-piperidine-4-carboxylic acid (3-benzooxazol-2-yl-phenyl)-amide;
4-(aminomethyl)-N-(4-methylthiazol-2-yl)-1-(7H-pyrrolo[2,3-d]pyrimidin-4-yl)piperidine-4-carboxamide;
4-(aminomethyl)-N-(5-methylthiazol-2-yl)-1-(7H-pyrrolo[2,3-d]pyrimidin-4-yl)piperidine-4-carboxamide;
4-(aminomethyl)-N-(5-fluoropyridin-2-yl)-1-(7H-pyrrolo[2,3-d]pyrimidin-4-yl)piperidine-4-carboxamide;
4-(aminomethyl)-1-(7H-pyrrolo[2,3-d]pyrimidin-4-yl)-N-(5-(trifluoromethyl)pyridin-2-yl)piperidine-4-carboxamide;
4-(aminomethyl)-N-(benzo[d]thiazol-6-yl)-1-(7H-pyrrolo[2,3-d]pyrimidin-4-yl)piperidine-4-carboxamide;
4-(aminomethyl)-N-(benzo[d]thiazol-2-yl)-1-(7H-pyrrolo[2,3-d]pyrimidin-4-yl)piperidine-4-carboxamide;
4-(aminomethyl)-N-(3-methyl-1,2,4-thiadiazol-5-yl)-1-(7H-pyrrolo[2,3-d]pyrimidin-4-yl)piperidine-4-carboxamide;
4-(aminomethyl)-N-(6-(methylsulfonyl)benzo[d]thiazol-2-yl)-1-(7H-pyrrolo[2,3-d]pyrimidin-4-yl)piperidine-4-carboxamide;
4-(aminomethyl)-N-(4-(pyridin-3-yl)thiazol-2-yl)-1-(7H-pyrrolo[2,3-d]pyrimidin-4-yl)piperidine-4-carboxamide;
4-(aminomethyl)-N-(pyridin-2-yl)-1-(7H-pyrrolo[2,3-d]pyrimidin-4-yl)piperidine-4-carboxamide;
4-(aminomethyl)-1-(7H-pyrrolo[2,3-d]pyrimidin-4-yl)-N-(4-(trifluoromethyl)pyridin-2-yl)piperidine-4-carboxamide;
4-(aminomethyl)-N-(5-methylpyridin-2-yl)-1-(7H-pyrrolo[2,3-d]pyrimidin-4-yl)piperidine-4-carboxamide;
(4-(3-fluoro-5-(1-methyl-1H-pyrazol-4-yl)phenyl)-1-(7H-pyrrolo[2,3-d]pyrimidin-4-yl)piperidin-4-yl)methanamine;
(4-(3-fluoro-5-(5-fluoropyridin-3-yl)phenyl)-1-(7H-pyrrolo[2,3-d]pyrimidin-4-yl)piperidin-4-yl)methanamine;
4-(aminomethyl)-N-(5-methylthiazol-2-yl)-1-(9H-purin-6-yl)piperidine-4-carboxamide [4-[3-(1H-pyrazol-4-yl)phenyl]-1-(7H-pyrrolo[3,2-e]pyrimidin-4-yl)piperidin-4-yl]methanamine;
[4-[3-(4-methylpyridin-3-yl)phenyl]-1-(7H-pyrrolo[3,2-e]pyrimidin-4-yl)piperidin-4-yl]methanamine;
[4-(3-pyridin-4-ylphenyl)-1-(7H-pyrrolo[3,2-e]pyrimidin-4-yl)piperidin-4-yl]methanamine;
[4-(3-pyridin-3-ylphenyl)-1-(7H-pyrrolo[3,2-e]pyrimidin-4-yl)piperidin-4-yl]methanamine;
[4-(3-pyrimidin-5-ylphenyl)-1-(7H-pyrrolo[3,2-e]pyrimidin-4-yl)piperidin-4-yl]methanamine;
[4-[3-(furan-2-yl)phenyl]-1-(7H-pyrrolo[3,2-e]pyrimidin-4-yl)piperidin-4-yl]methanamine;
[4-(3-furan-3-ylphenyl)-1-(7H-pyrrolo[3,2-e]pyrimidin-4-yl)piperidin-4-yl]methanamine;
[4-[3-(1,2-oxazol-4-yl)phenyl]-1-(7H-pyrrolo[3,2-e]pyrimidin-4-yl)piperidin-4-yl]methanamine;
4-(aminomethyl)-N-(5-chloropyridin-2-yl)-1-(7H-pyrrolo[2,3-d]pyrimidin-4-yl)piperidine-4-carboxamide;
4-(aminomethyl)-1-(7H-pyrrolo[2,3-d]pyrimidin-4-yl)-N-(6-(trifluoromethyl)pyridin-3-yl)piperidine-4-carboxamide; and salts, tautomers and N-oxides thereof.

130. A compound according to claim 113 in the form of a salt or N-oxide.

131. A method:
for the prophylaxis or treatment of a disease state or condition mediated by protein kinase B, which method comprises administering to a subject in need thereof a compound as defined in claim 113; or
for treating a disease or condition comprising or arising from abnormal cell growth or abnormally arrested cell death in a mammal, the method comprising administering to the mammal a compound as defined in claim 113 in an amount effective to inhibit protein kinase B activity; or
of inhibiting protein kinase B, which method comprises contacting the kinase with a kinase-inhibiting compound as defined in claim 113; or
of modulating a cellular process by inhibiting the activity of a protein kinase B using a compound as defined in claim 113; or
for the prophylaxis or treatment of a disease state or condition mediated by protein kinase A, which method comprises administering to a subject in need thereof a compound as defined in claim 113; or
for treating a disease or condition comprising or arising from abnormal cell growth or abnormally arrested cell death in a mammal, the method comprising administering to the mammal a compound as defined in claim 113 in an amount effective to inhibit protein kinase A activity; or
of inhibiting protein kinase A, which method comprises contacting the kinase with a kinase-inhibiting compound as defined in claim 113; or
of modulating a cellular process by inhibiting the activity of a protein kinase A using a compound as defined in claim 113; or
for treating a disease or condition comprising or arising from abnormal cell growth or abnormally arrested cell death in a mammal, which method comprises administering to the mammal a compound as defined in claim 113 in an amount effective in inhibiting abnormal cell growth; or
for alleviating or reducing the incidence of a disease or condition comprising or arising from abnormal cell growth or abnormally arrested cell death in a mammal, which method comprises administering to the mammal a compound as defined in claim 113 in an amount effective in inhibiting abnormal cell growth; or
for the treatment or prophylaxis of any one of the disease states or conditions disclosed herein, which method comprises administering to a patient in need thereof a therapeutically effective amount of a compound as defined in claim 113; or
for alleviating or reducing the incidence of a disease state or condition disclosed herein, which method comprises administering to a patient in need thereof a therapeutically effective amount of a compound as defined in claim 113; or
for the diagnosis and treatment of a disease state or condition mediated by protein kinase B, which method comprises (i) screening a patient to determine whether a disease or condition from which the patient is or may be suffering is one which would be susceptible to treatment with a compound having activity against protein kinase B; and (ii) where it is indicated that the disease or condition from which the patient is thus susceptible, thereafter administering to the patient a compound as defined in claim 113; or
for the treatment or prophylaxis of a disease state or condition in a patient who has been screened and has been determined as suffering from, or being at risk of suffering from, a disease or condition which would be susceptible to treatment with a compound having activity against protein kinase B; or
for the diagnosis and treatment of a disease state or condition mediated by protein kinase A, which method comprises (i) screening a patient to determine whether a disease or condition from which the patient is or may be suffering is one which would be susceptible to treatment with a compound having activity against protein kinase A; and (ii) where it is indicated that the disease or condition from which the patient is thus susceptible, thereafter administering to the patient a compound as defined in claim 113; or
for the treatment or prophylaxis of a disease state or condition in a patient who has been screened and has been determined as suffering from, or being at risk of suffering from, a disease or condition which would be susceptible to treatment with a compound having activity against protein kinase A; or
for modulating protein kinase B and/or protein kinase A.

132. A pharmaceutical composition comprising a novel compound as defined in claim 113 and a pharmaceutically acceptable carrier.


SEQUENCE LISTING (2 sequences)

SEQ ID NO: 1 - protein, 9 aa, Artificial
  PKA specific peptide
GRTGRRNSI

SEQ ID NO: 2 - protein, 15 aa, Artificial
  AKTide-2T
HARKRERTYSFGHHA